(19) United States
(12) Patent Application Publication
(10) Pub. No.: US 2015/0225404 A1
(43) Pub. Date: Aug. 13, 2015

(54) PYRIDINE DERIVATIVE AND MEDICINE

(71) Applicant: NIPPON SHINYAKU CO., LTD., Kyoto-shi, Kyoto (JP)

(72) Inventors: Takashi Tsuji, Otsu-shi (JP); Masaaki Shirai, Kusatsu-shi (JP)

(73) Assignee: NIPPON SHINYAKU CO., LTD., Kyoto-shi, Kyoto (JP)

(21) Appl. No.: 14/424,469

(22) PCT Filed: Aug. 30, 2013
(86) PCT No.: PCT/JP2013/073438

§ 371 Date: Feb. 27, 2015

(30) Foreign Application Priority Data
Aug. 30, 2012 (JP) 2012-190548
Sep. 28, 2012 (JP) 2012-215947

(51) Int. Cl. C07D 487/04; C07D 401/12; C07D 401/04; C07D 417/14; C07D 405/14; C07D 471/04; C07D 413/14; C07D 401/14; C07D 409/14

(52) U.S. Cl. CPC C07D 487/04; C07D 401/14; C07D 401/12; C07D 401/04; C07D 409/14; C07D 405/14; C07D 471/04; C07D 413/14; C07D 417/14

(57) ABSTRACT

The main purpose of the invention is to provide a novel pyridine derivative or a pharmaceutically acceptable salt thereof. Examples of the invention include a pyridine derivative represented by general formula [1], and a pharmaceutically acceptable salt thereof. This compound or a pharmaceutically acceptable salt thereof exhibits mGluR5 inhibitory activity, and can therefore be used as an agent for the prevention or treatment of, e.g., pain (for example, acute pain, chronic pain, inflammatory pain, neuropathic pain, hyperalgesia, thermal hyperalgesia, allodynia, pain due to noxious thermal stimulation, pain due to noxious mechanical stimulation, pain in the lower urinary tract or reproductive organs, or migraine), pruritus, lower urinary tract symptoms or lower urinary tract dysfunctions, gastroesophageal reflux disease (GERD), gastroesophageal reflux associated with transient lower esophageal sphincter relaxation (TLESR), and diseases of the central nervous system.

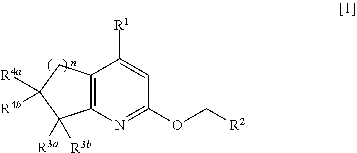

### CROSS REFERENCE TO RELATED APPLICATIONS

This patent application is a U.S. national stage application under 35 U.S.C. §371 of International Patent Application No. PCT/JP2013/073438 filed on Aug. 30, 2013, which claims the benefit of foreign priority to Japanese Patent Application No. JP 2012-190548 filed on Aug. 30, 2012, and to Japanese Patent Application No. JP 2012-215947 filed on Sep. 28, 2012. The International Application was published in Japanese on Mar. 6, 2014, as International Publication No. WO 2014/034898 A1 under PCT Article 21(2).

### FIELD OF THE INVENTION

The present invention relates to a novel pyridine derivative.

### BACKGROUND OF THE INVENTION

L-Glutamic acid is a principal excitatory neurotransmitter and plays an important role in a living body. Glutamate receptors are classified into two major groups. The first group is ionotropic glutamate receptors (iGluRs) and is composed of three subtypes, NMDA-type glutamate receptor, AMPA-type glutamate receptor, and kainate receptor. The second group is metabotropic glutamate receptors (mGluRs), which are G protein-coupled receptors (GPCR). As for mGluRs, eight subtypes are known at present (mGluR1 to mGluR8).

The eight subtypes of the metabotropic glutamate receptors are classified into three groups on the basis of homology of gene sequence, intracellular signaling pathway, and pharmacological characteristics. mGluR1 and mGluR5 belong to Group I which is coupled with Gq/11, and activation of these receptors induces intracellular Ca²⁺ mobilization. mGluR2 and mGluR3 belong to Group II, and mGluR4, mGluR6, mGlusR7, and mGluR8 belong to Group III. Each of them is coupled with Gi/o, and hence, activation of these receptors decreases intracellular cAMP.

In the nervous system, mGluR5 is expressed in a peripheral nervous system and a central nervous system (see Non-Patent Literature 1) and glial cells or the like (see Non-Patent Literature 2), and it is considered to positively regulate an excitatory synaptic transmission, e.g., by inducing a release of glutamic acid and other neurotransmitters from a presynaptic terminal (see Non-Patent Literature 3). For that reason, it is considered that an mGluR5 inhibitor will act to lower the excitation of a peripheral nerve and central nerve. In view of the fact that there are a lot of pathologies or diseases related to the excitation of a peripheral nerve or central nerve or glutamate neurotransmission, it is considered that the mGluR5 inhibitor may work as a therapeutic agent for these diseases.

### PRIOR ART LITERATURE

### Patent Literature

[Patent Literature 1] JP2012-131829

### Non-Patent Literature

[Non-Patent Literature 1] Neuroscience Research vol. 28, 49-57, 1997.
[Non-Patent Literature 2] Cell vol. 146, 785-798, 2011.
[Non-Patent Literature 3] Neuroscience Letters vol. 361 220-224, 2004.
[Non-Patent Literature 4] Neuropharmacology, vol. 40, 10-19, 2001.
[Non-Patent Literature 5] Expert Opin. Ther. Targets, vol. 6(3), 349-361, 2002.
[Non-Patent Literature 6] Psychopharmacology, vol. 179, 207-217, 2005.
[Non-Patent Literature 7] Current Drug Targets-CNS & Neurological Disorders, vol. 1, 283-296, 2002.
[Non-Patent Literature 8] Expert Opin Investig Drugs, vol. 19(4), 555-561, 2010.
[Non-Patent Literature 9] Molecular Pain, vol. 8, 20, 2012.
[Non-Patent Literature 10] J. Physiol., vol. 589(23), 5833-5843, 2011.
[Non-Patent Literature 11] Pharmacol. Rev., vol. 63, 35-58, 2011.
[Non-Patent Literature 12] Neuroscience Letters, vol. 450, 12-17, 2009.
[Non-Patent Literature 13] J. Neural Transm., vol. 118, 1703-1716, 2011.
[Non-Patent Literature 14] Neuropharmacology, vol. 44, 562-572, 2003.
[Non-Patent Literature 15] J. Neural. Transm., vol. 115, 1609-1619, 2008.
[Non-Patent Literature 16] PLoS Biol. Vol. 5(3), e52, March 2007.
[Non-Patent Literature 17] J. Physiol., 586(6), 1503-1508, 2008.
[Non-Patent Literature 18] CNS Neurol Disord Drug Targets, vol. 8(6), 475-491, 2009.
[Non-Patent Literature 19] Brain Res., vol. 1019(1-2), 246-254, 2004.
[Non-Patent Literature 20] Neuroscience Letters, vol. 420, 155-159, 2007.
[Non-Patent Literature 21] Neuropsychopharmacology, vol. 29, 921-928, 2004.
[Non-Patent Literature 22] Psychopharmacology vol. 225, 151-159, 2013.
[Non-Patent Literature 23] J. Pharmacol. Exp. Ther., vol. 313, 395-402, 2005.
[Non-Patent Literature 24] Biochimie., Vol. 94(11), 2366-2375, 2012.
[Non-Patent Literature 25] BJU INTERNATIONAL, vol. 102, 890-898, 2008.
[Non-Patent Literature 26] L. W. CROCK, et al., “MGluR5 is necessary for the full expression of both inflammatory and non-inflammatory bladder pain” [online], [retrieved November 13], 2011, Neuroscience 2011, Program No. 180.08/Poster No. 0021, [retrieved Aug. 21, 2013], searchable on the Internet <URL:http://www.abstractsonline.com/Plan/ViewAbstract.aspx? mID=2773&sKey=e4f6098c-83a5-4f51-9d69-2ec8dd0c4e29&cKey=389c9d2c-777b-4237-a079-925c7e9ed77d&mKey=8334be29-8911-4991-8c31-32b32dd5e6c8>.

### BRIEF SUMMARY OF THE INVENTION

### Problem to be Solved by the Invention

A main object of the present invention is to provide a novel pyridine derivative or a pharmaceutically acceptable salt thereof. Another main object of the present invention is to provide a pharmaceutical composition comprising the pyridine derivative or a pharmaceutically acceptable salt thereof as an active ingredient.

### Means for Solving the Problem

The present inventors found that a novel pyridine derivative or a pharmaceutically acceptable salt thereof, which is described below, has an excellent mGluR5 inhibitory activity, and thus completed the present invention.

The present invention includes the following. (A) A pyridine derivative represented by the following general formula [1] (hereinafter referred to as “the compound of the present invention”) or a pharmaceutically acceptable salt thereof:

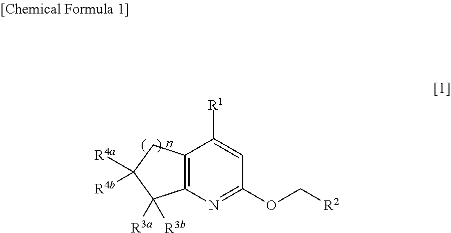

[wherein

R¹ represents phenyl, benzo[d][1,3]dioxolyl, or heteroaryl;

the heteroaryl of R¹ is bound through a carbon atom on the ring;

the phenyl, benzo[d][1,3]dioxolyl, or heteroaryl of R¹ may be substituted at a substitutable arbitrary position(s) with one or two same or different groups selected from the group consisting of

(i) halogen,

(ii) cyano,

(iii) hydroxy,

(iv) nitro,

(v) alkoxy,

(vi) cycloalkyl,

(vii) alkylsulfonate,

(viii) alkyl,

(ix) hydroxyalkyl,

(x) alkoxyalkyl,

(xi) monohalogenoalkyl,

(xii) dihalogenoalkyl,

(xiii) trihalogenoalkyl,

(xiv) amino which may be substituted at a substitutable arbitrary position(s) with one or two same or different groups selected from the group consisting of alkyl, aminoalkyl, alkoxycarbonylaminoalkyl, monoalkylaminoalkyl, dialkylaminoalkyl, and alkylsulfonyl,

(xv) saturated cyclic amino which may be substituted at a substitutable arbitrary position(s) with one or two oxos,

(xvi) alkylcarbonyl,

(xvii) alkoxycarbonyl,

(xviii) hydroxycarbonyl,

(xix) carbamoyl which may be substituted at a substitutable arbitrary position(s) with one or two same or different groups selected from the group consisting of alkyl, cycloalkyl, and (cycloalkyl)alkyl, and

(xx) a group represented by the following general formula [2]:

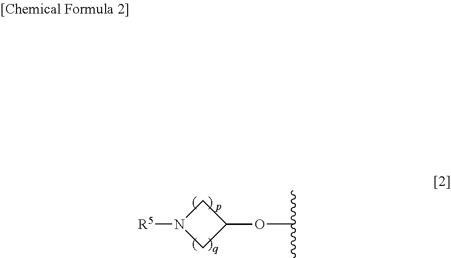

(wherein R⁵ represents hydrogen, alkyl, or alkylcarbonyl, and p and q may be the same as or different from each other and each represents 1 or 2); and

when nitrogen atom is contained as a ring constituting atom in the heteroaryl of R¹, oxygen atom may coordinate to the nitrogen atom;

R² represents phenyl or heteroaryl, and the heteroaryl of R² is bound through a carbon atom on the ring;

the phenyl or heteroaryl of R² may be substituted at a substitutable arbitrary position(s) with one or two same or different groups selected from the group consisting of cyano, halogen, cycloalkyl, alkoxy, carbamoyl, alkenyl, alkyl, hydroxyalkyl, alkoxyalkyl, monohalogenoalkyl, dihalogenoalkyl, trihalogenoalkyl, amino, monoalkylamino, and dialkylamino; and

when nitrogen atom is contained as a ring constituting atom in the heteroaryl of R², oxygen atom may coordinate to the nitrogen atom;

R^{3a} represents hydrogen, and R^{3b} represents hydrogen, alkyl, hydroxy, halogen, alkoxy, or alkylcarbonyloxy, or R^{3a} and R^{3b} taken together with the adjacent carbon atom represent a group represented by the following general formula [3] or [4]:

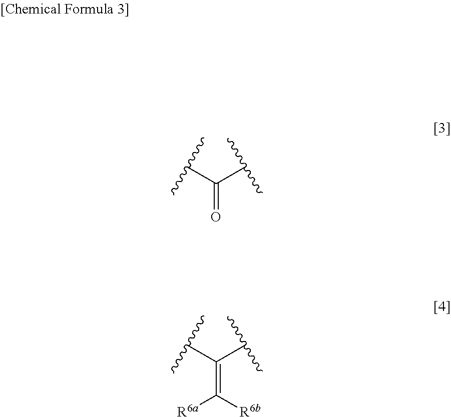

(wherein R^{6a} and R^{6b} may be the same as or different from each other and each represents hydrogen or alkyl);

R^{4a} and R^{4b} may be the same as or different from each other and each represents hydrogen or alkyl; and

n represents an integer of 1 to 3.]

(B) In the above general formula [I], the pyridine derivative or a pharmaceutically acceptable salt thereof according to (A), wherein R¹ is phenyl or heteroaryl.
(C) In the above general formula [I], the pyridine derivative or a pharmaceutically acceptable salt thereof according to (A), wherein R¹ is phenyl, pyridyl, pyrimidyl, pyrazinyl, or pyridazinyl.
(D) In the above general formula [I], the pyridine derivative or a pharmaceutically acceptable salt thereof according to (A), wherein R¹ is phenyl, pyridyl, pyrimidyl, pyrazinyl, or pyridazinyl, and R¹ may be substituted with one or two groups selected from the group consisting of alkyl, halogen, cyano, amino, monoalkylamino, dialkylamino, hydroxyalkyl, and alkylsulfonate.
(E) In the above general formula [I], the pyridine derivative or a pharmaceutically acceptable salt thereof according to (A), wherein R² is phenyl, pyridyl, pyrazinyl, pyrimidyl, oxazolyl, imidazolyl, or thiazolyl.
(F) In the above general formula [I], the pyridine derivative or a pharmaceutically acceptable salt thereof according to (A), wherein R² is phenyl, pyridyl, pyrazinyl, pyrimidyl, oxazolyl, imidazolyl, or thiazolyl, and R² may be substituted with one or two groups selected from the group consisting of alkyl, hydroxyalkyl, alkoxyalkyl, halogen, and cyano. (G) In the above general formula [I], the pyridine derivative or a pharmaceutically acceptable salt thereof according to (A), wherein R^{1a} is hydrogen, and R^{3b} is hydrogen, alkyl, halogen, hydroxy, or alkoxy. (H) In the above general formula [I], the pyridine derivative or a pharmaceutically acceptable salt thereof according to (A), wherein R^{3a}, R^{3b}, R^{4a}, and R^{4b} are each hydrogen.
(I) In the above general formula [I], the pyridine derivative or a pharmaceutically acceptable salt thereof according to (A), wherein R¹ is phenyl, pyridyl, pyrimidyl, pyrazinyl, or pyridazinyl; R² is phenyl, pyridyl, pyrazinyl, pyrimidyl, oxazolyl, imidazolyl, or thiazolyl; and R^{3a}, R^{3b}, R^{4a}, and R^{4b} are each hydrogen. (J) In the above general formula [I], the pyridine derivative or a pharmaceutically acceptable salt thereof according to (A), wherein R¹ is phenyl, pyridyl, pyrimidyl, pyrazinyl, or pyridazinyl, and R¹ may be substituted with one or two groups selected from the group consisting of alkyl, halogen, cyano, amino, monoalkylamino, dialkylamino, hydroxyalkyl, and alkylsulfonate; R² is phenyl, pyridyl, pyrazinyl, pyrimidyl, oxazolyl, imidazolyl, or thiazolyl, and R² may be substituted with one or two groups selected from the group consisting of alkyl, hydroxyalkyl, alkoxyalkyl, halogen, and cyano; and R^{3a}, R^{3b}, R^{4a}, and R^{4b} are each hydrogen.

Further, within the present invention, the compounds as defined according to any one of the following [1] to [95], or pharmaceutically acceptable salts thereof, are preferred.
(1)
4-(pyridin-3-yl)-2-(pyridin-2-ylmethoxy)-6,7-dihydro-5H-cyclopenta[b]pyridine,
(2)
3-[2-(pyridin-2-ylmethoxy)-6,7-dihydro-5H-cyclopenta[b]pyridin-4-yl]pyridine-2-carbonitrile,
(3)
5-[2-(pyridin-2-ylmethoxy)-6,7-dihydro-5H-cyclopenta[b]pyridin-4-yl]pyridin-3-yl methanesulfonate,
(4)
5-{2-[(5-fluoropyridin-2-yl)methoxy]-6,7-dihydro-5H-cyclopenta[b]pyridin-4-yl}pyridine-2-carbonitrile, (5) 2-(benzyloxy)-4-(pyrimidin-5-yl)-6,7-dihydro-5H-cyclopenta[b]pyridine,
(6)
3-({[4-(pyrimidin-5-yl)-6,7-dihydro-5H-cyclopenta[b]pyridin-2-yl]oxy}methyl)benzonitrile,
(7)
2-[(3-fluoropyridin-2-yl)methoxy]-4-(pyrimidin-5-yl)-6,7-dihydro-5H-cyclopenta[b]pyridine,
(8)
2-[(3,5-difluoropyridin-2-yl)methoxy]-4-(pyrimidin-5-yl)-6,7-dihydro-5H-cyclopenta[b]pyridine,
(9)
2-[(6-methylpyridin-3-yl)methoxy]-4-(pyrimidin-5-yl)-6,7-dihydro-5H-cyclopenta[b]pyridine,
(10)
4-(2-methylpyrimidin-5-yl)-2-(pyridin-2-ylmethoxy)-6,7-dihydro-5H-cyclopenta[b]pyridine,
(11)
5-[2-(pyridin-2-ylmethoxy)-6,7-dihydro-5H-cyclopenta[b]pyridin-4-yl]pyrimidine-2-carbonitrile,
(12)
2-[(3-fluoropyridin-2-yl)methoxy]-4-(2-methylpyrimidin-5-yl)-6,7-dihydro-5H-cyclopenta[b]pyridine,
(13)
2-[(3,5-difluoropyridin-2-yl)methoxy]-4-(2-methylpyrimidin-5-yl)-6,7-dihydro-5H-cyclopenta[b]pyridine,
(14)
2-[(3,6-difluoropyridin-2-yl)methoxy]-4-(2-methylpyrimidin-5-yl)-6,7-dihydro-5H-cyclopenta[b]pyridine,
(15)
5-{2-[(3,5-difluoropyridin-2-yl)methoxy]-6,7-dihydro-5H-cyclopenta[b]pyridin-4-yl}pyrimidine-2-carboni trite,
(16)
2-[(6-methylpyridin-2-yl)methoxy]-4-(2-methylpyrimidin-5-yl)-6,7-dihydro-5H-cyclopenta[b]pyridine,
(17)
6-({[4-(2-methylpyrimidin-5-yl)-6,7-dihydro-5H-cyclopenta[b]pyridin-2-yl]oxy}methyl)pyridine-2-carboni trite,
(18)
6-({[4-(2-methylpyrimidin-5-yl)-6,7-dihydro-5H-cyclopenta[b]pyridin-2-yl]oxy}methyl)pyridine-3-carboni trite,
(19)
5-{2-[(3-fluoropyridin-2-yl)methoxy]-6,7-dihydro-5H-cyclopenta[b]pyridin-4-yl}pyrimidine-2-carbonitril e,
(20)
2-fluoro-4-[2-(pyridin-2-ylmethoxy)-6,7-dihydro-5H-cyclopenta[b]pyridin-4-yl]benzonitrile,
(21)
3-fluoro-5-[2-(pyridin-2-ylmethoxy)-6,7-dihydro-5H-cyclopenta[b]pyridin-4-yl]phenyl methanesulfonate,
(22)
4-(pyridin-3-yl)-2-(pyrimidin-2-ylmethoxy)-5,6,7,8-tetrahydroquinoline,
(23)
2-[(4-methyl-1,3-oxazol-2-yl)methoxy]-4-(pyridin-3-yl)-5,6,7,8-tetrahydroquinoline,
(24)
4-(pyridin-3-yl)-2-(1,3-thiazol-2-ylmethoxy)-5,6,7,8-tetrahydroquinoline,
(25)
2-[(1-methyl-1H-imidazol-2-yl)methoxy]-4-(pyridin-3-yl)-5,6,7,8-tetrahydroquinoline,
(26)
4-(4-methylpyridin-3-yl)-2-(pyridin-2-ylmethoxy)-5,6,7,8-tetrahydroquinoline,
(27)
5-[2-(pyridin-2-ylmethoxy)-5,6,7,8-tetrahydroquinolin-4-yl]pyridine-2-carbonitrile,
(28)
{5-[2-(pyridin-2-ylmethoxy)-5,6,7,8-tetrahydroquinolin-4-yl]pyridin-3-yl}methanol,
(29)
N-methyl-5-[2-(pyridin-2-ylmethoxy)-5,6,7,8-tetrahydroquinolin-4-yl]pyridin-3-amine,
(30)
N,N-dimethyl-5-[2-(pyridin-2-ylmethoxy)-5,6,7,8-tetrahydroquinolin-4-yl]pyridin-3-amine,
(31)
5-{2-[(3-fluoropyridin-2-yl)methoxy]-5,6,7,8-tetrahydroquinolin-4-yl}pyridine-2-carbonitrile,
(32)
2-(pyridin-2-ylmethoxy)-4-(pyrimidin-5-yl)-5,6,7,8-tetrahydroquinoline,
(33)
2-[(3-fluoropyridin-2-yl)methoxy]-4-(pyrimidin-5-yl)-5,6,7,8-tetrahydroquinoline,
(34)
2-[(3,6-difluoropyridin-2-yl)methoxy]-4-(pyrimidin-5-yl)-5,6,7,8-tetrahydroquinoline,
(35)
6-({[4-(pyrimidin-5-yl)-5,6,7,8-tetrahydroquinolin-2-yl]oxy}methyl)pyridine-2-carbontrile,
(36)
2-[(6-methylpyridin-2-yl)methoxy]-4-(pyrimidin-5-yl)-5,6,7,8-tetrahydroquinoline,
(37)
[6-({[4-(pyrimidin-5-yl)-5,6,7,8-tetrahydroquinolin-2-yl]oxy}methyl)pyridin-2-yl]methanol,
(38)
5-[2-(pyridin-2-ylmethoxy)-5,6,7,8-tetrahydroquinolin-4-yl]pyrimidin-2-amine,
(39)
5-[2-(pyridin-2-ylmethoxy)-5,6,7,8-tetrahydroquinolin-4-yl]pyrimidine-2-carbonitrile,
(40)
2-[(6-methylpyridin-2-yl)methoxy]-4-(2-methylpyrimidin-5-yl)-5,6,7,8-tetrahydroquinoline,
(41)
6-({[4-(2-methylpyrimidin-5-yl)-5,6,7,8-tetrahydroquinolin-2-yl]oxy}methyl)pyridine-2-carbonitrile,
(42)
6-({[4-(2-methylpyrimidin-5-yl)-5,6,7,8-tetrahydroquinolin-2-yl]oxy}methyl)pyridine-3-carbonitrile,
(43)
2-[(5-fluoropyridin-2-yl)methoxy]-4-(2-methylpyrimidin-5-yl)-5,6,7,8-tetrahydroquinoline,
(44)
2-[(3-fluoropyridin-2-yl)methoxy]-4-(2-methylpyrimidin-5-yl)-5,6,7,8-tetrahydroquinoline,
(45)
2-[(3,5-difluoropyridin-2-yl)methoxy]-4-(2-methylpyrimidin-5-yl)-5,6,7,8-tetrahydroquinoline,
(46)
5-{2-[(3-fluoropyridin-2-yl)methoxy]-5,6,7,8-tetrahydroquinolin-4-yl}pyrimidin-2-amine,
(47)
3-[2-(pyridin-2-ylmethoxy)-5,6,7,8-tetrahydroquinolin-4-yl]pyrazine-2-carbonitrile,
(48)
(6-{2-[(5-fluoropyridin-2-yl)methoxy]-5,6,7,8-tetrahydroquinolin-4-yl}pyrazin-2-yl)methanol,
(49)
(6-{2-[(3-fluoropyridin-2-yl)methoxy]-5,6,7,8-tetrahydroquinolin-4-yl}pyrazin-2-yl)methanol,
(50)
7-ethyl-2-(pyridin-2-ylmethoxy)-4-(pyrimidin-5-yl)-6,7-dihydro-5H-cyclopenta[b]pyridine,
(51)
3-({[4-(pyridin-3-yl)-6,7-dihydro-5H-cyclopenta[b]pyridin-2-yl]oxy}methyl)benzonitrile,
(52)
5-{2-[(3,5-difluoropyridin-2-yl)methoxy]-6,7-dihydro-5H-cyclopenta[b]pyridin-4-yl}pyridine-2-carbonitr ile,
(53)
4-[({4-[5-(hydroxymethyl)pyridin-3-yl]-6,7-dihydro-5H-cyclopenta[b]pyridin-2-yl}oxy)methyl]benzonitrile,
(54)
(5-{2-[(4-fluorobenzyl)oxy]-6,7-dihydro-5H-cyclopenta[b]pyridin-4-yl}pyridin-3-yl)methanol,
(55)
(5-{2-[(3-fluorobenzyl)oxy]-6,7-dihydro-5H-cyclopenta[b]pyridin-4-yl}pyridin-3-yl)methanol,
(56)
6-({[4-(5-fluoropyridin-3-yl)-6,7-dihydro-5H-cyclopenta[b]pyridin-2-yl]oxy}methyl)pyridine-3-carbonitr ile,
(57)
3-({[4-(2-fluoropyridin-4-yl)-6,7-dihydro-5H-cyclopenta[b]pyridin-2-yl]oxy}methyl)benzonitrile,
(58)
6-({[4-(2-fluoropyridin-4-yl)-6,7-dihydro-5H-cyclopenta[b]pyridin-2-yl]oxy}methyl)pyridine-3-carbonitr ile,
(59)
3-[({4-[5-(hydroxymethyl)pyridin-3-yl]-6,7-dihydro-5H-cyclopenta[b]pyridin-2-yl}oxy)methyl]benzonitrile,
(60)
6-({[4-(pyrimidin-5-yl)-6,7-dihydro-5H-cyclopenta[b]pyridin-2-yl]oxy}methyl)pyridine-3-carbonitrile,
(61)
2-[(3,6-difluoropyridin-2-yl)methoxy]-4-(pyrimidin-5-yl)-6,7-dihydro-5H-cyclopenta[b]pyridine,
(62)
2-[(2-methylpyridin-4-yl)methoxy]-4-(pyrimidin-5-yl)-6,7-dihydro-5H-cyclopenta[b]pyridine,
(63)
2-[(4-fluorobenzyl)oxy]-4-(pyrimidin-5-yl)-6,7-dihydro-5H-cyclopenta[b]pyridine,
(64)
4-({[4-(pyrimidin-5-yl)-6,7-dihydro-5H-cyclopenta[b ]pyridin-2-yl]oxy}methyl)benzonitrile,
(65)
5-[2-(pyridin-2-ylmethoxy)-6,7-dihydro-5H-cyclopenta[b]pyridin-4-yl]pyrimidin-2-amine,
(66)
2-[(4-fluorobenzyl)oxy]-4-(2-methylpyrimidin-5-yl)-6,7-dihydro-5H-cyclopenta[b]pyridine,
(67)
4-({[4-(2-methylpyrimidin-5-yl)-6,7-dihydro-5H-cyclopenta[b]pyridin-2-yl]oxy}methyl)benzonitrile,
(68)
2-[(2-methylpyridin-4-yl)methoxy]-4-(2-methylpyrimidin-5-yl)-6,7-dihydro-5H-cyclopenta[b]pyridine,
(69)
5-{2-[(3-fluorobenzyl)oxy]-6,7-dihydro-5H-cyclopenta[b]pyridin-4-yl}pyrimidine-2-amine,
(70)
4-({[4-(2-aminopyrimidin-5-yl)-6,7-dihydro-5H-cyclopenta[b]pyridin-2-yl]oxy}methyl)benzonitrile,
(71)
3-({[4-(2-aminopyrimidin-5-yl)-6,7-dihydro-5H-cyclopenta[b]pyridin-2-yl]oxy}methyl)benzonitrile,
(72)
2-[(3-fluorobenzyl)oxy]-4-(2-methylpyrimidin-5-yl)-6,7-dihydro-5H-cyclopenta[b]pyridine,
(73)
2-({[4-(2-methylpyrimidin-5-yl)-6,7-dihydro-5H-cyclopenta[b]pyridin-2-yl]oxy}methyl)-1,3-oxazole-4-carbonitrile,
(74)
{6-[2-(pyridin-2-ylmethoxy)-6,7-dihydro-5H-cyclopenta[b]pyridin-4-yl]pyrazin-2-yl}methanol,
(75)
(6-{2-[(5-fluoropyridin-2-yl)methoxy]-6,7-dihydro-5H-cyclopenta[b]pyridin-4-yl}pyrazin-2-yl)methanol,
(76)
3-[({4-[6-(hydroxymethyl)pyrazin-2-yl]-6,7-dihydro-5H-cyclopenta[b]pyridin-2-yl}oxy)methyl]benzonitrile,
(77)
4-(pyridazin-3-yl)-2-(pyridin-2-ylmethoxy)-6,7-dihydro-5H-cyclopenta[b]pyridine,
(78)
2-[(3-fluoropyridin-2-yl)methoxy]-4-(pyridazin-3-yl)-6,7-dihydro-5H-cyclopenta[b]pyridine,
(79)
4-({[4-(pyridazin-3-yl)-6,7-dihydro-5H-cyclopenta[b]pyridin-2-yl]oxy}methyl)benzonitrile,
(80)
6-({[4-(1-methyl-1H-pyrazol-5-yl)-6,7-dihydro-5H-cyclopenta[b]pyridin-2-yl]oxy}methyl)pyridine-3-carbo nitrile,
(81)
6-({[4-(pyridazin-3-yl)-6,7-dihydro-5H-cyclopenta[b]pyridin-2-yl]oxy}methyl)pyridine-3-carbonitrile,
(82)
6-[({4-[5-(hydroxymethyl)pyridin-3-yl]-5,6,7,8-tetrahydroquinolin-2-yl}oxy)methyl]pyridine-3-carbonitrile,
(83)
2-[(3,5-difluoropyridin-2-yl)methoxy]-4-(pyrimidin-5-yl)-5,6,7,8-tetrahydroquinoline,
(84)
4-({[4-(pyrimidin-5-yl)-5,6,7,8-tetrahydroquinolin-2-yl]oxy}methyl)benzonitrile,
(85)
3-({[4-(pyrimidin-5-yl)-5,6,7,8-tetrahydroquinolin-2-yl]oxy}methyl)benzonitrile,
(86)
2-[(4-fluorobenzyl)oxy]-4-(pyrimidin-5-yl)-5,6,7,8-tetrahydroquinoline,
(87)
2-[(3-fluorobenzyl)oxy]-4-(pyrimidin-5-yl)-5,6,7,8-tetrahydroquinoline,
(88)
4-({[4-(pyrimidin-5-yl)-5,6,7,8-tetrahydroquinolin-2-yl]oxy}methyl)pyridine-2-carbonitrile,
(89)
5-{2-[(6-fluoropyridin-2-yl)methoxy]-5,6,7,8-tetrahydroquinolin-4-yl}pyrimidin-2-amine,
(90)
3-({[4-(2-methylpyrimidin-5-yl)-5,6,7,8-tetrahydroquinolin-2-yl]oxy}methyl)benzonitrile,
(91)
4-({[4-(2-methylpyrimidin-5-yl)-5,6,7,8-tetrahydroquinolin-2-yl]oxy}methyl)benzonitrile,
(92)
2-[(2-methylpyridin-4-yl)methoxy]-4-(2-methylpyrimidin-5-yl)-5,6,7,8-tetrahydroquinoline,
(93)
4-({[4-(2-methylpyrimidin-5-yl)-5,6,7,8-tetrahydroquinolin-2-yl]oxy}methyl)pyridine-2-carbonitrile,
(94)
6-[({4-[6-(hydroxymethyl)pyrazin-2-yl]-5,6,7,8-tetrahydroquinolin-2-yl}oxy)methyl]pyridine-3-carbonitr ile, and
(95)
4-(pyridazin-3-yl)-2-(pyridin-2-ylmethoxy)-5,6,7,8-tetrahydroquinoline.

Specific terms used in this specification are hereunder described in detail.

Examples of “halogen” include fluorine, chlorine, bromine, and iodine.

“Alkyl” may be a linear or branched one having 1 to 8 carbon atoms, and specific examples thereof may include methyl, ethyl, n-propyl, isopropyl, n-butyl, isobutyl, sec-butyl, tert-butyl, n-pentyl, isopentyl, n-hexyl, isohexyl, n-heptyl, isoheptyl, and n-octyl. Among these, an alkyl having 1 to 6 carbon atoms is preferred, and an alkyl having 1 to 3 carbon atoms is more preferred.

Examples of an alkyl moiety in “alkylsulfonate”, “alkylsulfonyl”, “aminoalkyl”, “monoalkylaminoalkyl”, “dialkylaminoalkyl”, “(cycloalkyl)alkyl”, “alkylcarbonyl”, “alkylcarbonyloxy”, “alkoxycarbonylaminoalkyl”, “hydroxyalkyl”, “alkoxyalkyl”, “monohalogenoalkyl”, “dihalogenoalkyl”, “trihalogenoalkyl”, “monoalkylamino”, or “dialkylamino” may include the same as those described above for the “alkyl”.

“Alkoxy” may be a linear or branched one having 1 to 8 carbon atoms, and specific examples thereof may include methoxy, ethoxy, n-propoxy, isopropoxy, n-butoxy, isobutoxy, sec-butoxy, tert-butoxy, n-pentyloxy, n-hexyloxy, n-heptyloxy, and n-octyloxy.

Examples of an alkoxy moiety in “alkoxycarbonyl”, “alkoxycarbonylaminoalkyl”, or “alkoxyalkyl” may include the same as those described above for the “alkoxy”.

Examples of “heteroaryl” may include a monocyclic or bicyclic, 5- to 10-membered aromatic heterocyclic group having 1 to 3 heteroatoms selected from the group consisting of nitrogen atom, oxygen atom, and sulfur atom as a ring constituting atom. Among these, a monocyclic or bicyclic, 5- to 10-membered aromatic heterocyclic group which contains at least one nitrogen atom as a ring constituting atom and which may further have one or two heteroatoms selected from the group consisting of nitrogen atom, oxygen atom, and sulfur atom is preferred. Specific examples thereof may include furyl (for example, 2-furyl and 3-furyl), thienyl (for example, 2-thienyl and 3-thienyl), pyrrolyl (for example, 1-pyrrolyl, 2-pyrrolyl, and 3-pyrrolyl), imidazolyl (for example, 2-imidazolyl and 4-imidazolyl), pyrazolyl (for example, 3-pyrazolyl and 4-pyrazolyl), triazolyl (for example, 1,2,4-triazol-3-yl and 1,2,4-triazol-4-yl), tetrazolyl (for example, 2-tetrazolyl and 5-tetrazolyl), oxazolyl (for example, 2-oxazolyl, 4-oxazolyl, and 5-oxazolyl), triazolyl (for example, 2-thiazolyl, 4-thiazolyl, and 5-thiazolyl), pyridyl (for example, 2-pyridyl, 3-pyridyl, and 4-pyridyl), pyridazinyl (for example, 3-pyridazinyl and 4-pyridazinyl), pyrimidinyl (for example, 2-pyrimidinyl, 4-pyrimidinyl, and 5-pyrimidinyl), pyrazinyl (for example, 2-pyrazinyl), benzothiazolyl (for example, benzothiazol-2-yl, benzothiazol-4-yl, benzothiazol-5-yl, benzothiazol-6-yl, and benzothiazol-7-yl), indolyl (for example, indol-3-yl, indol-4-yl, indol-5-yl, indol-6-yl, and indol-7-yl), benzothiophenyl (for example, 1-benzothiophen-2-yl, 1-benzothiophen-3-yl, 1-benzothiophenyl-4-yl, 1-benzothiophen-5-yl, 1-benzothiophen-6-yl, and 1-benzothiophen-7-yl), quinolyl (for example, quinolin-2-yl, quinolin-3-yl, quinolin-4-yl, quinoline-5-yl, quinolin-6-yl, quinolin-7-yl, and quinolin-8-yl), benzo[d]imidazolyl (for example, benzo[d]imidazol-2-yl, benzo[d]imidazol-4-yl, benzo[d]imidazol-5-yl, benzo[d]imidazol-6-yl, and benzo[d]imidazol-7-yl), imidazo[1,2-a]pyridyl (for example, imidazo[1,2-a]pyridin-2-yl, imidazo[1,2-a]pyridin-3-yl, imidazo[1,2-a]pyridin-5-yl, imidazo[1,2-a]pyridin-6-yl, imidazo[1,2-a]pyridin-7-yl, and imidazo[1,2-a]pyridin-8-yl), imidazo[1,2-b]pyridazinyl (for example, imidazo[1,2-b]pyridazin-2-yl, imidazo[1,2-b]pyridazin-3-yl, imidazo[1,2-b]pyridazin-6-yl, imidazo[1,2-b]pyridazin-7-yl, and imidazo[1,2-b]pyridazin-8-yl), isoxazolyl (for example, 3-isoxazolyl, 4-isoxazolyl, and 5-isoxazolyl), and isothiazolyl (for example, 3-isothiazolyl, 4-isothiazolyl, and 5-isothiazolyl). In addition, in such a heteroaryl containing nitrogen atom as a ring constituting atom, oxygen atom may coordinate to the nitrogen atom.

Examples of “benzo[d][1,3]dioxolyl” may include benzo[d][1,3]dioxol-4-yl and benzo[d][1,3]dioxol-5-yl.

Examples of “saturated cyclic amino” may include 4- to 7-membered saturated cyclic amino having one or two nitrogen atoms, which may have one oxygen atom or sulfur atom as a ring constituting atom. Specific examples thereof may include 1-azetidinyl, 1-pyrrolidinyl, 1-imidazolidinyl, piperidino, 1-piperazinyl, 1-tetrahydropyrimidinyl, morpholino, and thiomorpholino. In addition, the saturated cyclic amino may be substituted with one or two oxos, and examples of the saturated cyclic amino substituted with one or two oxos may include (thiomorpholine 1,1-dioxide)-4-yl and 2-oxo-azetidin-1-yl.

“Cycloalkyl” may be one having 3 to 8 carbon atoms, and specific examples thereof may include cyclopropyl, cyclobutyl, cyclopentyl, cyclohexyl, cycloheptyl, and cyclooctyl.

Examples of a cycloalkyl moiety of the “(cycloalkyl)alkyl” may include the same as those described above for the “cycloalkyl”.

Examples of the “alkenyl” may include a linear or branched alkenyl having 2 to 6 carbon atoms. Specific examples thereof may include vinyl, allyl, butenyl, and hexenyl.

### DETAILED DESCRIPTION OF THE INVENTION

The compound of the present invention can be produced according to, for example, the following method, the Examples as described later, or a known method from a known compound or an intermediate which can be easily synthesized. In the production of the compound of the present invention, in the case where a starting material has a substituent which affects a reaction, the reaction is generally performed after the starting material is protected with an appropriate protective group according to a known method in advance. The protective group can be removed by a known method after the reaction.

### Production Method 1:

Production method (1) of a compound represented by the following general formula [1A] (hereinafter referred to as “compound [1A]”), which is the compound of the present invention represented by the general formula [1], wherein R^{3a} is hydrogen, and R^{3b} is hydrogen, alkyl, halogen, or alkoxy

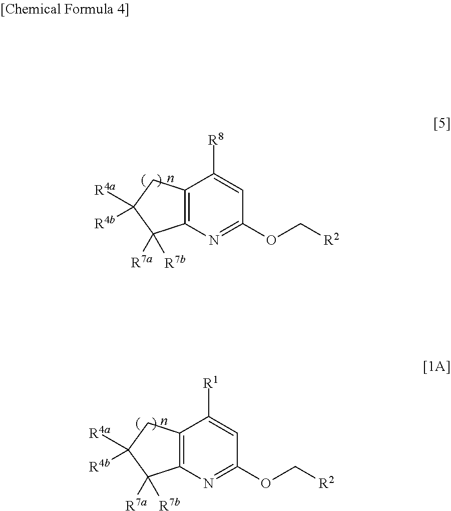

(R¹, R², R^{4a}, R^{4b}, and n mean the same as described above. R^{7a} represents hydrogen, and R^{7b} represents hydrogen, alkyl, halogen, or alkoxy. R⁸ represents halogen, trifluoromethanesulfonate, or a group represented by the following general formula [6] (hereinafter referred to as “substituent [6]”).

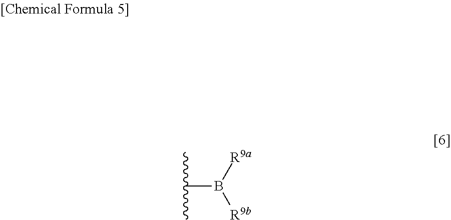

(R^{9a} and R^{9b} each represent hydroxy, or R^{9a} and R^{9b} together represent —O—C(CH₃)₂—C(CH₃)₂—O—, —O—(CH₂)₃—O—, or —O—CH₂—C(CH₃)₂—CH₂—O—.))

This reaction is a cross-coupling reaction of a compound represented by the foregoing general formula [5] (hereinafter referred to as “compound [5]”) with (i) a compound represented by the following general formula [7A] (hereinafter referred to as “compound [7A]”), which is commercially available or may be produced by a known method, (ii) a compound represented by the following general formula [7B] (hereinafter referred to as “compound [7B]”), or (iii) a compound represented by the following general formula [8] (hereinafter referred to as “compound[8]”), which is commercially available or may be produced by a known method, using a palladium catalyst, and this reaction can be, for example, performed in the presence of an appropriate base and/or an inorganic salt in an appropriate solvent.

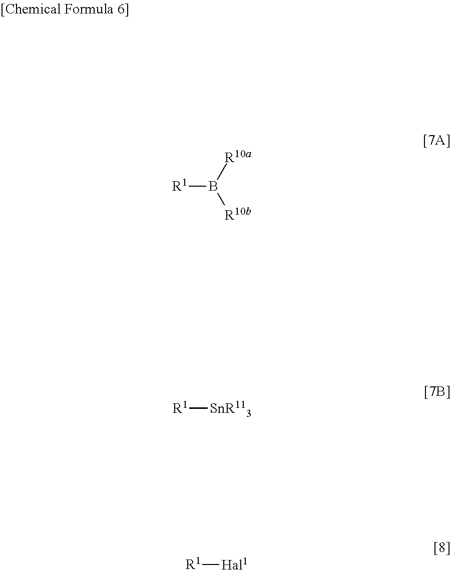

(R¹ means the same as described above. R^{10a} and R^{10b} each represent hydroxy, or R^{10a} and R^{10b} together represent —O—C(CH₃)₂—C(CH₃)₂—O—, —O—(CH₂)₃—O—, or —O—CH₂—C(CH₃)₂—CH₂—O—. R¹¹ represents methyl or n-butyl. Hal¹ represents halogen.)

In detail, the compound [1A] can be produced by a cross-coupling reaction of the compound [5], wherein R⁸ is halogen or trifluoromethanesulfonate, with the compound [7A] or compound [7B], or the compound [5], wherein R⁸ is the substituent [6], and the compound [8]. In addition, if desired, a ligand may be added, and a microwave reaction apparatus (for example, a microwave synthesis system “Initiator” (available from Biotage Japan Ltd.)) may be used.

An amount of the compound [7A], compound [7B], or compound [8] to be used is suitably in a range of, for example, 1 to 3-fold molar amount with respect to the compound [5]. Examples of the usable palladium catalyst may include a tris(dibenzylideneacetone)bispalladium chloroform adduct (hereinafter referred to as “Pd₂(dba)₃.CHCl₃”), tris(dibenzylideneacetone)bispalladium (hereinafter referred to as “Pd₂(dba)₃”), tetrakistriphenylphosphine palladium (hereinafter referred to as “Pd(PPh₃)₄”), a [1,1′-bis(diphenylphosphino)ferrocene]-dichloropalladium(II) dichloromethane adduct (hereinafter referred to as “Pd(dppf)Cl₂.CH₂Cl₂”), bis(triphenylphosphine)palladium(II) dichloride (hereinafter referred to as “PdCl₂(PPh₃)₂”), [1,1′-bis(di-tert-butylphosphino)ferrocene]palladium(II) dichloride (hereinafter referred to as “Pd(dtbpf)Cl₂”), bis(tricyclohexylphosphine)palladium(II) dichloride (hereinafter referred to as “PdCl₂(PCy₃)₂”), and palladium acetate (hereinafter referred to as “Pd(OAc)₂”). An amount of such a palladium catalyst is suitably in a range of, for example, 0.01 to 0.3-fold molar amount with respect to the compound [5]. Examples of the usable ligand may include 1,1′-bis(diphenylphosphino)ferrocene (hereinafter referred to as “dppf”), 4,5-bis(diphenylphosphino)-9,9-dimethylxanthene (hereinafter referred to as “Xantphos”), 2-dicyclohexylphosphino-2′,4′,6′-triisopropylbiphenyl (hereinafter referred to as “X-Phos”), 2,2′-bis(diphenylphosphino)-1,1′-binaphthyl (hereinafter referred to as “BINAP”), 2-dicyclohexylphosphino-2′,6′-dimethoxybiphenyl (hereinafter referred to as “S-Phos”), 9,9-dimethyl-4,5-bis[di(tert-butylphosphino)]xanthene (hereinafter referred to as “t-Bu-X-Phos”), 2-(di-tert-butylphosphino)biphenyl, bis[2-(diphenylphosphino)phenyl]ether (hereinafter referred to as “DPEphos”), tri-tert-butylphosphine, and tricyclohexylphosphine. An amount of such a ligand is suitably in a range of, for example, 1 to 5-fold molar amount with respect to the palladium catalyst. Examples of the usable base may include inorganic bases such as sodium tert-butoxide (hereinafter referred to as “NaO-t-Bu”), potassium tert-butoxide (hereinafter referred to as “KO-t-Bu”), sodium carbonate, sodium hydrogen carbonate, potassium carbonate, tripotassium phosphate, cesium carbonate, and potassium acetate.

An amount of such a base is suitably in a range of, for example, 1 to 3-fold molar amount with respect to the compound [5]. Examples of the usual inorganic salt may include lithium chloride (hereinafter referred to as “LiCl”) and cesium fluoride. An amount of such an inorganic salt is suitably in a range of, for example, 1 to 3-fold molar amount with respect to the compound [5]. Although the usable solvent is not particularly limited so long as it does not participate in the reaction, examples thereof include: hydrocarbons such as toluene and xylene; ethers such as 1,4-dioxane, tetrahydrofuran (hereinafter referred to as “THF”), and 1,2-dimethoxyethane (hereinafter referred to as “DME”); amides such as N,N-dimethylformamide (hereinafter referred to as “DMF”), N,N-dimethylacetamide (hereinafter referred to as “DMA”), and N-methylpyrrolidone (hereinafter referred to as “NMP”); alcohols such as ethanol and propanol; water; and mixed solvents thereof. A reaction temperature is suitably in a range of 20° C. to 200° C. Although a reaction time varies depending on the type of a starting material to be used, the reaction temperature, or the like, in general, it is suitably in a range of 10 minutes to 24 hours.

Incidentally, the compound [5] that is a starting material can be, for example, produced according to the following Production Method A of Compound [5] to Production Method C of Compound [5].

### Production Method a of Compound [5]:

Production method (1) of a compound represented by the following general formula [5Aa] (hereinafter referred to as “compound [5Aa]”), which is the compound [5], wherein R⁸ is halogen

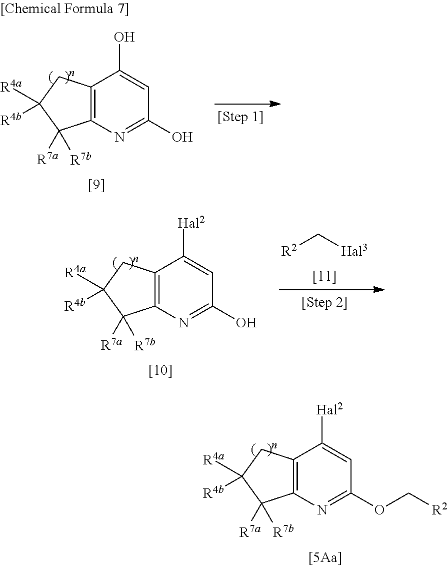

(R², R^{4a}, R^{4b}, R^{7a}, R^{7b}, and n mean the same as described above. Hal² and Hal³ each represent halogen.)

### Step 1

This reaction is a monohalogenation reaction of a compound represented by the foregoing general formula [9] (hereinafter referred to as “compound [9]”), and a compound represented by the foregoing general formula [10] (hereinafter referred to as “compound [10]”) can be, for example, produced by allowing the compound [9] to react with a halogenating agent according to a method which is known itself as a halogenation reaction. In general, this reaction can be performed without using a solvent or in an appropriate solvent at a temperature ranging from 20° C. to 200° C. In addition, if desired, a base may be added.

Examples of the usable halogenating agent may include phosphorusoxychloride, phosphorus oxybromide, phosphorus pentachloride, and phosphorus tribromide. An amount of such a halogenating agent is preferably in a range of, for example, 1 to 3-fold molar amount with respect to the compound [9]. Examples of the usable base may include organic bases such as N,N-dimethylaniline, N,N-diethylaniline, triethylamine (hereinafter referred to as “NEt₃”), and diisopropylethylamine (hereinafter referred to as “DIPEA”). Although the usable solvent is not particularly limited so long as it does not participate in the reaction, examples thereof may include: hydrocarbons such as toluene, xylene, and benzene; ethers such as 1,4-dioxane and DME; and acetonitrile (hereinafter referred to as “MeCN”). A reaction temperature is preferably in a range of 20° C. to 100° C. Although a reaction time varies depending on the type of a starting material to be used or the reaction temperature, in general, it is suitably in a range of 1 hour to 24 hours.

### Step 2

The compound [5Aa] can be produced by allowing the compound [10] to react with a compound represented by the foregoing general formula [11] (hereinafter referred to as “compound [11]”), which is commercially available or can be produced by a known method. This reaction can be performed in the presence of an appropriate base in an appropriate solvent at a temperature ranging from 0° C. to 200° C. In addition, in the case of performing this reaction in the presence of a base, if desired, an additive may be added.

An amount of the compound [11] to be used is suitably in a range of, for example, 1 to 3-fold molar amount with respect to the compound [10]. Examples of the usable base include silver carbonate, potassium carbonate, cesium carbonate, potassium iodide, and sodium hydride. An amount of such a base is suitably in a range of, for example, 1 to 5-fold molar amount with respect to the compound [10]. Examples of the usable additive may include tetra-n-butylammonium iodide, tetra-n-butylammonium bromide, and 18-crown-6-ether. An amount of such an additive is suitably in a range of, for example, 1 to 5-fold molar amount with respect to the compound [10]. Although the usable solvent is not particularly limited so long as it does not participate in the reaction, examples thereof may include: hydrocarbons such as toluene and xylene; ethers such as diethyl ether (hereinafter referred to as “Et₂O”), THF, 1,4-dioxane, DME, and cyclopentylmethyl ether (hereinafter referred to as “CPME”); amides such as DMF, DMA, and NMP; MeCN; acetone; and mixed solvents thereof. Although a reaction time varies depending on the type of a starting material to be used, a reaction temperature, or the like, in general, it is suitably in a range of 1 hour to 24 hours.

Incidentally, the compound [9] that is a starting material can be, for example, produced according to the following production method.

### Production Method of Compound [9]

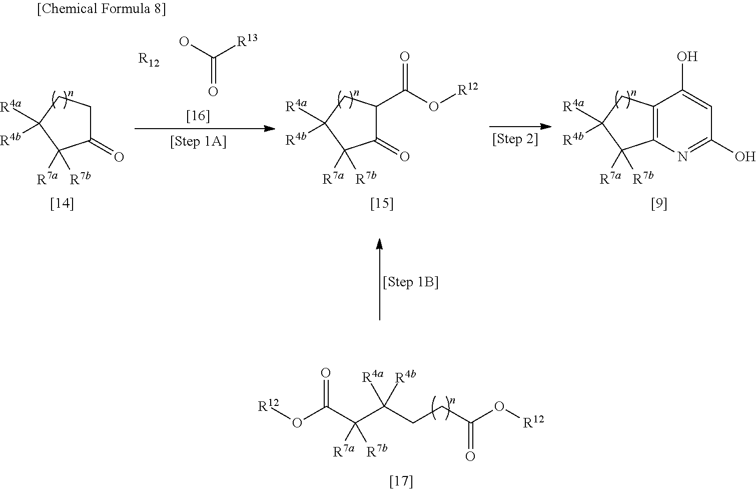

(R^{4a}, R^{4b}, R^{7a}, R^{7b}, and n mean the same as described above. R¹² represents alkyl. R¹³ represents OR¹² (R¹² means the same as described above) or cyano.)

### Step 1A

A compound represented by the foregoing general formula [15] (hereinafter referred to as “compound [15]”) can be, for example, produced by allowing a compound represented by the foregoing general formula [14] (hereinafter referred to as “compound [14]”), which is commercially available or can be produced by a known method, to react with a compound represented by the foregoing general formula [16] (hereinafter referred to as “compound [16]”), which is commercially available or can be produced by a known method, according to a method described in Journal of Organic Chemistry, 1991, vol. 56, pp. 6199-6205, US2005/38052, US2006/293364, or WO2009/47255.

### Step 1B

The compound [15] can be, for example, produced from a compound represented by the foregoing general formula [17] (hereinafter referred to as “compound [17]”), which is commercially available or may be produced by a known method, according to a method described in Organic Mass Spectrometry, 1988, vol. 23, pp. 719-722, Canadian Journal of Chemistry, 1997, vol. 75, pp. 965-974, or W02006/35061.

### Step 2

The compound [9] can be, for example, produced from the compound [15] according to a method described in Helvetica Chimica Acta, 1945, vol. 28, pp. 1684-1690 or Helvetica Chimica Acta, 1944, vol. 27, pp. 1854-1858.

### Production Method B of Compound [5]:

Production method (2) of the compound [5Aa], which is the compound [5], wherein R⁸ is halogen

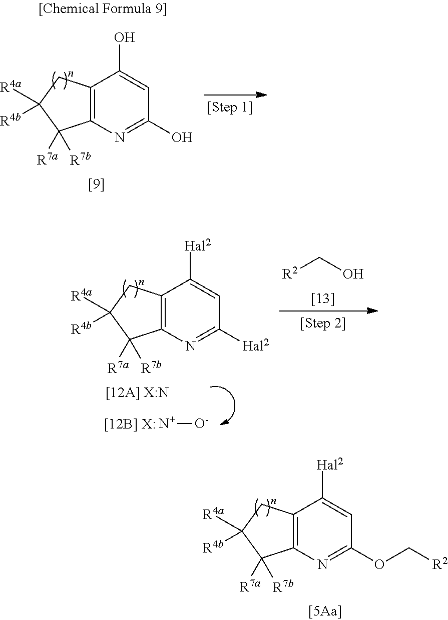

(R², R^{4a}, R^{4b}, R^{7a}, R^{7b}, Hal² and n mean the same as described above. X represents N or N⁺—O⁻.) Step 1

This reaction is a dihalogenation reaction of the compound [9], and a compound represented by the foregoing general formula [12A] (hereinafter referred to as “compound [12A]”) can be, for example, produced by allowing the compound [9] to react with a halogenating agent according to a method which is known itself as a halogenation reaction. In general, this reaction can be performed without using a solvent or in an appropriate solvent at a temperature ranging from 20° C. to 200° C. In addition, if desired, a base may be added.

Examples of the usable halogenating agent may include phosphorusoxychloride, phosphorus oxybromide, phosphorus pentachloride, and phosphorus tribromide. An amount of such a halogenating agent is preferably in a range of, for example, 1 to 3-fold molar amount with respect to the compound [9]. Examples of the usable base may include organic bases such as N,N-dimethylaniline, N,N-diethylaniline, NEt₃, and DIPEA. Although the usable solvent is not particularly limited so long as it does not participate in the reaction, examples thereof may include: hydrocarbons such as toluene, xylene, and benzene; ethers such as 1,4-dioxane and dimethoxyethane; and MeCN. A reaction temperature is preferably in a range of 100° C. to 200° C. Although a reaction time varies depending on the type of a starting material to be used or the reaction temperature, in general, it is suitably in a range of 1 hour to 24 hours.

Incidentally, a compound represented by the foregoing general formula [12B] (hereinafter referred to as “compound [12B]”) can be produced by allowing the compound [12A] to react with an oxidizing agent. In general, this reaction can be performed in an appropriate solvent in the presence of an oxidizing agent, for example, 3-chloroperbenzoic acid (hereinafter referred to as “m-CPBA”) or hydrogen peroxide, at a temperature ranging from 0° C. to 100° C. In addition, if desired, an additive may be added.

Examples of the usable additive may include sodium hydrogen carbonate and trifluoroacetic anhydride (hereinafter referred to as “TFAA”). Although the usable solvent is not particularly limited so long as it does not participate in the reaction, examples thereof may include: hydrocarbons such as toluene, xylene, and benzene; ethers such as 1,4-dioxane and DME; halogenated hydrocarbons such as dichloromethane (hereinafter referred to as “CH₂Cl₂”) and chloroform; MeCN; water; and mixed solvents thereof. Although a reaction time varies depending on the type of a starting material to be used or the reaction temperature, in general, it is suitably in a range of 1 hour to 24 hours.

### Step 2

The compound [5Aa] can be produced by allowing the compound [12A] to react with a compound represented by the foregoing general formula [13] (hereinafter referred to as “compound [13]”), which is commercially available or can be produced by a known method. In general, this reaction can be performed in the presence of an appropriate base in an appropriate solvent at a temperature ranging from 0° C. to 200° C.

An amount of the compound [13] to be used is suitably in a range of, for example, 1 to 3-fold molar amount with respect to the compound [12A]. Examples of the usable base may include NaO-t-Bu, KO-t-Bu, potassium carbonate, cesium carbonate, sodium hydroxide, and sodium hydride. An amount of such a base is suitably in a range of, for example, 1 to 5-fold molar amount with respect to the compound [12A]. Although the usable solvent is not particularly limited so long as it does not participate in the reaction, examples thereof may include: hydrocarbons such as toluene and xylene; ethers such as Et₂O, THF, 1,4-dioxane, and DME; and amides such as DMF, DMA, and NMP. Although a reaction time varies depending on the type of a starting material to be used, the reaction temperature, or the like, in general, it is suitably in a range of 1 to 24 hours.

In addition, the compound [5Aa] can be produced by allowing the compound [12B] to react with the compound [13] which is commercially available or may be produced by a known method in the presence of an appropriate base in an appropriate solvent at a temperature ranging from 0° C. to 200° C., followed by further performing a reduction reaction. In addition, if desired, an additive may be added.

An amount of the compound [13] to be used is suitably in a range of, for example, 1 to 3-fold molar amount with respect to the compound [12B]. Examples of the usable base may include NaO-t-Bu, KO-t-Bu, potassium carbonate, cesium carbonate, sodium hydroxide, and sodium hydride. An amount of such abase is suitably in a range of, for example, 1 to 5-fold molar amount with respect to the compound [12B]. Although the usable solvent is not particularly limited so long as it does not participate in the reaction, examples thereof may include: halogenated hydrocarbons such as CH₂Cl₂ and chloroform; hydrocarbons such as hexane, toluene, and xylene; ethers such as Et₂O, THF, 1,4-dioxane, and DME; and amides such as DMF, DMA, and NMP. Examples of a usable reducing agent include phosphorus trichloride, phosphorus tribromide, lithium aluminum hydride, sodium borohydride (hereinafter referred to as “NaBH₄”), zinc, and iron. An amount of such a reducing agent is suitably in a range of, for example, 1 to 5-fold molar amount with respect to the compound [12B]. Examples of the usable additive include ammonium chloride and acetic acid. Although a reaction time varies depending on the type of a starting material to be used, the reaction temperature, or the like, in general, it is suitably in a range of 1 hour to 24 hours.

### Production Method C of Compound [5]:

Production Method of a compound represented by the following general formula [5AB] (hereinafter referred to as “compound [5AB]”), which is the compound [5], wherein R⁸ is trifluoromethanesulfonate, and a compound represented by the following general formula [5Qb] (hereinafter referred to as “compound [5B]”), which is the compound [5], wherein R⁸ is the substituent [6]

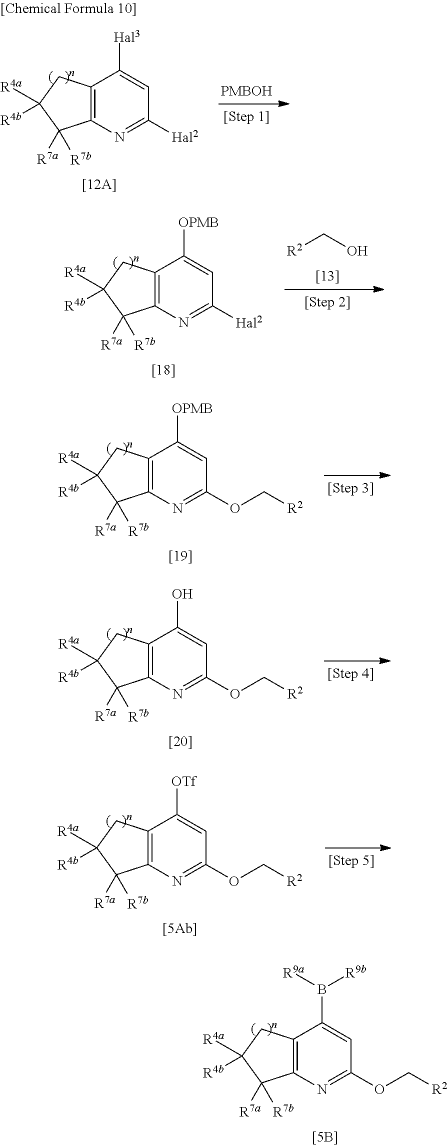

(R², R^{4a}, R^{4b}, R^{7a}, R^{7b}, R^{9a}, R^{9b}, Hal², and n mean the same as described above. PMB represents p-methoxybenzyl, and Tf represents trifluoromethanesulfonyl.)

### Step 1

A compound represented by the foregoing general formula [18] (hereinafter referred to as “compound [18]”) can be produced by allowing the compound [12A] to react with p-methoxybenzyl alcohol. Therefore, this reaction can be performed according to a method which is known itself as a p-methoxybenzyl alcoholation reaction. This reaction can be performed in the presence of p-methoxybenzyl alcohol in an appropriate solvent using a base such as sodium hydride at a temperature ranging from 0° C. to 150° C. In addition, if desired, for example, 15-crown-5-ether may be added.

An amount of the p-methoxybenzyl alcohol to be used is suitably in a range of, for example, 1 to 2-fold molar amount with respect to the compound [12A]. Although the usable solvent is not particularly limited so long as it does not participate in the reaction, examples thereof may include: hydrocarbons such as toluene and xylene; ethers such as Et₂O, THF, 1,4-dioxane, and DME; and amides such as DMF, DMA, and NMP. Although a reaction time varies depending on the type of a starting material to be used, the reaction temperature, or the like, in general, it is suitably in a range of 1 hour to 24 hours.

### Step 2

A compound [19] can be produced by allowing the compound [18] to react with the compound [13] which is commercially available or can be produced by a known method.

In general, this reaction can be, for example, performed in the presence of a palladium catalyst and an appropriate base or inorganic salt in an appropriate solvent by optionally adding a ligand at a temperature ranging from 20° C. to 200° C. In addition, if desired, this reaction can be performed using a microwave reaction apparatus.

An amount of the compound [13] to be used is suitably in a range of, for example, 1 to 3-fold molar amount with respect to the compound [18]. Examples of the usable palladium catalyst may include Pd₂(dba)₃.CHCl₃, Pd₂(dba)₃, Pd(PPh₃)₄, Pd(dPpf)Cl₂.CH₂Cl₂, PdCl₂(PPh₃)₂, Pd(dtbpf)Cl₂, PdCl₂(PCy₃)₂, and Pd(OAc)₂. An amount of such a palladium catalyst is suitably in a range of, for example, 0.01 to 0.3-fold molar amount with respect to the compound [18]. Examples of the usable ligand may include dppf, Xantphos, X-Phos, BINAP, S-Phos, t-Bu-X-Phos, 2-(di-tert-butylphosphino)biphenyl, DPEphos, tri-tert-butylphosphine, and tricyclohexylphosphine. An amount of such a ligand is suitably in a range of, for example, 1 to 5-fold molar amount with respect to the palladium catalyst. Examples of the usable base may include inorganic bases such as NaO-t-Bu, KO-t-Bu, sodium carbonate, sodium hydrogen carbonate, potassium carbonate, tripotassium phosphate, cesium carbonate, and potassium acetate. An amount of such a base is suitably in a range of, for example, 1 to 3-fold molar amount with respect to the compound [18]. Examples of the usable inorganic salt may include LiCl and cesium fluoride. An amount of such an inorganic salt is suitably in a range of, for example, 1 to 3-fold molar amount with respect to the compound [18]. Although the usable solvent is not particularly limited so long as it does not participate in the reaction, examples thereof may include: hydrocarbons such as toluene and xylene; ethers such as 1,4-dioxane, THF, and DMF; amides such as DMF, DMA, and NMP; alcohols such as ethanol and propanol; water; and mixed solvents thereof. Although a reaction time varies depending on the type of a starting material to be used, the reaction temperature, or the like, in general, it is suitably in a range of 1 hour to 24 hours.

### Step 3

A compound represented by the foregoing general formula [20] (hereinafter referred to as “compound [20]”) can be produced by deprotection of p-methoxybenzyl of the compound [19]. Therefore, this reaction can be performed according to a known method which is known itself as the deprotection of p-methoxybenzyl. This reaction can be performed in the presence of a deprotecting agent of p-methoxybenzyl in an appropriate solvent at a temperature ranging from 0° C. to 100° C.

Examples of the usable deprotecting agent of p-methoxybenzyl may include trifluoroacetic acid (hereinafter referred to as “TFA”), hydrochloric acid, AMBERLYST-15, cerium(IV) ammonium nitrate, and iodine. Although the usable solvent is not particularly limited so long as it does not participate in the reaction, examples thereof may include: halogenated hydrocarbons such as CH₂Cl₂ and chloroform; hydrocarbons such as toluene and xylene; ethers such as Et₂O, THF, 1,4-dioxane, and DME; alcohols such as methanol, ethanol, and propanol; MeCN; water; and mixed solvents thereof. Although a reaction time varies depending on the type of a starting material to be used, the reaction temperature, or the like, in general, it is suitably in a range of 1 to 24 hours.

### Step 4

The compound [5Ab] can be produced by allowing the compound [20] to react with a trifluoromethanesulfonylating agent. Therefore, this reaction can be performed according to a method which is known itself as a trifluoromethanesulfonylation reaction. In general, this reaction is performed in an appropriate solvent in the presence of a base at −78° C. to 20° C.

Examples of the usable trifluoromethanesulfonylating agent may include trifluoromethanesulfonic anhydride (hereinafter referred to as “Tf₂O”) and N-phenylbis(trifluoromethanesulfonimide) (hereinafter referred to as “Tf₂NPh”). Examples of the usable base may include Et₃N, pyridine, DIPEA, 2,6-lutidine, N,N-dimethylaminopyridine (hereinafter referred to as “DMAP”), and mixtures thereof. Although the usable solvent is not particularly limited so long as it does not participate in the reaction, for example, halogenated hydrocarbons such as CH₂Cl₂ and chloroform are preferred. Although a reaction time varies depending on the type of a starting material to be used, the reaction temperature, or the like, in general, it is suitably in a range of 1 to 24 hours.

### Step 5

The compound [5B] can be produced by subjecting the compound [5Ab] to boration. Therefore, this reaction can be performed according to a method which is known itself as a boration reaction. In general, this reaction can be performed in the presence of a palladium catalyst and an appropriate base or inorganic salt in an appropriate solvent by optionally adding a ligand at a temperature ranging from 20° C. to 200° C. In addition, if desired, this reaction can be performed using a microwave reaction apparatus.

Examples of the usable borating agent include bis(pinacolato)diboron, pinacol borane, and the like. An amount of such a borating agent is suitably in a range of, for example, 1 to 2-fold molar amount with respect to the compound [5Ab]. Examples of the usable palladium catalyst may include Pd₂(dba)₂.CHCl₃, Pd₂(dba)₃, Pd(PPh₃)₄, Pd(dppf)Cl₂.CH₂Cl₂, PdCl₂(PPh₃)₂, Pd(dtbpf)Cl₂, PdCl₂(PCy₃)₂, and Pd(OAc)₂. An amount of such a palladium catalyst is suitably in a range of, for example, 0.01 to 0.3-fold molar amount with respect to the compound [5Ab]. Examples of the usable ligand may include dppf, Xantphos, X-Phos, BINAP, S-Phos, t-Bu-X-Phos, 2-(di-tert-butylphosphino)biphenyl, DPEphos, tri-tert-butylphosphine, and tricyclohexylphosphine. An amount of such a ligand is suitably in a range of, for example, 1 to 5-fold molar amount with respect to the palladium catalyst. Examples of the usable base may include: inorganic bases such as potassium carbonate, tripotassium phosphate, cesium carbonate, and potassium acetate; and organic bases such as NEt₃ and DIPEA. An amount of such a base is suitably in a range of, for example, 1 to 3-fold molar amount with respect to the compound [5Ab]. Examples of the usable inorganic salt may include LiCl and cesium fluoride. An amount of such an inorganic salt is suitably in a range of, for example, 1 to 3-fold molar amount with respect to the compound [5Ab]. Although the usable solvent is not particularly limited so long as it does not participate in the reaction, examples thereof may include: halogenated hydrocarbons such as 1,2-dichloroethane and chloroform; hydrocarbons such as toluene and xylene; ethers such as 1,4-dioxane, THF, and DME; amides such as DMF, DMA, and NMP; alcohols such as ethanol and propanol; water; and mixed solvents thereof. Although a reaction time varies depending on the type of a starting material to be used, the reaction temperature, or the like, in general, it is suitably in a range of 1 hour to 24 hours.

### Production Method 2:

Production Method (2) of the compound [1A]

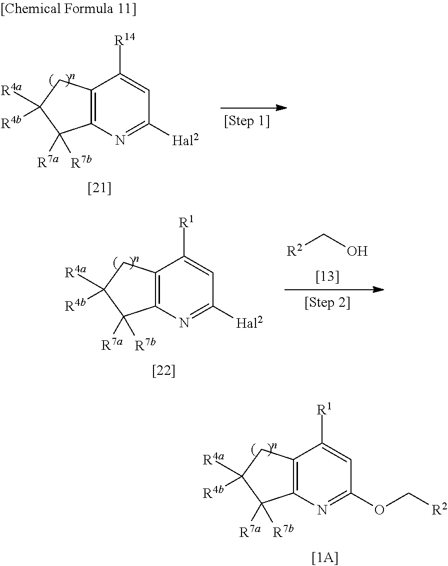

(R¹, R², R^{4a}, R^{4b}, R^{7a}, R^{7b}, Hal², and n mean the same as described above. R¹⁴ represents halogen, trifluoromethanesulfonate, or the substituent [6].)

### Step 1

This reaction is a cross-coupling reaction of a compound represented by the foregoing general formula [21] (hereinafter referred to as “compound [21]”) with the compound [7A], the compound [7B], or the compound [8] using a palladium catalyst, and this reaction can be, for example, performed in the presence of an appropriate base and/or inorganic salt in an appropriate solvent.

A compound represented by the foregoing general formula [22] (hereinafter referred to as “compound [22]”) can be, for example, produced by subjecting the compound [21] wherein R¹⁴ is halogen or trifluoromethanesulfonate and the compound [7A] or compound [7B], or the compound [21] wherein R¹⁴ is the substituent [6] and the compound [8], to a cross-coupling reaction. In addition, a microwave reaction apparatus may be used.

An amount of the compound [7A], compound [7B], or compound [8] to be used is suitably in a range of, for example, 1 to 3-fold molar amount with respect to the compound [21]. Examples of the usable palladium catalyst may include Pd₂(dba)₃.CHCl₂, Pd₂(dba)₃, Pd(PPh₂)₄, Pd(dppf)Cl₂.CH₂Cl₂, PdCl₂(PPh₂)₂, Pd(dtbpf)Cl₂, PdCl₂(PCy₃)₂, and Pd(OAc)₂. An amount of such a palladium catalyst is suitably in a range of, for example, 0.01 to 0.3-fold molar amount with respect to the compound [21]. Examples of the usable base may include inorganic bases such as potassium carbonate, sodium carbonate, sodium hydrogen carbonate, tripotassium phosphate, cesium carbonate, and potassium acetate. An amount of such a base is suitably in a range of, for example, 1 to 3-fold molar amount with respect to the compound [21]. Examples of the usable inorganic salt may include LiCl and cesium fluoride. An amount of such an inorganic salt is suitably in a range of, for example, 1 to 3-fold molar amount with respect to the compound [21]. Although the usable solvent is not particularly limited so long as it does not participate in the reaction, examples thereof may include: hydrocarbons such as toluene and xylene; ethers such as 1,4-dioxane, THF, and DME; amides such as DMF, DMA, and NMP; alcohols such as ethanol and propanol; MeCN; water; and mixed solvents thereof. A reaction temperature is suitably in a range of 20° C. to 200° C. Although a reaction time varies depending on the type of a starting material to be used, the reaction temperature, or the like, in general, it is suitably in a range of 0.5 to 24 hours.

### Step 2

The compound [1A] can be, for example, produced by allowing the compound [22] to react with the compound [13] which is commercially available or can be produced by a known method according to the method described in Step 2 of Production Method C as described above.

Incidentally, the compound [21] that is a starting compound can be, for example, produced according to the following production method.

### Production Method of Compound [21]:

Production method of a compound represented by the following general formula [21A] (hereinafter referred to as “compound [21A]”), which is the compound [21], wherein R¹⁴ is trifluoromethanesulfonate, and a compound represented by the following general formula [21B] (hereinafter referred to as “compound [21B]”), which is the compound [21], wherein R¹⁴ is the substituent [6]

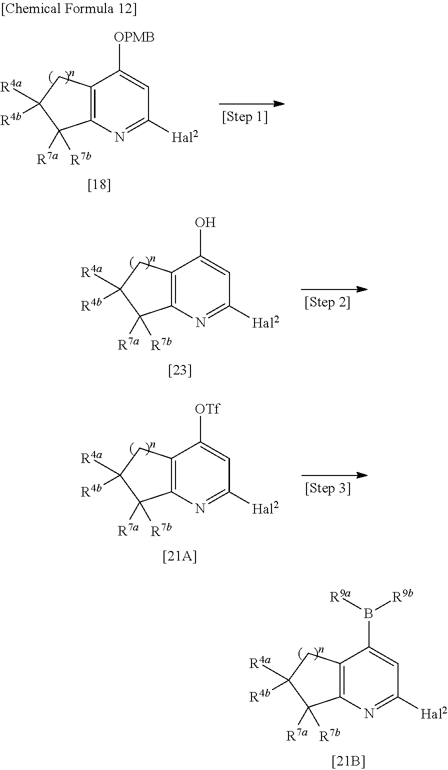

(R^{4a}, R^{4b}, R^{7a}, R^{7b}, R^{9a}, R^{9b}, Hal², PMB, Tf, and n mean the same as described above.)

### Step 1

This reaction is a p-methoxybenzyl deprotection reaction, and a compound represented by the foregoing general formula [23] (hereinafter referred to as “compound [23]”) can be, for example, produced by deprotection of p-methoxybenzyl of the compound [18] according to the method described in Step 3 of Production Method C of the compound [5] as described above.

### Step 2

This reaction is a trifluoromethanesulfonylation reaction, and the compound [21A] can be, for example, produced by allowing the compound [23] to react with a trifluoromethanesulfonylating agent according to the method described in Step 4 of Production Method C of the compound [5] as described above.

### Step 3

This reaction is a boration reaction, and the compound [21B] can be, for example, produced by subjecting the compound [21A] to boration according to the method described in Step 5 of Production Method C of the compound [5] as described above.

Incidentally, the compound [21] wherein R¹⁴ is halogen can be produced according to the method described in Step 1 of Production Method B of the compound [5] as described above.

### Production Method 3:

Production method of a compound represented by the following general formula [1B] (hereinafter referred to as “compound [1B]”), which is the compound of the present invention represented by the general formula (1), wherein R^{3a} is hydrogen, and R^{3b} is alkylcarbonyloxy; a compound represented by the following general formula [1C] (hereinafter referred to as “compound [1C]”), which is the compound of the present invention represented by the general formula (1), wherein R^{3a} is hydrogen, and R^{3b} is hydroxy; a compound represented by the following general formula [1D] (hereinafter referred to as “compound [1D]”), which is the compound of the present invention represented by the general formula (1), wherein R^{3a} and R^{3b} taken together with the adjacent carbon atom represent the group [3]; and a compound represented by the following general formula [1E] (hereinafter referred to as “compound [1E]”), which is the compound of the present invention represented by the general formula (1), wherein R^{3a} and R^{3b} taken together with the adjacent carbon atom represent the group [4]

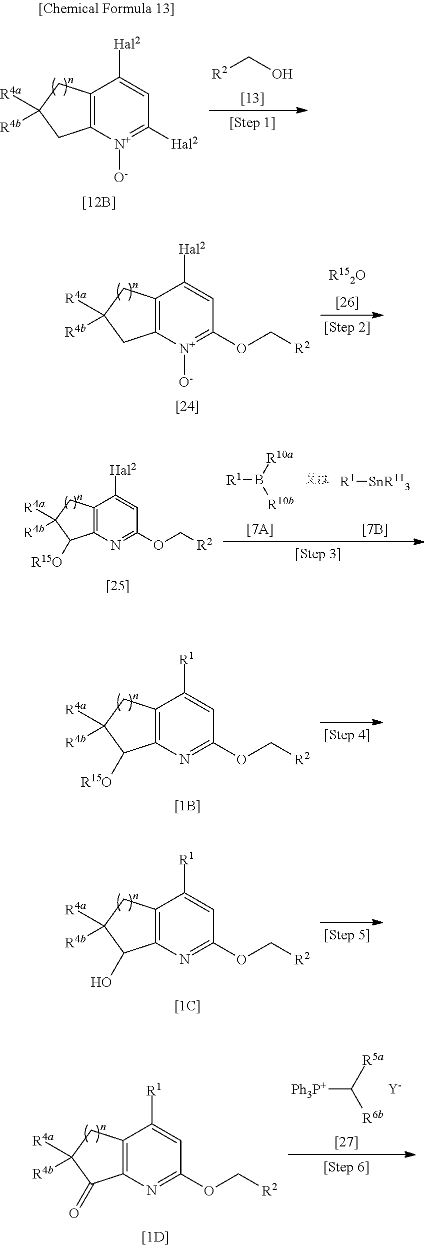

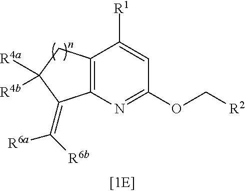

(R¹, R², R^{4a}, R^{4b}, R^{6a}, R^{6b}, R^{10a}, R^{10b}, R¹¹, Hal², and n mean the same as described above. R¹⁵ represents alkylcarbonyl. Ph represents phenyl. Y represents halogen.)

### Step 1

A compound represented by the foregoing general formula [24] (hereinafter referred to as “compound [24]”) can be produced by allowing the compound [12B] to react with the compound [13] which is commercially available or can be produced according to a known method. In general, this reaction can be performed in the presence of an appropriate base in an appropriate solvent at a temperature ranging from 0° C. to 200° C.

An amount of the compound [13] to be used is suitably in a range of, for example, 1 to 3-fold molar amount with respect to the compound [12B]. Examples of the usable base may include NaO-t-Bu, KO-t-Bu, potassium carbonate, cesium carbonate, sodium hydroxide, and sodium hydride. An amount of such abase is suitably in a range of, for example, 1 to 5-fold molar amount with respect to the compound [12B]. Although the usable solvent is not particularly limited so long as it does not participate in the reaction, examples thereof may include: hydrocarbons such as toluene and xylene; ethers such as Et₂O, THF, 1,4-dioxane, and DME; and amides such as DMF, DMA, and NMP. Although a reaction time varies depending on the type of a starting material to be used, the reaction temperature, or the like, in general, it is suitably in a range of 1 to 24 hours.

### Step 2

A compound represented by the foregoing general formula [25] (hereinafter referred to as “compound [25]”) can be produced by allowing the compound [24] to react with a compound represented by the foregoing general formula [26] (hereinafter referred to as “compound [26]”): R¹⁵₂O (R¹⁵ means the same as described above). In general, this reaction can be performed in the presence of the compound [26] in an appropriate solvent at a temperature ranging from 20° C. to 150° C. In addition, if desired, a base may be added.

Examples of the usable base may include sodium carbonate, sodium hydrogen carbonate, and sodium hydroxide. Although the usable solvent is not particularly limited so long as it does not participate in the reaction, examples thereof may include: halogenated hydrocarbons such as CH₂Cl₂, 1,2-dichloroethane, and chloroform; hydrocarbons such as benzene, toluene, and xylene; ethers such as Et₂O, THF, 1,4-dioxane, and DME; ethyl acetate; water; and mixed solvents thereof. Although a reaction time varies depending on the type of a starting material to be used, the reaction temperature, or the like, in general, it is suitably in a range of 0.5 to 24 hours.

### Step 3

This reaction is a cross-coupling reaction of the compound [25] with the compound [7A] or compound [7B] which is commercially available or can be produced by a known method using a palladium catalyst, and the compound [1B] can be, for example, produced according to the method described in Production Method 1 as described above.

### Step 4

This reaction is an alkylcarbonyl deprotection reaction, and the compound [1C] can be, for example, produced by allowing the compound [1B] to react with an appropriate base. In general, this reaction can be performed in the presence of an appropriate base in an appropriate solvent at a temperature ranging from 0° C. to 100° C.

Examples of the usable base may include sodium hydroxide, potassium hydroxide, lithium hydroxide, potassium carbonate, sodium carbonate, ammonia, sodium methoxide, and potassium methoxide. Although the usable solvent is not particularly limited so long as it does not participate in the reaction, examples thereof may include: halogenated hydrocarbons such as CH₂Cl₂ and chloroform; ethers such as Et₂O, THF, 1,4-dioxane, and DME; alcohols such as methanol, ethanol, and propanol; MeCN; water; and mixed solvents thereof. Although a reaction time varies depending on the type of a starting material to be used, the reaction temperature, or the like, in general, it is suitably in a range of 1 to 24 hours.

### Step 5

This reaction is an oxidation reaction of a secondary hydroxyl group, and the compound [1D] can be, for example, produced by allowing the compound [1C] to react with an oxidizing agent according to a method described in “Dai 4-han Jikken Kagaku Kouza, Vol. 23 (Oxidation Reaction)”, Maruzen (1991), pp. 37-78 and pp. 299-346, edited by The Chemical Society of Japan; Tetrahedron, 1978, vol. 34, pp. 1651-1660; or Tetrahedron Letters, 1994, vol. 35, pp. 8019-8022.

Examples of the usable oxidizing agent may include Jones reagent, Sarett reagent, Collins reagent, and Dess-Martin reagent.

### Step 6

This reaction is the Wittig reaction, and the compound [1E] can be produced by allowing the compound [1D] to react with a compound represented by the foregoing general formula [27] which is commercially available or can be produced by a known method, according to a method described in Organic Reactions, 1965, vol. 14, p. 270.

Although the compound of the present invention may be used as a medicinal agent as it is, it may also be used in a form of a pharmaceutically acceptable salt according to a known method. Examples of such a salt may include: salts of mineral acids such as hydrochloric acid, hydrobromic acid, sulfuric acid, and phosphoric acid; salts of organic acids such as acetic acid, citric acid, oxalic acid, tartaric acid, maleic acid, succinic acid, fumaric acid, p-toluenesulfonic acid, benzenesulfonic acid, methanesulfonic acid, trifluoroacetic acid, and methanesulfonamide; salts of alkali metals or alkaline earth metals such as sodium, potassium, and calcium; and salts of organic amine compounds such as L-arginine, choline, L-lysine, t-butylamine, ethylenediamine, ammonia, dimethylaminoethanol, N-methylglucamine, tromethamine, and hydroxyethyl morpholine.

For example, a hydrochloride of the compound of the present invention can be obtained by dissolving the compound of the present invention in a solution of hydrogen chloride (hereinafter referred to as “HCl”) in an alcohol, ethyl acetate, or Et₂O.

Although some of the compounds of the present invention have asymmetric carbon, all of their respective optical isomers and mixtures thereof fall within the scope of the present invention. For example, an optical isomer can be produced by optically resolving a racemate with an optically active acid (e.g., tartaric acid, dibenzoyltartaric acid, mandelic acid, 10-camphor sulfonic acid, etc.) while utilizing basicity thereof according to a known method, or by using an optically active compound having been prepared in advance as a starting material. Besides, the optical isomer can also be produced by optical resolution using a chiral column or asymmetric synthesis.

In addition, among the compounds of the present invention, with respect to those which may form a tautomer, all of their respective tautomers and mixtures thereof fall within the scope of the present invention.

The compound of the present invention or a pharmaceutically acceptable salt thereof has mGluR5 inhibitory activity as shown in the Test Examples as described later.

Accordingly, the compound of the present invention or a pharmaceutically acceptable salt thereof can be used as a preventive agent or therapeutic agent for diseases in which mGluR5 participates.

Examples of a disease to which the compound of the present invention or a pharmaceutically acceptable salt thereof is applicable may include the following:

(1) pain
(2) pruritus (see, for example, Patent Literature 1)
(3) a lower urinary tract symptom or lower urinary tract dysfunction
(4) gastroesophageal reflux disease (GERD) (see, for example, Non-Patent Literature 11) or gastroesophageal reflux disease associated with transient lower esophageal sphincter relaxation (TLESR) (see, for example, Non-Patent Literature 11),
(5) central nervous system disease
(6) drug-induced liver damage (see, for example, Non-Patent Literature 11)
(7) hypoxia-induced liver damage (see, for example, Non-Patent Literature 11)
(8) obesity (see, for example, Non-Patent Literature 23)
(9) diabetes (see, for example, Non-Patent Literature 11)
(10) oral squamous cell carcinoma (see, for example, Non-Patent Literature 11)
(11) liver cancer (see, for example, Non-Patent Literature 24)

Examples of “pain” may include acute pain (see, for example, Non-Patent Literature 7), chronic pain (see, for example, Non-Patent Literature 7), inflammatory pain (see, for example, Non-Patent Literature 4 and 7), neuropathic pain (see, for example, Non-Patent Literature 5 and 7), hyperalgesia (see, for example, Non-Patent Literature 4 and 6), thermal hyperalgesia, allodynia (see, for example, Non-Patent Literature 6), pain by noxious thermal stimulation (see, for example, Non-Patent Literature 7), pain by noxious mechanical stimulation (see, for example, Non-Patent Literature 7), pain in the lower urinary tract or reproductive organs or migraine (see, for example, Non-Patent Literature 8).

Examples of “acute pain” may include herpetic pain, pain after tooth extraction, postoperative pain or a pain associated with ureteral calculus.

Examples of “chronic pain” may include cancer pain, pain associated with fibromyalgia, pain in complex regional pain syndrome or pain in postoperative scar pain syndrome.

Examples of “inflammatory pain” may include pain associated with rheumatoid arthritis or pain associated with osteoarthritis.

Examples of “neuropathic pain” may include trigeminal neuralgia, postherpetic neuralgia, pain associated with painful diabetic neuropathy or pain associated with carcinomatous neuropathy.

Examples of “pain in the lower urinary tract or reproductive organs” may include pain associated with bacterial cystitis (see, for example, Non-Patent Literature 9, 12, 25 and 26), pain associated with interstitial cystitis (see, for example, Non-Patent Literature 9, 12, 25 and 26), pain associated with cystitis, pain associated with acute prostatitis, pain associated with chronic prostatitis, pain associated with pelvic pain syndrome (see, for example, Non-Patent Literature 10), coital pain, bladder pain, urethral pain, vulvodynia, vaginal pain, scrotal pain, perineal pain or pelvic pain.

Examples of “central nervous system disease” may include L-dopa-induced dyskinesia (see, for example, Non-Patent Literature 13 and 15), psychosis (see, for example, Non-Patent Literature 14), anxiety (see, for example, Non-Patent Literature 6, 14 and 15), depression (see, for example, Non-Patent Literature 14), Alzheimer's disease (see, for example, Non-Patent Literature 16), fragile X syndrome (see, for example, Non-Patent Literature 17), Parkinson's disease, Huntington's disease (see, for example, Non-Patent Literature 18), morphine tolerance (see, for example, Non-Patent Literature 20), alcohol dependence (see, for example, Non-Patent Literature 21) or food addiction (see, for example, Non-Patent Literature 9, 12, 25 and 22).

Examples of “lower urinary tract symptom or lower urinary tract dysfunction” may include frequent urination, diurnal frequency, nocturia, urinary urgency, urinary incontinence, bladder perception, symptom associated with pelvic organ prolapse, neurogenic bladder, lower urinary tract obstruction, discomfort in bladder, discomfort in lower urinary tract or discomfort in genital tract.

Examples of “disease accompanied by lower urinary tract symptom or lower urinary tract dysfunction” may include overactive bladder (see, for example, Non-Patent Literature 12), cystitis, urethritis, bacterial cystitis (see, for example, Non-Patent Literature 9, 12, 25 and 26), interstitial cystitis (see, for example, Non-Patent Literature 9, 12, 25 and 26), prostatic hyperplasia, prostate cancer, urolithiasis, acute prostatitis, chronic prostatitis, bladder tumor, poorly compliant bladder, pelvic organ prolapse, uterine fibroid, polyuria, stress urinary incontinence, cerebrovascular accident, Parkinson disease, multiple system atrophy, brain tumor, dementia, spinal cord injury, multiple sclerosis, Degenerative Myelopathy, spinal vascular disorder, spina bifida, peripheral neuropathy or pelvic pain syndrome (see, for example, Non-Patent Literature 10).

When administered as a medicinal agent, the compound of the present invention or a pharmaceutically acceptable salt thereof is administered as it is or as a pharmaceutical composition containing, for example, 0.001% to 99.5%, and preferably 0.1% to 90% thereof, in a pharmaceutically acceptable non-toxic and inactive carrier to a mammal including human.

A diluent, a bulking agent, and one or more of other formulation additives in the form of a solid, a semi-solid, or a liquid can be used as a carrier. It is desirable that the pharmaceutical composition of the present invention is administered in a unit dosage form. The pharmaceutical composition may be administered by intra-tissue administration, oral administration, intravenous administration, local administration (e.g., dermal administration, ocular instillation, intraperitoneal administration, intrathoracic administration, etc.), or transrectal administration. As a matter of course, the composition is administered in a dosage form suitable for these administration routes.

It is desirable that the dosage as a medicinal agent is adjusted while taking into consideration conditions of the patient such as age, body weight, nature, severity, and the like of the disease, route of administration, the compound of the present invention to be administered, whether or not the compound is a salt, the kind of the salt, and the like. For the oral administration, a daily dosage of the compound of the present invention or a pharmaceutically acceptable salt thereof as an active ingredient for adult is typically within the range of 10 mg to 3,000 mg, and preferably from 30 mg to 600 mg per adult. However, a dosage below the foregoing range may be sufficient in some cases, or a dosage above the foregoing range may be needed in other cases. In general, a daily dosage is administered once per day or may be administered by divided doses. Alternatively, a daily dosage can be administered intravenously by bolus administration or continuous infusion within 24 hours.

### EXAMPLES

The present invention is hereunder described in more detail by reference to the following Examples, Test Examples, and Formulation Example, but it should not be construed that the present invention is limited thereto.

Incidentally, the measurement conditions for high-performance liquid chromatography mass spectrometry are as follows.

Analytical instrument: ACUITY UPLC MS/PDA System (available from Waters)
Mass spectrometer: Waters 3100 MS detector
Photodiode array detector: ACUITY PDA detector (UV detection wavelength: 210 to 400 nm)

### Column: Acuity BEH C18, 1.7 μm, 2.1×50 mm

Flow rate: 0.5 mL/min
Column temperature: 40° C.

### Solvent:

Solvent A: 0.1% formic acid/water (v/v)

Solvent B: 0.1% formic acid/MeCN (v/v)

### Example 1

### 4-(Pyridin-3-yl)-2-(pyridin-2-ylmethoxy)-6,7-dihydro-5H-cyclopenta[b]pyridine dihydrochloride

### [Step 1]

Production of 4-chloro-2-(pyridin-2-ylmethoxy)-6,7-dihydro-5H-cyclopenta[b]pyridine

Toluene (14 mL) was added to pyridine-2-ylmethanol (0.924 mL), followed by the addition of sodium hydride (60%, dispersion in oil, 640 mg) (hereinafter referred to as “NaH”) under ice water cooling and the mixture was stirred for 30 minutes. After the reaction mixture was allowed to return to room temperature, 2,4-dichloro-6,7-dihydro-5H-cyclopenta[b]pyridine (see, for example, Helvetica Chimica Acta, 1945, vol. 28, p. 1684-1690) (2 g) was added, and the reaction mixture was stirred at 170° C. for 1 hour. After the reaction mixture was allowed to return to room temperature, and filtered through Celite, the filtrate was evaporated under reduced pressure. The resulting residue was purified by silica gel column chromatography to give the title compound (1.06 g) as a white solid. [Rf value (TLC silica gel plate 60F₂₅₄, developing solvent; hexane: ethyl acetate=2:1): 0.3]

### [Step 2]

Production of 4-(pyridin-3-yl)-2-(pyridin-2-ylmethoxy)-6,7-dihydro-5H-cyclopenta[b]pyridine dihydrochloride

To 4-chloro-2-(pyridin-2-ylmethoxy)-6,7-dihydro-5H-cyclopenta[b]pyridine (20 mg), pyridin-3-yl boronic acid (19 mg), Pd(dppf)Cl₂.CH₂Cl₂ (5 mg), and potassium carbonate (32 mg) was added 1,4-dioxane/water (3/1, 1.9 mL), and the mixture was stirred at 100° C. for 5 hours. After the solvent was evaporated under reduced pressure, the residue was purified by silica gel column chromatography to give 4-(pyridin-3-yl)-2-(pyridin-2-ylmethoxy)-6,7-dihydro-5H-cyclopenta[b]pyridine (16 mg).

The resulting compound was dissolved in ethyl acetate (10 mL) and 1 N HCl/Et₂O solution (0.26 mL) was added, and the mixture was stirred for 0.5 hour. The resulting precipitate was collected by filtration to give the title compound (10 mg) as a white amorphous form.

[MS (ESI) m/z 304.3 (M+H)⁺]

### Example 2

### 4-(2-Fluoropyridin-3-yl)-2-(pyridin-2-ylmethoxy)-6,7-dihydro-5H-cyclopenta[b]pyridine hydrochloride

To 4-chloro-2-(pyridin-2-ylmethoxy)-6,7-dihydro-5H-cyclopenta[b]pyridine (100 mg), 2-fluoropyridin-3-ylboronic acid (70 mg), Pd(OAc)₂ (9 mg), X-Phos (37 mg) and potassium. phosphate (244 mg) was added 1,4-dioxane/water (3/1, 2 mL), and the mixture was stirred at 100° C. for 6 hours. After the reaction mixture was allowed to return to room temperature, diluted with ethyl acetate, and filtered through Celite, the filtrate was evaporated under reduced pressure. The resulting residue was purified by silica gel column chromatography to give 4-(2-fluoropyridin-3-yl)-2-(pyridin-2-ylmethoxy)-6,7-dihydro-5H-cyclopenta[b]pyridine (48 mg).

[MS (ESI) m/z 322.0 (M+H)⁺]

The resulting compound was dissolved in ethyl acetate, 1 N HCl/Et₂O solution (0.12 mL) was added, and the mixture was stirred for 0.5 hour. The resulting precipitate was collected by filtration to give the title compound (10 mg) as a white powder.

[MS (ESI) m/z 322.0 (M+H)⁺]

### Example 3

### 3-[2-(Pyridin-2-ylmethoxy)-6,7-dihydro-5H-cyclopenta[b]pyridin-4-yl]pyridine-2-carbonitrile

To a solution of 4-(2-fluoropyridin-3-yl)-2-(pyridin-2-ylmethoxy)-6,7-dihydro-5H-cyclopenta[b]pyridine (8 mg) in DMSO (0.2 mL) was added NaCN (4.9 mg), and the mixture was stirred at 150° C. for 24 hours. After the reaction mixture was allowed to return to room temperature, added with water, and filtered through Celite, the filtrate was evaporated under reduced pressure. The resulting precipitate was collected by filtration to give the title compound (5 mg) as a white powder.

[MS (ESI) m/z 329.2 (M+H)⁺]

### Example 4

### 5-[2-(Pyridin-2-ylmethoxy)-6,7-dihydro-5H-cyclopenta[b]pyridin-4-yl]pyridine-2-carbonitrile hydrochloride

### [Step 1]

Production of 2-(pyridin-2-ylmethoxy)-6,7-dihydro-5H-cyclopenta[b]pyridin-4-ol

To 4-chloro-2-(pyridin-2-ylmethoxy)-6,7-dihydro-5H-cyclopenta[b]pyridine (5 g), potassium hydroxide (3.23 g), Pd₂(dba)₃ (352 mg) and t-Bu-XPhos (408 mg) were added 1,4-dioxane (20 mL) and water (20 mL), and the mixture was degassed, then stirred under argon (hereinafter referred to as “Ar”) atmosphere at 100° C. for 2 hours. The reaction mixture was filtered through Celite, and the filtrate was evaporated under reduced pressure. The residue was neutralized with hydrochloric acid and then the mixture was weakly basified with sodium hydrogen carbonate. The resulting mixture was extracted with water and ethyl acetate. The organic layer was washed with saturated brine, dried over anhydrous sodium sulfate, and filtered off, and the solvent was evaporated under reduced pressure. The resulting residue was purified by silica gel column chromatography to give the title compound (4.55 g).

### [Step 2]

Production of 2-(pyridin-2-ylmethoxy)-6,7-dihydro-5H-cyclopenta[b]pyridin-4-yl trifluoromethanesulfonate

To 2-(pyridin-2-ylmethoxy)-6,7-dihydro-5H-cyclopenta[b]pyridin-4-ol (4.55 g) were added CH₂Cl₂(100 mL), Et₃N (5.3 mL) and Tf₂NPh (8.05 g), and the mixture was stirred at room temperature for 5 hours. After the solvent of the reaction mixture was evaporated under reduced pressure, the residue was extracted with water and ethyl acetate. The organic layer was washed with saturated brine, dried over anhydrous sodium sulfate, and filtered off, and the solvent was evaporated under reduced pressure. The resulting residue was purified by silica gel column chromatography to give the title compound (6.46 g).

### [Step 3]

Production of 5-[2-(pyridin-2-ylmethoxy)-6,7-dihydro-5H-cyclopenta[b]pyridin-4-yl]pyridine-2-carbonitrile hydrochloride

To 2-(pyridin-2-ylmethoxy)-6,7-dihydro-5H-cyclopenta[b]pyridin-4-yl trifluoromethanesulfonate (100 mg), 5-(4,4,5,5-tetramethyl-1,3,2-dioxaborolan-2-yl)pyridine-2-carbonitrile (74 mg), Pd (dppf)Cl₂.CH₂Cl₂ (22 mg) and potassium carbonate (111 mg) was added 1,4-dioxane/water (3/1, 4 mL), and the mixture was degassed, and then stirred under Ar atmosphere at 80° C. for 1 hour. After the reaction mixture was allowed to return to room temperature, the mixture was added with water and ethyl acetate, and subjected to extraction. The organic layer was washed with saturated brine, dried over anhydrous sodium sulfate, and filtered off, and the solvent was evaporated under reduced pressure. The resulting residue was purified by silica gel column chromatography to give 5-[2-(pyridin-2-ylmethoxy)-6,7-dihydro-5H-cyclopenta[b]pyridin-4-yl]pyridine-2-carbonitrile (97 mg).

The resulting compound was dissolved in ethyl acetate (3 mL), and added with 1 N HCl/Et₂O solution (0.32 mL) under ice water cooling, and the mixture was stirred for 0.5 hour under ice water cooling. The resulting precipitate was collected by filtration to give the title compound (70 mg) as a pale yellow powder.

[MS (ESI) m/z 329.5 (M+H)⁺]

### Example 5

### 5-[2-(Pyridin-2-ylmethoxy)-6,7-dihydro-5H-cyclopenta[b]pyridin-4-yl]pyridin-3-ol

### [Step 1]

Production of 2-(pyridin-2-ylmethoxy)-4-(4,4,5,5-tetramethyl-1,3,2-dioxaborolan-2-yl)-6,7-dihydro-5H-cyclopenta[b]pyridine

To 2-(pyridin-2-ylmethoxy)-6,7-dihydro-5H-cyclopenta[b ]pyridin-4-yl trifluoromethanesulfonate (273 mg), bis(pinacolato)diboron (1.87 g), potassium acetate (1.97 g) and Pd(dppf)Cl₂.CH₂Cl₂ (273 mg) was added 1,4-dioxane (50 mL), and the mixture was degassed, and then stirred under Ar atmosphere at 100° C. for 1 hour. After the reaction mixture was allowed to return to room temperature, diluted with ethyl acetate, and filtered through Celite, the filtrate was evaporated under reduced pressure. The resulting residue was purified by silica gel column chromatography to give the title compound (48 mg) as a white solid.

### [Step 2]

Production of 5-[2-(pyridin-2-ylmethoxy)-6,7-dihydro-5H-cyclopenta[b]pyridin-4-yl]pyridin-3-ol

To 2-(pyridin-2-ylmethoxy)-4-(4,4,5,5-tetramethyl-1,3,2-dioxaborolan-2-yl)-6,7-dihydro-5H-cyclopenta[b]pyridine (100 mg), 5-bromopyridin-3-ol (74 mg), Pd(dppf)Cl₂.CH₂Cl₂ (19 mg) and potassium carbonate (118 mg) was added 1,4-dioxane/water (3/1, 1.4 mL), and the mixture was degassed and stirred under Ar atmosphere at 100° C. for 2 hours. After the reaction mixture was allowed to return to room temperature, diluted with ethyl acetate, and filtered through Celite, the filtrate was evaporated under reduced pressure. The resulting residue was purified by silica gel column chromatography to give the title compound (16 mg) as a pale yellow powder.

[MS (ESI) m/z 320.3 (M+H)⁺]

### Example 6

### 5-[2-(Pyridin-2-ylmethoxy)-6,7-dihydro-5H-cyclopenta[b]pyridin-4-yl]pyridin-3-yl methanesulfonate

To a solution of 5-[2-(pyridin-2-ylmethoxy)-6,7-dihydro-5H-cyclopent a[b]pyridin-4-yl]pyridin-3-ol (13 mg) in CH₂Cl₂ (1 mL) were added Et₃N (0.023 mL) and methanesulfonyl chloride (hereinafter referred to as “MsCl”) (0.007 mL) sequentially, and the mixture was stirred at room temperature overnight. The reaction mixture was purified by silica gel column chromatography to give the title compound (4 mg) as colorless oil.

[MS (ESI) m/z 398.3 (M+H)⁺]

### Example 7

N-{3-Fluoro-5-[2-(pyridin-2-ylmethoxy)-6,7-dihydro-5H-cyclopenta[b]pyridin-4-yl]phenyl methanesulfonamide}methanesulfonamide

### [Step 1]

Production of 3-fluoro-5-[2-(pyridin-2-ylmethoxy)-6,7-dihydro-5H-cyclopenta[b]pyridin-4-yl]phenol

To 2-(pyridin-2-ylmethoxy)-4-(4,4,5,5-tetramethyl-1,3, 2-dioxaborolan-2-yl)-6,7-dihydro-5H-cyclopenta[b]pyridine (100 mg), 3-bromo-5-fluorophenol (82 mg), Pd(OAc)₂ (2.6 mg) and S-Phos (9.4 mg) was added ethanol (1.4 mL), and the mixture was degassed, and stirred under Ar atmosphere at 100° C. for 2 hours. After the reaction mixture was allowed to return to room temperature, diluted with ethyl acetate, and filtered through Celite, the filtrate was evaporated under reduced pressure. The resulting residue was purified by silica gel column chromatography to give the title compound (63 mg) as a white powder.

### [Step 2]

Production of 3-fluoro-5-[2-(pyridin-2-ylmethoxy)-6,7-dihydro-5H-cyclopenta[b]pyridin-4-yl]phenyl trifluoromethanesulfonate

To 3-fluoro-5-[2-(pyridin-2-ylmethoxy)-6,7-dihydro-5H-cyclopenta[b]pyridin-4-yl]phenol (36 mg) and pyridine (4 mg) was added CH₂Cl₂ (2 mL). The mixture was stirred under Ar atmosphere under ice water cooling, and then added with Tf₂O (0.035 mL) at the same temperature for 2 hours. The solvent was removed under reduced pressure, and the resulting residue was purified by silica gel column chromatography to give the title compound (45 mg) as colorless oil.

### [Step 3]

Production of N-{3-fluoro-5-[2-(pyridin-2-ylmethoxy)-6,7-dihydro-5H-cyclopenta[b]pyridin-4-yl]phenyl methanesulfonamide}methanesulfonamide

To 3-fluoro-5-[2-(pyridin-2-ylmethoxy)-6,7-dihydro-5H-cyclopenta[b]pyridin-4-yl]phenyl trifluoromethanesulfonate (10 mg), methanesulfonamide (18 mg), Pd₂(dba)₃.CHCl₃ (10 mg), t-Bu-X-Phos (8 mg) and potassium phosphate (41 mg) was added toluene (1 mL), and the mixture was degassed, and stirred under Ar atmosphere at 100° C. overnight. After the reaction mixture was allowed to return to room temperature, diluted with ethyl acetate, and filtered through Celite, the filtrate was evaporated under reduced pressure. The resulting residue was purified by silica gel column chromatography to give the title compound (40 mg) as a white powder.

[MS (ESI) m/z 414.4 (M+H)⁺]

### Example 8

### 6-({[4-(2-Fluoropyridin-3-yl)-6,7-dihydro-5H-cyclopenta[b]pyridin-2-yl]oxy}methyl)pyridine-2-carbonitrile

### [Step 1]

Production of 2-[(1-oxidopyridin-2-yl)methoxy)-6,7-dihydro-5H-cyclopenta[b]pyridin-4-yl trifluoromethanesulfonate

To a solution of 2-(pyridin-2-ylmethoxy)-6,7-dihydro-5H-cyclopenta[b]pyridin-4-yl trifluoromethanesulfonate in CH₂Cl₂ (70 mL) was added m-CPBA (with abs. 25% water, 2.9 g) under ice water cooling, and the mixture was stirred at room temperature for 1 hour. The solvent of the reaction mixture was removed under reduced pressure, and the resulting residue was purified by silica gel column chromatography to give the title compound (3.52 g).

### [Step 2]

Production of 2-[(6-cyanopyridin-2-yl)methoxy]-6,7-dihydro-5H-cyclopenta[b]pyridin-4-yl trifluoromethanesulfonate

To a solution of 2-[(1-oxidopyridin-2-yl)methoxy)-6,7-dihydro-5H-cyclopenta[b]pyridin-4-yl trifluoromethanesulfonate (3.52 g) in CH₂Cl₂ (9 mL) were added N,N-dimethylcarbamoyl chloride (1.93 g) and trimethylsilyl cyanide (1.78 g), and the mixture was stirred at room temperature for 1 hour. The solvent was removed under reduced pressure, and the resulting residue was purified by silica gel column chromatography to give the title compound (3.93 g) as colorless oil.

### [Step 3]

Production of 6-({[4-(4,4,5,5-tetramethyl-1,3,2-dioxaborolan-2-yl)-6,7-dihydro-5H-cyclopenta[b]pyridin-2-yl]oxy}methyl)pyridine-2-carbonitrile

The title compound was prepared as a white solid according to the procedure described in Example 5, Step 1, using 2-[(6-cyanopyridin-2-yl)methoxy]-6,7-dihydro-5H-cyclopenta[b]pyridin-4-yl trifluoromethanesulfonate instead of 2-(pyridin-2-ylmethoxy)-6,7-dihydro-5H-cyclopenta[b]pyridin-4-yl trifluoromethanesulfonate.

### [Step 4]

Production of 6-({[4-(2-fluoropyridin-3-yl)-6,7-dihydro-5H-cyclopenta[b]pyridin-2-yl]oxy}methyl)pyridine-2-carbonitrile

To 6-({[4-(4,4,5,5-tetramethyl-1,3,2-dioxaborolan-2-yl)-6,7-dihydro-5H-cyclopenta[b]pyridin-2-yl]oxy}methyl)pyridine-2-carbonitrile (100 mg), 2-fluoro-3 iodopyridine (71 mg), Pd(dppf)Cl₂.CH₂Cl₂ (17 mg) and potassium carbonate (110 mg) was added 1,4-dioxane/water (3/1, 2.6 mL), and the mixture was degassed, and then stirred under Ar atmosphere at 100° C. for 4 hours. After the reaction mixture was allowed to return to room temperature, diluted with ethyl acetate, dried over anhydrous sodium sulfate, and filtered through Celite, the filtrate was evaporated under reduced pressure. The resulting residue was purified by silica gel column chromatography to give the title compound (65 mg) as a white solid.

[MS (ESI) m/z 347.4 (M+H)⁺]

### Example 9

### 5-{2-[(5-Fluoropyridin-2-yl)methoxy]-6,7-dihydro-5H-cyclopenta[b]pyridin-4-yl}pyridine-2-carbonitrile

### [Step 1]

Production of 2-chloro-4-(pyridin-2-ylmethoxy)-6,7-dihydro-5H-cyclopenta[b]pyridine

To pyridine-2-ylmethanol (0.694 mL) was added toluene (12 mL) and then NaH (60% dispersion in oil, 480 mg) was added under ice water cooling, and the mixture was stirred for 30 minutes. After the reaction mixture was allowed to return to room temperature, 2,4-dichloro-6,7-dihydro-5H-cyclopenta[b]pyridine (1.5 g) was added to the mixture, and the mixture was stirred at 170° C. for 1 hour. After the reaction mixture was allowed to return to room temperature, it was added with water and ethyl acetate, and subjected to extraction. The organic layer was washed with saturated brine, and dried over anhydrous sodium sulfate, and the solvent was evaporated under reduced pressure. The resulting residue was purified by silica gel column chromatography to give the title compound (623 mg) as a white solid.

[Rf value (TLC silica gel plate 60F₂₅₄, developing solvent:hexane:ethyl acetate=1:1):0.3]

### [Step 2]

Production of 2-chloro-6,7-dihydro-5H-cyclopenta[b]pyridin-4-ol

To 2-chloro-4-(pyridin-2-ylmethoxy)-6,7-dihydro-5H-cyclopenta[b]pyridine (608 mg) and sulfuric acid (0.24 mL) were added MeCN (15 mL) and sodium iodide (hereinafter referred to as “NaI”) (1.33 g), and the mixture was heated under reflux for 12 hours. After the solvent of the reaction mixture was removed under reduced pressure, the resulting residue was added with ethyl acetate and aqueous sodium thiosulfate solution, and subjected to extraction. The organic layer was washed with saturated brine, dried over anhydrous sodium sulfate, and filtered off, and the solvent was evaporated under reduced pressure to give the title compound (470 mg) as a white solid.

### [Step 3]

Production of 2-chloro-6,7-dihydro-5H-cyclopenta[b]pyridin-4-yl trifluoromethanesulfonate

To 2-chloro-6,7-dihydro-5H-cyclopenta[b]pyridin-4-ol (269 mg), 2,6-lutidine (0.34 mL) and DMAP (18 mg) was added CH₂Cl₂ (15 mL), and the mixture was added with Tf₂O (0.3 mL) under ice water cooling, and stirred at room temperature for 3 hours. After the solvent of the reaction mixture was removed under reduced pressure, the resulting residue was added with Et₂O and water, and subjected to extraction. The organic layer was dried over anhydrous sodium sulfate, and filtered, and the solvent was evaporated under reduced pressure. The resulting residue was purified by silica gel column chromatography to give the title compound (439 mg) as colorless oil.

### [Step 4]

Production of 2-chloro-4-(4,4,5,5-tetramethyl-1,3,2-dioxaborolan-2-yl)-6,7-dihydro-5H-cyclopenta[b]pyridine

The title compound was prepared as a white powder according to the procedure described in Example 5, Step 1, using 2-chloro-6,7-dihydro-5H-cyclopenta[b]pyridin-4-yl trifluoromethanesulfonate instead of 2-(pyridin-2-ylmethoxy)-6,7-dihydro-5H-cyclopenta[b]pyridin-4-yl trifluoromethanesulfonate.

### [Step 5]

Production of 5-(2-chloro-6,7-dihydro-5H-cyclopenta[b]pyridin-4-yl) pyridine-2-carbonitrile

The title compound was prepared as a white powder according to the procedure described in Example 5, Step 2, using 2-chloro-4-(4,4,5,5-tetramethyl-1,3,2-dioxaborolan-2-yl)-6,7-dihydro-5H-cyclopenta[b]pyridine instead of 2-(pyridin-2-ylmethoxy)-4-(4,4,5,5-tetramethyl-1,3,2-dioxaborolan-2-yl)-6,7-dihydro-5H-cyclopenta[b]pyridine, and using 5-bromopyridine-2-carbonitrile instead of 5-bromopyridin-3-ol.

### [Step 6]

Production of 5-{2-[(5-fluoropyridin-2-yl)methoxy]-6,7-dihydro-5H-cyclopenta[b]pyridin-4-yl}pyridine-2-carbonitrile

To 5-(2-chloro-6,7-dihydro-5H-cyclopenta[b]pyridin-4-yl)pyridine-2-carbonitrile (60 mg), (5-fluoropyridin-2-yl)methanol (36 mg), t-Bu-X-Phos (24 mg), NaO-t-Bu (45 mg), Pd₂(dba)₃.CHCl₃ (10 mg) and molecular sieve 4A (hereinafter referred to as “MS4A”) was added toluene (1 mL), and the mixture was degassed, and then stirred under Ar atmosphere at 100° C. for 1 hour. After the reaction mixture was allowed to return to room temperature, diluted with ethyl acetate, and filtered through Celite, the filtrate was evaporated under reduced pressure. The resulting residue was dissolved in CH₂Cl₂ (3 mL), and then Et₃N (0.1 mL) and TFAA (0.05 mL) were added to the mixture under ice water cooling under Ar atmosphere, and the mixture was stirred at the same temperature for 2 hours. The resulting mixture was added with water and ethyl acetate, and subjected to extraction. The organic layer was washed with saturated brine, dried over anhydrous sodium sulfate, and filtered off, and the solvent was evaporated under reduced pressure. The resulting residue was purified by silica gel column chromatography to give the title compound (11 mg).

[MS (ESI) m/z 347.3 (M+H)⁺]

### Example 10

### 5-{2-[(6-Methylpyridin-2-yl)methoxy]-6,7-dihydro-5H-cyclopenta[b]pyridin-4-yl}pyridine-2-carbonitrile

The title compound was prepared as a white solid according to the procedure described in Example 9, Step 6, using (6-methylpyridin-2-yl)methanol instead of (5-fluoropyridin-2-yl)methanol.

[MS (ESI) m/z 343.4 (M+H)⁺]

### Example 11

### 2-(Benzyloxy)-4-(pyrimidin-5-yl)-6,7-dihydro-5H-cyclopenta[b]pyridine

### [Step 1]

Production of 2-chloro-4-(pyrimidin-5-yl)-6,7-dihydro-5H-cyclopenta[b]pyridine

To 2-chloro-6,7-dihydro-5H-cyclopenta[b]pyridin-4-yl trifluoromethanesulfonate (439 mg), Pd(dppf)Cl₂.CH₂Cl₂ (59 mg), potassium carbonate (603 mg) and pyrimidin-5-yl boronic acid (198 mg) was added THF/water (3/1, 16 mL), and the mixture was degassed, and then stirred under Ar atmosphere at 60° C. for 1 hour. The solvent of the reaction mixture was evaporated under reduced pressure, diluted with ethyl acetate, and filtered through Celite, and the filtrate was evaporated under reduced pressure. The resulting residue was purified by silica gel column chromatography to give the title compound (315 mg).

### [Step 2]

Production of 2-(benzyloxy)-4-(pyrimidin-5-yl)-6,7-dihydro-5H-cyclopenta[b]pyridine

To 2-chloro-4-(pyrimidin-5-yl)-6,7-dihydro-5H-cyclopenta[b]pyridine (50 mg), benzylalcohol (35 mg), t-Bu-X-Phos (15 mg), NaO-t-Bu (41 mg) and Pd₂ (dba)₃.CHCl₃ (9 mg) was added toluene (2 mL), and the mixture was degassed, and then stirred under Ar atmosphere at 100° C. for 1 hour. After the reaction mixture was allowed to return to room temperature, diluted with ethyl acetate, and filtered through a short column, the solvent was evaporated under reduced pressure. The resulting residue was purified by silica gel column chromatography to give the title compound (11 mg).

[MS (ESI) m/z 304.4 (M+H)⁺]

### Example 12

### 2-(Pyridin-2-ylmethoxy)-4-(pyrimidin-5-yl)-6,7-dihydro-5H-cyclopenta[b]pyridine hydrochloride

To 4-chloro-2-(pyridin-2-ylmethoxy)-6,7-dihydro-5H-cyclopenta[b]pyridine (100 mg), pyrimidin-5-yl boronic acid (143 mg), Pd(OAc)₂ (9 mg), S-Phos (31 mg) and potassium carbonate (158 mg) was added 1,4-dioxane/water (3/1, 1.9 mL), and the mixture was degassed, and then stirred under Ar atmosphere at 100° C. for 6 hours. After the reaction mixture was allowed to return to room temperature, diluted with ethyl acetate, dried over anhydrous sodium sulfate, and filtered through Celite, the solvent was evaporated under reduced pressure. The resulting residue was purified by silica gel column chromatography to give 2-(pyridin-2-ylmethoxy)-4-(pyrimidin-5-yl)-6,7-dihydro-5H-cyclopenta[b]pyridine (76 mg).

The resulting compound was dissolved in ethyl acetate (4.8 mL), 1 N HCl/Et₂O solution (0.24 mL) was added under ice water cooling, and the mixture was stirred at the same temperature for 1 hour. The resulting precipitate was collected by filtration to give the title compound (47 mg) as a white powder.

Elementary analysis as C₁₈H₁₆N₄O.HCl+0.2H₂O

Calcd. (%) C: 62.77.; H: 5.09.; N: 16.27.

Found. (%) C: 62.81.; H: 4.85.; N: 15.80.

### Example 13

### 2-(Pyridin-3-ylmethoxy)-4-(pyrimidin-5-yl)-6,7-dihydro-5H-cyclopenta[b]pyridine

The title compound was prepared as a white powder according to the procedure described in Example 11, Step 2, using pyridin-3-ylmethanol instead of benzylalcohol.

[MS (ESI) m/z 305.4 (M+H)⁺]

### Example 14

### 2-(Pyridin-4-ylmethoxy)-4-(pyrimidin-5-yl)-6,7-dihydro-5H-cyclopenta[b]pyridine

The title compound was prepared as a white powder according to the procedure described in Example 11, Step 2, using pyridin-4-ylmethanol instead of benzylalcohol.

[MS (ESI) m/z 305.4 (M+H)⁺]

### Example 15

### 2-[(3-Fluorobenzyl)oxy]-4-(pyrimidin-5-yl)-6,7-dihydro-5H-cyclopenta[b]pyridine

The title compound was prepared as a white powder according to the procedure described in Example 11, Step 2, using 3-fluorobenzylalcohol instead of benzylalcohol.

[MS (ESI) m/z 322.3 (M+H)⁺]

### Example 16

### 2-[(2-Fluorobenzyl)oxy]-4-(pyrimidin-5-yl)-6,7-dihydro-5H-cyclopenta[b]pyridine

The title compound was prepared as a white powder according to the procedure described in Example 11, Step 2, using 2-fluorobenzylalcohol instead of benzylalcohol.

[MS (ESI) m/z 322.3 (M+H)⁺]

### Example 17

### 2-[(2,6-Difluorobenzyl)oxy]-4-(pyrimidin-5-yl)-6,7-dihydro-5H-cyclopenta[b]pyridine

The title compound was prepared as a white powder according to the procedure described in Example 11, Step 2, using 2,6-difluorobenzylalcohol instead of benzylalcohol.

[MS (ESI) m/z 340.3 (M+H)⁺]

### Example 18

### 2-[(2,4-Difluorobenzyl)oxy]-4-(pyrimidin-5-yl)-6,7-dihydro-5H-cyclopenta[b]pyridine

The title compound was prepared as a white powder according to the procedure described in Example 11, Step 2, using 2,4-difluorobenzylalcohol instead of benzylalcohol.

[MS (ESI) m/z 340.3 (M+H)⁺]

### Example 19

### 2-[(3,5-Difluorobenzyl)oxy]-4-(pyrimidin-5-yl)-6,7-dihydro-5H-cyclopenta[b]pyridine

The title compound was prepared as a white powder according to the procedure described in Example 11, Step 2, using 3,5-difluorobenzylalcohol instead of benzylalcohol.

[MS (ESI) m/z 340.3 (M+H)⁺]

### Example 20

### 2-[(3,4-Difluorobenzyl)oxy]-4-(pyrimidin-5-yl)-6,7-dihydro-5H-cyclopenta[b]pyridine

The title compound was prepared as a white powder according to the procedure described in Example 11, Step 2, using 3,4-difluorobenzylalcohol instead of benzylalcohol.

[MS (ESI) m/z 340.3 (M+H)⁺]

### Example 21

### 2-[(4-Chloro-2-fluorobenzyl)oxy]-4-(pyrimidin-5-yl)-6,7-dihydro-5H-cyclopenta[b]pyridine

The title compound was prepared as a white powder according to the procedure described in Example 11, Step 2, using 4-chloro-2-fluorobenzylalcohol instead of benzylalcohol.

[MS (ESI) m/z 356.3 (M+H)⁺]

### Example 22

### 2-({[4-(Pyrimidin-5-yl)-6,7-dihydro-5H-cyclopenta[b]pyridin-2-yl]oxy}methyl)benzonitrile

### [Step 1]

Production of 2-[(2-bromobenzyl)oxy]-4-(pyrimidin-5-yl)-6,7-dihydro-5H-cyclopenta[b]pyridine

To a solution of 2-bromobenzylalcohol (97 mg) in DMF (3 mL) were added KO-t-Bu (72 mg) and 2-chloro-4-(pyrimidin-5-yl)-6,7-dihydro-5H-cyclopenta[b]pyridine (100 mg), and the mixture was stirred at 70° C. for 2 hours. After the reaction mixture was allowed to return to room temperature, the mixture was added with water and Et₂O, and subjected to extraction. The organic layer was dried over anhydrous sodium sulfate, and filtered off, and the solvent was evaporated under reduced pressure. The resulting residue was purified by silica gel column chromatography to give the title compound (47 mg) as a white solid.

### [Step 2]

Production of 2-({[4-(pyrimidin-5-yl)-6,7-dihydro-5H-cyclopenta[b]pyridin-2-yl]oxy}methyl)benzonitrile

To 2-[(2-bromobenzyl)oxy]-4-(pyrimidin-5-yl)-6,7-dihydro-5H-cyclopenta[b]pyridine (45 mg), Pd(dppf)Cl₂ CH₂.Cl₂ (10 mg), zinc cyanide (83 mg) and zinc powder (0.9 mg) was added DMF (1 mL), and the mixture was reacted under microwave irradiation at 180° C. for 1 hour. After the reaction mixture was allowed to return to room temperature, diluted with ethyl acetate, and filtered through Celite, the filtrate was evaporated under reduced pressure. The resulting residue was purified by silica gel column chromatography to give the title compound (24 mg) as a white solid. [MS (ESI) m/z 329.3 (M+H)⁺]

### Example 23

### 3-({[4-(Pyrimidin-5-yl)-6,7-dihydro-5H-cyclopenta[b]pyridin-2-yl]oxy}methyl)benzonitrile

The title compound was prepared as a white powder according to the procedure described in Example 11, Step 2, using 3-(hydroxymethyl)benzonitrile instead of benzylalcohol.

[MS (ESI) m/z 329.3 (M+H)⁺]

### Example 24

### 2-[(6-Fluoropyridin-2-yl)methoxy]-4-(pyrimidin-5-yl)-6,7-dihydro-5H-cyclopenta[b]pyridine

The title compound was prepared according to the procedure described in Example 11, Step 2, using (6-fluoropyridin-2-yl)methanol instead of benzylalcohol, and using cesium carbonate instead of NaO-t-Bu.

[MS (ESI) m/z 323.4 (M+H)⁺]

### Example 25

### 2-[(5-Fluoropyridin-2-yl)methoxy]-4-(pyrimidin-5-yl)-6,7-dihydro-5H-cyclopenta[b]pyridine

The title compound was prepared as a white powder according to the procedure described in Example 11, Step 2, using (5-fluoropyridin-2-yl)methanol instead of benzylalcohol, and using cesium carbonate instead of NaO-t-Bu.

[MS (ESI) m/z 323.4 (M+H)⁺]

### Example 26

### 2-[(3-Fluoropyridin-2-yl)methoxy]-4-(pyrimidin-5-yl)-6,7-dihydro-5H-cyclopenta[b]pyridine

To 2-chloro-4-(pyrimidin-5-yl)-6,7-dihydro-5H-cyclopenta[b]pyridine (70 mg), (3-fluoropyridin-2-yl)methanol (46 mg), t-Bu-X-Phos (31 mg), Pd₂(dba)₃.CHCl₃ (19 mg) and cesium carbonate (295 mg) was added toluene (3 mL), and the mixture was degassed, and then stirred under Ar atmosphere at 100° C. for 5 hours. After the reaction mixture was allowed to return to room temperature, diluted with ethyl acetate, and filtered through a short column, the solvent was evaporated under reduced pressure. The resulting residue was purified by silica gel column chromatography to give the title compound (47 mg) as a white solid.

[MS (ESI) m/z 323.4 (M+H)⁺]

### Example 27

### 2-[(3,5-Difluoropyridin-2-yl)methoxy]-4-(pyrimidin-5-yl)-6,7-dihydro-5H-cyclopenta[b]pyridine

The title compound was prepared as a white solid according to the procedure described in Example 11, Step 2, using (3,5-difluoropyridin-2-yl)methanol instead of benzylalcohol. [MS (ESI) m/z 341.3 (M+H)⁺]

### Example 28

### 2-[(6-Methylpyridin-2-yl)methoxy]-4-(pyrimidin-5-yl)-6,7-dihydro-5H-cyclopenta[b]pyridine

The title compound was prepared as a white powder according to the procedure described in Example 11, Step 2, using (6-methylpyridin-2-yl)methanol instead of benzylalcohol.

[MS (ESI) m/z 319.4 (M+H)⁺]

### Example 29

### 2-({[4-(Pyrimidin-5-yl)-6,7-dihydro-5H-cyclopenta[b]pyridin-2-yl]oxy}methyl)quinoline

The title compound was prepared as a white solid according to the procedure described in Example 11, Step 2, using quinolin-2-ylmethanol instead of benzylalcohol.

[MS (ESI) m/z 425.5 (M+H)⁺]

### Example 30

### 2-[(5-Fluoropyridin-3-yl)methoxy]-4-(pyrimidin-5-yl)-6,7-dihydro-5H-cyclopenta[b]pyridine

The title compound was prepared as a white powder according to the procedure described in Example 11, Step 2, using (5-fluoropyridin-3-yl)methanol instead of benzylalcohol.

[MS (ESI) m/z 323.4 (M+H)⁺]

### Example 31

### 2-[(6-Methylpyridin-3-yl)methoxy]-4-(pyrimidin-5-yl)-6,7-dihydro-5H-cyclopenta[b]pyridine

The title compound was prepared as yellow oil according to the procedure described in Example 11, Step 2, using (6-methylpyridin-3-yl)methanol instead of benzylalcohol.

[MS (ESI) m/z 319.4 (M+H)]

### Example 32

### 2-[(3-Fluoropyridin-4-yl)methoxy]-4-(pyrimidin-5-yl)-6,7-dihydro-5H-cyclopenta[b]pyridine

The title compound was prepared as a white powder according to the procedure described in Example 11, Step 2, using (3-fluoropyridin-4-yl)methanol instead of benzylalcohol.

[MS (ESI) m/z 323.3 (M+H)⁺]

### Example 33

### 2-[(2-Cyclopropylpyridin-4-yl)methoxy]-4-(pyrimidin-5-yl)-6,7-dihydro-5H-cyclopenta[b]pyridine

### [Step 1]

Production of methyl 2-cyclopropylpyridine-4-carboxylate

To methyl 2-chloropyrimidine-4-carboxylate (200 mg), cyclopropylboronic acid (150 mg), PdCl₂(PCy₃)₂ (172 mg) and potassium phosphate (742 mg) was added toluene (0.9 mL), and the mixture was degassed, and then stirred under Ar atmosphere at 100° C. overnight. After the reaction mixture was allowed to return to room temperature, diluted with ethyl acetate, and filtered through Celite, the solvent was evaporated under reduced pressure. The resulting residue was purified by silica gel column chromatography to give the title compound as yellow oil (218 mg).

### [Step 2]

Production of (2-cyclopropylpyridin-4-yl)methanol

To a solution of methyl 2-cyclopropylpyridine-4-carboxylate (218 mg) in methanol (8 mL) was added NaBH₄ (140 mg) under Ar atmosphere and the mixture was stirred at room temperature overnight. The reaction mixture was added with water and ethyl acetate, and subjected to extraction. The organic layer was washed with saturated brine, dried over anhydrous sodium sulfate, and filtered off, and the solvent was evaporated under reduced pressure. The resulting residue was purified by silica gel column chromatography to give the title compound (51 mg) as pale yellow oil.

### [Step 3]

Production of 2-[(2-cyclopropylpyridin-4-yl)methoxy]-4-(pyrimidin-5-yl)-6,7-dihydro-5H-cyclopenta[b]pyridine

The title compound was prepared as a white powder according to the procedure described in Example 11, Step 2, using (2-cyclopropylpyridin-4-yl)methanol instead of benzylalcohol.

[MS (ESI) m/z 345.3 (M+H)⁺]

### Example 34

### 4-(Pyrimidin-5-yl)-2-(thiophen-2-ylmethoxy)-6,7-dihydro-5H-cyclopenta[b]pyridine

The title compound was prepared as a white solid according to the procedure described in Example 11, Step 2, using thiophen-2-ylmethanol instead of benzylalcohol.

[MS (ESI) m/z 310.3 (M+H)⁺]

### Example 35

### 2-[(5-Chlorothiophen-2-yl)methoxy]-4-(pyrimidin-5-yl)-6,7-dihydro-5H-cyclopenta[b]pyridine

The title compound was prepared as a white solid according to the procedure described in Example 11, Step 2, using (5-chlorothiophen-2-yl)methanol instead of benzylalcohol.

[MS (ESI) m/z 344.2 (M+H)⁺]

### Example 36

### 4-(2-Methylpyrimidin-5-yl)-2-(pyridin-2-ylmethoxy)-6,7-dihydro-5H-cyclopenta[b]pyridine hydrochloride

4-(2-Methylpyrimidin-5-yl)-2-(pyridin-2-ylmeth oxy)-6,7-dihydro-5H-cyclopenta[b]pyridine (429 mg) was prepared according to the procedure described in Example 5, Step 2, using 5-bromo-2-methylpyrimidine instead of 5-bromopyridin-3-ol. The resulting compound was dissolved in ethyl acetate, 1 N HCl/Et₂O solution (1.48 mL) was added under ice water cooling, and the mixture was stirred at the same temperature for 0.5 hour. The resulting precipitate was collected by filtration to give the title compound (451 mg) as a white powder.

[MS (ESI) m/z 319.4 (M+H)⁺]

Elementary analysis as C₁₉H₁₈N₄O.HCl+.3H₂O

Calcd. (%) C: 63.35.; H: 5.48.; N: 15.55.

Found. (%) C: 63.26.; H: 5.35.; N: 15.39.

### Example 37

### 5-[2-(Pyridin-2-ylmethoxy)-6,7-dihydro-5H-cyclopenta[b]pyridin-4-yl]pyrimidine-2-carbonitrile hydrochloride

5-[2-(Pyridin-2-ylmethoxy)-6,7-dihydro-5H-cyclopenta[b]pyridin-4-yl]pyrimidine-2-carbonitrile was prepared (65 mg) according to the procedure described in Example 5, Step 2, using 5-bromopyrimidine-2 carbonitrile instead of 5-bromopyrimidin-3-ol. The resulting compound was dissolved in ethyl acetate (2 mL), 1 N HCl/Et₂O solution (0.22 mL) was added under ice water cooling, and then the mixture was stirred at the same temperature for 0.5 hour. The resulting precipitate was collected by filtration to give the title compound (63 mg) as a white powder.

[MS (ESI) m/z 330.3 (M+H)⁺]

### Example 38

### 2-(Benzyloxy)-4-(2-methylpyrimidin-5-yl)-6,7-dihydro-5H-cyclopenta[b]pyridine

### [Step 1]

Production of 2-chloro-4-(2-methylpyrimidin-5-yl)-6,7-dihydro-5H-cyclopenta[b]pyridine

The title compound was prepared as a white solid according to the procedure described in Example 5, Step 2, using 2-chloro-4-(4,4,5,5-tetramethyl-1,3,2-dioxaborolan-2-yl)-6,7-dihydro-5H-cyclopenta[b]pyridine instead of 2-(pyridin-2-ylmethoxy)-4-(4,4,5,5-tetramethyl-1,3,2-dioxaborolan-2-yl)-6,7-dihydro-5H-cyclopenta[b]pyridine, and using 5-bromo-2-methylpyrimidine instead of 5-bromopyridin-3-ol, and using THF/water (3/1) instead of 1,4-dioxane/water (3/1).

### [Step 2]

Production of 2-(benzyloxy)-4-(2-methylpyrimidin-5-yl)-6,7-dihydro-5H-cyclopenta[b]pyridine

The title compound was prepared as a white solid according to the procedure described in Example 11, Step 2, using 2-chloro-4-(2-methylpyrimidin-5-yl)-6,7-dihydro-5H-cyclopenta[b]pyridine instead of 2-chloro-4-(pyrimidin-5-yl)-6,7-dihydro-5H-cyclopenta[b]pyridine, and using cesium carbonate instead of NaO-t-Bu.

[MS (ESI) m/z 318.3 (M+H)⁺]

### Example 39

### 2-[(6-Fluoropyridin-2-yl)methoxy]-4-(2-methylpyrimidin-5-yl)-6,7-dihydro-5H-cyclopenta[b]pyridine

To 2-chloro-4-(2-methylpyrimidin-5-yl)-6,7-dihydro-5H-cyclopenta[b]pyridine (100 mg), (6-fluoropyridin-2-yl)methanol (62 mg), t-Bu-X-Phos (41 mg), NaO-t-Bu (78 mg), Pd₂(dba)₃.CHCl₃ (25 mg) and MS4A was added toluene (2 mL), and the mixture was degassed, and then stirred under Ar atmosphere at 100° C. for 4 hours. After the reaction mixture was allowed to return to room temperature, diluted with ethyl acetate, and filtered through Celite, the filtrate was evaporated under reduced pressure. The resulting residue was purified by silica gel column chromatography to give the title compound (22 mg) as a white powder.

[MS (ESI) m/z 337.4 (M+H)⁺]

### Example 40

### 2-[(5-Fluoropyridin-2-yl)methoxy]-4-(2-methylpyrimidin-5-yl)-6,7-dihydro-5H-cyclopenta[b]pyridine hydrochloride

2-[(5-Fluoropyridin-2-yl)methoxy]-4-(2-methylpyrimidin-5-yl)-6,7-dihydro-5H-cyclopenta[b]pyridine (46 mg) was prepared according to the procedure described in Example 39 using (5-fluoropyridin-2-yl)methanol instead of (6-fluoropyridin-2-yl)methanol.

[MS (ESI) m/z 337.4 (M+H)⁺]

The resulting compound was dissolved in ethyl acetate (2.6 mL), 1 N HCl/Et₂O solution (0.22 mL) was added under ice water cooling, and then the mixture was stirred at the same temperature for 0.5 hour. The resulting precipitate was collected by filtration to give the title compound (24 mg) as a white powder.

[MS (ESI) m/z 337.4 (M+H)+]

### Example 41

### 2-[(3-Fluoropyridin-2-yl)methoxy]-4-(2-methylpyrimidin-5-yl)-6,7-dihydro-5H-cyclopenta[b]pyridine

The title compound was prepared as a white powder according to the procedure described in Example 39 using (3-fluoropyridin-2-yl)methanol instead of (6-fluoropyridin-2-yl)methanol.

[MS (ESI) m/z 337.4 (M+H)⁺]

### Example 42

### 2-[(3,5-Difluoropyridin-2-yl)methoxy]-4-(2-methylpyrimidin-5-yl)-6,7-dihydro-5H-cyclopenta[b]pyridine

The title compound was prepared as a white powder according to the procedure described in Example 39 using (3,5-difluoropyridin-2-yl)methanol instead of (6-fluoropyridin-2-yl)methanol.

[MS (ESI) m/z 355.4 (M+H)⁺]

### Example 43

### 2-[(3,6-Difluoropyridin-2-yl)methoxy]-4-(2-methylpyrimidin-5-yl)-6,7-dihydro-5H-cyclopenta[b]pyridine

The title compound was prepared as a white powder according to the procedure described in Example 39 using (3,6-difluoropyridin-2-yl)methanol instead of (6-fluoropyridin-2-yl)methanol.

[MS (ESI) m/z 355.3 (M+H)⁺]

### Example 44

### 5-{2-[(3,5-Difluoropyridin-2-yl)methoxy]-6,7-dihydro-5H-cyclopenta[b]pyridin-4-yl}pyrimidine-2-carbonitrile

### [Step 1]

Production of 2,4-dichloro-6,7-dihydro-5H-cyclopenta[b]pyridine 1-oxide

To a solution of 2,4-dichloro-6,7-dihydro-5H-cyclopenta[b]pyridine (1 g) in CH₂Cl₂ (20 mL) was added m-CPBA (with abs. 25% water, 1.69 g), and the mixture was stirred at room temperature for 24 hours. The resulting residue was added with ethyl acetate, aqueous sodium hydrogen carbonate solution, and aqueous sodium thiosulfate solution, and subjected to extraction. The organic layer was dried over anhydrous sodium sulfate, and filtered off, and the solvent was evaporated under reduced pressure. The resulting residue was purified by silica gel column chromatography to give the title compound (852 mg) as a white powder.

### [Step 2]

Production of 4-chloro-2-[(3,5-difluoropyridin-2-yl)methoxy]-6,7-dihydro-5H-cyclopenta[b]pyridine

To 2,4-dichloro-6,7-dihydro-5H-cyclopenta[b]pyridine 1-oxide (200 mg) and (3,5-difluoropyridin-2-yl)methanol (185 mg) was added THF (8 mL) and then NaH (60% dispersion in oil, 59 mg) was added under ice water cooling, and then the mixture was stirred at room temperature for 3 hours. After that, phosphorus trichloride (175 mg) was added to the mixture under ice water cooling, and the mixture was stirred for 30 minutes. The reaction mixture was added with aqueous sodium bicarbonate solution, water and ethyl acetate, and subjected to extraction. The organic layer was dried over anhydrous sodium sulfate, and filtered off, and the solvent was evaporated under reduced pressure. The resulting residue was purified by silica gel column chromatography to give the title compound (63 mg) as a white powder.

### [Step 3]

Production of 2-[(3,5-difluoropyridin-2-yl)methoxy]-6,7-dihydro-5H-cyclopenta[b]pyridin-4-ol

To 4-chloro-2-[(3,5-difluoropyridin-2-yl)methoxy]-6,7-dihydro-5H-cyclopenta[b]pyridine (6.2 mg), potassium hydroxide (29 mg), Pd₂(dba)₃ (10 mg) and t-Bu-X-Phos (9 mg) were added 1,4-dioxane (1 mL) and water (1 mL), and the mixture was degassed, and then stirred under Ar atmosphere at 100° C. for 1 minute. After the reaction mixture was allowed to return to room temperature, and added with ethyl acetate, saturated aqueous ammonium chloride solution and water, it was subjected to extraction. The organic layer was dried over anhydrous sodium sulfate, and filtered off, and the solvent was evaporated under reduced pressure. The resulting residue was purified by silica gel column chromatography to give the title compound (67 mg) as pale brown oil.

### [Step 4]

Production of 2-[(3,5-difluoropyridin-2-yl)methoxy]-6,7-dihydro-5H-cyclopenta[b]pyridin-4-yl trifluoromethanesulfonate

To a solution of 2-[(3,5-difluoropyridin-2-yl)methoxy]-6,7-dihydro-5H-cyclopenta[b]pyridin-4-ol (65 mg) in CH₂Cl₂ (2 mL) were sequentially added Et₃N (5.3 mL), Tf₂NPh (92 mg) and DMAP (3 mg), and the mixture was stirred at room temperature for 2 hours. The reaction mixture was extracted with water and ethyl acetate. The organic layer was washed with saturated brine, dried over magnesium sulfate, and filtered off, and the solvent was evaporated under reduced pressure. The resulting residue was purified by silica gel column chromatography to give the title compound (70 mg).

### [Step 5]

Production of 5-{2-[(3,5-difluoropyridin-2-yl)methoxy]-6,7-dihydro-5H-cyclopenta[b]pyridin-4-yl}pyrimidine-2-carbonitrile

To 2-[(3,5-difluoropyridin-2-yl)methoxy]-6,7-dihydro-5H-cyclopenta[b]pyridin-4-yl trifluoromethanesulfonate (50 mg), 5-(4,4,5,5-tetramethyl-1,3,2-dioxaborolan-2-yl)pyrimidine-2-carbonitrile (31 mg), Pd(dppf)Cl₂—CH₂Cl₂ (10 mg) and potassium carbonate (50 mg) was added 1,4-dioxane/water (3/1, 1 mL), and the mixture was degassed, and then stirred under Ar atmosphere at 70° C. for 1 hour. After the reaction mixture was allowed to return to room temperature, the mixture was added with water and ethyl acetate, and subjected to extraction. The organic layer was dried over anhydrous sodium sulfate, and filtered off, and the solvent was evaporated under reduced pressure. The resulting residue was purified by silica gel column chromatography to give the title compound (31 mg) as a white solid.

[MS (ESI) m/z 366.3 (M+H)⁺]

### Example 45

### 2-[(6-Methylpyridin-2-yl)methoxy]-4-(2-methylpyrimidin-5-yl)-6,7-dihydro-5H-cyclopenta[b]pyridine hydrochloride

To 2-chloro-4-(2-methylpyrimidin-5-yl)-6,7-dihydro-5H-cyclopenta[b]pyridine (100 mg), (6-methylpyridin-2-yl)methanol (52 mg), t-Bu-X-Phos (55 mg), cesium carbonate (398 mg) and Pd₂(dba)₃.CHCl₃ (34 mg) was added toluene (2.2 mL), and the mixture was degassed, and then stirred under Ar atmosphere at 100° C. for 6 hours. After the reaction mixture was allowed to return to room temperature, diluted with ethyl acetate, and filtered through Celite, the filtrate was evaporated under reduced pressure. The resulting residue was purified by silica gel column chromatography to give 2-[(6-methylpyridin-2-yl)methoxy]-4-(2-methylpyrimidin-5-yl)-6,7-dihydro-5H-cyclopenta[b]pyridine (49 mg). The resulting compound was dissolved in ethyl acetate (2 mL), 1 N HCl/Et₂O solution (0.147 mL) was added, and then the mixture was stirred under ice water cooling for 3 hours. The resulting precipitate was collected by filtration to give the title compound (43 mg) as a white powder.

[MS (ESI) m/z 333.4 (M+H)⁺]

### Example 46

### 6-({[4-(2-Methylpyrimidin-5-yl)-6,7-dihydro-5H-cyclopenta[b]pyridin-2-yl]oxy}methyl)pyridine-2-carbonitrile

The title compound was prepared according to the procedure described in Example 8, Step 4, using 5-bromo-2-methylpyrimidine instead of 2-fluoro-3-iodopyridine.

[MS (ESI) m/z 344.4 (M+H)⁺]

### Example 47

### 6-({[4-(2-Methylpyrimidin-5-yl)-6,7-dihydro-5H-cyclopenta[b]pyridin-2-yl]oxy}methyl)pyridine-3-carbonitrile

To 2-chloro-4-(2-methylpyrimidin-5-yl)-6,7-dihydro-5H-cyclopenta[b]pyridine (100 mg), 6-(hydroxymethyl)pyridine-3-carbonitrile (57 mg), t-Bu-X-Phos (55 mg), cesium carbonate (398 mg) and Pd₂(dba)₃.CHCl₃ (34 mg) was added toluene (2.2 mL), and the mixture was degassed, and then stirred under Ar atmosphere at 100° C. for 6 hours. After the reaction mixture was allowed to return to room temperature, diluted with ethyl acetate, and filtered through Celite, the filtrate was evaporated under reduced pressure.

The resulting residue was purified by silica gel column chromatography to give the title compound (30 mg) as a white powder.

[MS (ESI) m/z 344.4 (M+H)⁺]

### Example 48

### 5-{2-[(6-Fluoropyridin-2-yl)methoxy]-6,7-dihydro-5H-cyclopenta[b]pyridin-4-yl}pyrimidine-2-carbonitrile

### [Step 1]

Production of 5-(2-chloro-6,7-dihydro-5H-cyclopenta[b]pyridin-4-yl) pyrimidine-2-carbonitrile

The title compound was prepared according to the procedure described in Example 11, Step 1, using 5-(4,4,5,5-tetramethyl-1,3,2-dioxaborolan-2-yl)pyrimidine-2-carbonitrile instead of pyrimidin-5-ylboronic acid.

### [Step 2]

Production of 5-{2-[(6-fluoropyridin-2-yl)methoxy]-6,7-dihydro-5H-cyclopenta[b]pyridin-4-yl}pyrimidine-2-carbonitrile

The title compound was prepared as a white powder according to the procedure described in Example 47 using 5-(2-chloro-6,7-dihydro-5H-cyclopenta[b]pyridin-4-yl) pyrimidine-2-carbonitrile instead of 2-chloro-4-(2-methylpyrimidin-5-yl)-6,7-dihydro-5H-cyclopenta[b]pyridine, and using (6-fluoropyridin-2-yl)methanol instead of 6-(hydroxymethyl)pyridine-3-carbonitrile.

[MS (ESI) m/z 348.3 (M+H)⁺]

### Example 49

### 5-{2-[(5-Fluoropyridin-2-yl)methoxy]-6,7-dihydro-5H-cyclopenta[b]pyridin-4-yl}pyrimidine-2-carbonitrile

The title compound was prepared as a yellow solid according to the procedure described in Example 48, Step 2, using (5-fluoropyridin-2-yl)methanol instead of (6-fluoropyridin-2-yl)methanol.

[MS (ESI) m/z 348.4 (M+H)⁺]

### Example 50

### 5-{2-[(3-Fluoropyridin-2-yl)methoxy]-6,7-dihydro-5H-cyclopenta[b]pyridin-4-yl}pyrimidine-2-carbonitrile

### [Step 1]

Production of 4-chloro-2-[(3-fluoropyridin-2-yl)methoxy]-6,7-dihydro-5H-cyclopenta[b]pyridine

The title compound was prepared as a white powder according to the procedure described in Example 44, Step 2, using (3-fluoropyridin-2-yl)methanol instead of (3,5-difluoropyridin-2-yl)methanol.

### [Step 2]

Production of 2-[(3-fluoropyridin-2-yl)methoxy]-6,7-dihydro-5H-cyclopenta[b]pyridin-4-ol

The title compound was prepared as a white solid according to the procedure described in Example 44, Step 3, using 4-chloro-2-[(3-fluoropyridin-2-yl)methoxy]-6,7-dihydro-5H-cyclopenta[b]pyridine instead of 4-chloro-2-[(3,5-difluoropyridin-2-yl)methoxy]-6,7-dihydro-5H-cyclopenta[b]pyridine.

### [Step 3]

Production of 2-[(3-fluoropyridin-2-yl)methoxy]-6,7-dihydro-5H-cyclopenta[b]pyridin-4-yl trifluoromethanesulfonate

To a solution of 2-[(3-fluoropyridin-2-yl)methoxy]-6,7-dihydro-5H-cyclopenta[b]pyridin-4-ol (157 mg) and 2,6-lutidine (0.11 mL) in CH₂Cl₂ (6 mL) was added Tf₂O (0.12 mL) under ice water cooling, and the mixture was stirred at room temperature for 1 hour. The reaction mixture was added with water and Et₂O, and subjected to extraction. The organic layer was washed with saturated brine, dried over anhydrous sodium sulfate, and filtered off, and the solvent was evaporated under reduced pressure. The resulting residue was purified by silica gel column chromatography to give the title compound (220 mg) as colorless oil.

### [Step 4]

Production of 5-{2-[(3-fluoropyridin-2-yl)methoxy]-6,7-dihydro-5H-cyclopenta[b]pyridin-4-yl}pyrimidine-2-carbonitrile

The title compound was prepared as a white solid according to the procedure described in Example 44, Step 5, using 2-[(3-fluoropyridin-2-yl)methoxy]-6,7-dihydro-5H-cyclopenta[b]pyridin-4-yl trifluoromethanesulfonate instead of 2-[(3,5-difluoropyridin-2-yl)methoxy]-6,7-dihydro-5H-cyclopenta[b]pyridin-4-yl trifluoromethanesulfonate.

[MS (ESI) m/z 348.3 (M+H)⁺]

### Example 51

### 5-{2-[(6-Cyanopyridin-2-yl)methoxy]-6,7-dihydro-5H-cyclopenta[b]pyridin-4-yl}pyrimidine-2-carbonitrile

The title compound was prepared according to the procedure described in Example 8, Step 4, using 5-bromopyrimidine-2-carbonitrile instead of 2-fluoro-3-iodopyridine.

[MS (ESI) m/z 355.3 (M+H)⁺]

### Example 52

### 6-({[4-(2-Aminopyrimidin-5-yl)-6,7-dihydro-5H-cyclopenta[b]pyridin-2-yl]oxy}methyl)pyridine-2-carbonitrile hydrochloride

6-({[4-(2-Aminopyrimidin-5-yl)-6,7-dihydro-5H-cyclopenta[b]pyridin-2-yl]oxy}methyl)pyridine-2-carbonitrile (46 mg) was prepared according to the procedure described in Example 8, Step 4, using 5-bromopyrimidine-2-amine instead of 2-fluoro-3-iodopyridine.

[MS (ESI) m/z 345.4 (M+H)⁺]

The resulting compound was dissolved in Et₂O (2.6 mL), 1 N HCl/Et₂O solution (0.134 mL) was added, and then the mixture was stirred for 0.5 hour. The resulting precipitate was collected by filtration to give the title compound (41 mg) as a white powder.

[MS (ESI) m/z 345.4 (M+H)⁺]

### Example 53

### 4-(Pyrazin-2-yl)-2-(pyridin-2-ylmethoxy)-6,7-dihydro-5H-cyclopenta[b]pyridine hydrochloride

To 4-chloro-2-(pyridin-2-ylmethoxy)-6,7-dihydro-5H-cyclopenta[b]pyridine (50 mg), 2-(tributylstannyl)pyrazine (211 mg) and Pd(PPh₃)₄ (88 mg) was added DMF (0.9 mL), and the mixture was reacted under microwave irradiation at 180° C. for 30 minutes. After the reaction mixture was allowed to return to room temperature, diluted with ethyl acetate, and filtered through Celite, the filtrate was evaporated under reduced pressure. The resulting residue was purified by silica gel column chromatography to give 4-(pyrazin-2-yl)-2-(pyridin-2-ylmethoxy)-6,7-dihydro-5H-cyclopenta[b]pyridine (94 mg). The resulting compound was dissolved in ethyl acetate (3 mL), 1 N HCl/Et₂O solution (0.3 mL) was added under ice water cooling, and then the mixture was stirred at the same temperature for 2 hours under ice water cooling. The resulting precipitate was collected by filtration to give the title compound (86 mg) as a white powder.

[MS (ESI) m/z 305.2 (M+H)⁺]

Elementary analysis as C₁₈H₁₆N₄O.HCl+0.8H₂O

Calcd. (%) C: 60.86.; H: 5.28.; N: 15.77.

Found. (%) C: 60.84.; H: 5.18.; N: 15.57.

### Example 54

### 6-[2-(Pyridin-2-ylmethoxy)-6,7-dihydro-5H-cyclopenta[b]pyridin-4-yl]pyrazine-2-carbonitrile hydrochloride

### [Step 1]

Production of 4-(6-chloropyrazin-2-yl)-2-(pyridin-2-ylmethoxy)-6,7-dihydro-5H-cyclopenta[b]pyridine

The title compound was prepared according to the procedure described in Example 5, Step 2, using 2,6-dichloropyrazine instead of 5-bromopyridin-3-ol.

### [Step 2]

Production of 6-[2-(pyridin-2-ylmethoxy)-6,7-dihydro-5H-cyclopenta[b]pyridin-4-yl]pyrazine-2-carbonitrile hydrochloride

To 4-(6-chloropyrazin-2-yl)-2-(pyridin-2-ylmethoxy)-6,7-dihydro-5H-cyclopenta[b]pyridine (100 mg), Pd (dppf) Cl₂.CH₂Cl₂ (24 mg), zinc cyanide (21 mg) and zinc powder (2 mg) was added DMF (2 mL), and then the mixture was reacted under microwave irradiation at 190° C. for 1 hour. After the reaction mixture was allowed to return to room temperature, it was added with water and ethyl acetate, and subjected to extraction. The organic layer was dried over anhydrous sodium sulfate, and filtered off, and the solvent was evaporated under reduced pressure. The resulting residue was purified by silica gel column chromatography to give 6-[2-(pyridin-2-ylmethoxy)-6,7-dihydro-5H-cyclopenta[b]pyridin-4-yl]pyrazine-2-carbonitrile (16 mg). The resulting compound was dissolved in ethyl acetate (1 mL), 1 N HCl/Et₂O solution (0.055 mL) was added under ice water cooling, and then the mixture was stirred at the same temperature for 0.5 hour. The resulting precipitate was collected by filtration to give the title compound (18 mg) as a white powder.

[MS (ESI) m/z 330.3 (M+H)⁺]

### Example 55

### 2-{6-[2-(Pyridin-2-ylmethoxy)-6,7-dihydro-5H-cyclopenta[b]pyridin-4-yl]pyrazin-2-yl}propan-2-ol

### [Step 1]

Production of ethyl 6-[2-(pyridin-2-ylmethoxy)-6,7-dihydro-5H-cyclopent a[b]pyridin-4-yl]pyrazine-2-carboxylate

A mixture of 4-(6-chloropyrazin-2-yl)-2-(pyridin-2-ylmethoxy)-6,7-dihydro-5H-cyclopenta[b]pyridine (170 mg), Pd(dppf)Cl₂.CH₂Cl₂ (32 mg) and DIPEA (0.26 mL) in ethanol (2.5 mL) and DMF (2.5 mL) was stirred under carbon monooxide atmosphere at 100° C. overnight. After the reaction mixture was allowed to return to room temperature, diluted with Et₂O, and filtered through Celite, the filtrate was evaporated under reduced pressure. The resulting residue was purified by silica gel column chromatography to give the title compound (66 mg)

### [Step 2]

Production of 2-{6-[2-(pyridin-2-ylmethoxy)-6,7-dihydro-5H-cyclopenta[b]pyridin-4-yl]pyrazin-2-yl}propan-2-ol

To a solution of ethyl 6-[2-(pyridin-2-ylmethoxy)-6,7-dihydro-5H-cyclopent a[b]pyridin-4-yl]pyrazine-2-carboxylate (66 mg) in THF (3.5 mL) was added dropwise methylmagnesium iodide (in Et₂O solution, 0.83 mL) under Ar atmosphere under ice water cooling, and the mixture was stirred at room temperature for 5 hours. The reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (5 mg) as a white powder.

[MS (ESI) m/z 363.4 (M+H)⁺]

### Example 56

### 6-[({4-[6-(Hydroxymethyl)pyrazin-2-yl]-6,7-dihydro-5H-cyclopenta[b]pyridin-2-yl}oxy)methyl]pyridine-2-carbonitrile

To 6-({[4-(4,4,5,5-tetramethyl-1,3,2-dioxaborolan-2-yl)-6,7-dihydro-5H-cyclopenta[b]pyridin-2-yl]oxy}meth yl)pyridine-2-carbonitrile (100 mg), (6-chloropyrazin-2-yl)methanol (46 mg), Pd₂(dba)₃ (12 mg), S-Phos (22 mg) and potassium carbonate (110 mg) was added 1,4-dioxane/water (3/1, 4 mL), and the mixture was degassed, and then stirred under Ar atmosphere at 100° C. for 2 hours. After the reaction mixture was allowed to return to room temperature, it was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (72 mg) as a white powder.

[MS (ESI) m/z 360.3 (M+H)⁺]

### Example 57

### 6-({[4-(6-Methylpyrazin-2-yl)-6,7-dihydro-5H-cyclopenta[b]pyridin-2-yl]oxy}methyl)pyridine-2-carbonitrile

The title compound was prepared as a white powder according to the procedure described in Example 56 using 2-chloro-6-methylpyrazine instead of (6-chloropyrazin-2-yl)methanol.

[MS (ESI) m/z 344.3 (M+H)⁺]

### Example 58

### 4-(3-Fluorophenyl)-2-(pyridin-2-ylmethoxy)-6,7-dihydro-5H-cyclopenta[b]pyridine

To 4-chloro-2-(pyridin-2-ylmethoxy)-6,7-dihydro-5H-cyclopenta[b]pyridine (100 mg), (3-fluorophenyl)boronic acid (70 mg), Pd(OAc)₂ (1.7 mg), X-Phos (7.3 mg) and potassium carbonate (106 mg) was added ethanol (1.9 mL), and the mixture was degassed, and then stirred under Ar atmosphere at 95° C. for 2 hours. After the reaction mixture was allowed to return to room temperature, diluted with ethyl acetate, and filtered through Celite, the filtrate was evaporated under reduced pressure. The resulting residue was purified by silica gel column chromatography to give the title compound (106 mg) as a white solid.

[MS (ESI) m/z 322.3 (M+H)⁺]

### Example 59

### 4-(4-Fluorophenyl)-2-(pyridin-2-ylmethoxy)-6,7-dihydro-5H-cyclopenta[b]pyridine

The title compound was prepared as a beige powder according to the procedure described in Example 58 using (4-fluorophenyl)boronic acid instead of (3-fluorophenyl)boronic acid.

[MS (ESI) m/z 321.2 (M+H)⁺]

### Example 60

### 4-(4-Methylphenyl)-2-(pyridin-2-ylmethoxy)-6,7-dihydro-5H-cyclopenta[b]pyridine

The title compound was prepared as a white powder according to the procedure described in Example 58 using (4-methylphenyl)boronic acid instead of (3-fluorophenyl)boronic acid.

[MS (ESI) m/z 317.3 (M+H)⁺]

### Example 61

### 2-(Pyridin-2-ylmethoxy)-4-[3-(trifluoromethyl)phenyl]-6,7-dihydro-5H-cyclopenta[b]pyridine

The title compound was prepared as colorless oil according to the procedure described in Example 58 using [3-(trifluoromethyl)phenyl]boronic acid instead of (3-fluorophenyl)boronic acid.

[MS (ESI) m/z 372.2 (M+H)⁺]

### Example 62

### 2-(Pyridin-2-ylmethoxy)-4-[2-(trifluoromethyl)phenyl]-6,7-dihydro-5H-cyclopenta[b]pyridine

The title compound was prepared as colorless oil according to the procedure described in Example 58 using [2-(trifluoromethyl)phenyl]boronic acid instead of (3-fluorophenyl)boronic acid.

[MS (ESI) m/z 371.2 (M+H)⁺]

### Example 63

### 4-(3-Nitrophenyl)-2-(pyridin-2-ylmethoxy)-6,7-dihydro-5H-cyclopenta[b]pyridine

The title compound was prepared as a white powder according to the procedure described in Example 58 using (3-nitrophenyl)boronic acid instead of (3-fluorophenyl)boronic acid.

[MS (ESI) m/z 349.3 (M+H)⁺]

### Example 64

### 4-(3-Fluoro-4-methylphenyl)-2-(pyridin-2-ylmethoxy)-6,7-dihydro-5H-cyclopenta[b]pyridine

The title compound was prepared as colorless oil according to the procedure described in Example 5, Step 2, using 4-bromo-2-fluoro-1-methylbenzene instead of 5-bromopyridin-3-ol.

[MS (ESI) m/z 335.3 (M+H)⁺]

### Example 65

### 4-(3-Fluoro-5-methylphenyl)-2-(pyridin-2-ylmethoxy)-6,7-dihydro-5H-cyclopenta[b]pyridine

The title compound was prepared as a white powder according to the procedure described in Example 5, Step 2, using 1-bromo-3-fluoro-5-methylbenzene instead of 5-bromopyridin-3-ol.

[MS (ESI) m/z 335.4 (M+H)⁺]

### Example 66

### 2-Fluoro-4-[2-(pyridin-2-ylmethoxy)-6,7-dihydro-5H-cyclopenta[b]pyridin-4-yl]benzonitrile

The title compound was prepared as a white powder according to the procedure described in Example 5, Step 2, using 4-bromo-2-fluorobenzonitrile instead of 5-bromopyridin-3-ol.

[MS (ESI) m/z 346.3 (M+H)⁺]

### Example 67

### 2-Fluoro-4-[2-(pyridin-2-ylmethoxy)-6,7-dihydro-5H-cyclopenta[b]pyridin-4-yl]benzamide

To 2-fluoro-4-[2-(pyridin-2-ylmethoxy)-6,7-dihydro-5H-cyclopenta[b]pyridin-4-yl]benzonitrile (25 mg) were added tert-butanol (1 mL), and further added excess amounts of potassium fluoride on alumina, and the mixture was stirred under at 95° C. overnight. After the reaction mixture was allowed to return to room temperature, diluted with ethyl acetate, and filtered through Celite, the filtrate was evaporated under reduced pressure. The resulting residue was purified by silica gel column chromatography to give the title compound (15 mg) as a white powder.

[MS (ESI) m/z 364.3 (M+H)⁺]

### Example 68

### 3-Fluoro-5-[2-(pyridin-2-ylmethoxy)-6,7-dihydro-5H-cyclopenta[b]pyridin-4-yl]phenol

The title compound was prepared as a white powder according to the procedure described in Example 7, Step 1.

[MS (ESI) m/z 337.3 (M+H)⁺]

### Example 69

### 3-Fluoro-5-[2-(pyridin-2-ylmethoxy)-6,7-dihydro-5H-cyclopenta[b]pyridin-4-yl]phenyl methanesulfonate

To a solution of 3-fluoro-5-[2-(pyridin-2-ylmethoxy)-6,7-dihydro-5H-cyclopenta[b]pyridin-4-yl]phenol (16 mg) in CH₂Cl₂ (1 mL) was added MsCl (0.008 mL), and the mixture was stirred at room temperature overnight. The reaction mixture was purified by silica gel column chromatography to give the title compound (4 mg) as colorless oil.

[MS (ESI) m/z 415.3 (M+H)⁺]

### Example 70

### 4-(3,4-Dimethoxyphenyl)-2-(pyridin-2-ylmethoxy)-6,7-dihydro-5H-cyclopenta[b]pyridine

The title compound was prepared as colorless oil according to the procedure described in Example 58 using (3,4-dimethoxyphenyl)boronic acid instead of (3-fluorophenyl)boronic acid.

[MS (ESI) m/z 363.3 (M+H)⁺]

### Example 71

### 4-(5-Methylpyridin-2-yl)-2-(pyridin-2-ylmethoxy)-6,7-dihydro-5H-cyclopenta[b]pyridine

To 4-chloro-2-(pyridin-2-ylmethoxy)-6,7-dihydro-5H-cyclopenta[b]pyridine (100 mg), 5-methyl-2-(tributylstannyl)pyridine (146 mg) and Pd(PPh₃)₄ (44 mg) was added DMF (0.5 mL), and the mixture was reacted under microwave irradiation at 160° C. for 1 hour. After the reaction mixture was allowed to return to room temperature, diluted with ethyl acetate, and filtered through Celite, the filtrate was evaporated under reduced pressure. The resulting residue was purified by silica gel column chromatography to give the title compound (24 mg) as a white powder.

[MS (ESI) m/z 318.3 (M+H)⁺]

### Example 72

### 4-(6-Methylpyridin-2-yl)-2-(pyridin-2-ylmethoxy)-6,7-dihydro-5H-cyclopenta[b]pyridine

To 4-chloro-2-(pyridin-2-ylmethoxy)-6,7-dihydro-5H-cyclopenta[b]pyridine (100 mg), 2-methyl-6-(tributylstannyl)pyridine (147 mg) and Pd(PPh₃)₄ (44 mg) was added 1,4-dioxane (0.5 mL), and the mixture was reacted under microwave irradiation at 160° C. for 1 hour. After the reaction mixture was allowed to return to room temperature, diluted with ethyl acetate, and filtered through Celite, the filtrate was evaporated under reduced pressure. The resulting residue was purified by silica gel column chromatography to give the title compound (24 mg) as a white powder.

[MS (ESI) m/z 318.3 (M+H)⁺]

### Example 73

### 4-(2-Fluoropyridin-4-yl)-2-(pyridin-2-ylmethoxy)-6,7-dihydro-5H-cyclopenta[b]pyridine

The title compound was prepared as a white powder according to the procedure described in Example 58 using (2-fluoropyridin-2-yl)boronic acid instead of (3-fluorophenyl)boronic acid.

[MS (ESI) m/z 322.3 (M+H)⁺]

### Example 74

### 4-(Furan-2-yl)-2-(pyridin-2-ylmethoxy)-6,7-dihydro-5H-cyclopenta[b]pyridine

The title compound was prepared as a white solid according to the procedure described in Example 58 using (furan-2-yl)boronic acid instead of (3-fluorophenyl)boronic acid.

[MS (ESI) m/z 294.3 (M+H)⁺]

### Example 75

### 4-(1-Methyl-1H-pyrrol-2-yl)-2-(pyridin-2-ylmethoxy)-6,7-dihydro-5H-cyclopenta[b]pyridine

The title compound was prepared as a white powder according to the procedure described in Example 72 using 1-methyl-2-(tributylstannanyl)-1H-pyrrole instead of 2-methyl-6-(tributylstannanyl)pyridine.

[MS (ESI) m/z 306.3 (M+H)⁺]

### Example 76

### 3-[2-(Pyridin-2-ylmethoxy)-6,7-dihydro-5H-cyclopent a[b]pyridin-4-yl]quinoline

The title compound was prepared as a beige powder according to the procedure described in Example 58 using quinolin-3-ylboronic acid instead of (3-fluorophenyl)boronic acid.

[MS (ESI) m/z 354.3 (M+H)⁺]

### Example 77

### 1-Methyl-6-[2-(pyridin-2-ylmethoxy)-6,7-dihydro-5H-cyclopenta[b]pyridin-4-yl]-1H-benzimidazole

The title compound was prepared as colorless oil according to the procedure described in Example 58 using 1-methyl-6-(4,4,5,5-tetramethyl-1,3,2-dioxaborolan-2-yl)-1H-benzimidazole (see, for example, WO2009/14637) instead of (3-fluorophenyl)boronic acid, and using Pd₂(dba)₃ instead of Pd(OAc)₂

[MS (ESI) m/z 357.3 (M+H)⁺]

### Example 78

### 1-Methyl-5-[2-(pyridin-2-ylmethoxy)-6,7-dihydro-5H-cyclopenta[b]pyridin-4-yl]-1H-benzimidazole hydrochloride

1-Methyl-5-[2-(pyridin-2-ylmethoxy)-6,7-dihydr o-5H-cyclopenta[b]pyridin-4-yl]-1H-benzimidazole (129 mg) was prepared as colorless oil according to the procedure described in Example 58 using (1-methyl-1H-benzimidazol-5-yl)boronic acid instead of (3-fluorophenyl)boronic acid.

[MS (ESI) m/z 357.3 (M+H)⁺]

The resulting compound was dissolved in ethyl acetate (4 mL), followed by the addition of 1 N HCl/Et₂O solution (0.353 mL), and the mixture was stirred for 0.5 hour. The resulting precipitate was collected by filtration to give the title compound (128 mg) as a white powder.

[MS (ESI) m/z 357.3 (M+H)⁺]

### Example 79

### 4-(1,3-Benzodioxol-5-yl)-2-(pyridin-2-ylmethoxy)-6,7-dihydro-5H-cyclopenta[b]pyridine

The title compound was prepared as a beige powder according to the procedure described in Example 58 using 1,3-benzodioxol-5-ylboronic acid instead of (3-fluorophenyl)boronic acid.

[MS (ESI) m/z 347.3 (M+H)⁺]

### Example 80

### 6-[2-(Pyridin-2-ylmethoxy)-6,7-dihydro-5H-cyclopent a[b]pyridin-4-yl]imidazo[1,2-a]pyridine

The title compound was prepared as a brown powder according to the procedure described in Example 5, Step 2, using 6-bromoimidazo[1,2-a]pyridine instead of 5-bromopyridin-3-ol.

[MS (ESI) m/z 343.4 (M+H)⁺]

### Example 81

### 6-[2-(Pyridin-2-ylmethoxy)-6,7-dihydro-5H-cyclopent a[b]pyridin-4-yl]imidazo[1,2-b]pyridazine

The title compound was prepared as a white powder according to the procedure described in Example 5, Step 2, using 6-chloroimidazo[1,2-b]pyridazine instead of 5-bromopyridin-3-ol.

[MS (ESI) m/z 344.4 (M+H)⁺]

### Example 82

### 4-(Pyridin-3-yl)-2-(pyridin-2-ylmethoxy)-5,6,7,8-tetrahydroquinoline

### [Step 1]

Production of 4-chloro-2-(pyridin-2-ylmethoxy)-5,6,7,8-tetrahydroquinoline

To pyridin-2-ylmethanol (119 mg) was added toluene (2 mL), followed by the addition of NaH (60% dispersion in oil, 48 mg) under ice water cooling, and the mixture was stirred for 30 minutes. After the reaction mixture was allowed to return to room temperature, 2,4-dichloro-5,6,7,8-tetrahydroquinoline (see, for example, Helvetica Chimica Acta, 1945, vol. 28, p. 1684-1690) (200 mg) was added to the mixture, and the mixture was stirred at 100° C. for 3 hours. After the reaction mixture was allowed to return to room temperature, it was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (44 mg) as colorless oil.

[Rf value (TLC silica gel plate 60F₂₅₄, developing solvent:hexane:ethyl acetate=2:1):0.5]

### [Step 2]

Production of 4-(pyridin-3-yl)-2-(pyridin-2-ylmethoxy)-5,6,7,8-tetrahydroquinoline

To 4-chloro-2-(pyridin-2-ylmethoxy)-5,6,7,8-tetrahydroquinoline (22 mg), pyridin-3-yl boronic acid (15 mg), Pd(OAc)₂ (2 mg), S-Phos (7 mg) and potassium phosphate (51 mg) was added 1,4-dioxane/water (3/1, 2 mL), and the mixture was stirred at 100° C. for 2 hours. After the reaction mixture was allowed to return to room temperature, diluted with ethyl acetate, and filtered through Celite, the filtrate was evaporated under reduced pressure. The resulting residue was purified by silica gel column chromatography to give the title compound (17 mg) as colorless oil.

[MS (ESI) m/z 318.3 (M+H)+]

### Example 83

### 2-[(6-Methylpyridin-2-yl)methoxy]-4-(pyridin-3-yl)-5,6,7,8-tetrahydroquinoline

### [Step 1]

Production of 2-chloro-4-(pyridin-2-ylmethoxy)-5,6,7,8-tetrahydroquinoline

The title compound was prepared according to the procedure described in Example 9, Step 1, using 2,4-dichloro-5,6,7,8-tetrahydroquinoline instead of 2,4-dichloro-6,7-dihydro-5H-cyclopenta[b]pyridine. [Rf value (TLC silica gel plate 60F₂₅₄, developing solvent:hexane:ethyl acetate=2:1):0.3]

### [Step 2]

Production of 2-chloro-5,6,7,8-tetrahydroquinolin-4-ol

The title compound was prepared according to the procedure described in Example 9, Step 2, using 2-chloro-4-(pyridin-2-ylmethoxy)-5,6,7,8-tetrahydroquinoline instead of 2-chloro-4-(pyridin-2-ylmethoxy)-6,7-dihydro-5H-cyclopenta[b]pyridine.

### [Step 3]

Production of 2-chloro-5,6,7,8-tetrahydroquinolin-4-yl trifluoromethanesulfonate

The title compound was prepared according to the procedure described in Example 9, Step 3, using 2-chloro-5,6,7,8-tetrahydroquinolin-4-ol instead of 2-chloro-6,7-dihydro-5H-cyclopenta[b]pyridin-4-ol.

### [Step 4]

Production of 2-chloro-4-(pyridin-3-yl)-5,6,7,8-tetrahydroquinoline

To 2-chloro-5,6,7,8-tetrahydroquinolin-4-yl trifluoromethanesulfonate (200 mg), pyridin-3-ylboronic acid (86 mg), Pd(PPh₃)₄ (73 mg) and potassium carbonate (262 mg) was added THF/water (3/1, 8 mL), and the mixture was degassed, and then stirred under Ar atmosphere at 60° C. for 3 hours. After the reaction mixture was allowed to return to room temperature, the reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (144 mg).

### [Step 5]

Production of 2-[(6-methylpyridin-2-yl)methoxy]-4-(pyridin-3-yl)-5,6,7,8-tetrahydroquinoline

(6-Methylpyridin-2-yl)methanol (15 mg) and NaH (60% dispersion in oil, 10 mg) were dissolved in DMSO (1 mL), and the mixture was stirred for 30 minutes. Then, 2-chloro-4-(pyridin-3-yl)-5,6,7,8-tetrahydroquinoline (20 mg) was added to the mixture, and the mixture was stirred at 100° C. for 1 hour. After the reaction mixture was allowed to return to room temperature, the reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (5 mg) as pale yellow oil.

[MS (ESI) m/z 332.2 (M+H)+]

### Example 84

### 2-[(5-Methylpyridin-2-yl)methoxy]-4-(pyridin-3-yl)-5,6,7,8-tetrahydroquinoline

### [Step 1]

Production of 4-(pyridin-3-yl)-5,6,7,8-tetrahydroquinolin-2-ol

To a solution of 4-(pyridin-3-yl)-2-(pyridin-2-ylmethoxy)-5,6,7,8-tetrahydroquinoline (640 mg) and NaI (1.21 g) in MeCN (20 mL) and water (2 mL) was added sulfuric acid (0.22 mL), and the mixture was heated under reflux for 1 hour. After the reaction mixture was allowed to return to room temperature, it was added with aqueous sodium hydrogen carbonate solution and aqueous sodium thiosulfate solution, and then the solvent was evaporated under reduced pressure. The resulting residue was diluted with chloroform/methanol (7/1), and filtered through Celite, and the solvent was evaporated under reduced pressure. The residue was purified by silica gel column chromatography to give the title compound (336 mg)

### [Step 2]

Production of 2-[(5-methylpyridin-2-yl)methoxy]-4-(pyridin-3-yl)-5,6,7,8-tetrahydroquinoline

To 4-(pyridin-3-yl)-5,6,7,8-tetrahydroquinolin-2-ol (20 mg), 2-(chloromethyl)-5-methylpyridine hydrochloride (32 mg) and silver(I) carbonate (73 mg) was added DMF (1 mL), and the mixture was stirred at 100° C. for 3 hours. After the reaction mixture was allowed to return to room temperature, diluted with ethyl acetate, and filtered through Celite, the filtrate was evaporated under reduced pressure. The resulting residue was purified by silica gel column chromatography to give the title compound (7.5 mg) as colorless oil.

[MS (ESI) m/z 332.1 (M+H)⁺]

### Example 85

### 2-[(4-Methylpyridin-2-yl)methoxy]-4-(pyridin-3-yl)-5,6,7,8-tetrahydroquinoline

To 2-chloro-4-(pyridin-3-yl)-5,6,7,8-tetrahydroquinoline (30 mg), (4-methylpyridin-2-yl)methanol (23 mg), Pd₂(dba)₃ (7 mg), t-Bu-X-Phos (8 mg) and cesium carbonate (80 mg) was added toluene (1.5 mL), and the mixture was degassed, and then stirred under Ar atmosphere at 100° C. for 2 hours. After the reaction mixture was allowed to return to room temperature, the mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (29 mg) as colorless oil.

[MS (ESI) m/z 331.9 (M+H)⁺]

### Example 86

### 2-[(6-Fluoropyridin-2-yl)methoxy]-4-(pyridin-3-yl)-5,6,7,8-tetrahydroquinoline

To 2-chloro-4-(pyridin-3-yl)-5,6,7,8-tetrahydroquinoline (65 mg), (6-fluoropyridin-2-yl)methanol (34 mg), Pd₂(dba)₃.CHCl₃ (17 mg), t-Bu-X-Phos (18 mg) and cesium carbonate (174 mg) was added toluene (2.6 mL), and the mixture was degassed, and then stirred at 100° C. for 4 hours under Ar atmosphere. The mixture was filtered through Celite and the solvent was evaporated under reduced pressure. The resulting residue was purified by silica gel column chromatography to give the title compound (26 mg) as colorless oil.

[MS (ESI) m/z 336.2 (M+H)⁺]

### Example 87

### 2-[(6-Methoxypyridin-2-yl)methoxy]-4-(pyridin-3-yl)-5,6,7,8-tetrahydroquinoline hydrochloride

2-[(6-Methoxypyridin-2-yl)methoxy]-4-(pyridin-3-yl)-5,6,7,8-tetrahydroquinoline (16 mg) was prepared according to the procedure described in Example 86 using (6-methoxypyridin-2-yl)methanol instead of (6-fluoropyridin-2-yl)methanol, and using NaO-t-Bu instead of cesium carbonate.

[MS (ESI) m/z 348.2 (M+H)⁺]

The resulting compound was dissolved in ethyl acetate (1 mL), 1 N HCl/Et₂O solution (0.046 mL) was added to the solution under ice water cooling, and the mixture was stirred under ice water cooling for 0.5 hour.

The resulting precipitate was collected by filtration to give the title compound (4 mg) as a pale green powder.

[MS (ESI) m/z 348.2 (M+H)⁺]

### Example 88

### [6-({[4-(Pyridin-3-yl)-5,6,7,8-tetrahydroquinolin-2-yl]oxy}methyl)pyridin-2-yl]methanol

The title compound was prepared as a white solid according to the procedure described in Example 86 using (pyridine-2,6-diyl)bismethanol instead of (6-fluoropyridin-2-yl)methanol and using NaO-t-Bu instead of cesium carbonate.

[MS (ESI) m/z 348.2 (M+H)+]

### Example 89

### 2-{[6-(Methoxymethyl)pyridin-2-yl]methoxy}-4-(pyrid in-3-yl)-5,6,7,8-tetrahydroquinoline

To [6-({[4-(pyridin-3-yl)-5,6,7,8-tetrahydroquinolin-2-yl]oxy}methyl)pyridin-2-yl]methanol (7 mg) was added THF (0.5 mL), followed by the addition of NaH (60% dispersion in oil, 2.4 mg) under ice water cooling, and the mixture was stirred for 30 minutes and then was added with methyl iodide (hereinafter referred to as “MeI”) (0.0015 mL) and stirred at room temperature for 4 hours. Additionally, NaH (60% dispersion in oil, 2.4 mg) was added to the solution, and the mixture was further stirred for 2 hours. The reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (4 mg) as pale green oil.

[MS (ESI) m/z 362.3 (M+H)⁺]

### Example 90

### 4-(Pyridin-3-yl)-2-(pyrimidin-2-ylmethoxy)-5,6,7,8-tetrahydroquinoline

The title compound was prepared as colorless oil according to the procedure described in Example 86 using pyrimidin-2-ylmethanol instead of (6-fluoropyridin-2-yl)methanol.

[MS (ESI) m/z 319.2 (M+H)⁺]

### Example 91

### 2-(1,3-Oxazol-2-ylmethoxy)-4-(pyridin-3-yl)-5,6,7,8-tetrahydroquinoline

To a solution of 2-chloro-4-(pyridin-3-yl)-5,6,7,8-tetrahydroquinoline (20 mg) and 1,3-oxazol-2-ylmethanol (12 mg) in DMSO (1 mL) was added NaH (60% dispersion in oil, 7 mg), and the mixture was stirred at 100° C. for 3 hours. After the reaction mixture was allowed to return to room temperature, the reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (11 mg) as a white powder.

[MS (ESI) m/z 308.3 (M+H)⁺]

### Example 92

### 2-[(4-Methyl-1,3-oxazol-2-yl)methoxy]-4-(pyridin-3-yl)-5,6,7,8-tetrahydroquinoline

The title compound was prepared as colorless oil according to the procedure described in Example 85 using (4-methyl-1,3-oxazol-2-yl)methanol instead of (4-methylpyridin-2-yl)methanol.

[MS (ESI) m/z 322.2 (M+H)⁺]

### Example 93

### 2-[(2-Methyl-1,3-oxazol-4-yl)methoxy]-4-(pyridin-3-yl)-5,6,7,8-tetrahydroquinoline hydrochloride

2-[(2-Methyl-1,3-oxazol-4-yl)methoxy]-4-(pyrid in-3-yl)-5,6,7,8-tetrahydroquinoline (25 mg) was prepared according to the procedure described in Example 85 using (2-methyl-1,3-oxazol-4-yl)methanol instead of (4-methylpyridin-2-yl)methanol. The resulting compound was dissolved in ethyl acetate, 1 N HCl/Et₂O solution (0.078 mL) was added under ice water cooling, and then the mixture was stirred at the same temperature. The resulting precipitate was collected by filtration to give the title compound (20 mg) as a white powder.

[MS (ESI) m/z 322.3 (M+H)⁺]

### Example 94

### 4-(Pyridin-3-yl)-2-(1,3-thiazol-2-ylmethoxy)-5,6,7,8-tetrahydroquinoline hydrochloride

The title compound was prepared as a white powder according to the procedure described in Example 87 using 1,3-thiazol-2-ylmethanol instead of (6-methoxypyridin-2-yl)methanol, and using 1,4-dioxane instead of toluene.

[MS (ESI) m/z 324.1 (M+H)⁺]

### Example 95

### 2-[(4-Methyl-1,3-thiazol-2-yl)methoxy]-4-(pyridin-3-yl)-5,6,7,8-tetrahydroquinoline

The title compound was prepared as colorless oil according to the procedure described in Example 85 using (4-methyl-1,3-thiazol-2-yl)methanol instead of (4-methylpyridin-2-yl)methanol, and using NaO-t-Bu instead of cesium carbonate.

[MS (ESI) m/z 338.0 (M+H)⁺]

### Example 96

### 2-[(2-Methyl-1,3-thiazol-4-yl)methoxy]-4-(pyridin-3-yl)-5,6,7,8-tetrahydroquinoline

The title compound was prepared as colorless oil according to the procedure described in Example 86 using (2-methyl-1,3-thiazol-4-yl)methanol instead of (4-methylpyridin-2-yl)methanol.

[MS (ESI) m/z 338.0 (M+H)⁺]

### Example 97

### 2-(1H-Imidazol-2-ylmethoxy)-4-(pyridin-3-yl)-5,6,7,8-tetrahydroquinoline

### [Step 1]

Production of 4-(pyridin-3-yl)-2-[(1-{[2-(trimethylsilyl)ethoxy]m ethyl}-1H-imidazol-2-yl)methoxy]-5,6,7,8-tetrahydroquinoline

The title compound was prepared according to the procedure described in Example 85 using (1-{[2-(trimethylsilyl)ethoxy]methyl}-1H-imidazol-2-yl)methanol (see, for example, WO2007/000582) instead of (6-fluoropyridin-2-yl)methanol and using NaO-t-Bu instead of cesium carbonate.

### [Step 2]

Production of 2-(1H-imidazol-2-ylmethoxy)-4-(pyridin-3-yl)-5,6,7,8-tetrahydroquinoline

To 4-(pyridin-3-yl)-2-[(1-{[2-(trimethylsilyl)ethoxy]methyl}-1H-imidazol-2-yl)methoxy]-5,6,7,8-tetrahydroquinoline (90 mg) were added CH₂Cl₂ (2 mL), ethanol (0.01 mL) and TFA (0.5 mL), and the mixture was stirred at 40° C. for 10 hours and further stirred at 60° C. for 2 hours. Additionally, the reaction mixture was added with TFA (0.45 mL), and was further stirred at room temperature for 2 hours. The reaction mixture was added with water, ethyl acetate and potassium carbonate, and extracted with chloroform. The organic layer was dried over anhydrous sodium sulfate, and filtered off, and the filtrate was evaporated under reduced pressure. The resulting residue was purified by silica gel column chromatography to give the title compound (35 mg) as colorless oil.

[MS (ESI) m/z 307.2 (M+H)⁺]

### Example 98

### 2-[(1-Methyl-1H-imidazol-2-yl)methoxy]-4-(pyridin-3-yl)-5,6,7,8-tetrahydroquinoline

To a solution of 2-(1H-imidazol-2-ylmethoxy)-4-(pyridin-3-yl)-5,6,7, 8-tetrahydroquinoline (10 mg) in DMSO (0.5 mL) were added potassium carbonate (14 mg) and MeI (0.003 mL), and the mixture was stirred at room temperature overnight. The reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (8 mg) as colorless oil.

[MS (ESI) m/z 321.2 (M+H)⁺]

### Example 99

### 4-(4-Methylpyridin-3-yl)-2-(pyridin-2-ylmethoxy)-5,6,7,8-tetrahydroquinoline

To 4-chloro-2-(pyridin-2-ylmethoxy)-5,6,7,8-tetrahydroquinoline (40 mg), (4-methylpyridin-3-yl)boronic acid (40 mg), Pd(dppf)Cl₂.CH₂Cl₂ (6 mg) and potassium carbonate (104 mg) was added 1,4-dioxane/water (3/1, 0.6 mL), and the mixture was stirred at 180° C. for 15 minutes. After the reaction mixture was allowed to return to room temperature, diluted with ethyl acetate, and filtered through Celite, the filtrate was evaporated under reduced pressure. The resulting residue was purified by silica gel column chromatography to give the title compound (16 mg) as colorless oil.

[MS (ESI) m/z 332.3 (M+H)⁺]

### Example 100

### 4-(2-Methylpyridin-3-yl)-2-(pyridin-2-ylmethoxy)-5,6,7,8-tetrahydroquinoline hydrochloride

To 4-chloro-2-(pyridin-2-ylmethoxy)-5,6,7,8-tetrahydroquinoline, 2-methyl-3-(4,4,5,5-tetramethyl-1,3,2-dioxaborolan-2-yl)pyridine, Pd₂(dba)₃.CHCl₃ (19 mg), t-Bu-X-Phos (19 mg) and potassium carbonate (76 mg) was added 1,4-dioxane/water (3/1, 1.8 mL), and the mixture was degassed, and then stirred under Ar atmosphere at 80° C. for 5 hours. After the reaction mixture was allowed to return to room temperature, diluted with ethyl acetate, dried over anhydrous sodium sulfate, and filtered through Celite, the filtrate was evaporated under reduced pressure. The resulting residue was purified by silica gel column chromatography to give 4-(2-methylpyridin-3-yl)-2-(pyridin-2-ylmethoxy)-5,6,7,8-tetrahydroquinoline. The resulting compound was dissolved in ethyl acetate, followed by the addition of 1 N HCl/Et₂O solution (0.03 mL), and the mixture was stirred at room temperature overnight. The resulting precipitate was collected by filtration to give the title compound as a white powder.

[MS (ESI) m/z 332.3 (M+H)⁺]

### Example 101

### 4-(6-Fluoropyridin-3-yl)-2-(pyridin-2-ylmethoxy)-5,6,7,8-tetrahydroquinoline dihydrochloride

To 5-bromo-2-fluoropyridine (67 mg), bis(pinacolato)diboron (116 mg), Pd₂ (dba)₃.CHCl₃ (20 mg), X-Phos (36 mg) and potassium carbonate (80 mg) was added 1,4-dioxane (3.8 mL), and the mixture was degassed, and then stirred under Ar atmosphere at 100° C. for 2 hours. After the reaction mixture was allowed to return to room temperature, diluted with ethyl acetate, and filtered through Celite, the filtrate was evaporated under reduced pressure. The resulting residue was purified by silica gel column chromatography to give 2-fluoro-5-(4,4,5,5-tetramethyl-1,3,2-dioxaborolan-2-yl)pyridine. To the resulting compound were added 4-chloro-2-(pyridin-2-ylmethoxy)-5,6,7,8-tetrahydroquinoline (70 mg), Pd(OAc)₂ (6 mg), S-Phos (21 mg), potassium carbonate (104 mg) and 1,4-dioxane/water (3/1, 1.8 mL), and the mixture was degassed, and then stirred under Ar atmosphere at 100° C. for 5 hours. After the reaction mixture was allowed to return to room temperature, diluted with ethyl acetate, dried over anhydrous sodium sulfate, and filtered through Celite, the filtrate was evaporated under reduced pressure. The resulting residue was purified by silica gel column chromatography to give 4-(6-fluoropyridin-3-yl)-2-(pyridin-2-ylmethoxy)-5,6,7,8-tetrahydroquinoline (90 mg).

[MS (ESI) m/z 336.3 (M+H)⁺]

The resulting compound was dissolved in ethyl acetate, followed by the addition of 1 N HCl/Et₂O (0.268 mL), and the resulting precipitate was collected by filtration to give the title compound (38 mg) as a white powder.

[MS (ESI) m/z 336.3 (M+H)⁺]

### Example 102

### 4-(5-Methoxypyridin-3-yl)-2-(pyridin-2-ylmethoxy)-5,6,7,8-tetrahydroquinoline hydrochloride

The title compound was prepared as a white powder according to the procedure described in Example 101 using 3-bromo-5-methoxypyridine instead of 5-bromo-2-fluoropyridine.

[MS (ESI) m/z 348.2 (M+H)⁺]

### Example 103

### 4-(2-Methoxypyridin-3-yl)-2-(pyridin-2-ylmethoxy)-5,6,7,8-tetrahydroquinoline hydrochloride

### [Step 1]

Production of 4-(2-fluoropyridin-3-yl)-2-(pyridin-2-ylmethoxy)-5,6,7,8-tetrahydroquinoline

To 4-chloro-2-(pyridin-2-ylmethoxy)-5,6,7,8-tetrahydro quinoline (170 mg), 2-fluoro-3-(4,4,5,5-tetramethyl-1,3,2-dioxaborolan-2-yl)pyridine (207 mg), Pd(OAc)₂ (14 mg), S-Phos (51 mg) and potassium carbonate (257 mg) was added 1,4-dioxane/water (3/1, 6 mL), and the mixture was degassed, and then stirred under Ar atmosphere at 100° C. for 4 hours. After the reaction mixture was allowed to return to room temperature, diluted with ethyl acetate, dried over anhydrous sodium sulfate, and filtered through Celite, the solvent was evaporated under reduced pressure. The resulting residue was purified by silica gel column chromatography to give the title compound (124 mg) as a white powder.

### [Step 2]

Production of 4-(2-methoxypyridin-3-yl)-2-(pyridin-2-ylmethoxy)-5,6,7,8-tetrahydroquinoline hydrochloride

To 4-(2-fluoropyridin-3-yl)-2-(pyridin-2-ylmethoxy)-5,6,7,8-tetrahydroquinoline (25 mg) were added methanol (2 mL) and KO-t-Bu (42 mg), and the mixture was heated under reflux for 12 hours. The reaction mixture was evaporated under reduced pressure. The resulting residue was purified by silica gel column chromatography to give 4-(2-methoxypyridin-3-yl)-2-(pyridin-2-ylmethoxy)-5,6,7,8-tetrahydroquinoline (25 mg) as colorless oil. The resulting compound was dissolved in ethyl acetate (2 mL), followed by the addition of 1 N HCl/Et₂O solution (0.075 mL), and the mixture was stirred at room temperature for 30 minutes. The resulting precipitate was collected by filtration to give the title compound (4 mg) as a white solid.

[MS (ESI) m/z 348.2 (M+H)⁺]

### Example 104

### 5-[2-(Pyridin-2-ylmethoxy)-5,6,7,8-tetrahydroquinolin-4-yl]pyridine-2-carbonitrile hydrochloride

5-[2-(Pyridin-2-ylmethoxy)-5,6,7,8-tetrahydroquinolin-4-yl]pyridine-2-carbonitrile was prepared as colorless oil according to the procedure described in Example 103, Step 1, using 5-(4,4,5,5-tetramethyl-1,3,2-dioxaborolan-2-yl)pyridine-2-carbonitrile instead of 2-fluoro-3-(4,4,5,5-tetramethyl-1,3,2-dioxaborolan-2-yl)pyridine. The resulting compound (12 mg) was dissolved in ethyl acetate (2 mL), followed by the addition of 1 N HCl/Et₂O solution (0.035 mL), and the resulting precipitate was collected by filtration to give the title compound (9.1 mg) as a white powder.

[MS (ESI) m/z 344.2 (M+H)⁺]

### Example 105

### 4-(4-Chloropyridin-3-yl)-2-(pyridin-2-ylmethoxy)-5,6,7,8-tetrahydroquinoline hydrochloride

### [Step 1]

Production of 2-(pyridin-2-ylmethoxy)-5,6,7,8-tetrahydroquinolin-4-ol

To 4-chloro-2-(pyridin-2-ylmethoxy)-5,6,7,8-tetrahydroquinoline (1 g), potassium hydroxide (613 mg), Pd₂ (dba)₃ (33 mg) and t-Bu-X-Phos (38 mg) were added 1,4-dioxane (5 mL) and water (1 mL), and the mixture was degassed, and then stirred under Ar atmosphere at 100° C. for 2 hours. After the reaction mixture was allowed to return to room temperature, the mixture was added with ethyl acetate and water, and subjected to extraction. The organic layer was dried over anhydrous magnesium sulfate, and filtered off, and the filtrate was evaporated under reduced pressure. The resulting residue was purified by silica gel column chromatography to give the title compound (739 mg).

### [Step 2]

Production of 2-(pyridin-2-ylmethoxy)-5,6,7,8-tetrahydroquinolin-4-yl trifluoromethanesulfonate

The title compound was prepared according to the procedure described in Example 4, Step 2, using 2-(pyridin-2-ylmethoxy)-5,6,7,8-tetrahydroquinolin-4-ol instead of 2-(pyridin-2-ylmethoxy)-6,7-dihydro-5H-cyclopenta[b]pyridin-4-ol.

### [Step 3]

Production of 4-(4-chloropyridin-3-yl)-2-(pyridin-2-ylmethoxy)-5,6,7,8-tetrahydroquinoline hydrochloride

To 2-(pyridin-2-ylmethoxy)-5,6,7,8-tetrahydroquinolin-4-yl trifluoromethanesulfonate (40 mg), 4-chloro-3-(tributylstannanyl)pyridine (60 mg), LiCl (13 mg), Pd(PPh₃)₄ (24 mg) and copper iodide (3.9 mg) was added DMF (0.6 mL), and the mixture was reacted under microwave irradiation at 160° C. for 30 minutes. After the reaction mixture was allowed to return to room temperature, diluted with ethyl acetate, and filtered through Celite, the filtrate was evaporated under reduced pressure. The resulting residue was purified by silica gel column chromatography to give 4-(4-chloropyridin-3-yl)-2-(pyridin-2-ylmethoxy)-5,6,7,8-tetrahydroquinoline (15 mg) as a white powder.

[MS (ESI) m/z 352.1 (M+H)⁺]

The resulting compound was dissolved in ethyl acetate, followed by addition of 1 N HCl/Et₂O solution (0.043 mL) under ice water cooling, and the mixture was stirred at room temperature overnight. The resulting precipitate was collected by filtration to give the title compound (7 mg) as a white powder.

[MS (ESI) m/z 352.1 (M+H)⁺]

### Example 106

### 4-(6-Methoxypyridin-3-yl)-2-(pyridin-2-ylmethoxy)-5,6,7,8-tetrahydroquinoline hydrochloride

To 4-(6-methoxypyridin-3-yl)-2-(pyridin-2-ylmethoxy)-5,6,7,8-tetrahydroquinoline (45 mg) were added methanol (4 mL) and NaH (60% dispersion in oil, 16 mg), and the mixture was stirred at room temperature for 72 hours, and then at 40° C. for 5 hours, and further at room temperature for 24 hours. The reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give 4-(6-methoxypyridin-3-yl)-2-(pyridin-2-ylmethoxy)-5,6,7,8-tetrahydroquinoline (13 mg). The resulting compound was dissolved in ethyl acetate, followed by the addition of 1 N HCl/Et₂O solution (0.039 mL) under ice water cooling, and the mixture was stirred at room temperature for 1 hour. The resulting precipitate was collected by filtration to give the title compound (5 mg) as a white powder.

[MS (ESI) m/z 348.2 (M+H)⁺]

### Example 107

### 4-(5-Fluoropyridin-3-yl)-2-(pyridin-2-ylmethoxy)-5,6,7,8-tetrahydroquinoline hydrochloride

The title compound was prepared as a white solid according to the procedure described in Example 100 using 3-fluoro-5-(4,4,5,5-tetramethyl-1,3,2-dioxaborolan-2-yl)pyridine instead of 2-methyl-3-(4,4,5,5-tetramethyl-1,3,2-dioxaborolan-2-yl)pyridine.

[MS (ESI) m/z 336.0 (M+H)⁺]

### Example 108

### 4-(2-Fluoro-5-methylpyridin-3-yl)-2-(pyridin-2-ylmethoxy)-5,6,7,8-tetrahydroquinoline

### [Step 1]

Production of 2-(pyridin-2-ylmethoxy)-4-(4,4,5,5-tetramethyl-1,3,2-dioxaborolan-2-yl)-5,6,7,8-tetrahydroquinoline

The title compound was prepared according to the procedure described in Example 5, Step 1, using 2-(pyridin-2-ylmethoxy)-5,6,7,8-tetrahydroquinolin-4-yl trifluoromethanesulfonate instead of 2-(pyridin-2-ylmethoxy)-6,7-dihydro-5H-cyclopenta[b]pyridin-4-yl trifluoromethanesulfonate.

### [Step 2]

Production of 4-(2-fluoro-5-methylpyridin-3-yl)-2-(pyridin-2-ylmethoxy)-5,6,7,8-tetrahydroquinoline

To 2-(pyridin-2-ylmethoxy)-4-(4,4,5,5-tetramethyl-1,3,2-dioxaborolan-2-yl)-5,6,7,8-tetrahydroquinoline (50 mg), 3-bromo-2-fluoro-5-methylpyridine (34 mg), Pd(dppf)Cl₂—CH₂.Cl₂ (9 mg) and potassium carbonate (57 mg) was added 1,4-dioxane/water (3/1, 1.3 mL), and the mixture was degassed, and then stirred under Ar atmosphere at 100° C. for 6 hours. After the reaction mixture was allowed to return to room temperature, diluted with AcOEt, dried over anhydrous sodium sulfate, and filtered through Celite, the filtrate was evaporated under reduced pressure. The resulting residue was purified by silica gel column chromatography to give the title compound (30 mg) as a white solid.

[MS (ESI) m/z 350.4 (M+H)⁺]

### Example 109

### 5-Methyl-3-[2-(pyridin-2-ylmethoxy)-5,6,7,8-tetrahydroquinolin-4-yl]pyridine-2-carbonitrile

To 4-(2-fluoro-5-methylpyridin-3-yl)-2-(pyridin-2-ylmethoxy)-5,6,7,8-tetrahydroquinoline (20 mg) and sodium cyanide (hereinafter referred to as “NaCN”) (6 mg) was added DMSO (0.6 mL), and the mixture was stirred at 150° C. for 4 hours. The reaction mixture was added with water and Et₂O, and subjected to extraction. The organic layer was washed with saturated brine, dried over anhydrous sodium sulfate, and filtered off, and the filtrate was evaporated under reduced pressure. The resulting residue was purified by silica gel column chromatography to give the title compound (10 mg) as colorless oil.

[MS (ESI) m/z 357.4 (M+H)⁺]

### Example 110

### 4-(5-Chloro-2-fluoropyridin-3-yl)-2-(pyridin-2-ylmethoxy)-5,6,7,8-tetrahydroquinoline hydrochloride

To 2-(pyridin-2-ylmethoxy)-4-(4,4,5,5-tetramethyl-1,3,2-dioxaborolan-2-yl)-5,6,7,8-tetrahydroquinoline (100 mg), 3-bromo-5-chloro-2-fluoropyridine (76 mg), Pd(dppf)Cl₂.CH₂Cl₂ (18 mg) and potassium carbonate (116 mg) was added 1,4-dioxane/water (3/1, 2.8 mL). The mixture was degassed, and then stirred under Ar atmosphere at 100° C. for 4 hours. After the reaction mixture was allowed to return to room temperature, diluted with ethyl acetate, and filtered through Celite, the filtrate was evaporated under reduced pressure. The resulting residue was purified by silica gel column chromatography to give the title compound (64 mg) as a white powder.

[MS (ESI) m/z 370.3 (M+H)⁺]

The resulting compound (25 mg) was dissolved in Et₂O (2 mL), followed by the addition of 1 N HCl/Et₂O solution (0.067 mL), and the resulting precipitate was collected by filtration to give the title compound (21 mg) as a white powder.

[MS (ESI) m/z 370.3 (M+H)⁺]

### Example 111

### 6-Fluoro-5-[2-(pyridin-2-ylmethoxy)-5,6,7,8-tetrahydroquinolin-4-yl]pyridine-3-carbonitrile

To 4-(5-chloro-2-fluoropyridin-3-yl)-2-(pyridin-2-ylmethoxy)-5,6,7,8-tetrahydroquinoline (25 mg), zinc cyanide (17 mg), Pd₂(dba)₃.CHCl₃ (4.2 mg) and S-Phos (7 mg) were added DMF (0.6 mL) and water (0.006 mL), and the mixture was reacted under microwave irradiation at 180° C. for 30 minutes. After the reaction mixture was allowed to return to room temperature, it was added with water and ethyl acetate, and subjected to extraction. The organic layer was washed with saturated brine and saturated aqueous sodium hydrogen carbonate solution, dried over anhydrous sodium sulfate, and filtered off, and the filtrate was evaporated under reduced pressure. The resulting residue was purified by silica gel column chromatography to give the title compound (8 mg) as a colorless solid.

[MS (ESI) m/z 361.4 (M+H)⁺]

### Example 112

### 3-[2-(Pyridin-2-ylmethoxy)-5,6,7,8-tetrahydroquinolin-4-yl]pyridine-2-carboxamide

### [Step 1]

Production of 3-[2-(pyridin-2-ylmethoxy)-5,6,7,8-tetrahydroquinolin-4-yl]pyridine-2-carbonitrile

The title compound was prepared as yellow oil according to the procedure described in Example 109 using 4-(2-fluoropyridin-3-yl)-2-(pyridin-2-ylmethoxy)-5,6,7,8-tetrahydroquinoline instead of 4-(2-fluoro-5-methylpyridin-3-yl)-2-(pyridin-2-ylmethoxy)-5,6,7,8-tetrahydroquinoline.

### [Step 2]

Production of 3-[2-(pyridin-2-ylmethoxy)-5,6,7,8-tetrahydroquinolin-4-yl]pyridine-2-carboxamide

To 3-[2-(pyridin-2-ylmethoxy)-5,6,7,8-tetrahydroquinolin-4-yl]pyridine-2-carbonitrile (10 mg) was added tert-butanol (1 mL), and further added potassium fluoride on alumina (100 mg), and the mixture was stirred at 90° C. for 4 hours. After the reaction mixture was allowed to return to room temperature, diluted with ethyl acetate, and filtered through Celite, the filtrate was evaporated under reduced pressure. The resulting residue was purified by silica gel column chromatography to give the title compound (9 mg) as a white solid.

[MS (ESI) m/z 362.3 (M+H)⁺]

### Example 113

### 5-[2-(Pyridin-2-ylmethoxy)-5,6,7,8-tetrahydroquinolin-4-yl]pyridine-3-carbonitrile hydrochloride

The title compound was prepared as a white powder according to the procedure described in Example 101 using 5-bromonicotinonitrile instead of 5-bromo-2-fluoropyridine.

[MS (ESI) m/z 344.3 (M+H)⁺]

Elementary analysis as C₂₁H₁₈N₄O.HCl+0.1H₂O

Calcd. (%) C: 66.26.; H: 5.08.; N: 14.72.

Found. (%) C: 66.22.; H: 5.04.; N: 14.55.

### Example 114

### 2-(Pyridin-2-ylmethoxy)-4-[5-(trifluoromethyl)pyridin-3-yl]-5,6,7,8-tetrahydroquinoline hydrochloride

To 3-bromo-5-(trifluoromethyl)pyridine (86 mg), bis(pinacolato)diboron (116 mg), Pd₂ (dba) 3 CHCl₃ (20 mg), X-Phos (36 mg) and potassium carbonate (80 mg) was added 1,4-dioxane (3.8 mL), and the mixture was degassed, and then stirred under Ar atmosphere at 100° C. for 2 hours. After the reaction mixture was allowed to return to room temperature, diluted with ethyl acetate, and filtered through Celite, the filtrate was evaporated to give 3-(4,4,5,5-tetramethyl-1,3,2-dioxaborolan-2-yl)-5-(trifluoromethyl)pyridine. To the resulting compound (70 mg) were added Pd(OAc)₂ (6 mg), S-Phos (21 mg), potassium carbonate (104 mg) and 1,4-dioxane/water (3/1, 1.8 mL). The mixture was degassed, and then stirred under Ar atmosphere at 100° C. for 5 hours. After the reaction mixture was allowed to return to room temperature, diluted with ethyl acetate, dried over anhydrous sodium sulfate, and filtered through Celite, the filtrate was evaporated under reduced pressure. The resulting residue was purified by silica gel column chromatography to give 2-(pyridin-2-ylmethoxy)-4-[5-(trifluoromethyl)pyridin-3-yl]-5,6,7,8-tetrahydroquinoline (90 mg).

[MS (ESI) m/z 386.1 (M+H)⁺]

The resulting compound was dissolved in ethyl acetate (5.8 mL), followed by the addition of 1 N HCl/Et₂O solution (0.288 mL), and the mixture was stirred under ice water cooling for 1 hour. The resulting precipitate was collected by filtration to give the title compound (78 mg) as a white powder.

[MS (ESI) m/z 386.3 (M+H)⁺]

### Example 115

### 1-{5-[2-(Pyridin-2-ylmethoxy)-5,6,7,8-tetrahydroquinolin-4-yl]pyridin-3-yl}ethanone dihydrochloride

The title compound was prepared as a white powder according to the procedure described in Example 101 using 1-(5-bromopyridin-3-yl)ethanone instead of 5-bromo-2-fluoropyridine.

[MS (ESI) m/z 360.2 (M+H)⁺]

Elementary analysis as C₂₂H₂₁N₃O₂ 2 HCl+.7H₂O

Calcd. (%) C: 59.38.; H: 5.53.; N: 9.44.

Found. (%) C: 59.13.; H: 5.17.; N: 9.31.

### Example 116

### Methyl 5-[2-(pyridin-2-ylmethoxy)-5,6,7,8-tetrahydroquinolin-4-yl]pyridine-3-carboxylate

To 4-chloro-2-(pyridin-2-ylmethoxy)-5,6,7,8-tetrahydro quinoline (59 mg), ethyl 5-(4,4,5,5-tetramethyl-1,3,2-dioxaborolan-2-yl)pyridine-3-carboxylate (78 mg), Pd₂(dba)₃.CHCl₃ (8.2 mg) and potassium carbonate (140 mg) was added 1,4-dioxane/water (3/1, 1 mL), and the mixture was stirred at 100° C. for 24 hours. After the reaction mixture was allowed to return to room temperature, diluted with ethyl acetate, and filtered through Celite, the filtrate was evaporated under reduced pressure. The resulting residue was purified by silica gel column chromatography with ethyl acetate/methanol as eluting solvent to give the title compound (3 mg) as colorless oil.

[MS (ESI) m/z 376.3 (M+H)⁺]

### Example 117

### 5-[2-(Pyridin-2-ylmethoxy)-5,6,7,8-tetrahydroquinolin-4-yl]pyridine-3-carboxylic acid

### [Step 1]

Production of ethyl 5-[2-(pyridin-2-ylmethoxy)-5,6,7,8-tetrahydroquinolin-4-yl]pyridine-3-carboxylate

The title compound was prepared as pale yellow oil according to the procedure described in Example 103, Step 1, using ethyl 5-(4,4,5,5-tetramethyl-1,3,2-dioxaborolan-2-yl)pyridine-3-carboxylate instead of 2-fluoro-3-(4,4,5,5-tetramethyl-1,3,2-dioxaborolan-2-yl)pyridine.

### [Step 2]

Production of 5-[2-(pyridin-2-ylmethoxy)-5,6,7,8-tetrahydroquinolin-4-yl]pyridine-3-carboxylic acid

To ethyl 5-[2-(pyridin-2-ylmethoxy)-5,6,7,8-tetrahydroquinolin-4-yl]pyridine-3-carboxylate (13 mg) was added dimethylamine/water (40%, 1 mL), and the mixture was reacted under microwave irradiation at 140° C. for 0.5 hour. The reaction mixture was purified by silica gel column chromatography to give the title compound (6 mg) as a white powder.

[MS (ESI) m/z 362.2 (M+H)⁺]

### Example 118

### N-Methyl-5-[2-(pyridin-2-ylmethoxy)-5,6,7,8-tetrahydroquinolin-4-yl]pyridine-3-carboxamide hydrochloride

A mixture of ethyl 5-[2-(pyridin-2-ylmethoxy)-5,6,7,8-tetrahydroquinolin-4-yl]pyridine-3-carboxylate (15 mg) and methylamine/methanol (1 mL) was reacted under microwave irradiation at 140° C. for 0.5 hour. The solvent of the reaction mixture was evaporated under reduced pressure and the resulting residue was purified by silica gel column chromatography to give N-methyl-5-[2-(pyridin-2-ylmethoxy)-5,6,7,8-tetrahydroquinolin-4-yl]pyridine-3-carboxamide (14 mg) as colorless oil.

[MS (ESI) m/z 375.3 (M+H)⁺]

The resulting compound was dissolved in ethyl acetate (1 mL), followed by addition of 1 N HCl/Et₂O solution (0.039 mL), and then the resulting precipitate was collected by filtration to give the title compound (9 mg) as a white powder.

[MS (ESI) m/z 375.3 (M+H)⁺]

### Example 119

### N-Cyclopropyl-5-[2-(pyridin-2-ylmethoxy)-5,6,7,8-tetrahydroquinolin-4-yl]pyridine-3-carboxamide hydrochloride

To 5-[2-(pyridin-2-ylmethoxy)-5,6,7,8-tetrahydroquinolin-4-yl]pyridine-3-carboxylic acid (20 mg), cyclopropylamine (16 mg), 2-(7-aza-1H-benzotriazole-1-yl)-1,1,3,3-tetramethyl uronium hexafluorophosphate (hereinafter referred to as “HATU”) (44 mg) and Et₃N (0.04 mL) was added DMF (4 mL), and then the mixture was stirred at room temperature for 24 hours. The reaction mixture was added with water and ethyl acetate, and subjected to extraction. The organic layer was washed with saturated brine, dried over anhydrous sodium sulfate, and filtered off, and the filtrate was evaporated under reduced pressure. The resulting residue was purified by silica gel column chromatography to give N-cyclopropyl-5-[2-(pyridin-2-ylmethoxy)-5,6,7,8-tetrahydroquinolin-4-yl]pyridine-3-carboxamide (17 mg) as a white powder.

[MS (ESI) m/z 401.3 (M+H)⁺]

The resulting compound was dissolved in ethyl acetate (2 mL), followed by addition of 1 N HCl/Et₂O solution (0.039 mL), and then the solvent of the reaction mixture was evaporated under reduced pressure. Ethyl acetate was added to the resulting residue, and the insoluble portion was collected by filtration to give the title compound (5 mg) as a white powder.

[MS (ESI) m/z 401.3 (M+H)⁺]

### Example 120

### N,N-Dimethyl-5-[2-(pyridin-2-ylmethoxy)-5,6,7,8-tetrahydroquinolin-4-yl]pyridine-3-carboxamide hydrochloride

To ethyl 5-[2-(pyridin-2-ylmethoxy)-5,6,7,8-tetrahydroquinolin-4-yl]pyridine-3-carboxylate (12 mg) was added dimethylamine/water (0.5 mL), and the mixture was reacted under microwave irradiation at 140° C. for 15 minutes. The reaction mixture was filtered through Celite, and the filtrate was evaporated under reduced pressure. To the resulting residue were added dimethylamine, HATU, Et₃N and methanol, and then the mixture was reacted under microwave irradiation at 140° C. for 15 minutes. The reaction mixture was filtered through Celite, and the filtrate was evaporated under reduced pressure. The resulting residue was purified by silica gel column chromatography to give N,N-dimethyl-5-[2-(pyridin-2-ylmethoxy)-5,6,7,8-tetrahydroquinolin-4-yl]pyridine-3-carboxamide (14 mg) as colorless oil.

[MS (ESI) m/z 389.3 (M+H)⁺]

The resulting compound was dissolved in ethyl acetate, followed by the addition of 1 N HCl/Et₂O solution (0.036 mL). The resulting precipitate was collected by filtration to give the title compound (4 mg) as a white powder.

[MS (ESI) m/z 389.3 (M+H)⁺]

### Example 121

### N-(Cyclopropylmethyl)-5-[2-(pyridin-2-ylmethoxy)-5,6,7,8-tetrahydroquinolin-4-yl]pyridine-3-carboxamide

To 5-[2-(pyridin-2-ylmethoxy)-5,6,7,8-tetrahydroquinolin-4-yl]pyridine-3-carboxylic acid (25 mg), cyclopropylmethylamine (49 mg), HATU (52 mg) and DIPEA (0.12 mL) were added DMF (4 mL) and MeCN (0.004 mL), and the mixture was stirred at 40° C. for 24 hours. Cyclopropylmethylamine (49 mg), HATU (52 mg) and DIPEA (0.12 mL) were further added, and the mixture was stirred at 40° C. for 24 hours. The reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (7 mg) as colorless oil.

[MS (ESI) m/z 415.3 (M+H)⁺]

### Example 122

### {5-[2-(Pyridin-2-ylmethoxy)-5,6,7,8-tetrahydroquinolin-4-yl]pyridin-3-yl}methanol hydrochloride

To ethyl 5-[2-(pyridin-2-ylmethoxy)-5,6,7,8-tetrahydroquinolin-4-yl]pyridine-3-carboxylate (35 mg) were added THF (1 mL), NaBH₄ (23 mg) and MeOH, and the mixture was stirred at 50° C. for 3 hours. The reaction mixture was added with saturated aqueous potassium carbonate solution, and stirred at room temperature for 1 hour. The reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give {5-[2-(pyridin-2-ylmethoxy)-5,6,7,8-tetrahydroquinolin-4-yl]pyridin-3-yl}methanol (23 mg) as colorless oil.

[MS (ESI) m/z 348.3 (M+H)⁺]

The resulting compound (10 mg) was dissolved in ethyl acetate (1 mL), followed by the addition of 1 N HCl/Et₂O solution (0.029 mL). The resulting precipitate was collected by filtration to give the title compound (6 mg) as a white powder.

[MS (ESI) m/z 348.3 (M+H)⁺]

### Example 123

### 4-[5-(Methoxymethyl)pyridin-3-yl]-2-(pyridin-2-ylmethoxy)-5,6,7,8-tetrahydroquinoline hydrochloride

To {5-[2-(pyridin-2-ylmethoxy)-5,6,7,8-tetrahydroquinolin-4-yl]pyridin-3-yl}methanol (10 mg) was added THF (0.5 mL), followed by the addition of NaH (60% dispersion in oil, 2.4 mg) under ice water cooling, and the mixture was stirred for 30 minutes. After that, methyl iodide (hereinafter referred to as “MeI”) (0.0015 mL) was added, and stirred at room temperature overnight. The reaction mixture was diluted with ethyl acetate, and filtered through Celite, and the filtrate was evaporated under reduced pressure. The resulting residue was purified by silica gel column chromatography to give 4-[5-(methoxymethyl)pyridin-3-yl]-2-(pyridin-2-ylmethoxy)-5,6,7,8-tetrahydroquinoline (12 mg) as yellow oil. The resulting compound was dissolved in ethyl acetate, followed by the addition of 1 N HCl/Et₂O solution (0.028 mL). The resulting precipitate was collected by filtration to give the title compound (5 mg) as a pale yellow powder.

[MS (ESI) m/z 362.3 (M+H)⁺]

### Example 124

### 5-[2-(Pyridin-2-ylmethoxy)-5,6,7,8-tetrahydroquinolin-4-yl]pyridin-3-amine

The title compound (47 mg) was prepared as a white solid according to the procedure described in Example 103, Step 1, using 5-(4,4,5,5-tetramethyl-1,3,2-dioxaborolan-2-yl)pyridin-3-amine instead of 2-fluoro-3-(4,4,5,5-tetramethyl-1,3,2-dioxaborolan-2-yl)pyridine.

[MS (ESI) m/z 333.2 (M+H)⁺]

### Example 125

### N-Methyl-5-[2-(pyridin-2-ylmethoxy)-5,6,7,8-tetrahydroquinolin-4-yl]pyridin-3-amine

To 5-[2-(pyridin-2-ylmethoxy)-5,6,7,8-tetrahydroquinolin-4-yl]pyridin-3-amine (24 mg), formaldehyde (0.008 mL) and sodium triacetoxyborohydride (61 mg) was added MeCN (1.5 mL), and the mixture was stirred at room temperature overnight. The reaction mixture was added with water, ethyl acetate and saturated aqueous sodium hydrogen carbonate solution, and subjected to extraction. The organic layer was dried over anhydrous sodium sulfate, and filtered off, and the filtrate was evaporated under reduced pressure. The resulting residue was purified by silica gel column chromatography to give the title compound (3 mg) as a white powder.

[MS (ESI) m/z 347.2 (M+H)⁺]

### Example 126

### N,N-Dimethyl-5-[2-(pyridin-2-ylmethoxy)-5,6,7,8-tetrahydroquinolin-4-yl]pyridin-3-amine

To 5-[2-(pyridin-2-ylmethoxy)-5,6,7,8-tetrahydroquinolin-4-yl]pyridin-3-amine (24 mg), formaldehyde (0.033 mL) and sodium triacetoxyborohydride (92 mg) was added MeCN (1.5 mL), and the mixture was stirred at room temperature overnight. The reaction mixture was added with water, ethyl acetate and saturated aqueous sodium hydrogen carbonate solution, and subjected to extraction. The organic layer was dried over anhydrous sodium sulfate, and filtered off, and the filtrate was evaporated under reduced pressure. The resulting residue was purified by silica gel column chromatography to give the title compound (8 mg) as pale yellow oil.

[MS (ESI) m/z 361.2 (M+H)⁺]

### Example 127

### tert-Butyl [2-({5-[2-(pyridin-2-ylmethoxy)-5,6,7,8-tetrahydroquinolin-4-yl]pyridin-3-yl}amino)ethyl]carbamate

To tert-butyl {2-[(5-bromopyridin-3-yl)amino]ethyl}carbamate (see, for example, US2003/187026)(120 mg), bis(pinacolato)diboron (116 mg), Pd₂ (dba) 3 CHCl₃ (20 mg), X-Phos (36 mg) and potassium acetate (80 mg) was added 1,4-dioxane (7.6 mL), and the mixture was stirred at 100° C. for 4 hours. After the reaction mixture was allowed to return to room temperature, diluted with ethyl acetate, and filtered through Celite, the filtrate was evaporated. To the resulting residue were added 4-chloro-2-(pyridin-2-ylmethoxy)-5,6,7,8-tetrahydroquinoline (35 mg), Pd(OAc)₂ (6 mg), S-Phos(21 mg), potassium carbonate (100 mg) and 1,4-dioxane/water (3/1, 3.6 mL). The mixture was stirred at 100° C. for 4 hours. After the reaction mixture was allowed to return to room temperature, diluted with ethyl acetate, dried over anhydrous sodium sulfate, and filtered through Celite, the filtrate was evaporated under reduced pressure. The resulting residue was purified by silica gel column chromatography to give the title compound (49 mg) as colorless oil.

[MS (ESI) m/z 476.3 (M+H)⁺]

### Example 128

### N-{5-[2-(Pyridin-2-ylmethoxy)-5,6,7,8-tetrahydroquinolin-4-yl]pyridin-3-yl}ethane-1,2-diamine

To tert-butyl [2-({5-[2-(pyridin-2-ylmethoxy)-5,6,7,8-tetrahydroquinolin-4-yl]pyridin-3-yl}amino)ethyl]carbamate (37 mg) was added THF (2 mL), followed by the addition of 10% hydrochloric acid (2 mL) under ice water cooling, and the reaction mixture was stirred at room temperature for 10 hours. The reaction mixture was added with water, CHCl₃ and potassium carbonate, and subjected to extraction. The organic layer was dried over anhydrous sodium sulfate, and filtered off, and the filtrate was evaporated under reduced pressure to give the title compound (24 mg) as pale yellow oil.

[MS (ESI) m/z 376.2 (M+H)⁺]

### Example 129

### N,N-Dimethyl-N′-{5-[2-(pyridin-2-ylmethoxy)-5,6,7,8-tetrahydroquinolin-4-yl]pyridin-3-yl}ethane-1,2-diamine

To N-{5-[2-(pyridin-2-ylmethoxy)-5,6,7,8-tetrahydroquinolin-4-yl]pyridin-3-yl}ethane-1,2-diamine (20 mg) were added MeCN (2 mL), Et₃N (0.074 mL) and methyl p-toluenesulfonate (60 mg), and the reaction mixture was stirred at 50° C. for 10 hours. The reaction mixture was added with saturated aqueous sodium hydrogen carbonate solution and chloroform, and subjected to extraction. The organic layer was dried over anhydrous sodium sulfate, and filtered off, and the filtrate was evaporated under reduced pressure to give the title compound (3 mg) as yellow oil.

[MS (ESI) m/z 404.3 (M+H)⁺]

### Example 130

### 4-(5-Cyclopropylpyridin-3-yl)-2-(pyridin-2-ylmethoxy)-5,6,7,8-tetrahydroquinoline hydrochloride

The title compound was prepared as a white powder according to the procedure described in Example 100 using 3-cyclopropyl-5-(4,4,5,5-tetramethyl-1,3,2-dioxaborolan-2-yl)pyridine instead of 2-methyl-3-(4,4,5,5-tetramethyl-1,3,2-dioxaborolan-2-yl)pyridine.

[MS (ESI) m/z 358.3 (M+H)⁺]

### Example 131

### 1-{5-[2-(Pyridin-2-ylmethoxy)-5,6,7,8-tetrahydroquinolin-4-yl]pyridin-3-yl}azetidin-2-one hydrochloride

### [Step 1]

Production of 1-(5-bromopyridin-3-yl)azetidin-2-one

To 3,5-dibromopyridine, azetidin-2-one, copper iodide and potassium carbonate was added toluene, and the mixture was stirred at 100° C. for 6 hours. After the reaction mixture was allowed to return to room temperature, diluted with ethyl acetate, dried over anhydrous sodium sulfate, and filtered through Celite, the filtrate was evaporated under reduced pressure to give the title compound as yellow oil.

### [Step 2]

Production of 1-{5-[2-(pyridin-2-ylmethoxy)-5,6,7,8-tetrahydroquinolin-4-yl]pyridin-3-yl}azetidin-2-one hydrochloride

To 1-(5-bromopyridin-3-yl)azetidin-2-one (62 mg), bis(pinacolato) diboron (83 mg), Pd₂ (dba)₃.CHCl₃ (14 mg), X-Phos (26 mg) and potassium acetate (80 mg) was added 1,4-dioxane (5.5 mL), and the mixture was stirred at 100° C. After the reaction mixture was allowed to return to room temperature, diluted with ethyl acetate, and filtered through Celite, the filtrate was evaporated under reduced pressure. To the resulting residue were added 4-chloro-2-(pyridin-2-ylmethoxy)-5,6,7,8-tetrahydroquinoline (56 mg), Pd₂(dba)₃.CHCl₃ (14 mg), t-Bu-X-Phos (23 mg), potassium carbonate and 1,4-dioxane/water (3/1, 3.6 mL). The mixture was stirred at 100° C. for 5 hours. After the reaction mixture was allowed to return to room temperature, diluted with ethyl acetate, dried over anhydrous sodium sulfate, and filtered through Celite, the filtrate was evaporated under reduced pressure. The resulting residue was purified by silica gel column chromatography to give 1-{5-[2-(pyridin-2-ylmethoxy)-5,6,7,8-tetrahydroquinolin-4-yl]pyridin-3-yl}azetidin-2-one (58 mg) as yellow oil.

[MS (ESI) m/z 387.2 (M+H)⁺]

The resulting compound was dissolved in ethyl acetate, followed by the addition of 1 N HCl/Et₂O solution (0.148 mL), and the resulting precipitate was collected by filtration to give the title compound (63 mg) as a white powder.

[MS (ESI) m/z 387.2 (M+H)⁺]

### Example 132

### N-{5-[2-(Pyridin-2-ylmethoxy)-5,6,7,8-tetrahydroquinolin-4-yl]pyridin-3-yl}acetamide hydrochloride

To 5-[2-(pyridin-2-ylmethoxy)-5,6,7,8-tetrahydroquinolin-4-yl]pyridin-3-amine (20 mg) were added THF (1 mL), Et₃N (0.025 mL) and acetyl chloride (0.007 mL), and the mixture was stirred at room temperature for 3 hours. The reaction mixture was added with saturated aqueous sodium hydrogen carbonate solution and ethyl acetate, and subjected to extraction. The organic layer was dried over anhydrous sodium sulfate, and filtered off, and the filtrate was evaporated under reduced pressure. The resulting residue was purified by silica gel column chromatography to give N-{5-[2-(pyridin-2-ylmethoxy)-5,6,7,8-tetrahydroquinolin-4-yl]pyridin-3-yl}acetamide (20 mg) as colorless oil. The resulting compound was dissolved in ethyl acetate, followed by the addition of 1 N HCl/Et₂O solution (0.148 mL), and the resulting precipitate was collected by filtration to give the title compound (9 mg) as a white powder.

[MS (ESI) m/z 375.2 (M+H)⁺]

### Example 133

### 4-[5-(1,1-Dioxidothiomorpholin-4-yl)pyridin-3-yl]-2-(pyridin-2-ylmethoxy)-5,6,7,8-tetrahydroquinoline hydrochloride

### [Step 1]

Production of 4-(5-bromopyridin-3-yl)thiomorpholine 1,1-dioxide

To 3,5-dibromopyridine (500 mg), thiomorpholine 1,1-dioxide (343 mg), Pd₂(dba)₃.CHCl₃ (110 mg), 4,5-bis(diphenylphosphino)-9,9-dimethylxanthene (123 mg) and NaO-t-Bu (305 mg) was added toluene (6 mL) under Ar atmosphere and the reaction mixture was stirred at 100° C. for 1 hour. The mixture was filtered through Celite, and the filtrate was evaporated under reduced pressure. The resulting residue was purified by silica gel column chromatography to give the title compound (324 mg) as a brown solid.

### [Step 2]

Production of 4-[5-(1,1-dioxidothiomorpholin-4-yl)pyridin-3-yl]-2-(pyridin-2-ylmethoxy)-5,6,7,8-tetrahydroquinoline hydrochloride

4-[5-(1,1-Dioxidothiomorpholin-4-yl)pyridin-3-yl]-2-(pyridin-2-ylmethoxy)-5,6,7,8-tetrahydroquino line was prepared as yellow oil according to the procedure described in Example 127 using 4-(5-bromopyridin-3-yl)thiomorpholine 1,1-dioxide instead of tert-butyl {2-[(5-bromopyridin-3-yl)amino]ethyl}carbamate.

[MS (ESI) m/z 451.2 (M+H)⁺]

The resulting compound (92 mg) was dissolved in ethyl acetate, followed by the addition of 1 N HCl/Et₂O solution (0.204 mL), and the resulting precipitate was collected by filtration to give the title compound (50 mg) as a white powder.

[MS (ESI) m/z 451.2 (M+H)⁺]

### Example 134

### 4-[5-(Azetidin-3-yloxy)pyridin-3-yl]-2-(pyridin-2-ylmethoxy)-5,6,7,8-tetrahydroquinoline hydrochloride

### [Step 1]

Production of tert-butyl 3-[(5-bromopyridin-3-yl)oxy]azetidine-1-carboxylate

To 5-bromopyridin-3-ol (500 mg), tert-butyl 3-hydroxyazetidine-1-carboxylate (747 mg) and PPh₃ (1.13 g) were added toluene (15 mL) and diethyl azodicarboxylate (2.2 M in toluene solution, 2 mL), and the mixture was stirred at 90° C. for 2 hours. The solvent of the reaction mixture was evaporated under reduced pressure. The resulting residue was purified by silica gel column chromatography to give the title compound (820 mg) as a pink solid.

### [Step 2]

Production of tert-butyl 3-({5-[2-(pyridin-2-ylmethoxy)-5,6,7,8-tetrahydroquinolin-4-yl]pyridin-3-yl}oxy)azetidine-1-carboxylate

The title compound was prepared as yellow oil according to the procedure described in Example 127 using tert-butyl 3-[(5-bromopyridin-3-yl)oxy]azetidine-1-carboxylate instead of tert-butyl {2-[(5-bromopyridin-3-yl)amino]ethyl}carbamate.

### [Step 3]

Production of 4-[5-(azetidin-3-yloxy)pyridin-3-yl]-2-(pyridin-2-ylmethoxy)-5,6,7,8-tetrahydroquinoline hydrochloride

To tert-butyl 3-({5-[2-(pyridin-2-ylmethoxy)-5,6,7,8-tetrahydroquinolin-4-yl]pyridin-3-yl}oxy)azetidine-1-carboxylate (130 mg) were added THF (2.7 mL) and 10% hydrochloric acid (2.7 mL), and the mixture was stirred at room temperature overnight. The reaction mixture was neutralized by addition of potassium carbonate, and then extracted with CHCl₃. The organic layer was dried over anhydrous sodium sulfate, and filtered off, and the filtrate was evaporated under reduced pressure. The resulting residue was purified by silica gel column chromatography to give 4-[5-(azetidin-3-yloxy)pyridin-3-yl]-2-(pyridin-2-ylmethoxy)-5,6,7,8-tetrahydroquinoline.

[MS (ESI) m/z 389.2 (M+H)⁺]

The resulting compound (18 mg) was dissolved in ethyl acetate, followed by the addition of 1 N HCl/Et₂O solution (0.046 mL), and the resulting precipitate was collected by filtration to give the title compound (11 mg) as a white powder.

[MS (ESI) m/z 389.2 (M+H)⁺]

### Example 135

### 1-[3-({5-[2-(Pyridin-2-ylmethoxy)-5,6,7,8-tetrahydroquinolin-4-yl]pyridin-3-yl}oxy)azetidin-1-yl]ethanone

To 4-[5-(azetidin-3-yloxy)pyridin-3-yl]-2-(pyridin-2-ylmethoxy)-5,6,7,8-tetrahydroquinoline (34 mg) were added THF (1 mL), Et₃N (0.037 mL) and acetyl chloride (0.01 mL), and then the mixture was stirred at room temperature overnight. The reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (30 mg) as a white amorphous.

[MS (ESI) m/z 431.2 (M+H)⁺]

### Example 136

### 4-{5-[(1-Methylazetidin-3-yl)oxy]pyridin-3-yl}-2-(pyridin-2-ylmethoxy)-5,6,7,8-tetrahydroquinoline

To 4-[5-(azetidin-3-yloxy)pyridin-3-yl]-2-(pyridin-2-ylmethoxy)-5,6,7,8-tetrahydroquinoline (34 mg) were added THF (1 mL) and cesium carbonate (51 mg), followed by addition of MeI (0.0065 mL) under ice water cooling, and the mixture was stirred at room temperature for 12 hours. The reaction mixture was filtered through Celite, and the filtrate was evaporated under reduced pressure. The resulting residue was purified by silica gel column chromatography to give the title compound (10 mg) as colorless oil.

[MS (ESI) m/z 403.2 (M+H)⁺]

### Example 137

### 4-(2-Fluoropyridin-3-yl)-2-[(6-fluoropyridin-2-yl)methoxy]-5,6,7,8-tetrahydroquinoline

### [Step 1]

Production of 2-chloro-4-(4,4,5,5-tetramethyl-1,3,2-dioxaborolan-2-yl)-5,6,7,8-tetrahydroquinoline

The title compound was prepared as a white solid according to the procedure described in Example 9, Step 4, using 2-chloro-5,6,7,8-tetrahydroquinolin-4-yl trifluoromethanesulfonate instead of 2-chloro-6,7-dihydro-5H-cyclopenta[b]pyridin-4-yl trifluoromethanesulfonate.

### [Step 2]

Production of 2-chloro-4-(2-fluoropyridin-3-yl)-5,6,7,8-tetrahydroquinoline

To 2-chloro-4-(4,4,5,5-tetramethyl-1,3,2-dioxaborolan-2-yl)-5,6,7,8-tetrahydroquinoline (400 mg), 2-fluoro-3-iodopyridine (365 mg), Pd(dppf)Cl₂—CH₂Cl₂ (89 mg) and potassium carbonate (565 mg) was added THF/water (3/1, 6.2 mL), and the mixture was degassed, and then stirred under Ar atmosphere at 60° C. for 4 hours. After the reaction mixture was allowed to return to room temperature, diluted with ethyl acetate, and filtered through Celite, the filtrate was evaporated under reduced pressure. The resulting residue was purified by silica gel column chromatography to give the title compound (262 mg) as a white powder.

### [Step 3]

Production of 4-(2-fluoropyridin-3-yl)-2-[(6-fluoropyridin-2-yl)methoxy]-5,6,7,8-tetrahydroquinoline

To 2-chloro-4-(2-fluoropyridin-3-yl)-5,6,7,8-tetrahydroquinoline (62 mg), (6-fluoropyridin-2-yl)methanol (36 mg), Pd₂(dba)₃.CHCl₃ (15 mg), t-Bu-X-Phos (24 mg) and cesium carbonate (231 mg) was added toluene (2.6 mL), and the mixture was degassed, and then stirred under Ar atmosphere at 100° C. for 4 hours. After the reaction mixture was allowed to return to room temperature, diluted with ethyl acetate, and filtered through Celite, the filtrate was evaporated under reduced pressure. The resulting residue was purified by silica gel column chromatography to give the title compound as colorless oil.

[MS (ESI) m/z 354.4 (M+H)⁺]

### Example 138

### 4-(2-Fluoropyridin-3-yl)-2-[(5-fluoropyridin-2-yl)methoxy]-5,6,7,8-tetrahydroquinoline hydrochloride

4-(2-Fluoropyridin-3-yl)-2-[(5-fluoropyridin-2-yl)methoxy]-5,6,7,8-tetrahydroquinoline was prepared as colorless oil according to the procedure described in Example 137, Step 3, using (5-fluoropyridin-2-yl)methanol instead of (6-fluoropyridin-2-yl)methanol. The resulting compound (96 mg) was dissolved in ethyl acetate (2.7 mL), followed by the addition of 1 N HCl/Et₂O solution (0.3 mL), and the mixture was stirred at room temperature for 0.5 hour. The resulting precipitate was collected by filtration to give the title compound (113 mg) as a white powder.

[MS (ESI) m/z 354.4 (M+H)⁺]

### Example 139

### 4-(2-Fluoropyridin-3-yl)-2-[(3-fluoropyridin-2-yl)methoxy]-5,6,7,8-tetrahydroquinoline

The title compound was prepared as colorless oil according to the procedure described in Example 137, Step 3, using (3-fluoropyridin-2-yl)methanol instead of (6-fluoropyridin-2-yl)methanol.

[MS (ESI) m/z 354.4 (M+H)⁺]

### Example 140

### 5-{2-[(6-Fluoropyridin-2-yl)methoxy]-5,6,7,8-tetrahydroquinolin-4-yl}pyridine-2-carbonitrile

### [Step 1]

Production of 5-(2-chloro-5,6,7,8-tetrahydroquinolin-4-yl)pyridine-2-carbonitrile

The title compound was prepared as a white powder according to the procedure described in Example 137, Step 2, using 5-bromopyridine-2-carbonitrile instead of 2-fluoro-3-iodopyridine and using 1,4-dioxane instead of THF.

### [Step 2]

Production of 5-{2-[(6-fluoropyridin-2-yl)methoxy]-5,6,7,8-tetrahydroquinolin-4-yl}pyridine-2-carbonitrile

To 5-(2-chloro-5,6,7,8-tetrahydroquinolin-4-yl)pyridine-2-carbonitrile (90 mg), (6-fluoropyridin-2-yl)methanol (55 mg), Pd₂(dba)₃ (31 mg), t-Bu-X-Phos (28 mg) and cesium carbonate (218 mg) was added toluene (3 mL), and the mixture was degassed, and then stirred under Ar atmosphere at 100° C. for 8 hours. After the reaction mixture was allowed to return to room temperature, diluted with ethyl acetate, and filtered through Celite, the filtrate was evaporated under reduced pressure. The resulting residue was purified by silica gel column chromatography to give the title compound (57 mg) as a white powder.

[MS (ESI) m/z 361.3 (M+H)⁺]

### Example 141

### 5-{2-[(3-Fluoropyridin-2-yl)methoxy]-5,6,7,8-tetrahydroquinolin-4-yl}pyridine-2-carbonitrile

The title compound was prepared as colorless oil according to the procedure described in Example 140, Step 2, using (3-fluoropyridin-2-yl)methanol instead of (6-fluoropyridin-2-yl)methanol, and using NaO-t-Bu instead of cesium carbonate.

[MS (ESI) m/z 361.3 (M+H)⁺]

### Example 142

### 5-{2-[(3,5-Difluoropyridin-2-yl)methoxy]-5,6,7,8-tetrahydroquinolin-4-yl}pyridine-2-carbonitrile

The title compound was prepared as colorless oil according to the procedure described in Example 140, Step 2, using (3,5-difluoropyridin-2-yl)methanol instead of (6-fluoropyridin-2-yl)methanol, and using NaO-t-Bu instead of cesium carbonate.

[MS (ESI) m/z 379.3 (M+H)⁺]

### Example 143

### 2-(Pyridin-2-ylmethoxy)-4-(pyrimidin-5-yl)-5,6,7,8-tetrahydroquinoline hydrochloride

2-(Pyridin-2-ylmethoxy)-4-(pyrimidin-5-yl)-5,6,7,8-tetrahydroquinoline was prepared as a pale yellow solid according to the procedure described in Example 82, Step 2, using pyrimidin-5-ylboronic acid instead of pyridin-3-ylboronic acid, and using potassium carbonate instead of potassium phosphate. The resulting compound (100 mg) was dissolved in ethyl acetate (6.2 mL), followed by addition of 1 N HCl/Et₂O solution (0.3 mL) under ice water cooling, and the mixture was stirred for 1 hour. The resulting precipitate was collected by filtration to give the title compound (91 mg) as a white powder.

[MS (ESI) m/z 318.9 (M+H)⁺]

Elementary analysis as C₁₉H₁₈N₄O.HCl+.1H₂O

Calcd. (%) C: 63.99.; H: 5.43.; N: 15.71.

Found. (%) C: 63.83.; H: 5.42.; N: 15.53.

### Example 144

### 2-[(6-Fluoropyridin-2-yl)methoxy]-4-(pyrimidin-5-yl)-5,6,7,8-tetrahydroquinoline

### [Step 1]

Production of 2-chloro-4-(pyrimidin-5-yl)-5,6,7,8-tetrahydroquinoline

The title compound was prepared as a beige powder according to the procedure described in Example 83, Step 4, using pyrimidin-5-ylboronic acid instead of pyridin-3-ylboronic acid and using Pd(dppf)Cl₂.CH₂Cl₂ instead of Pd(PPh₃)₄.

### [Step 2]

Production of 2-[(6-fluoropyridin-2-yl)methoxy]-4-(pyrimidin-5-yl)-5,6,7,8-tetrahydroquinoline

To 2-chloro-4-(pyrimidin-5-yl)-5,6,7,8-tetrahydroquinoline (71 mg), (6-fluoropyridin-2-yl)methanol (41 mg), Pd₂(dba)₃.CHCl₃ (20 mg), t-Bu-X-Phos (33 mg) and cesium carbonate (315 mg) was added toluene (2.9 mL), and the mixture was degassed, and then stirred under Ar atmosphere at 100° C. for 14 hours. After the reaction mixture was allowed to return to room temperature, diluted with ethyl acetate, and filtered through Celite, the filtrate was evaporated under reduced pressure. The resulting residue was purified by silica gel column chromatography to give the title compound (59 mg) as a pale yellow powder.

[MS (ESI) m/z 337.3 (M+H)⁺]

### Example 145

### 2-[(5-Fluoropyridin-2-yl)methoxy]-4-(pyrimidin-5-yl)-5,6,7,8-tetrahydroquinoline

The title compound was prepared as yellow oil according to the procedure described in Example 144, Step 2, using (5-fluoropyridin-2-yl)methanol instead of (6-fluoropyridin-2-yl)methanol.

[MS (ESI) m/z 337.4 (M+H)⁺]

### Example 146

### 2-[(4-Fluoropyridin-2-yl)methoxy]-4-(pyrimidin-5-yl)-5,6,7,8-tetrahydroquinoline

### [Step 1]

Production of ethyl 4-fluoropyridine-2-carboxylate

To 2-chloro-4-fluoropyridine (300 mg), Pd(dppf)Cl₂.CH₂Cl₂ (149 mg) and DIPEA (1.18 mL) were added ethanol (2 mL) and DMF (2 mL), and the mixture was stirred under carbon monooxide atmosphere at 80° C. overnight. After the reaction mixture was allowed to return to room temperature, and evaporated under reduced pressure, the reaction mixture was added with ethyl acetate and water, and subjected to extraction. The organic layer was dried over anhydrous sodium sulfate, and filtered off, and the filtrate was evaporated under reduced pressure. The resulting residue was purified by silica gel column chromatography to give the title compound (254 mg) as brown oil.

### [Step 2]

Production of (4-fluoropyridin-2-yl)methanol

To ethyl 4-fluoropyridine-2-carboxylate (254 mg) were added MeOH and NaBH₄ (170 mg) sequentially, and then the mixture was stirred for 4 hours. After the solvent of the reaction mixture was evaporated under reduced pressure, the residue was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (126 mg) as colorless oil.

### [Step 3]

Production of 2-[(4-fluoropyridin-2-yl)methoxy]-4-(pyrimidin-5-yl)-5,6,7,8-tetrahydroquinoline

The title compound was prepared as a white powder according to the procedure described in Example 144, Step 2, using (4-fluoropyridin-2-yl)methanol instead of (6-fluoropyridin-2-yl)methanol.

[MS (ESI) m/z 337.3 (M+H)+]

### Example 147

### 2-[(3-Fluoropyridin-2-yl)methoxy]-4-(pyrimidin-5-yl)-5,6,7,8-tetrahydroquinoline

The title compound was prepared as yellow oil according to the procedure described in Example 144, Step 2, using (3-fluoropyridin-2-yl)methanol instead of (6-fluoropyridin-2-yl)methanol.

[MS (ESI) m/z 337.2 (M+H)⁺]

### Example 148

### 2-[(3,6-Difluoropyridin-2-yl)methoxy]-4-(pyrimidin-5-yl)-5,6,7,8-tetrahydroquinoline

### [Step 1]

Production of (3,6-difluoropyridin-2-yl)methanol

To a solution of methyl 3,6-difluoropyridine-2-carboxylate (300 mg) in MeOH (5 mL) was added NaBH₄ (170 mg) under ice water cooling, and then the reaction mixture was stirred at room temperature for 4 hours. Hydrochloric acid was added to the reaction mixture under ice water cooling, and the solvent was evaporated under reduced pressure. The resulting residue was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (105 mg) as colorless oil.

### [Step 2]

Production of 2-[(3,6-difluoropyridin-2-yl)methoxy]-4-(pyrimidin-5-yl)-5,6,7,8-tetrahydroquinoline

The title compound was prepared as a pale yellow powder according to the procedure described in Example 144, Step 2, using (3,6-difluoropyridin-2-yl)methanol instead of (6-fluoropyridin-2-yl)methanol and using Pd₂(dba)₃ instead of Pd₂(dba)₃.CHCl₃.

[MS (ESI) m/z 355.4 (M+H)⁺]

### Example 149

### 6-({[4-(Pyrimidin-5-yl)-5,6,7,8-tetrahydroquinolin-2-yl]oxy}methyl)pyridine-2-carbonitrile

The title compound was prepared as a pale yellow powder according to the procedure described in Example 144, Step 2, using 6-(hydroxymethyl)pyridine-2-carbonitrile instead of (6-fluoropyridin-2-yl)methanol.

[MS (ESI) m/z 344.4 (M+H)⁺]

### Example 150

### 6-({[4-(Pyrimidin-5-yl)-5,6,7,8-tetrahydroquinolin-2-yl]oxy}methyl)pyridine-3-carbonitrile

To 2-chloro-4-(pyrimidin-5-yl)-5,6,7,8-tetrahydroquino line (80 mg), 6-(hydroxymethyl)pyridine-3-carbonitrile (46 mg), Pd₂(dba)₃ (30 mg), t-Bu-X-Phos (55 mg), NaO-t-Bu (63 mg) and MS4A (150 mg) was added toluene (2 mL), and the mixture was degassed, and then stirred under Ar atmosphere at 100° C. for 5 hours. After the reaction mixture was allowed to return to room temperature, diluted with ethyl acetate, washed with water and saturated brine, dried over anhydrous magnesium sulfate, and filtered off, the filtrate was evaporated under reduced pressure. The resulting residue was purified by silica gel column chromatography to give the title compound (20 mg) as a pale yellow powder.

[MS (ESI) m/z 344.4 (M+H)⁺]

### Example 151

### 2-({[4-(Pyrimidin-5-yl)-5,6,7,8-tetrahydroquinolin-2-yl]oxy}methyl)pyridine-4-carbonitrile

The title compound was prepared as a white powder according to the procedure described in Example 150 using 2-(hydroxymethyl)pyridine-4-carbonitrile instead of 6-(hydroxymethyl)pyridine-3-carbonitrile.

[MS (ESI) m/z 344.3 (M+H)⁺]

### Example 152

### 2-[(6-Methylpyridin-2-yl)methoxy]-4-(pyrimidin-5-yl)-5,6,7,8-tetrahydroquinoline

### [Step 1]

Production of 4-(pyrimidin-5-yl)-5,6,7,8-tetrahydroquinolin-2-ol

To 2-(pyridin-2-ylmethoxy)-4-(pyrimidin-5-yl)-5,6,7,8-tetrahydroquinoline (550 mg) were added THF (12 mL) and 15% hydrochloric acid (3 mL), and the mixture was heated under reflux for 3 days. After the reaction mixture was allowed to return to room temperature, the mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (240 mg).

### [Step 2]

Production of 2-[(6-methylpyridin-2-yl)methoxy]-4-(pyrimidin-5-yl)-5,6,7,8-tetrahydroquinoline

To 4-(pyrimidin-5-yl)-5,6,7,8-tetrahydroquinolin-2-ol (20 mg), 2-(chloromethyl)-6-methylpyridine (25 mg) and silver carbonate (24 mg) was added toluene (1 mL), and the mixture was reacted under microwave irradiation at 160° C. for 50 minutes. The reaction mixture was filtered through Celite, and the filtrate was evaporated under reduced pressure. The resulting residue was purified by silica gel column chromatography to give the title compound (7 mg) as a white powder.

[MS (ESI) m/z 333.3 (M+H)⁺]

### Example 153

### 2-[(3-Methylpyridin-2-yl)methoxy]-4-(pyrimidin-5-yl)-5,6,7,8-tetrahydroquinoline

The title compound was prepared as a white powder according to the procedure described in Example 144, Step 2, using (3-methylpyridin-2-yl)methanol instead of (6-fluoropyridin-2-yl)methanol.

[MS (ESI) m/z 333.4 (M+H)⁺]

### Example 154

### 4-(Pyrimidin-5-yl)-2-{[6-(trifluoromethyl)pyridin-2-yl]methoxy}-5,6,7,8-tetrahydroquinoline hydrochloride

To 2-chloro-4-(pyrimidin-5-yl)-5,6,7,8-tetrahydroquinoline (30 mg), [6-(trifluoromethyl)pyridin-2-yl]methanol (28 mg), Pd₂(dba)₃.CHCl₃ (8.3 mg), t-Bu-X-Phos (8.3 mg) and cesium carbonate (80 mg) was added toluene (1.6 mL), and the mixture was degassed, and then stirred under Ar atmosphere at 100° C. overnight. After the reaction mixture was allowed to return to room temperature, diluted with ethyl acetate, and filtered through Celite, the filtrate was evaporated under reduced pressure. The resulting residue was purified by silica gel column chromatography to give the title compound (37 mg) as a pink solid.

[MS (ESI) m/z 388.2 (M+H)⁺]

Et₂O and 1 N HCl/Et₂O solution were added to the resulting compound, and then the mixture was evaporated under reduced pressure to give the title compound (26 mg) as a white powder.

[MS (ESI) m/z 388.2 (M+H)⁺]

### Example 155

### 2-[(3-Methoxypyridin-2-yl)methoxy]-4-(pyrimidin-5-y 1)-5,6,7,8-tetrahydroquinoline

To 2-[(3-fluoropyridin-2-yl)methoxy]-4-(pyrimidin-5-yl)-5,6,7,8-tetrahydroquinoline (5 mg) were added methanol (1 mL) and NaH (60% dispersion in oil, excess amount), and the mixture was stirred at room temperature overnight and then at 50° C. for 2 days, and reacted under microwave irradiation at 120° C. for 1 hour, and then at 100° C. for 3 days. The solvent of the reaction mixture was evaporated under reduced pressure and the resulting residue was purified by silica gel column chromatography to give the title compound (3 mg) as colorless oil.

[MS (ESI) m/z 349.3 (M+H)⁺]

### Example 156

### 6-({[4-(Pyrimidin-5-yl)-5,6,7,8-tetrahydroquinolin-2-yl]oxy}methyl)pyridine-2-carboxamide

To 6-({[4-(pyrimidin-5-yl)-5,6,7,8-tetrahydroquinolin-2-yl]oxy}methyl)pyridine-2-carbonitrile (20 mg) was added tert-butanol (1 mL), followed by the addition of potassium fluoride on alumina (10 mg), and the mixture was stirred at 90° C. for 4 hours. After the reaction mixture was allowed to return to room temperature, diluted with ethyl acetate, and filtered through Celite, the filtrate was evaporated under reduced pressure. The resulting residue was purified by silica gel column chromatography to give the title compound (25 mg) as a white powder.

[MS (ESI) m/z 362.3 (M+H)⁺]

### Example 157

### 6-({[4-(Pyrimidin-5-yl)-5,6,7,8-tetrahydroquinolin-2-yl]oxy}methyl)pyridin-2-amine

To 2-[(6-fluoropyridin-2-yl)methoxy]-4-(pyrimidin-5-yl)-5,6,7,8-tetrahydroquinoline (20 mg) was added concentrated ammonia/methanol solution (1 mL), and the mixture was stirred in a sealed tube at 110° C. for 2 days. The solvent of the reaction mixture was evaporated under reduced pressure, and the resulting residue was purified by silica gel column chromatography to give the title compound (2.3 mg) as yellow oil.

[MS (ESI) m/z 334.3 (M+H)⁺]

### Example 158

### N-Methyl-6-({[4-(pyrimidin-5-yl)-5,6,7,8-tetrahydroquinolin-2-yl]oxy}methyl)pyridin-2-amine

The title compound was prepared as a white powder according to the procedure described in Example 157 using 2 N monomethylamine/methanol solution instead of concentrated ammonia/methanol solution.

[MS (ESI) m/z 348.4 (M+H)⁺]

### Example 159

### [6-({[4-(Pyrimidin-5-yl)-5,6,7,8-tetrahydroquinolin-2-yl]oxy}methyl)pyridin-2-yl]methanol

The title compound was prepared as a pale yellow powder according to the procedure described in Example 144, Step 2, using (pyridine-2,6-diyl)bismethanol instead of (6-fluoropyridin-2-yl)methanol.

[MS (ESI) m/z 349.4 (M+H)+]

### Example 160

### 2-{[6-(Methoxymethyl)pyridin-2-yl]methoxy}-4-(pyrimidin-5-yl)-5,6,7,8-tetrahydroquinoline hydrochloride

To [6-({[4-(pyrimidin-5-yl)-5,6,7,8-tetrahydroquinolin-2-yl]oxy}methyl)pyridin-2-yl]methanol (50 mg) was added THF (1 mL), followed by the addition of NaH (60% dispersion in oil, 10 mg) under ice water cooling, and then the mixture was stirred at room temperature for 10 minutes. MeI (0.013 mL) was added to the mixture under ice water cooling, and then stirred at room temperature for 2 hours. After the reaction mixture was diluted with ethyl acetate, washed with water and saturated brine, dried over anhydrous magnesium sulfate, and filtered off, the filtrate was evaporated under reduced pressure. The resulting residue was purified by silica gel column chromatography to give the title compound (41 mg) as a white powder. To the resulting compound was added Et₂O (2 mL), followed by addition of 1 N HCl/Et₂O solution (0.31 mL) under ice water cooling, and the mixture was stirred at room temperature for 0.5 hour. The resulting precipitate was collected by filtration to give the title compound (12 mg) as a white powder.

[MS (ESI) m/z 363.4 (M+H)⁺]

### Example 161

### 2-[(6-Ethenylpyridin-2-yl)methoxy]-4-(pyrimidin-5-yl)-5,6,7,8-tetrahydroquinoline

### [Step 1]

Production of 2-bromo-6-({[tert-butyl(dimethyl)silyl]oxy}methyl)pyridine

To (6-bromopyridin-2-yl)methanol (2 g) were added CH₂Cl₂ (50 mL), tert-butylchlorodimethylsilane (1.6 g), imidazole (54 mg) and DIPEA (5.5 mL), and the mixture was stirred at room temperature overnight. After the reaction mixture was diluted with Et₂O, washed with water and saturated brine, dried over anhydrous magnesium sulfate, and filtered off, the filtrate was evaporated under reduced pressure. The resulting residue was purified by silica gel column chromatography to give the title compound (3.11 g) as colorless oil.

### [Step 2]

Production of 2-({[tert-butyl(dimethyl)silyl]oxy}methyl)-6-ethenylpyridine

To 2-bromo-6-({[tert-butyl(dimethyl)silyl]oxy}methyl)pyridine (500 mg) were added 1,4-dioxane (3 mL), tributylvinyltin (1.05 g) and Pd(PPh₃)₄ (96 mg) under Ar atmosphere, and the mixture was reacted under microwave irradiation at 120° C. for 20 minutes. Pd(PPh₃)₄ (96 mg) was further added to the reaction mixture, and the mixture was further reacted under microwave irradiation at 120° C. for 1 hour. After the reaction mixture was allowed to return to room temperature, the mixture was diluted with ethyl acetate, washed with water and saturated brine, dried over anhydrous magnesium sulfate, and filtered off, and the filtrate was evaporated under reduced pressure. The resulting residue was purified by silica gel column chromatography to give the title compound (416 mg) as colorless oil.

### [Step 3]

Production of (6-ethenylpyridin-2-yl)methanol

To 2-({[tert-butyl(dimethyl)silyl]oxy}methyl)-6-ethenylpyridine (150 mg) was added THF (3 mL), followed by the addition of 1.0 M tetrabutylammonium fluoride (hereinafter referred to as “TBAF”) in THF solution (0.35 mL) under ice water cooling, and the mixture was stirred at room temperature for 3 hours. After the reaction mixture was diluted with ethyl acetate, washed with water and saturated brine, dried over anhydrous magnesium sulfate, and filtered off, the filtrate was evaporated under reduced pressure. The resulting residue was purified by silica gel column chromatography to give the title compound (68 mg) as colorless oil.

### [Step 4]

Production of 2-[(6-ethenylpyridin-2-yl)methoxy]-4-(pyrimidin-5-yl)-5,6,7,8-tetrahydroquinoline

The title compound was prepared as a white powder according to the procedure described in Example 144, Step 2, using (6-ethenylpyridin-2-yl)methanol instead of (6-fluoropyridin-2-yl)methanol.

[MS (ESI) m/z 345.4 (M+H)+]

### Example 162

### 2-[(6-Propylpyridin-2-yl)methoxy]-4-(pyrimidin-5-yl)-5,6,7,8-tetrahydroquinoline hydrochloride

The title compound was prepared as a white powder according to the procedure described in Example 144, Step 2, using (6-propylpyridin-2-yl)methanol instead of (6-fluoropyridin-2-yl)methanol, and using Pd₂(dba)₃ instead of Pd₂(dba)₃.CHCl₃. To the resulting compound (64 mg) was added Et₂O (4 mL), followed by the addition of 1 N HCl/Et₂O solution (0.178 mL) under ice water cooling, and the mixture was stirred at room temperature for 0.5 hour. The resulting precipitate was collected by filtration to give the title compound (57 mg) as a white powder.

[MS (ESI) m/z 361.5 (M+H)⁺]

### Example 163

### 2-(Pyrazin-2-ylmethoxy)-4-(pyrimidin-5-yl)-5,6,7,8-tetrahydroquinoline

The title compound was prepared as colorless oil according to the procedure described in Example 152, Step 2, using 2-(chloromethyl)pyrazine instead of 2-(chloromethyl)-6-methylpyridine.

[MS (ESI) m/z 320.2 (M+H)⁺]

### Example 164

### 4-(Pyrimidin-5-yl)-2-(1,3-thiazol-2-ylmethoxy)-5,6,7,8-tetrahydroquinoline

The title compound was prepared as yellow oil according to the procedure described in Example 144, Step 2, using 1,3-thiazol-2-ylmethanol instead of (6-fluoropyridin-2-yl)methanol.

[MS (ESI) m/z 325.2 (M+H)⁺]

### Example 165

### 2-(1,3-Oxazol-2-ylmethoxy)-4-(pyrimidin-5-yl)-5,6,7,8-tetrahydroquinoline

The title compound was prepared as a white solid according to the procedure described in Example 144, Step 2, using 1,3-oxazol-2-ylmethanol instead of (6-fluoropyridin-2-yl)methanol.

[MS (ESI) m/z 310.2 (M+H)⁺]

### Example 166

### 2-[(1-Oxidopyridin-2-yl)methoxy]-4-(pyrimidin-5-yl)-5,6,7,8-tetrahydroquinoline

To 2-(pyridin-2-ylmethoxy)-4-(pyrimidin-5-yl)-5,6,7,8-tetrahydroquinoline (63 mg) was added chloroform (100 mL), followed by the addition of m-CPBA (with abs. 25% water, 46 mg) under ice water cooling, and the mixture was stirred at the same temperature for 1 hour, and then stirred at room temperature overnight. The reaction mixture was purified by silica gel column chromatography to give the title compound (12 mg) as a white powder.

[MS (ESI) m/z 335.3 (M+H)⁺]

### Example 167

### 4-(2-Fluoropyrimidin-5-yl)-2-(pyridin-2-ylmethoxy)-5,6,7,8-tetrahydroquinoline hydrochloride

### [Step 1]

Production of 2-fluoro-5-(trimethylstannanyl)pyrimidine

To 5-bromo-2-fluoropyrimidine (300 mg), hexamethylditin (841 mg) and Pd(PPh₃)₄ (202 mg) was added 1,4-dioxane (33 mL), and the mixture was stirred under Ar atmosphere at 100° C. for 10 hours, and stirred at room temperature for 2 days. The solvent of the reaction mixture was evaporated under reduced pressure. The resulting residue was purified by silica gel column chromatography to give the title compound (361 mg) as colorless oil.

### [Step 2]

Production of 4-(2-fluoropyrimidin-5-yl)-2-(pyridin-2-ylmethoxy)-5,6,7,8-tetrahydroquinoline hydrochloride

To 4-chloro-2-(pyridin-2-ylmethoxy)-5,6,7,8-tetrahydroquinoline (100 mg), 2-fluoro-5-(trimethylstannanyl)pyrimidine (95 mg), LiCl (46 mg) and Pd(PPh₃)₄ (42 mg) was added DMF (0.6 mL), and the mixture was reacted under microwave irradiation at 180° C. for 1 hour. After the reaction mixture was allowed to return to room temperature, diluted with ethyl acetate, and filtered through Celite, the filtrate was evaporated under reduced pressure. The resulting residue was purified by silica gel column chromatography to give 4-(2-fluoropyrimidin-5-yl)-2-(pyridin-2-ylmethoxy)-5,6,7,8-tetrahydroquinoline (14 mg) as colorless oil. The resulting compound was dissolved in ethyl acetate, followed by the addition of 1 N HCl/Et₂O solution and the resulting precipitate was collected by filtration to give the title compound (12 mg) as a white powder.

[MS (ESI) m/z 337.1 (M+H)⁺]

### Example 168

### 4-(2-Methylpyrimidin-5-yl)-2-(pyridin-2-ylmethoxy)-5,6,7,8-tetrahydroquinoline

To 2-(pyridin-2-ylmethoxy)-5,6,7,8-tetrahydroquinolin-4-yl trifluoromethanesulfonate (50 mg), 2-methyl-5-(trimethylsilyl)pyrimidine (33 mg) and Pd(PPh₃)₄ (15 mg) was added NMP (1 mL), and the mixture was stirred at 100° C. overnight. After the reaction mixture was allowed to return to room temperature, the mixture was added with Et₂O and water, and subjected to extraction. The organic layer was dried over anhydrous sodium sulfate, and filtered off, and the filtrate was evaporated under reduced pressure. The resulting residue was purified by silica gel column chromatography to give the title compound (12 mg). To the resulting compound was added Et₂O (1 mL) and 1 N HCl/Et₂O solution (0.036 mL). The reaction mixture was washed by decantation, and the solvent was evaporated under reduced pressure to give the title compound (11 mg) as a white powder.

[MS (ESI) m/z 333.4 (M+H)⁺]

### Example 169

### 5-[2-(Pyridin-2-ylmethoxy)-5,6,7,8-tetrahydroquinol in-4-yl]pyrimidin-2-amine

To 4-(2-fluoropyrimidin-5-yl)-2-(pyridin-2-ylmethoxy)-5,6,7,8-tetrahydroquinoline (8 mg) was added concentrated ammonia/methanol solution (2 mL), and the mixture was stirred at 40° C. for 3 hours. The solvent of the reaction mixture was evaporated under reduced pressure, and the resulting residue was purified by silica gel column chromatography to give the title compound (5 mg) as a white powder.

[MS (ESI) m/z 334.3 (M+H)⁺]

### Example 170

### N-Methyl-5-[2-(pyridin-2-ylmethoxy)-5,6,7,8-tetrahydroquinolin-4-yl]pyrimidin-2-amine

The title compound was prepared as a white powder according to the procedure described in Example 169 using 2 N monomethylamine/methanol solution instead of concentrated ammonia/methanol solution.

[MS (ESI) m/z 348.3 (M+H)⁺]

### Example 171

### N,N-Dimethyl-5-[2-(pyridin-2-ylmethoxy)-5,6,7,8-tetrahydroquinolin-4-yl]pyrimidin-2-amine

To 4-chloro-2-(pyridin-2-ylmethoxy)-5,6,7,8-tetrahydroquinoline (100 mg), 2-fluoro-5-(trimethylstannanyl)pyrimidine (95 mg), LiCl (46 mg) and Pd(PPh₃)₄ (42 mg) was added DMF (0.6 mL), and the mixture was reacted under microwave irradiation at 180° C. for 1 hour. After the reaction mixture was allowed to return to room temperature, diluted with ethyl acetate, and filtered through Celite, the filtrate was evaporated under reduced pressure. The resulting residue was purified by silica gel column chromatography to give the title compound (4.8 mg) as colorless oil.

[MS (ESI) m/z 362.2 (M+H)⁺]

### Example 172

### 5-[2-(Pyridin-2-ylmethoxy)-5,6,7,8-tetrahydroquinolin-4-yl]pyrimidin-2-ol

To 4-(2-fluoropyrimidin-5-yl)-2-(pyridin-2-ylmethoxy)-5,6,7,8-tetrahydroquinoline (6 mg) were added t-butyl alcohol (0.5 mL) and sodium hydroxide solution (0.5 mL), and the mixture was stirred at 90° C. for 4 hours. After the reaction mixture was allowed to return to room temperature, added with chloroform and water, and neutralized with saturated aqueous ammonium chloride solution, it was subjected to extraction. The organic layer was dried over anhydrous sodium sulfate, and filtered off, and the filtrate was evaporated under reduced pressure. The resulting residue was purified by silica gel column chromatography to give the title compound (7.5 mg) as a white powder.

[MS (ESI) m/z 335.3 (M+H)⁺]

### Example 173

### 5-[2-(Pyridin-2-ylmethoxy)-5,6,7,8-tetrahydroquinolin-4-yl]pyrimidine-2-carbonitrile hydrochloride

5-[2-(Pyridin-2-ylmethoxy)-5,6,7,8-tetrahydroquinolin-4-yl]pyrimidine-2-carbonitrile was prepared as a white solid according to the procedure described in Example 108, Step 2, using 5-bromopyrimidine-2-carbonitrile instead of 3-bromo-2-fluoro-5-methylpyridine. The resulting compound (831 mg) was dissolved in ethyl acetate (25 mL), followed by the addition of 1 N HCl/Et₂O solution (2.16 mL) under ice water cooling. The resulting precipitate was collected by filtration to give the title compound (697 mg) as a white powder.

[MS (ESI) m/z 344.4 (M+H)⁺]

Elementary analysis as C₂₀H₁₇N₅O.HCl+water

Calcd. (%) C: 60.38.; H: 5.07.; N: 17.60.

Found. (%) C: 60.98.; H: 5.14.; N: 16.93.

### Example 174

### 5-[2-(Pyridin-2-ylmethoxy)-5,6,7,8-tetrahydroquinolin-4-yl]pyrimidine-4-carbonitrile hydrochloride

### [Step 1]

Production of 5-iodo-4-{[2-(trimethylsilyl)ethoxy]methoxy}pyrimidine

To 5-iodopyrimidin-4-ol (850 mg) were added DMF (9 mL), DIPEA (2 mL) and DMAP (46 mg). 2-(Chloromethyl)ethyltrimethylsilane (1 mL) was added to the mixture under ice water cooling, and the mixture was stirred at room temperature overnight. The reaction mixture was added with Et₂O and water, and subjected to extraction. The organic layer was dried over anhydrous sodium sulfate, and filtered off, and the filtrate was evaporated under reduced pressure. The resulting residue was purified by silica gel column chromatography to give the title compound (1 g) as a yellow solid.

### [Step 2]

Production of 2-(pyridin-2-ylmethoxy)-4-(4-{[2-(trimethylsilyl)ethoxy]methoxy}pyrimidin-5-yl)-5,6,7,8-tetrahydroquinoline

The title compound was prepared as yellow oil according to the procedure described in Example 108, Step 2, using 5-iodo-4-{[2-(trimethylsilyl)ethoxy]methoxy}pyrimidine instead of 3-bromo-2-fluoro-5-methylpyridine.

### [Step 3]

Production of 5-[2-(pyridin-2-ylmethoxy)-5,6,7,8-tetrahydroquinolin-4-yl]pyrimidin-4-ol

To 2-(pyridin-2-ylmethoxy)-4-(4-{[2-(trimethylsilyl)ethoxy]methoxy}pyrimidin-5-yl)-5,6,7,8-tetrahydroquinoline (237 mg) were added CH₂Cl₂ (3.4 mL) and ethanol (0.034 mL), followed by the addition of TFA (0.5 mL) under ice water cooling, and then the mixture was stirred at room temperature for 3 hours. After the reaction mixture was neutralized with water and potassium carbonate, it was extracted with chloroform. The organic layer was dried over anhydrous sodium sulfate, and filtered off, and the filtrate was evaporated under reduced pressure. Et₂O was added to the residue, and the resulting suspension was stirred for a while, and then filtered to give the title compound (110 mg) as a white powder.

### [Step 4]

Production of 4-(4-chloropyrimidin-5-yl)-2-(pyridin-2-ylmethoxy)-5,6,7,8-tetrahydroquinoline

To 5-[2-(pyridin-2-ylmethoxy)-5,6,7,8-tetrahydroquinol in-4-yl]pyrimidin-4-ol (69 mg) were added dichloroethane (2 mL) and PPh₃ (109 mg), and the mixture was stirred for 1 hour. Carbon tetrachloride (0.059 mL) was added to the mixture, and then the mixture was stirred at 60° C. for 6 hours. The solvent of the reaction mixture was evaporated under reduced pressure. The resulting residue was purified by silica gel column chromatography to give the title compound (24 mg) as colorless oil.

[MS (ESI) m/z 353.3 (M+H)⁺]

### [Step 5]

Production of 5-[2-(pyridin-2-ylmethoxy)-5,6,7,8-tetrahydroquinol in-4-yl]pyrimidine-4-carbonitrile hydrochloride

To 4-(4-chloropyrimidin-5-yl)-2-(pyridin-2-ylmethoxy)-5,6,7,8-tetrahydroquinoline (30 mg), Pd(dppf)Cl₂.CH₂Cl₂ (3.5 mg) and zinc cyanide (10 mg) was added DMF (0.5 mL), and the mixture was reacted under microwave irradiation at 180° C. for 1 hour. After the reaction mixture was allowed to return to room temperature, diluted with ethyl acetate, and filtered through Celite, the filtrate was evaporated under reduced pressure. The resulting residue was purified by silica gel column chromatography to give the title compound (22 mg) as colorless oil.

[MS (ESI) m/z 344.4 (M+H)⁺]

The resulting compound was dissolved in ethyl acetate (2 mL), followed by addition of 1 N HCl/Et₂O solution (0.064 mL), and the resulting precipitate was collected by filtration to give the title compound (17 mg) as a white powder.

[MS (ESI) m/z 344.4 (M+H)⁺]

### Example 175

### 5-[2-(Pyridin-2-ylmethoxy)-5,6,7,8-tetrahydroquinolin-4-yl]pyrimidin-4-ol

The title compound was prepared as a white powder according to the procedure described in Example 174, Step 3.

[MS (ESI) m/z 335.3 (M+H)⁺]

### Example 176

### 4-(4-Chloropyrimidin-5-yl)-2-(pyridin-2-ylmethoxy)-5,6,7,8-tetrahydroquinoline hydrochloride

4-(4-Chloropyrimidin-5-yl)-2-(pyridin-2-ylmethoxy)-5,6,7,8-tetrahydroquinoline (24 mg) was dissolved in Et₂O (1 mL), followed by the addition of 1 N HCl/Et₂O solution (0.034 mL), and the resulting precipitate was collected by filtration to give the title compound (7 mg) as a white powder.

[MS (ESI) m/z 353.3 (M+H)⁺]

### Example 177

### 5-[2-(Pyridin-2-ylmethoxy)-5,6,7,8-tetrahydroquinolin-4-yl]pyrimidin-4-amine

To 4-(4-chloropyrimidin-5-yl)-2-(pyridin-2-ylmethoxy)-5,6,7,8-tetrahydroquinoline (30 mg) was added concentrated ammonia/methanol solution (2 mL), and the mixture was stirred in a sealed tube at 60° C. overnight. The solvent of the reaction mixture was evaporated under reduced pressure, and the resulting residue was purified by silica gel column chromatography to give the title compound (5.6 mg) as a white powder.

[MS (ESI) m/z 334.4 (M+H)⁺]

### Example 178

### 4-(4-Methoxypyrimidin-5-yl)-2-(pyridin-2-ylmethoxy)-5,6,7,8-tetrahydroquinoline

To 4-(4-chloropyrimidin-5-yl)-2-(pyridin-2-ylmethoxy)-5,6,7,8-tetrahydroquinoline (30 mg) was added concentrated ammonia/methanol solution (2 mL), and the mixture was stirred in a sealed tube at 60° C. overnight. The solvent of the reaction mixture was evaporated under reduced pressure, and the resulting residue was purified by silica gel column chromatography to give the title compound (7.8 mg) as a white powder.

[MS (ESI) m/z 349.4 (M+H)⁺]

### Example 179

### 2-(Benzyloxy)-4-(2-methylpyrimidin-5-yl)-5,6,7,8-tetrahydroquinoline

### [Step 1]

Production of 2-chloro-4-(2-methylpyrimidin-5-yl)-5,6,7,8-tetrahydroquinoline

The title compound was prepared as a white powder according to the procedure described in Example 137, Step 2, using 5-bromo-2-methylpyrimidine instead of 2-fluoro-3-iodopyridine.

### [Step 2]

Production of 2-(benzyloxy)-4-(2-methylpyrimidin-5-yl)-5,6,7,8-tetrahydroquinoline

To 2-chloro-4-(2-methylpyrimidin-5-yl)-5,6,7,8-tetrahydroquinoline (30 mg), benzylalcohol (0.016 mL), Pd₂(dba)₃ (11 mg), t-Bu-X-Phos (12 mg) and cesium carbonate (94 mg) was added toluene (1.2 mL), and the mixture was degassed, and then stirred under Ar atmosphere at 100° C. for 6 hours. After the reaction mixture was allowed to return to room temperature, the solvent of the reaction mixture was evaporated under reduced pressure. The resulting residue was purified by silica gel column chromatography to give the title compound (22 mg) as a white powder.

[MS (ESI) m/z 332.4 (M+H)⁺]

### Example 180

### 4-(2-Methylpyrimidin-5-yl)-2-(pyridin-4-ylmethoxy)-5,6,7,8-tetrahydroquinoline

The title compound was prepared as a white solid according to the procedure described in Example 179, Step 2, using pyridin-4-ylmethanol instead of benzyl alcohol.

[MS (ESI) m/z 333.4 (M+H)⁺]

### Example 181

### 4-(2-Methylpyrimidin-5-yl)-2-(pyridin-3-ylmethoxy)-5,6,7,8-tetrahydroquinoline

The title compound was prepared as a white solid according to the procedure described in Example 179, Step 2, using pyridin-3-ylmethanol instead of benzyl alcohol.

[MS (ESI) m/z 333.4 (M+H)⁺]

### Example 182

### 2-[(6-Methylpyridin-2-yl)methoxy]-4-(2-methylpyrimidin-5-yl)-5,6,7,8-tetrahydroquinoline hydrochloride

2-[(6-Methylpyridin-2-yl)methoxy]-4-(2-methylpyrimidin-5-yl)-5,6,7,8-tetrahydroquinoline was prepared as a white powder according to the procedure described in Example 179, Step 2, using (6-methylpyridin-2-yl)methanol instead of benzyl alcohol. To the resulting compound (44 mg) was added Et₂O (3 mL), followed by the addition of 1 N HCl/Et₂O solution (0.14 mL) under ice water cooling, and the mixture was stirred at room temperature for 0.5 hour. The resulting precipitate was collected by filtration to give the title compound (30 mg) as a white powder.

[MS (ESI) m/z 347.4 (M+H)⁺]

### Example 183

### 6-({[4-(2-Methylpyrimidin-5-yl)-5,6,7,8-tetrahydroquinolin-2-yl]oxy}methyl)pyridine-2-carbonitrile

### [Step 1]

Production of 2-[(1-oxidopyridin-2-yl)methoxy]-5,6,7,8-tetrahydroquinolin-4-yl trifluoromethanesulfonate

The title compound was prepared according to the procedure described in Example 8, Step 1, using 2-(pyridin-2-ylmethoxy)-5,6,7,8-tetrahydroquinolin-4-yl trifluoromethanesulfonate instead of 2-(pyridin-2-ylmethoxy)-6,7-dihydro-5H-cyclopenta[b]pyridin-4-yl trifluoromethanesulfonate.

### [Step 2]

Production of 2-[(6-cyanopyridin-2-yl)methoxy]-5,6,7,8-tetrahydroquinolin-4-yl trifluoromethanesulfonate

The title compound was prepared according to the procedure described in Example 8, Step 2, using 2-[(1-oxidopyridin-2-yl)methoxy]-5,6,7,8-tetrahydroquinolin-4-yl trifluoromethanesulfonate instead of 2-[(1-oxidopyridin-2-yl)methoxy)-6,7-dihydro-5H-cyclopenta[b]pyridin-4-yl trifluoromethanesulfonate.

### [Step 3]

Production of 6-({[4-(4,4,5,5-tetramethyl-1,3,2-dioxaborolan-2-yl)-5,6,7,8-tetrahydroquinolin-2-yl]oxy}methyl)pyridine-2-carbonitrile

The title compound was prepared as a white solid according to the procedure described in Example 8, Step 3, using 2-[(6-cyanopyridin-2-yl)methoxy]-5,6,7,8-tetrahydroquinolin-4-yl trifluoromethanesulfonate instead of 2-[(6-cyanopyridin-2-yl)methoxy]-6,7-dihydro-5H-cyclopenta[b]pyridin-4-yl trifluoromethanesulfonate.

### [Step 4]

Production of 6-({[4-(2-methylpyrimidin-5-yl)-5,6,7,8-tetrahydroquinolin-2-yl]oxy}methyl)pyridine-2-carbonitrile

The title compound was prepared as a white powder according to the procedure described in Example 108, Step 2, using 6-({[4-(4,4,5,5-tetramethyl-1,3,2-dioxaborolan-2-yl)-5,6,7,8-tetrahydroquinolin-2-yl]oxy}methyl)pyridine-2-carbonitrile instead of 2-(pyridin-2-ylmethoxy)-4-(4,4,5,5-tetramethyl-1,3,2-dioxaborolan-2-yl)-5,6,7,8-tetrahydroquinoline, and using 5-bromo-2-methylpyrimidine instead of 3-bromo-2-fluoro-5-methylpyridine.

[MS (ESI) m/z 358.4 (M+H)⁺]

### Example 184

### 6-({[4-(2-Methylpyrimidin-5-yl)-5,6,7,8-tetrahydroquinolin-2-yl]oxy}methyl)pyridine-3-carbonitrile hydrochloride

6-({[4-(2-Methylpyrimidin-5-yl)-5,6,7,8-tetrahydroquinolin-2-yl]oxy}methyl)pyridine-3-carbonitrile was prepared as a white powder according to the procedure described in Example 179, Step 2, using 6-(hydroxymethyl)pyridine-3-carbonitrile instead of benzyl alcohol. To the resulting compound (44 mg) was added ethyl acetate (3 mL), followed by addition of 1 N HCl/Et₂O solution (0.18 mL) under ice water cooling, and the mixture was stirred for 0.5 hour. The resulting precipitate was collected by filtration to give the title compound (18 mg) as a white powder.

[MS (ESI) m/z 358.4 (M+H)⁺]

### Example 185

### 2-[(6-Fluoropyridin-2-yl)methoxy]-4-(2-methylpyrimidin-5-yl)-5,6,7,8-tetrahydroquinoline

The title compound was prepared as a white powder according to the procedure described in Example 179, Step 2, using (6-fluoropyridin-2-yl)methanol instead of benzyl alcohol.

[MS (ESI) m/z 351.3 (M+H)⁺]

### Example 186

### 2-[(5-Fluoropyridin-2-yl)methoxy]-4-(2-methylpyrimidin-5-yl)-5,6,7,8-tetrahydroquinoline

The title compound was prepared as a white powder according to the procedure described in Example 179, Step 2, using (5-fluoropyridin-2-yl)methanol instead of benzyl alcohol.

[MS (ESI) m/z 351.4 (M+H)⁺]

### Example 187

### 2-[(3-Fluoropyridin-2-yl)methoxy]-4-(2-methylpyrimidin-5-yl)-5,6,7,8-tetrahydroquinoline

To 2-chloro-4-(2-methylpyrimidin-5-yl)-5,6,7,8-tetrahydroquinoline (60 mg), (3-fluoropyridin-2-yl)methanol (38 mL), Pd₂(dba)₃ (21 mg), t-Bu-X-Phos (24 mg) and cesium carbonate (150 mg) was added toluene (2 mL), and the mixture was degassed, and then stirred under Ar atmosphere at 100° C. for 11 hours. After the reaction mixture was allowed to return to room temperature, the reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography and by recycling preparative gel permeation chromatography (Japan Analytical Industry, Co. Ltd., LC-9201) to give the title compound (27 mg) as a white powder.

[MS (ESI) m/z 351.3 (M+H)⁺]

### Example 188

### 2-[(3,5-Difluoropyridin-2-yl)methoxy]-4-(2-methylpyrimidin-5-yl)-5,6,7,8-tetrahydroquinolin e

The title compound was prepared as a pale yellow powder according to the procedure described in Example 150 using 2-chloro-4-(2-methylpyrimidin-5-yl)-5,6,7,8-tetrahydroquinoline instead of 2-chloro-4-(pyrimidin-5-yl)-5,6,7,8-tetrahydroquino line, and using (3,5-difluoropyridin-2-yl)methanol instead of 6-(hydroxymethyl)pyridine-3-carbonitrile

[MS (ESI) m/z 369.4 (M+H)⁺]

### Example 189

### 2-[(3,6-Difluoropyridin-2-yl)methoxy]-4-(2-methylpyrimidin-5-yl)-5,6,7,8-tetrahydroquinolin e

The title compound was prepared as a white powder according to the procedure described in Example 179, Step 2, using (3,6-difluoropyridin-2-yl)methanol instead of benzyl alcohol.

[MS (ESI) m/z 369.3 (M+H)⁺]

### Example 190

### 2-[(3-Fluoropyridin-2-yl)methoxy]-4-(2-methoxypyrimidin-5-yl)-5,6,7,8-tetrahydroquinoline

### [Step 1]

Production of 2-chloro-4-(2-methoxypyrimidin-5-yl)-5,6,7,8-tetrahydroquinoline

The title compound was prepared as a white powder according to the procedure described in Example 137, Step 2, using 5-bromo-2-methoxypyrimidine instead of 2-fluoro-3-iodopyridine, and using 1,4-dioxane instead of THF.

### [Step 2]

Production of 2-[(3-fluoropyridin-2-yl)methoxy]-4-(2-methoxypyrimidin-5-yl)-5,6,7,8-tetrahydroquinoline

The title compound was prepared as a white powder according to the procedure described in Example 179, Step 2, using 2-chloro-4-(2-methoxypyrimidin-5-yl)-5,6,7,8-tetrahydroquinoline instead of 2-chloro-4-(2-methylpyrimidin-5-yl)-5,6,7,8-tetrahydroquinoline, and using (3-fluoropyridin-2-yl)methanol instead of benzyl alcohol.

[MS (ESI) m/z 367.4 (M+H)⁺]

### Example 191

### 5-{2-[(3-Fluoropyridin-2-yl)methoxy]-5,6,7,8-tetrahydroquinolin-4-yl}pyrimidin-2-amine hydrochloride

### [Step 1]

Production of 2-chloro-4-[(4-methoxybenzyl)oxy]-5,6,7,8-tetrahydroquinoline

To 4-methoxybenzylalcohol (2.05 g) were added DMF (50 mL) and NaH (60% dispersion in oil, 891 mg) under ice water cooling, and the mixture was stirred for 30 minutes. After the reaction mixture was allowed to return to room temperature, 2,4-dichloro-5,6,7,8-tetrahydroquinoline (see, for example, Helvetica Chimica Acta, 1945, vol. 28, p. 1684-1690) (3 g) was added to the mixture, and the mixture was stirred at 60° C. for 4 hours. After the reaction mixture was allowed to return to room temperature, the reaction mixture was added with water and ethyl acetate, and subjected to extraction. The organic layer was washed with saturated brine, dried over anhydrous sodium sulfate, and filtered off, and the filtrate was evaporated under reduced pressure. The resulting residue was purified by silica gel column chromatography to give the title compound (3.31 g) as colorless oil.

### [Step 2]

Production of 2-[(3-fluoropyridin-2-yl)methoxy]-4-[(4-methoxybenzyl) oxy]-5,6,7,8-tetrahydroquinoline

To 2-chloro-4-[(4-methoxybenzyl)oxy]-5,6,7,8-tetrahydroquinoline (200 mg), (3-fluoropyridin-2-yl)methanol (109 mL), Pd₂(dba)₃ (60 mg), t-Bu-X-Phos (67 mg) and potassium phosphate (280 mg) was added toluene (5 mL), and the mixture was degassed, and then stirred under Ar atmosphere at 100° C. for 4 hours. The reaction mixture was added with NaO-t-Bu (127 mg), and further stirred at 100° C. for 2 hours. The solvent of the reaction mixture was evaporated under reduced pressure and the resulting residue was purified by silica gel column chromatography to give the title compound (113 mg) as red oil.

### [Step 3]

Production of 2-[(3-fluoropyridin-2-yl)methoxy]-5,6,7,8-tetrahydroquinolin-4-ol

To 2-[(3-fluoropyridin-2-yl)methoxy]-4-[(4-methoxybenzyl) oxy]-5,6,7,8-tetrahydroquinoline (270 mg) and anisole (148 mg) was added CH₂Cl₂ (3 mL), followed by the addition of TFA (3 mL) under ice water cooling, and then the mixture was stirred at room temperature for 2 hours. After toluene was added to the reaction mixture, the solvent of the reaction mixture was evaporated under reduced pressure. The resulting residue was purified by silica gel column chromatography to give the title compound (149 mg) as a white powder.

### [Step 4]

Production of 2-[(3-fluoropyridin-2-yl)methoxy]-5,6,7,8-tetrahydroquinolin-4-yl trifluoromethanesulfonate

To 2-[(3-fluoropyridin-2-yl)methoxy]-5,6,7,8-tetrahydroquinolin-4-ol (147 mg) were added CH₂Cl₂ (5 mL), Et₃N (5.3 mL), DMAP (6.5 mg) and Tf₂NPh (92 mg) under ice water cooling, and the mixture was stirred at room temperature overnight. The reaction mixture was added with water and ethyl acetate, and subjected to extraction. The organic layer was washed with saturated brine, dried over anhydrous sodium sulfate, and filtered off, and the filtrate was evaporated under reduced pressure. The resulting residue was purified by silica gel column chromatography to give the title compound (220 mg) as colorless oil.

### [Step 5]

Production of 5-{2-[(3-fluoropyridin-2-yl)methoxy]-5,6,7,8-tetrahydroquinolin-4-yl}pyrimidin-2-amine hydrochloride

5-{2-[(3-Fluoropyridin-2-yl)methoxy]-5,6,7,8-tetrahydroquinolin-4-yl}pyrimidin-2-amine was prepared as a white powder according to the procedure described in Example 44, Step 5, using 2-[(3-fluoropyridin-2-yl)methoxy]-5,6,7,8-tetrahydroquinolin-4-yl trifluoromethanesulfonate instead of 2-[(3,5-difluoropyridin-2-yl)methoxy]-6,7-dihydro-5H-cyclopenta[b]pyridin-4-yl trifluoromethanesulfonate, and using 5-(4,4,5,5-tetramethyl-1,3,2-dioxaborolan-2-yl)pyrimidin-2-amine instead of 5-(4,4,5,5-tetramethyl-1,3,2-dioxaborolan-2-yl)pyrimidine-2-carbonitrile. To the resulting compound (50 mg) were added chloroform (3 mL) and methanol (1 mL), followed by the addition of 1 N HCl/Et₂O solution (0.156 mL), and the mixture was stirred for 0.5 hour. Ethyl acetate was added to the reaction mixture, and the resulting precipitate was collected by filtration to give the title compound (30 mg) as a pale yellow powder.

[MS (ESI) m/z 352.3 (M+H)⁺]

### Example 192

### 5-{2-[(5-Fluoropyridin-2-yl)methoxy]-5,6,7,8-tetrahydroquinolin-4-yl}pyrimidin-2-amine hydrochloride

### [Step 1]

Production of 2-[(5-fluoropyridin-2-yl)methoxy]-4-[(4-methoxybenzyl) oxy]-5,6,7,8-tetrahydroquinoline

To 2-chloro-4-[(4-methoxybenzyl)oxy]-5,6,7,8-tetrahydroquinoline (450 mg), (5-fluoropyridin-2-yl)methanol (226 mL), Pd₂(dba)₃ (136 mg), t-Bu-X-Phos (151 mg) and NaO-t-Bu (285 mg) was added toluene (10 mL), and the mixture was degassed, and then stirred under Ar atmosphere at 100° C. for 2 hours. After the reaction mixture was allowed to return to room temperature, the reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (287 mg) as a yellow solid.

### [Step 2]

Production of 2-[(5-fluoropyridin-2-yl)methoxy]-5,6,7,8-tetrahydroquinolin-4-ol

The title compound was prepared as a white powder according to the procedure described in Example 191, Step 3, using 2-[(5-fluoropyridin-2-yl)methoxy]-4-[(4-methoxybenzyl) oxy]-5,6,7,8-tetrahydroquinoline instead of 2-[(3-fluoropyridin-2-yl)methoxy]-4-[(4-methoxybenzyl) oxy]-5,6,7,8-tetrahydroquinoline.

### [Step 3]

Production of 2-[(5-fluoropyridin-2-yl)methoxy]-5,6,7,8-tetrahydroquinolin-4-yl trifluoromethanesulfonate

The title compound was prepared as colorless oil according to the procedure described in Example 191, Step 4, using 2-[(5-fluoropyridin-2-yl)methoxy]-5,6,7,8-tetrahydroquinolin-4-ol instead of 2-[(3-fluoropyridin-2-yl)methoxy]-5,6,7,8-tetrahydroquinolin-4-ol.

### [Step 4]

Production of 5-{2-[(5-fluoropyridin-2-yl)methoxy]-5,6,7,8-tetrahydroquinolin-4-yl}pyrimidin-2-amine hydrochloride

The title compound (10 mg) was prepared as a white powder according to the procedure described in Example 4, Step 3, using 2-[(5-fluoropyridin-2-yl)methoxy]-5,6,7,8-tetrahydroquinolin-4-yl trifluoromethanesulfonate instead of 2-(pyridin-2-ylmethoxy)-6,7-dihydro-5H-cyclopenta[b]pyridin-4-yl trifluoromethanesulfonate, and using 5-(4,4,5,5-tetramethyl-1,3,2-dioxaborolan-2-yl)pyrimidin-2-amine instead of 5-(4,4,5,5-tetramethyl-1,3,2-dioxaborolan-2-yl)pyridine-2-carbonitrile

[MS (ESI) m/z 352.3 (M+H)⁺]

### Example 193

### 5-{2-[(6-Methylpyridin-2-yl)methoxy]-5,6,7,8-tetrahydroquinolin-4-yl}pyrimidine-2-carbonitrile

### [Step 1]

Production of 4-chloro-5,6,7,8-tetrahydroquinolin-2-ol

To 5,6,7,8-tetrahydroquinolin-2,4-diol (see, for example, Helvetica Chimica Acta, 1945, vol. 28, p. 1684-1690) (65 g) was added phosphoryl chloride (150 mL) and the mixture was stirred at 180° C. for 8 hours in a sealed tube. After the mixture was cooled under ice water cooling, toluene was added to the reaction mixture, and it was evaporated under reduced pressure to remove solvent and phosphory chloride. The residue was neutralized with ethyl acetate and saturated aqueous potassium carbonate solution, and filtered through Celite, and the Celite was washed with methanol. The resulting filtrate was evaporated under reduced pressure to give the title compound (16.6 g) as a white powder.

### [Step 2]

Production of 4-chloro-5,6,7,8-tetrahydroquinolin-2-yl trifluoromethanesulfonate

The title compound was prepared as colorless oil according to the procedure described in Example 50, Step 3, using 4-chloro-5,6,7,8-tetrahydroquinolin-2-ol instead of 2-[(3-fluoropyridin-2-yl)methoxy]-6,7-dihydro-5H-cyclopenta[b]pyridin-4-ol.

### [Step 3]

Production of 4-chloro-2-[(6-methylpyridin-2-yl)methoxy]-5,6,7,8-tetrahydroquinoline

To 4-chloro-5,6,7,8-tetrahydroquinolin-2-yl trifluoromethanesulfonate (300 mg), (6-methylpyridin-2-yl)methanol (117 mL), Pd₂(dba)₃.CHCl₃ (59 mg), t-Bu-X-Phos (97 mg) and cesium carbonate (929 mg) was added toluene (9 mL), and the mixture was degassed, and then stirred under Ar atmosphere at 100° C. for 5 hours. After the reaction mixture was allowed to return to room temperature, diluted with ethyl acetate, and filtered through Celite, the filtrate was evaporated under reduced pressure. The resulting residue was purified by silica gel column chromatography to give the title compound (90 mg).

### [Step 4]

Production of 2-[(6-methylpyridin-2-yl)methoxy]-5,6,7,8-tetrahydroquinolin-4-ol

To 4-chloro-2-[(6-methylpyridin-2-yl)methoxy]-5,6,7,8-tetrahydroquinoline (90 mg) were added DMSO (1 mL), water (1 mL) and potassium hydroxide (52 mg), and the mixture was stirred under Ar atmosphere at 100° C. for 12 hours. t-Bu-X-Phos (32 mg) and Pd₂(dba)₃.CHCl₃ (19 mg) were added to the reaction mixture, and the mixture was further stirred at 100° C. for 4 hours. After the reaction mixture was allowed to return to room temperature, diluted with ethyl acetate, and filtered through Celite, the filtrate was evaporated under reduced pressure. The resulting residue was purified by silica gel column chromatography to give the title compound (40 mg) as a yellow solid.

### [Step 5]

Production of 2-[(6-methylpyridin-2-yl)methoxy]-5,6,7,8-tetrahydroquinolin-4-yl trifluoromethanesulfonate

The title compound was prepared according to the procedure described in Example 50, Step 3, using 2-[(6-methylpyridin-2-yl)methoxy]-5,6,7,8-tetrahydroquinolin-4-ol instead of 2-[(3-fluoropyridin-2-yl)methoxy]-6,7-dihydro-5H-cyclopenta[b]pyridin-4-ol.

### [Step 6]

Production of 5-{2-[(6-methylpyridin-2-yl)methoxy]-5,6,7,8-tetrahydroquinolin-4-yl}pyrimidine-2-carbonitrile

The title compound was prepared as a white solid according to the procedure described in Example 44, Step 5, using 2-[(6-methylpyridin-2-yl)methoxy]-5,6,7,8-tetrahydroquinolin-4-yl trifluoromethanesulfonate instead of 2-[(3,5-difluoropyridin-2-yl)methoxy]-6,7-dihydro-5H-cyclopenta[b]pyridin-4-yl trifluoromethanesulfonate.

[MS (ESI) m/z 358.4 (M+H)⁺]

### Example 194

### 4-(Pyrazin-2-yl)-2-(pyridin-2-ylmethoxy)-5,6,7,8-tetrahydroquinoline hydrochloride

To 4-chloro-2-(pyridin-2-ylmethoxy)-5,6,7,8-tetrahydroquinoline (50 mg), 2-(tributylstannanyl)pyrazine (100 mg) and Pd(PPh₃)₄ (41 mg) was added DMF (0.6 mL), and the mixture was reacted under microwave irradiation at 180° C. for 15 minutes. After the reaction mixture was allowed to return to room temperature, diluted with ethyl acetate, and filtered through Celite, the filtrate was evaporated under reduced pressure. The resulting residue was purified by silica gel column chromatography to give 4-(pyrazin-2-yl)-2-(pyridin-2-ylmethoxy)-5,6,7,8-tetrahydroquinoline (20 mg).

[MS (ESI) m/z 319.3 (M+H)⁺]

The resulting compound was dissolved in ethyl acetate (2.5 mL), followed by the addition of 1 N HCl/Et₂O solution (0.063 mL) under ice water cooling, and the resulting precipitate was collected by filtration to give the title compound (15 mg) as a white solid.

[MS (ESI) m/z 319.3 (M+H)⁺]

### Example 195

### 4-(6-Chloropyrazin-2-yl)-2-(pyridin-2-ylmethoxy)-5,6,7,8-tetrahydroquinoline

The title compound was prepared as colorless oil according to the procedure described in Example 108, Step 2, using 2,6-dichloropyrazine instead of 3-bromo-2-fluoro-5-methylpyridine.

[MS (ESI) m/z 353.2 (M+H)⁺]

### Example 196

### 4-(5-Chloropyrazin-2-yl)-2-(pyridin-2-ylmethoxy)-5,6,7,8-tetrahydroquinoline hydrochloride

4-(5-Chloropyrazin-2-yl)-2-(pyridin-2-ylmethox y)-5,6,7,8-tetrahydroquinoline was prepared according to the procedure described in Example 108, Step 2, using 2,5-dichloropyrazine instead of 3-bromo-2-fluoro-5-methylpyridine. The resulting compound (20 mg) was dissolved in Et₂O, followed by addition of 1 N HCl/Et₂O solution (0.057 mL), and the resulting precipitate was collected by filtration to give the title compound (18 mg) as a white powder.

[MS (ESI) m/z 353.0 (M+H)⁺]

### Example 197

### 4-(3-Chloropyrazin-2-yl)-2-(pyridin-2-ylmethoxy)-5,6,7,8-tetrahydroquinoline hydrochloride

4-(3-Chloropyrazin-2-yl)-2-(pyridin-2-ylmethox y)-5,6,7,8-tetrahydroquinoline was prepared according to the procedure described in Example 108, Step 2, using 2,3-dichloropyrazine instead of 3-bromo-2-fluoro-5-methylpyridine. The resulting compound (20 mg) was dissolved in Et₂O, followed by the addition of 1 N HCl/Et₂O solution (0.057 mL), and the resulting precipitate was collected by filtration to give the title compound (15 mg) as a white powder.

[MS (ESI) m/z 353.3 (M+H)⁺]

### Example 198

### 3-[2-(Pyridin-2-ylmethoxy)-5,6,7,8-tetrahydroquinolin-4-yl]pyrazine-2-carbonitrile hydrochloride

The title compound was prepared as a white powder according to the procedure described in Example 54, Step 2, using 4-(3-chloropyrazin-2-yl)-2-(pyridin-2-ylmethoxy)-5,6,7,8-tetrahydroquinoline instead of 4-(6-chloropyrazin-2-yl)-2-(pyridin-2-ylmethoxy)-6,7-dihydro-5H-cyclopenta[b]pyridine.

[MS (ESI) m/z 344.6 (M+H)⁺]

### Example 199

### 4-(6-Fluoropyrazin-2-yl)-2-(pyridin-2-ylmethoxy)-5,6,7,8-tetrahydroquinoline hydrochloride

To 4-(6-chloropyrazin-2-yl)-2-(pyridin-2-ylmethoxy)-5,6,7,8-tetrahydroquinoline (20 mg), cesium fluoride (86 mg) and 1,4,7,10,13,16-hexaoxacyclooctadecane (7 mg) was added MeCN (1 mL), and the mixture was heated under reflux for 15 hours. After the reaction mixture was allowed to return to room temperature, the reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give 4-(6-fluoropyrazin-2-yl)-2-(pyridin-2-ylmethoxy)-5,6,7,8-tetrahydroquinoline (13 mg). The resulting compound was dissolved in Et₂O, followed by the addition of 1 N HCl/Et₂O solution (0.057 mL), and the resulting precipitate was collected by filtration to give the title compound (15 mg) as a white powder.

[MS (ESI) m/z 336.9 (M+H)⁺]

### Example 200

### 4-(3-Fluoropyrazin-2-yl)-2-(pyridin-2-ylmethoxy)-5,6,7,8-tetrahydroquinoline hydrochloride

The title compound was prepared as a white powder according to the procedure described in Example 199 using 4-(3-chloropyrazin-2-yl)-2-(pyridin-2-ylmethoxy)-5,6,7,8-tetrahydroquinoline instead of 4-(6-chloropyrazin-2-yl)-2-(pyridin-2-ylmethoxy)-5,6,7,8-tetrahydroquinoline.

[MS (ESI) m/z 336.9 (M+H)⁺]

### Example 201

### 4-(6-Methylpyrazin-2-yl)-2-(pyridin-2-ylmethoxy)-5,6,7,8-tetrahydroquinoline

The title compound was prepared as a white powder according to the procedure described in Example 124 using 2-methyl-6-(4,4,5,5-tetramethyl-1,3,2-dioxaborolan-2-yl)pyrazine instead of 5-(4,4,5,5-tetramethyl-1,3,2-dioxaborolan-2-yl)pyridin-3-amine.

[MS (ESI) m/z 333.3 (M+H)⁺]

### Example 202

### 4-(6-Ethylpyrazin-2-yl)-2-(pyridin-2-ylmethoxy)-5,6,7,8-tetrahydroquinoline

To 4-(6-chloropyrazin-2-yl)-2-(pyridin-2-ylmethoxy)-5,6,7,8-tetrahydroquinoline (20 mg) and Pd(dppf)Cl₂CH₂Cl₂ (5 mg) were added THF (1 mL) and 1M diethylzinc/hexane solution (0.074 mL), and the mixture was degassed, and then stirred under Ar atmosphere at 50° C. for 0.5 hour. After the reaction mixture was allowed to return to room temperature, the reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (19 mg) as colorless oil.

[MS (ESI) m/z 347.3 (M+H)⁺]

### Example 203

### 4-[6-(Propan-2-yl)pyrazin-2-yl]-2-(pyridin-2-ylmeth oxy)-5,6,7,8-tetrahydroquinoline

To 4-(6-chloropyrazin-2-yl)-2-(pyridin-2-ylmethoxy)-5,6,7,8-tetrahydroquinoline (20 mg) and dichloro[1,3-bis(diphenylphosphino)propane]nickel (II) (3 mg) were added THF (1 mL) and 0.5 M 2-propylzinc bromide/THF solution (0.17 mL), and the mixture was degassed, and then stirred under Ar atmosphere at 80° C. for 5 hours and then stirred at 100° C. for 2 hours. After the reaction mixture was allowed to return to room temperature, the reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (10 mg) as colorless oil.

[MS (ESI) m/z 361.4 (M+H)⁺]

### Example 204

### {6-[2-(Pyridin-2-ylmethoxy)-5,6,7,8-tetrahydroquinolin-4-yl]pyrazin-2-yl}methanol

### [Step 1]

Production of 6-[2-(pyridin-2-ylmethoxy)-5,6,7,8-tetrahydroquinolin-4-yl]pyrazine-2-carbaldehyde

The title compound was prepared according to the procedure described in Example 108, Step 2, using 6-chloropyrazine-2-carbaldehyde instead of 3-bromo-2-fluoro-5-methylpyridine.

### [Step 2]

Production of {6-[2-(pyridin-2-ylmethoxy)-5,6,7,8-tetrahydroquinolin-4-yl]pyrazin-2-yl}methanol

To 6-[2-(pyridin-2-ylmethoxy)-5,6,7,8-tetrahydroquinol in-4-yl]pyrazine-2-carbaldehyde (50 mg) was added MeOH (1.5 mL), followed by the addition of NaBH₄ (7 mg) under ice water cooling, and the mixture was stirred for 1 hour. Ethyl acetate, hydrochloric acid, aqueous sodium hydrogen carbonate solution and water were sequentially added to the reaction mixture, and the resulting mixture was subjected to extraction. The organic layer was dried over anhydrous sodium sulfate, and filtered off, and the filtrate was evaporated under reduced pressure. The resulting residue was purified by silica gel column chromatography to give the title compound (43 mg) as a white powder.

[MS (ESI) m/z 349.3 (M+H)⁺]

### Example 205

### 4-(6-Methoxypyrazin-2-yl)-2-(pyridin-2-ylmethoxy)-5,6,7,8-tetrahydroquinoline hydrochloride

To 4-(6-chloropyrazin-2-yl)-2-(pyridin-2-ylmethoxy)-5,6,7,8-tetrahydroquinoline (20 mg) was added 28% sodium methoxide/methanol solution (1 mL), and the mixture was stirred at 60° C. for 3 hours. After the reaction mixture was allowed to return to room temperature, the mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give 4-(6-methoxypyrazin-2-yl)-2-(pyridin-2-ylmethoxy)-5,6,7,8-tetrahydroquinoline (15 mg). The resulting compound was dissolved in Et₂O, followed by addition of 1 N HCl/Et₂O solution (0.039 mL), and the resulting precipitate was collected by filtration to give the title compound (13 mg) as a white powder.

[MS (ESI) m/z 349.3 (M+H)⁺]

### Example 206

### 6-[2-(Pyridin-2-ylmethoxy)-5,6,7,8-tetrahydroquinolin-4-yl]pyrazin-2-amine hydrochloride

To 4-(6-chloropyrazin-2-yl)-2-(pyridin-2-ylmethoxy)-5,6,7,8-tetrahydroquinoline (10 mg) was added 28% ammonia solution, and the mixture was reacted under microwave irradiation at 170° C. for 2 hours. The solvent of the reaction mixture was evaporated under reduced pressure, and the resulting residue was purified by silica gel column chromatography to give the title compound (5 mg) as a white powder.

[MS (ESI) m/z 334.3 (M+H)⁺]

### Example 207

### 5-[2-(Pyridin-2-ylmethoxy)-5,6,7,8-tetrahydroquinolin-4-yl]pyrazin-2-amine

The title compound was prepared (5 mg) as a white powder according to the procedure described in Example 206 using 4-(5-chloropyrazin-2-yl)-2-(pyridin-2-ylmethoxy)-5,6,7,8-tetrahydroquinoline instead of 4-(6-chloropyrazin-2-yl)-2-(pyridin-2-ylmethoxy)-5,6,7,8-tetrahydroquinoline.

[MS (ESI) m/z 334.3 (M+H)⁺]

### Example 208

### N-Methyl-6-[2-(pyridin-2-ylmethoxy)-5,6,7,8-tetrahydroquinolin-4-yl]pyrazin-2-amine hydrochloride

To 4-(6-chloropyrazin-2-yl)-2-(pyridin-2-ylmethoxy)-5,6,7,8-tetrahydroquinoline (10 mg) was added 2 N monomethylamine/methanol solution (1 mL), and the mixture was reacted under microwave irradiation at 190° C. for 1 hour. The reaction mixture was evaporated under reduced pressure, and the resulting residue was purified by silica gel column chromatography to give N-methyl-6-[2-(pyridin-2-ylmethoxy)-5,6,7,8-tetrahydroquinolin-4-yl]pyrazin-2-amine (10 mg) as a white solid. The resulting compound was dissolved in chloroform, followed by addition of 1 N HCl/Et₂O solution (0.029 mL), and the solvent of the reaction mixture was evaporated under reduced pressure. Et₂O was added to the resulting residue, and the insoluble portion was collected by filtration to give the title compound (10 mg) as a yellow powder.

[MS (ESI) m/z 348.2 (M+H)⁺]

### Example 209

### N,N-Dimethyl-6-[2-(pyridin-2-ylmethoxy)-5,6,7,8-tetrahydroquinolin-4-yl]pyrazin-2-amine hydrochloride

To 4-(6-chloropyrazin-2-yl)-2-(pyridin-2-ylmethoxy)-5,6,7,8-tetrahydroquinoline (10 mg), dimethylamine hydrochloride (12 mg) and cesium carbonate (46 mg) was added DMF (0.5 mL), and the mixture was stirred at 120° C. for 5 hours. After the reaction mixture was allowed to return to room temperature, diluted with ethyl acetate, and filtered through Celite, the filtrate of the reaction mixture was evaporated under reduced pressure. The resulting residue was purified by silica gel column chromatography to give N,N-dimethyl-6-[2-(pyridin-2-ylmethoxy)-5,6,7,8-tetrahydroquinolin-4-yl]pyrazin-2-amine (8 mg). The resulting compound was dissolved in chloroform, followed by the addition of 1 N HCl/Et₂O solution (0.022 mL), and the solvent was evaporated under reduced pressure. Et₂O was added to the resulting residue, and the insoluble portion was collected by filtration to give the title compound (8 mg) as a yellow powder.

[MS (ESI) m/z 362.4 (M+H)⁺]

### Example 210

### N-Methyl-1-{6-[2-(pyridin-2-ylmethoxy)-5,6,7,8-tetrahydroquinolin-4-yl]pyrazin-2-yl}methanamine

To 6-[2-(pyridin-2-ylmethoxy)-5,6,7,8-tetrahydroquinolin-4-yl]pyrazine-2-carbaldehyde (15 mg) were added CH₂Cl₂ (0.5 mL) and 2 N monomethylamine/THF solution (0.043 mL), and the mixture was stirred at room temperature for 0.5 hour. Sodium triacetoxyborohydride (18 mg) and acetic acid (0.007 mL) were added to the reaction mixture, and the mixture was further stirred at room temperature for 16 hours. The mixture was diluted with ethyl acetate, and filtered through Celite, and the filtrate was evaporated under reduced pressure. The resulting residue was purified by silica gel column chromatography to give the title compound (9 mg) as colorless oil.

[MS (ESI) m/z 362.4 (M+H)⁺]

### Example 211

### N,N-Dimethyl-1-{6-[2-(pyridin-2-ylmethoxy)-5,6,7,8-tetrahydroquinolin-4-yl]pyrazin-2-yl}methanamine

The title compound was prepared as colorless oil according to the procedure described in Example 210 using 2 N dimethylamine/THF solution instead of 2 N monomethylamine/THF solution.

[MS (ESI) m/z 376.2 (M+H)⁺]

### Example 212

### 2-[(6-Fluoropyridin-2-yl)methoxy]-4-(6-methylpyrazin-2-yl)-5,6,7,8-tetrahydroquinoline

### [Step 1]

Production of 2-chloro-4-(6-methylpyrazin-2-yl)-5,6,7,8-tetrahydroquinoline

The title compound was prepared as a white powder according to the procedure described in Example 137, Step 2, using 2-chloro-6-methylpyrazine instead of 2-fluoro-3-iodopyridine.

### [Step 2]

Production of 2-[(6-fluoropyridin-2-yl)methoxy]-4-(6-methylpyrazin-2-yl)-5,6,7,8-tetrahydroquinoline

The title compound was prepared as colorless oil according to the procedure described in Example 140, Step 2, using 2-chloro-4-(6-methylpyrazin-2-yl)-5,6,7,8-tetrahydroquinoline instead of 5-(2-chloro-5,6,7,8-tetrahydroquinolin-4-yl)pyridine-2-carbonitrile.

[MS (ESI) m/z 351.4 (M+H)⁺]

### Example 213

### 2-[(5-Fluoropyridin-2-yl)methoxy]-4-(6-methylpyrazin-2-yl)-5,6,7,8-tetrahydroquinoline

The title compound was prepared as a white powder according to the procedure described in Example 212, Step 2, using (5-fluoropyridin-2-yl)methanol instead of (6-fluoropyridin-2-yl)methanol.

[MS (ESI) m/z 351.4 (M+H)⁺]

### Example 214

### 2-[(3-Fluoropyridin-2-yl)methoxy]-4-(6-methylpyrazin-2-yl)-5,6,7,8-tetrahydroquinoline

The title compound was prepared as colorless oil according to the procedure described in Example 212, Step 2, using (3-fluoropyridin-2-yl)methanol instead of (6-fluoropyridin-2-yl)methanol.

[MS (ESI) m/z 351.4 (M+H)⁺]

### Example 215

### (6-{2-[(6-Fluoropyridin-2-yl)methoxy]-5,6,7,8-tetrahydroquinolin-4-yl}pyrazin-2-yl)methanol

### [Step 1]

Production of [6-(2-chloro-5,6,7,8-tetrahydroquinolin-4-yl)pyrazin-2-yl]methanol

The title compound was prepared as a yellow solid according to the procedure described in Example 137, Step 2, using (6-chloropyradin-2-yl)methanol instead of 2-fluoro-3-iodopyridine, and using 1,4-dioxane instead of THF.

### [Step 2]

Production of 4-[6-({[tert-butyl(dimethyl)silyl]oxy}methyl)pyrazin-2-yl]-2-chloro-5,6,7,8-tetrahydroquinoline

The title compound was prepared as colorless oil according to the procedure described in Example 161, Step 1, using [6-(2-chloro-5,6,7,8-tetrahydroquinolin-4-yl)pyrazin-2-yl]methanol instead of (6-bromopyridin-2-yl)methanol.

### [Step 3]

Production of 4-[6-({[tert-butyl(dimethyl)silyl]oxy}methyl)pyrazin-2-yl]-2-[(6-fluoropyridin-2-yl)methoxy]-5,6,7,8-tetrahydroquinoline

The title compound was prepared as yellow oil according to the procedure described in Example 140, Step 2, using 4-[6-({[tert-butyl(dimethyl)silyl]oxy}methyl)pyrazin-2-yl]-2-chloro-5,6,7,8-tetrahydroquinoline instead of 5-(2-chloro-5,6,7,8-tetrahydroquinolin-4-yl)pyridine-2-carbonitrile.

### [Step 4]

Production of (6-{2-[(6-fluoropyridin-2-yl)methoxy]-5,6,7,8-tetrahydroquinolin-4-yl}pyrazin-2-yl)methanol

To 4-[6-({[tert-butyl(dimethyl)silyl]oxy}methyl)pyrazin-2-yl]-2-[(6-fluoropyridin-2-yl)methoxy]-5,6,7,8-tetrahydroquinoline (60 mg) were added THF (2.4 mL) and 1.0 M TBAF/THF solution (0.375 mL), and the mixture was stirred at room temperature for 12 hours. The solvent of the reaction mixture was evaporated under reduced pressure. The resulting residue was purified by silica gel column chromatography to give the title compound (29 mg) as yellow oil.

[MS (ESI) m/z 367.4 (M+H)⁺]

### Example 216

### (6-{2-[(5-Fluoropyridin-2-yl)methoxy]-5,6,7,8-tetrahydroquinolin-4-yl}pyrazin-2-yl)methanol

[Step 1] Production of 4-[6-({[tert-butyl(dimethyl)silyl]oxy}methyl)pyrazin-2-yl]-2-[(5-fluoropyridin-2-yl)methoxy]-5,6,7,8-tetrahydroquinoline

To 4-[6-({[tert-butyl(dimethyl)silyl]oxy}methyl)pyrazin-2-yl]-2-chloro-5,6,7,8-tetrahydroquinoline (80 mg), (5-fluoropyridin-2-yl)methanol (31 mg), Pd₂(dba)₃ (11 mg), t-Bu-X-Phos (21 mg) and NaO-t-Bu (39 mg) was added toluene (2 mL), and the mixture was degassed, and then stirred under Ar atmosphere at 100° C. overnight. After the reaction mixture was allowed to return to room temperature, diluted with ethyl acetate, and filtered through Celite, the filtrate was evaporated under reduced pressure. The resulting residue was purified by silica gel column chromatography to give the title compound (59 mg) as yellow oil.

### [Step 2]

Production of (6-{2-[(5-fluoropyridin-2-yl)methoxy]-5,6,7,8-tetrahydroquinolin-4-yl}pyrazin-2-yl)methanol

The title compound was prepared as yellow oil according to the procedure described in Example 215, Step 4, using 4-[6-({[tert-butyl(dimethyl)silyl]oxy}methyl)pyrazin-2-yl]-2-[(5-fluoropyridin-2-yl)methoxy]-5,6,7,8-tetrahydroquinoline instead of 4-[6-({[tert-butyl(dimethyl)silyl]oxy}methyl)pyrazin-2-yl]-2-[(6-fluoropyridin-2-yl)methoxy]-5,6,7,8-tetrahydroquinoline.

[MS (ESI) m/z 367.4 (M+H)⁺]

### Example 217

### (6-{2-[(3-Fluoropyridin-2-yl)methoxy]-5,6,7,8-tetrahydroquinolin-4-yl}pyrazin-2-yl)methanol

### [Step 1]

Production of 4-[6-({[tert-butyl(dimethyl)silyl]oxy}methyl)pyrazin-2-yl]-2-[(3-fluoropyridin-2-yl)methoxy]-5,6,7,8-tetrahydroquinoline

The title compound was prepared according to the procedure described in Example 216, Step 1, using (3-fluoropyridin-2-yl)methanol instead of (5-fluoropyridin-2-yl)methanol.

### [Step 2]

Production of (6-{2-[(3-fluoropyridin-2-yl)methoxy]-5,6,7,8-tetrahydroquinolin-4-yl}pyrazin-2-yl)methanol

The title compound was prepared as yellow oil according to the procedure described in Example 215, Step 4, using 4-[6-({[tert-butyl(dimethyl)silyl]oxy}methyl)pyrazin-2-yl]-2-[(3-fluoropyridin-2-yl)methoxy]-5,6,7,8-tetrahydroquinoline instead of 4-[6-({[tert-butyl(dimethyl)silyl]oxy}methyl)pyrazin-2-yl]-2-[(6-fluoropyridin-2-yl)methoxy]-5,6,7,8-tetrahydroquinoline.

[MS (ESI) m/z 367.4 (M+H)⁺]

### Example 218

### (6-{2-[(3,5-Difluoropyridin-2-yl)methoxy]-5,6,7,8-tetrahydroquinolin-4-yl}pyrazin-2-yl)methanol

### [Step 1]

Production of 4-[6-({[tert-butyl(dimethyl)silyl]oxy}methyl)pyrazin-2-yl]-2-[(3,5-difluoropyridin-2-yl)methoxy]-5,6,7,8-tetrahydroquinoline

The title compound was prepared according to the procedure described in Example 216, Step 1, using (3,5-difluoropyridin-2-yl)methanol instead of (5-fluoropyridin-2-yl)methanol.

### [Step 2]

Production of (6-{2-[(3,5-difluoropyridin-2-yl)methoxy]-5,6,7,8-tetrahydroquinolin-4-yl}pyrazin-2-yl)methanol

The title compound was prepared as yellow oil according to the procedure described in Example 215, Step 4, using 4-[6-({[tert-butyl(dimethyl)silyl]oxy}methyl)pyrazin-2-yl]-2-[(3,5-difluoropyridin-2-yl)methoxy]-5,6,7,8-tetrahydroquinoline instead of 4-[6-({[tert-butyl(dimethyl)silyl]oxy}methyl)pyrazin-2-yl]-2-[(6-fluoropyridin-2-yl)methoxy]-5,6,7,8-tetrahydroquinoline.

[MS (ESI) m/z 385.4 (M+H)⁺]

### Example 219

### 6-({[4-(6-Methoxypyrazin-2-yl)-5,6,7,8-tetrahydroquinolin-2-yl]oxy}methyl)pyridine-2-carbonitrile

The title compound was prepared as a white powder according to the procedure described in Example 108, Step 2, using 6-({[4-(4,4,5,5-tetramethyl-1,3,2-dioxaborolan-2-yl)-5,6,7,8-tetrahydroquinolin-2-yl]oxy}methyl)pyridine-2-carbonitrile instead of 2-(pyridin-2-ylmethoxy)-4-(4,4,5,5-tetramethyl-1,3,2-dioxaborolan-2-yl)-5,6,7,8-tetrahydroquinoline, and using 2-chloro-6-methoxypyrazine instead of 3-bromo-2-fluoro-5-methylpyridine.

[MS (ESI) m/z 374.4 (M+H)⁺]

### Example 220

### 4-(3,4-Difluorophenyl)-2-(pyridin-2-ylmethoxy)-5,6,7,8-tetrahydroquinoline

The title compound was prepared as a white solid according to the procedure described in Example 108, Step 2, using 4-bromo-1,2-difluorobenzene instead of 3-bromo-2-fluoro-5-methylpyridine.

[MS (ESI) m/z 353.0 (M+H)⁺]

### Example 221

### 4-(3-Methoxyphenyl)-2-(pyridin-2-ylmethoxy)-5,6,7,8-tetrahydroquinoline

The title compound was prepared as a white solid according to the procedure described in Example 108, Step 2, using 1-bromo-3-methoxybenzene instead of 3-bromo-2-fluoro-5-methylpyridine.

[MS (ESI) m/z 348.3 (M+H)⁺]

### Example 222

### 4-(4-Methylpyridin-2-yl)-2-(pyridin-2-ylmethoxy)-5,6,7,8-tetrahydroquinoline

To 4-chloro-2-(pyridin-2-ylmethoxy)-5,6,7,8-tetrahydroquinoline (100 mg), 4-methyl-2-(tributylstannanyl)pyridine (139 mg) and Pd(PPh₃)₄ (42 mg) was added 1,4-dioxane (0.5 mL), and the mixture was reacted under microwave irradiation at 160° C. for 1 hour. After the reaction mixture was allowed to return to room temperature, diluted with ethyl acetate, and filtered through Celite, the filtrate was evaporated under reduced pressure. The resulting residue was purified by silica gel column chromatography to give the title compound (54 mg) as colorless oil.

[MS (ESI) m/z 332.3 (M+H)⁺]

### Example 223

### 4-(4-Fluoropyridin-2-yl)-2-(pyridin-2-ylmethoxy)-5,6,7,8-tetrahydroquinoline

The title compound was prepared as a white solid according to the procedure described in Example 108, Step 2, using 2-chloro-4-fluoropyridine instead of 3-bromo-2-fluoro-5-methylpyridine.

[MS (ESI) m/z 336.3 (M+H)⁺]

### Example 224

### 2-(Pyridin-2-ylmethoxy)-4-(1,3-thiazol-5-yl)-5,6,7,8-tetrahydroquinoline

To 4-chloro-2-(pyridin-2-ylmethoxy)-5,6,7,8-tetrahydro quinoline (30 mg), 5-(tributylstannanyl)-1,3-thiazole (82 mg) and Pd(PPh₃)₄ (25 mg) was added DMF (0.8 mL), and the mixture was stirred at 100° C. for 6 hours. The mixture was filtered through Celite, and the filtrate was evaporated under reduced pressure. The resulting residue was purified by silica gel column chromatography to give the title compound (13 mg) as colorless oil.

[MS (ESI) m/z 324.3 (M+H)⁺]

### Example 225

### 4-(2-Methyl-1,3-thiazol-5-yl)-2-(pyridin-2-ylmethoxy)-5,6,7,8-tetrahydroquinoline hydrochloride

4-(2-Methyl-1,3-thiazol-5-yl)-2-(pyridin-2-ylmethoxy)-5,6,7,8-tetrahydroquinoline was prepared as yellow oil according to the procedure described in Example 222 using 2-methyl-5-(tributylstannanyl)-1,3-thiazole instead of 4-methyl-2-(tributylstannanyl)pyridine, and using DMF instead of 1,4-dioxane. The resulting compound (68 mg) was dissolved in ethyl acetate (4 mL), followed by the addition of 1 N HCl/Et₂O solution (0.2 mL) under ice water cooling, and the mixture was stirred at the same temperature for 2 hours. The resulting precipitate was collected by filtration to give the title compound (65 mg) as a white powder.

[MS (ESI) m/z 338.2 (M+H)⁺]

### Example 226

### {5-[2-(Pyridin-2-ylmethoxy)-5,6,7,8-tetrahydroquinolin-4-yl]-1,3-thiazol-2-yl}methanol hydrochloride

To 2-(pyridin-2-ylmethoxy)-4-(1,3-thiazol-5-yl)-5,6,7,8-tetrahydroquinoline (84 mg) was added THF (2.6 mL), and 1.1 M lithium hexamethyldisilazide/THF solution (0.71 mL) was added to the mixture at −78° C., and then the mixture was stirred for 0.5 hour. Then, DMF (0.06 mL) was added to the reaction mixture, and the mixture was further stirred for 1 hour. The reaction mixture was added with water and ethyl acetate, and subjected to extraction. The organic layer was dried over anhydrous sodium sulfate, and filtered off, and the filtrate was evaporated under reduced pressure. The resulting residue was dissolved in MeOH (2 mL), and NaBH₄ (13 mg) was added to the mixture, and then the mixture was stirred for 1 hour. The reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give {5-[2-(pyridin-2-ylmethoxy)-5,6,7,8-tetrahydroquinolin-4-yl]-1,3-thiazol-2-yl}methanol (19 mg) as colorless oil.

The resulting compound (9 mg) was dissolved in ethyl acetate (2 mL), followed by the addition of 1 N HCl/Et₂O solution (0.04 mL), and the resulting precipitate was collected by filtration to give the title compound (4.3 mg) as a white powder.

[MS (ESI) m/z 354.1 (M+H)⁺]

### Example 227

4-[2-(Methoxymethyl)-1,3-thiazol-5-yl]-2-(pyridin-2-ylmethoxy)-5,6,7,8-tetrahydroquinoline

To {5-[2-(pyridin-2-ylmethoxy)-5,6,7,8-tetrahydroquinolin-4-yl]-1,3-thiazol-2-yl}methanol (10 mg) were added THF (1 mL), NaH (60% dispersion in oil, 3 mg), and MeI (0.0053 mL) sequentially under ice water cooling, and the mixture was stirred at room temperature for 4 hours.

The reaction mixture was purified by silica gel column chromatography to give the title compound (3 mg) as colorless oil.

[MS (ESI) m/z 368.3 (M+H)⁺]

### Example 228

### 4-{2-[(Propan-2-yloxy)methyl]-1,3-thiazol-5-yl}-2-(pyridin-2-ylmethoxy)-5,6,7,8-tetrahydroquinoline

To {5-[2-(pyridin-2-ylmethoxy)-5,6,7,8-tetrahydroquinolin-4-yl]-1,3-thiazol-2-yl}methanol (39 mg) were added CH₂Cl₂ (2 mL), carbon tetrabromide (43 mg), and PPh₃ (35 mg), and the mixture was stirred at room temperature for 3 hours. The reaction mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography. To the obtained product (colorless oil, 10 mg) was added 2-propanol (2 mL), followed by the addition of NaH (60% dispersion in oil, 4 mg) under ice water cooling, and the mixture was stirred at room temperature overnight. The solvent of the reaction mixture was evaporated under reduced pressure, and then the residue was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (6 mg) as colorless oil.

[MS (ESI) m/z 396.3 (M+H)⁺]

### Example 229

### 2-[2-(Pyridin-2-ylmethoxy)-5,6,7,8-tetrahydroquinolin-4-yl]-1,3-thiazole-5-carbonitrile

The title compound was prepared as a white solid according to the procedure described in Example 108, Step 2, using 2-bromo-1,3-thiazole-5-carbonitrile instead of 3-bromo-2-fluoro-5-methylpyridine.

[MS (ESI) m/z 349.3 (M+H)⁺]

### Example 230

### 6-(2-(Pyridin-2-ylmethoxy)-5,6,7,8-tetrahydroquinolin-4-yl)pyrazin-2-ol

To 4-(6-chloropyrazin-2-yl)-2-(pyridin-2-ylmethoxy)-5,6,7,8-tetrahydroquinoline (10 mg), potassium hydroxide (5 mg), Pd₂(dba)₃ (1.3 mg) and t-Bu-X-Phos (2 mg) were added 1,4-dioxane (0.5 mL) and water (0.5 mL), and the mixture was degassed, and then stirred under Ar atmosphere at 100° C. for 1 hour. After the reaction mixture was allowed to return to room temperature, the mixture was dried over anhydrous sodium sulfate, and filtered through Celite, and the filtrate was evaporated under reduced pressure. The resulting residue was purified by silica gel column chromatography to give the title compound (10 mg) as a white powder.

[MS (ESI) m/z 335.1 (M+H)⁺]

### Example 231

### 4-(1-Oxidopyrimidin-5-yl)-2-(pyridin-2-ylmethoxy)-5,6,7,8-tetrahydroquinoline

The title compound was prepared as a pale brown solid according to the procedure described in Example 108, Step 2, using 5-bromopyrimidine 1-oxide instead of 3-bromo-2-fluoro-5-methylpyridine.

[MS (ESI) m/z 335.3 (M+H)⁺]

### Example 232

### 4-(Pyridin-3-yl)-2-(pyridin-2-ylmethoxy)-6,7,8,9-tetrahydro-5H-cyclohepta[b]pyridine

### [Step 1]

Production of 4-chloro-2-(pyridin-2-ylmethoxy)-6,7,8,9-tetrahydro-5H-cyclohepta[b]pyridine

The title compound was prepared as yellow oil according to the procedure described in Example 1, Step 1, using 2,4-dichloro-6,7,8,9-tetrahydro-5H-cyclohepta[b]pyridine (see, for example, Helvetica Chimica Acta, 1944, vol. 27, p. 1854-1858) instead of 2,4-dichloro-6,7-dihydro-5H-cyclopenta[b]pyridine. [Rf value (TLC silica gel plate 60F₂₅₄, developing solvent:hexane:ethyl acetate=2:1):0.6]

### [Step 2]

Production of 2-(pyridin-2-ylmethoxy)-4-(pyrimidin-5-yl-6,7,8,9-tetrahydro-5H-cyclohepta[b]pyridine

To 4-chloro-2-(pyridin-2-ylmethoxy)-6,7,8,9-tetrahydro-5H-cyclohepta[b]pyridine (18 mg), pyridin-3-ylboronic acid (15 mg), Pd(dppf)Cl₂—CH₂Cl₂ (10 mg) and potassium carbonate (26 mg) was added 1,4-dioxane/water (3/1, 1 mL), and the mixture was stirred at 100° C. for 4 hours. After the reaction mixture was allowed to return to room temperature, diluted with ethyl acetate, and filtered through Celite, the filtrate was evaporated under reduced pressure. The resulting residue was purified by silica gel column chromatography to give the title compound (5 mg) as a white solid.

[MS (ESI) m/z 332.3 (M+H)⁺]

### Example 233

### 2-(Pyridin-2-ylmethoxy)-4-(pyrimidin-5-yl)-6,7,8,9-tetrahydro-5H-cyclohepta[b]pyridine hydrochloride

The title compound was prepared as a white powder according to the procedure described in Example 12 using 4-chloro-2-(pyridin-2-ylmethoxy)-6,7,8,9-tetrahydro-5H-cyclohepta[b]pyridine instead of 4-chloro-2-(pyridin-2-ylmethoxy)-6,7-dihydro-5H-cyclopenta[b]pyridine.

[MS (ESI) m/z 333.3 (M+H)⁺]

### Example 234

### 4-(Pyrazin-2-yl)-2-(pyridin-2-ylmethoxy)-6,7,8,9-tetrahydro-5H-cyclohepta[b]pyridine hydrochloride

To 4-chloro-2-(pyridin-2-ylmethoxy)-6,7,8,9-tetrahydro-5H-cyclohepta[b]pyridine (70 mg), 2-(tributylstannanyl)pyrazine (166 mg) and Pd(PPh₃)₄ (55 mg) was added DMF (0.6 mL), and the mixture was reacted under microwave irradiation at 180° C. for 30 minutes. After the reaction mixture was allowed to return to room temperature, diluted with ethyl acetate, and filtered through Celite, the filtrate was evaporated under reduced pressure. The resulting residue was purified by silica gel column chromatography to give 4-(pyrazin-2-yl)-2-(pyridin-2-ylmethoxy)-6,7,8,9-tetrahydro-5H-cyclohepta[b]pyridine (94 mg).

[MS (ESI) m/z 333.2 (M+H)⁺]

The resulting compound was dissolved in ethyl acetate (3.1 mL), followed by the addition of 1 N HCl/Et₂O solution (0.078 mL) under ice water cooling, and then stirred at the same temperature for 1 hour.

The resulting precipitate was collected by filtration to give the title compound (23 mg) as a white powder.

### Example 235

### 2-(Pyridin-2-ylmethoxy)-4-(pyrimidin-5-yl)-6,7-dihydro-5H-cyclopenta[b]pyridin-7-ol hydrochloride

### [Step 1]

Production of 4-chloro-2-(pyridin-2-ylmethoxy)-6,7-dihydro-5H-cyclopenta[b]pyridine 1-oxide

To 2,4-dichloro-6,7-dihydro-5H-cyclopenta[b]pyridine 1-oxide (1 g) and pyridin-2-ylmethanol (0.521 mL) were added DMF (10 mL) and NaH (60% dispersion in oil, 274 mg) under Ar atmosphere under ice water cooling, and the mixture was stirred for 1 hour. The reaction mixture was purified by silica gel column chromatography to give the title compound (1.28 g).

### [Step 2]

Production of 4-chloro-2-(pyridin-2-ylmethoxy)-6,7-dihydro-5H-cyclopenta[b]pyridin-7-yl acetate

To 4-chloro-2-(pyridin-2-ylmethoxy)-6,7-dihydro-5H-cyclopenta[b]pyridine 1-oxide (1.28 g) was added acetic anhydride (15 mL), and the mixture was stirred at 90° C. for 2 hours. After the reaction mixture was allowed to return to room temperature, excess acetic anhydride was evaporated under reduced pressure. The resulting residue was neutralized with Et₂O and saturated aqueous sodium hydrogen carbonate solution, and then extracted with Et₂O. The organic layer was dried over anhydrous sodium sulfate, and filtered off, and the filtrate was evaporated under reduced pressure. The resulting residue was purified by silica gel column chromatography to give the title compound (1.25 mg) as yellow oil.

### [Step 3]

Production of 2-(pyridin-2-ylmethoxy)-4-(pyrimidin-5-yl)-6,7-dihydro-5H-cyclopenta[b]pyridin-7-ol hydrochloride

2-(Pyridin-2-ylmethoxy)-4-(pyrimidin-5-yl)-6,7-dihydro-5H-cyclopenta[b]pyridin-7-ol (95 mg) was prepared as a white powder according to the procedure described in Example 58 using 4-chloro-2-(pyridin-2-ylmethoxy)-6,7-dihydro-5H-cyclopenta[b]pyridin-7-yl acetate instead of 4-chloro-2-(pyridin-2-ylmethoxy)-6,7-dihydro-5H-cyclopenta[b]pyridine, and using pyrimidin-5-ylboronic acid instead of (3-fluorophenyl)boronic acid. The resulting compound was dissolved in ethyl acetate (8 mL), followed by the addition of 1 N HCl/Et₂O solution (0.3 mL), and then stirred for 0.5 hour. The resulting precipitate was collected by filtration to give the title compound (93.4 mg) as a white powder.

[MS (ESI) m/z 321.2 (M+H)⁺]

### Example 236

### 2-(Pyridin-2-ylmethoxy)-4-(pyrimidin-5-yl)-5,6-dihydro-7H-cyclopenta[b]pyridin-7-one

To 2-(pyridin-2-ylmethoxy)-4-(pyrimidin-5-yl)-6,7-dihydro-5H-cyclopenta[b]pyridin-7-ol (3 g) and 1,1,1-triacetoxy-1,1-dihydro-1,2-benziodoxol-3(1H)-one (Dess-Martin reagent, 4.37 g) was added CH₂Cl₂ (100 mL), and the mixture was stirred under Ar atmosphere at room temperature overnight. Chloroform and saturated aqueous sodium hydrogen carbonate solution were added to the reaction mixture, and the resulting precipitate was removed by filtration, and the filtrate was extracted with chloroform. The organic layer was dried over anhydrous sodium sulfate, and filtered off, and the filtrate was evaporated under reduced pressure. The resulting residue was purified by silica gel column chromatography to give the title compound (2.2 g) as a pale yellow powder.

[MS (ESI) m/z 319.3 (M+H)⁺]

### Example 237

### 7-Methylidene-2-(pyridin-2-ylmethoxy)-4-(pyrimidin-5-yl)-6,7-dihydro-5H-cyclopenta[b]pyridine

To methyltriphenylphosphonium bromide (281 mg) were added THF (8 mL) and KO-t-Bu (90 mg) under ice water cooling, and the mixture was stirred under Ar atmosphere for 1 hour. 2-(Pyridin-2-ylmethoxy)-4-(pyrimidin-5-yl)-5,6-dihydro-7H-cyclopenta[b]pyridin-7-one (50 mg) was added to the reaction mixture, and the mixture was further stirred at room temperature for 2 hours. The mixture was added with water and ethyl acetate, and subjected to extraction. The organic layer was washed with saturated brine, dried over anhydrous sodium sulfate, and filtered off, and the filtrate was evaporated under reduced pressure. The resulting residue was purified by silica gel column chromatography to give the title compound (24 mg) as a white powder.

[MS (ESI) m/z 317.3 (M+H)⁺]

### Example 238

### 7-Methyl-2-(pyridin-2-ylmethoxy)-4-(pyrimidin-5-yl)-6,7-dihydro-5H-cyclopenta[b]pyridine

### [Step 1]

Production of 7-methyl-4-(pyrimidin-5-yl)-6,7-dihydro-5H-cyclopenta[b]pyridin-2-ol

To 7-methylidene-2-(pyridin-2-ylmethoxy)-4-(pyrimidin-5-yl)-6,7-dihydro-5H-cyclopenta[b]pyridine (24 mg) were added methanol (4 mL) and palladium-activated carbon ethylenediamine complex (50 mg), and the mixture was stirred under hydrogen atmosphere at room temperature for 2 days. The reaction mixture was filtered through Celite, and the solvent was evaporated under reduced pressure to give the title compound (17 mg).

### [Step 2]

Production of 7-methyl-2-(pyridin-2-ylmethoxy)-4-(pyrimidin-5-yl)-6,7-dihydro-5H-cyclopenta[b]pyridine

To 7-methyl-4-(pyrimidin-5-yl)-6,7-dihydro-5H-cyclopenta[b]pyridin-2-ol (17 mg) and 2-(bromomethyl)pyridine hydrobromide (38 mg) were added toluene (10 mL) and silver carbonate (21 mg), and the mixture was stirred under Ar atmosphere at 100° C. for 10 hours. After the reaction mixture was allowed to return to room temperature, diluted with ethyl acetate, and filtered through Celite, the filtrate was evaporated under reduced pressure. The resulting residue was purified by silica gel column chromatography to give the title compound (5 mg) as a white powder.

[MS (ESI) m/z 319.3 (M+H)⁺]

### Example 239

### 7-Ethylidene-2-(pyridin-2-ylmethoxy)-4-(pyrimidin-5-yl)-6,7-dihydro-5H-cyclopenta[b]pyridine

THF (11 mL) was added to ethyltriphenylphosphonium bromide (416 mg), followed by the addition of 2.7 M of n-butyllithium/THF (0.41 mL) under ice water cooling, and the mixture was stirred under Ar atmosphere for 1 hour. 2-(Pyridin-2-ylmethoxy)-4-(pyrimidin-5-yl)-5,6-dihydro-7H-cyclopenta[b]pyridin-7-one (70 mg) was added to the reaction mixture and the mixture was further stirred at room temperature for 2 hours. The mixture was added with water and ethyl acetate, and subjected to extraction. The organic layer was washed with saturated brine, dried over anhydrous sodium sulfate, and filtered off, and the filtrate was evaporated under reduced pressure. The resulting residue was purified by silica gel column chromatography to give the title compound (63 mg) as a white powder.

[MS (ESI) m/z 331.4 (M+H)⁺]

### Example 240

### 7-Ethyl-2-(pyridin-2-ylmethoxy)-4-(pyrimidin-5-yl)-6,7-dihydro-5H-cyclopenta[b]pyridine

To 7-ethylidene-2-(pyridin-2-ylmethoxy)-4-(pyrimidin-5-yl)-6,7-dihydro-5H-cyclopenta[b]pyridine (63 mg) were added methanol (4 mL) and palladium-activated carbon ethylenediamine complex (30 mg), and the mixture was stirred under hydrogen atmosphere at room temperature for one day. Palladium-activated carbon ethylenediamine complex (30 mg) was added to the reaction mixture, and the mixture was further stirred at room temperature for one day. The reaction mixture was filtered through Celite and the filtrate was evaporated under reduced pressure. The resulting residue was purified by silica gel column chromatography to give the title compound (6 mg) as colorless oil.

[MS (ESI) m/z 333.4 (M+H)⁺]

### Example 241

### 2-(Pyridin-2-ylmethoxy)-4-(pyrimidin-5-yl)-6,7-dihydro-5H-cyclopenta[b]pyridin-7-yl acetate hydrochloride

2-(Pyridin-2-ylmethoxy)-4-(pyrimidin-5-yl)-6,7-dihydro-5H-cyclopenta[b]pyridin-7-yl acetate (29 mg) was prepared as colorless oil according to the procedure described in Example 58 using 4-chloro-2-(pyridin-2-ylmethoxy)-6,7-dihydro-5H-cyclopenta[b]pyridin-7-yl acetate instead of 4-chloro-2-(pyridin-2-ylmethoxy)-6,7-dihydro-5H-cyclopenta[b]pyridine and using pyrimidin-5-ylboronic acid instead of (3-fluorophenyl)boronic acid.

[MS (ESI) m/z 363.1 (M+H)⁺]

The resulting compound was dissolved in ethyl acetate (1 mL), followed by the addition of 1 N HCl/Et₂O solution (0.08 mL), and the mixture was stirred for 0.5 hour. The resulting precipitate was collected by filtration to give the title compound (10 mg) as a white powder.

[MS (ESI) m/z 363.3 (M+H)⁺]

### Example 242

### 7-Methoxy-2-(pyridin-2-ylmethoxy)-4-(pyrimidin-5-yl)-6,7-dihydro-5H-cyclopenta[b]pyridine hydrochloride

The title compound was prepared as a white powder according to the procedure described in Example 123 using 2-(pyridin-2-ylmethoxy)-4-(pyrimidin-5-yl)-6,7-dihydro-5H-cyclopenta[b]pyridin-7-ol instead of {5-[2-(pyridin-2-ylmethoxy)-5,6,7,8-tetrahydroquinolin-4-yl]pyridin-3-yl}methanol.

[MS (ESI) m/z 335.3 (M+H)⁺]

### Example 243

### 6,6-Dimethyl-2-(pyridin-2-ylmethoxy)-4-(pyrimidin-5-yl)-5,6-dihydro-7H-cyclopenta[b]pyridin-7-one

To 2-(pyridin-2-ylmethoxy)-4-(pyrimidin-5-yl)-5,6-dihydro-7H-cyclopenta[b]pyridin-7-one (100 mg) was added THF (31 mL), and 1 M lithium hexamethyldisilazide/THF solution (0.75 mL) was added to the mixture at −78° C., and the mixture was stirred under Ar atmosphere for 1 hour. Then, MeI (0.11 mL) was added to the reaction mixture, and the mixture was further stirred at room temperature for 2 hours. The reaction mixture was added with water and ethyl acetate, and subjected to extraction. The organic layer was washed with saturated brine, dried over anhydrous sodium sulfate, and filtered off, and the filtrate was evaporated under reduced pressure. The resulting residue was purified by silica gel column chromatography to give the title compound (50 mg) as pale yellow oil. [MS (ESI) m/z 347.4 (M+H)⁺]

### Example 244

### 6,6-Dimethyl-2-(pyridin-2-ylmethoxy)-4-(pyrimidin-5-yl)-6,7-dihydro-5H-cyclopenta[b]pyridine

To 6,6-dimethyl-2-(pyridin-2-ylmethoxy)-4-(pyrimidin-5-yl)-5,6-dihydro-7H-cyclopenta[b]pyridin-7-one (90 mg) were added ethylene glycol (5 mL), hydrazine monohydrate (30 mg) and potassium hydroxide (29 mg), and then the mixture was stirred under Ar atmosphere at 120° C. for 2 hours. Potassium hydroxide (100 mg) was added to the reaction mixture, and the mixture was further stirred at 150° C. for 5 hours. The reaction mixture was added with water and ethyl acetate, and subjected to extraction. The organic layer was washed with saturated brine, dried over anhydrous sodium sulfate, and filtered off, and the filtrate was evaporated under reduced pressure. The resulting residue was purified by silica gel column chromatography to give the title compound (33 mg) as a white powder.

[MS (ESI) m/z 333.4 (M+H)⁺]

### Example 245

### 3-({[4-(Pyridin-3-yl)-6,7-dihydro-5H-cyclopenta[b]pyridin-2-yl]oxy}methyl)benzonitrile

### [Step 1]

Production of 4-chloro-6,7-dihydro-5H-cyclopenta[b]pyridin-2-ol

To N,N-dimethylaniline (12.68 g) was added phosphoryl chloride (5.263 mL), and then 6,7-dihydro-5H-cyclopenta[b]pyridine-2,4-diol (see, for example, Helvetica Chimica Acta, 1945, vol. 28, p. 1684-1690) (5 g) was added in portions and the mixture was stirred at room temperature overnight. 5 N Sodium hydroxide solution was added dropwise under water cooling, and the resulting precipitate was collected by filtration to give the title compound (3.31 g) as a white solid.

### [Step 2]

Production of 3-{[(4-chloro-6,7-dihydro-5H-cyclopenta[b]pyridin-2-yl)oxy]methyl}benzonitrile

To 4-chloro-6,7-dihydro-5H-cyclopenta[b]pyridin-2-ol (500 mg), 3-(bromomethyl)benzonitrile (867 mg) and silver carbonate (813 mg) was added CPME (17 mL), and then the mixture was stirred under Ar atmosphere at 100° C. for 4 hours. After the reaction mixture was allowed to return to room temperature, diluted with ethyl acetate, and filtered through Celite, the filtrate was evaporated under reduced pressure. The resulting residue was purified by silica gel column chromatography to give the title compound (960 mg) as colorless oil.

### [Step 3]

Production of 3-({[4-(pyridin-3-yl)-6,7-dihydro-5H-cyclopenta[b]pyridin-2-yl]oxy}methyl)benzonitrile

To 3-{[(4-chloro-6,7-dihydro-5H-cyclopenta[b]pyridin-2-yl)oxy]methyl}benzonitrile (43 mg), pyridin-3-ylboronic acid (18 mg), Pd(dtbpf)Cl₂ (3 mg) and potassium carbonate (44 mg) was added 1,4-dioxane/water (3/1, 1 mL), and the mixture was stirred at 100° C. for 2 hours. After the reaction mixture was allowed to return to room temperature, diluted with ethyl acetate, and filtered through Celite, the filtrate was evaporated under reduced pressure. The resulting residue was purified by silica gel column chromatography to give the title compound (25 mg) as a white powder.

[MS (ESI) m/z 328.6 (M+H)⁺]

### Example 246

### 5-{2-[(3,5-Difluoropyridin-2-yl)methoxy]-6,7-dihydro-5H-cyclopenta[b]pyridin-4-yl}pyridine-2-carbonitr ile

To 5-(2-chloro-6,7-dihydro-5H-cyclopenta[b]pyridin-4-yl) pyridine-2-carbonitrile (47 mg), (3,5-difluoropyridin-2-yl)methanol (32 mg), Pd₂(dba)₃.CHCl₃ (11 mg), cesium carbonate (180 mg) and t-Bu-X-Phos (19 mg) was added toluene (1 mL), and the mixture was degassed, and then stirred under Ar atmosphere at 100° C. for 4 hours. After the reaction mixture was allowed to return to room temperature, diluted with ethyl acetate, and filtered through Celite, the filtrate was evaporated under reduced pressure. The resulting residue was dissolved in CH₂Cl₂ (2 mL), and added with Et₃N (0.1 mL) and TFAA (0.05 mL) under ice water cooling, and the mixture was stirred at the same temperature for 2 hours. The reaction mixture was added with ethyl acetate and saturated aqueous sodium hydrogen carbonate solution, and subjected to extraction. The organic layer was washed with saturated brine, dried over anhydrous sodium sulfate, and filtered off, and the filtrate was evaporated under reduced pressure. The resulting residue was purified by silica gel column chromatography and by recycling preparative gel permeation chromatography (Japan Analytical Industry, Co. Ltd., LC-9201) to give the title compound (27 mg) as colorless oil.

[MS (ESI) m/z 365.3 (M+H)⁺]

### Example 247

### 4-[({4-[5-(Hydroxymethyl)pyridin-3-yl]-6,7-dihydro-5H-cyclopenta[b]pyridin-2-yl}oxy)methyl]benzonitrile

### [Step 1]

Production of 4-{[(4-chloro-6,7-dihydro-5H-cyclopenta[b]pyridin-2-yl)oxy]methyl}benzonitrile

To 4-chloro-6,7-dihydro-5H-cyclopenta[b]pyridin-2-ol (200 mg), 4-(bromomethyl)benzonitrile (300 mg), and silver carbonate (325 mg) was added toluene (10 mL), and the mixture was stirred at 100° C. for 2 hours. After the reaction mixture was allowed to return to room temperature, diluted with ethyl acetate, and filtered through Celite, the filtrate was evaporated under reduced pressure. The resulting residue was purified by silica gel column chromatography to give the title compound (343 mg) as a white solid.

### [Step 2]

Production of 4-[({4-[5-(hydroxymethyl)pyridin-3-yl]-6,7-dihydro-5H-cyclopenta[b]pyridin-2-yl}oxy)methyl]benzonitrile

To 4-{[(4-chloro-6,7-dihydro-5H-cyclopenta[b]pyridin-2-yl)oxy]methyl}benzonitrile (25 mg), [5-(4,4,5,5-tetramethyl-1,3,2-dioxaborolan-2-yl)pyridin-3-yl]methanol (31 mg), Pd(dtbpf)Cl₂ (3 mg) and potassium carbonate (36 mg) was added 1,4-dioxane/water (4/1, 1 mL), and the mixture was degassed, and then stirred under Ar atmosphere at 100° C. for 2 hours. The reaction mixture was purified by silica gel column chromatography to give the title compound (19 mg) as a white solid.

[MS (ESI) m/z 358.3 (M+H)⁺]

### Example 248

### (5-{2-[(4-Fluorobenzyl)oxy]-6,7-dihydro-5H-cyclopenta[b]pyridin-4-yl}pyridin-3-yl)methanol

### [Step 1]

Production of 4-chloro-2-[(4-fluorobenzyl)oxy]-6,7-dihydro-5H-cyclopenta[b]pyridine

The title compound was prepared as a white solid according to the procedure described in Example 247, Step 1, using 1-(bromomethyl)-4-fluorobenzene instead of 4-(bromomethyl)benzonitrile.

### [Step 2]

Production of 5-{2-[(4-fluorobenzyl)oxy]-6,7-dihydro-5H-cyclopenta[b]pyridin-4-yl}pyridin-3-yl)methanol

The title compound was prepared as a white solid according to the procedure described in Example 247, Step 2, using 4-chloro-2-[(4-fluorobenzyl)oxy]-6,7-dihydro-5H-cyclopenta[b]pyridine instead of 4-{[(4-chloro-6,7-dihydro-5H-cyclopenta[b]pyridin-2-yl)oxy]methyl}benzonitrile

[MS (ESI) m/z 351.3 (M+H)⁺]

### Example 249

### (5-{2-[(3-Fluorobenzyl)oxy]-6,7-dihydro-5H-cyclopenta[b]pyridin-4-yl}pyridin-3-yl)methanol

### [Step 1]

Production of 4-chloro-2-[(3-fluorobenzyl)oxy]-6,7-dihydro-5H-cyclopenta[b]pyridine

The title compound was prepared as a white solid according to the procedure described in Example 247, Step 1, using 1-(bromomethyl)-3-fluorobenzene instead of 4-(bromomethyl)benzonitrile.

### [Step 2]

Production of (5-{2-[(3-fluorobenzyl)oxy]-6,7-dihydro-5H-cyclopenta[b]pyridin-4-yl}pyridin-3-yl)methanol

The title compound was prepared as a white solid according to the procedure described in Example 247, Step 2, using 4-chloro-2-[(3-fluorobenzyl)oxy]-6,7-dihydro-5H-cyclopenta[b]pyridine instead of 4-{[(4-chloro-6,7-dihydro-5H-cyclopenta[b]pyridin-2-yl)oxy]methyl}benzonitrile

[MS (ESI) m/z 351.3 (M+H)⁺]

### Example 250

### 6-({[4-(5-Fluoropyridin-3-yl)-6,7-dihydro-5H-cyclopenta[b]pyridin-2-yl]oxy}methyl)pyridine-3-carbonitrile

### [Step 1]

Production of 6-{[(4-chloro-6,7-dihydro-5H-cyclopenta[b]pyridin-2-yl)oxy]methyl}pyridine-3-carbonitrile

The title compound was prepared as a pale yellow powder according to the procedure described in Example 247, Step 1, using 6-(bromomethyl)pyridine-3-carbonitrile instead of 4-(bromomethyl)benzonitrile.

### [Step 2]

Production of 6-({[4-(5-fluoropyridin-3-yl)-6,7-dihydro-5H-cyclopenta[b]pyridin-2-yl]oxy}methyl)pyridine-3-carbonitrile

The title compound was prepared as a white powder according to the procedure described in Example 247, Step 2, using 6-{[(4-chloro-6,7-dihydro-5H-cyclopenta[b]pyridin-2-yl)oxy]methyl}pyridine-3-carbonitrile instead of 4-{[(4-chloro-6,7-dihydro-5H-cyclopenta[b]pyridin-2-yl)oxy]methyl}benzonitrile, and using 3-fluoro-5-(4,4,5,5-tetramethyl-1,3,2-dioxaborolan-2-yl)pyridine instead of [5-(4,4,5,5-tetramethyl-1,3,2-dioxaborolan-2-yl)pyridin-3-yl]methanol

[MS (ESI) m/z 347.3 (M+H)⁺]

### Example 251

### 3-({[4-(2-Fluoropyridin-4-yl)-6,7-dihydro-5H-cyclopenta[b]pyridin-2-yl]oxy}methyl)benzonitrile

The title compound was prepared as colorless oil according to the procedure described in Example 245, Step 3, using 2-fluoro-4-(4,4,5,5-tetramethyl-1,3,2-dioxaborolan-2-yl)pyridine instead of pyridin-3-ylboronic acid.

[MS (ESI) m/z 346.3 (M+H)⁺]

### Example 252

### 6-({[4-(2-Fluoropyridin-4-yl)-6,7-dihydro-5H-cyclopenta[b]pyridin-2-yl]oxy}methyl)pyridine-3-carbonitrile

The title compound was prepared as a white powder according to the procedure described in Example 250, Step 2, using 2-fluoro-4-(4,4,5,5-tetramethyl-1,3,2-dioxaborolan-2-yl)pyridine instead of 3-fluoro-5-(4,4,5,5-tetramethyl-1,3,2-dioxaborolan-2-yl)pyridine.

[MS (ESI) m/z 347.3 (M+H)⁺]

### Example 253

### 3-[({4-[5-(Hydroxymethyl)pyridin-3-yl]-6,7-dihydro-5H-cyclopenta[b]pyridin-2-yl}oxy)methyl]benzonitrile

The title compound was prepared as a white solid according to the procedure described in Example 247, Step 2, using 3-{[(4-chloro-6,7-dihydro-5H-cyclopenta[b]pyridin-2-yl)oxy]methyl}benzonitrile instead of 4-{[(4-chloro-6,7-dihydro-5H-cyclopenta[b]pyridin-2-yl)oxy]methyl}benzonitrile.

[MS (ESI) m/z 358.4 (M+H)⁺]

### Example 254

### 6-({[4-(Pyrimidin-5-yl)-6,7-dihydro-5H-cyclopenta[b]pyridin-2-yl]oxy}methyl)pyridine-3-carbonitrile hydrochloride

6-({[4-(Pyrimidin-5-yl)-6,7-dihydro-5H-cyclopenta[b]pyridin-2-yl]oxy}methyl)pyridine-3-carbonitrile was prepared as a pale yellow solid (54 mg) according to the procedure described in Example 11, Step 2, using 6-(hydroxymethyl)pyridine-3-carbonitrile instead of benzylalcohol, and using cesium carbonate instead of NaO-t-Bu. The resulting compound was dissolved in ethyl acetate (10 mL), followed by the addition of 1 N HCl/Et₂O solution (0.164 mL) under ice water cooling, and the mixture was stirred at the same temperature for 0.5 hour.

The resulting precipitate was collected by filtration to give the title compound (44 mg) as a white powder.

[MS (ESI) m/z 330.4 (M+H)⁺]

Elementary analysis as C₁₉H₁₅N₅O.HCl+0.2H₂O

Calcd. (%) C: 61.77.; H: 4.48.; N: 18.96.

Found. (%) C: 61.59.; H: 4.30.; N: 18.70.

### Example 255

### 2-[(3,6-Difluoropyridin-2-yl)methoxy]-4-(pyrimidin-5-yl)-6,7-dihydro-5H-cyclopenta[b]pyridine

The title compound was prepared as a pale yellow powder according to the procedure described in Example 11, Step 2, using (3,6-difluoropyridin-2-yl)methanol instead of benzylalcohol, and using Pd₂(dba)₃ instead of Pd₂(dba)₃.CHCl₃, and using cesium carbonate instead of NaO-t-Bu.

[MS (ESI) m/z 341.3 (M+H)⁺]

### Example 256

### 2-[(2-Methylpyridin-4-yl)methoxy]-4-(pyrimidin-5-yl)-6,7-dihydro-5H-cyclopenta[b]pyridine

The title compound was prepared as a white powder according to the procedure described in Example 11, Step 2, using (2-methylpyridin-4-yl)methanol instead of benzylalcohol, and using Pd₂(dba)₃ instead of Pd₂(dba)₃.CHCl₃.

[MS (ESI) m/z 319.5 (M+H)⁺]

### Example 257

### 2-[(4-Fluorobenzyl)oxy]-4-(pyrimidin-5-yl)-6,7-dihydro-5H-cyclopenta[b]pyridine

The title compound was prepared as a pale yellow solid according to the procedure described in Example 11, Step 2, using (4-fluorophenyl)methanol instead of benzylalcohol, and using Pd₂(dba)₃ instead of Pd₂(dba)₃.CHCl₃, and using potassium phosphate instead of NaO-t-Bu.

[MS (ESI) m/z 322.2 (M+H)⁺]

### Example 258

### 4-({[4-(Pyrimidin-5-yl)-6,7-dihydro-5H-cyclopenta[b]pyridin-2-yl]oxy}methyl)benzonitrile

The title compound was prepared as a cream solid according to the procedure described in Example 247, Step 2, using pyrimidin-5-ylboronic acid instead of [5-(4,4,5,5-tetramethyl-1,3,2-dioxaborolan-2-yl)pyridin-3-yl]methanol.

[MS (ESI) m/z 329.4 (M+H)⁺]

### Example 259

### 5-[2-(Pyridin-2-ylmethoxy)-6,7-dihydro-5H-cyclopenta[b]pyridin-4-yl]pyrimidin-2-amine hydrochloride

### [Step 1]

Production of 4-(2-fluoropyrimidin-5-yl)-2-(pyridin-2-ylmethoxy)-6,7-dihydro-5H-cyclopenta[b]pyridine

To 2-(pyridin-2-ylmethoxy)-6,7-dihydro-5H-cyclopenta[b]pyridin-4-yl trifluoromethanesulfonate (83 mg), 2-fluoro-5-(trimethylstannanyl)pyrimidine (69 mg) and Pd(PPh₃)₄ (38 mg) was added NMP (2 mL), and the mixture was stirred at 100° C. for 4 hours. After the reaction mixture was allowed to return to room temperature, the mixture was extracted with water and ethyl acetate. The organic layer was washed with water and saturated brine in turn, dried over anhydrous sodium sulfate, and filtered off, and the filtrate was evaporated under reduced pressure. The resulting residue was purified by silica gel column chromatography to give the title compound (53 mg) as a white solid.

### [Step 2]

Production of 5-[2-(pyridin-2-ylmethoxy)-6,7-dihydro-5H-cyclopenta[b]pyridin-4-yl]pyrimidin-2-amine hydrochloride

To 4-(2-fluoropyrimidin-5-yl)-2-(pyridin-2-ylmethoxy)-6,7-dihydro-5H-cyclopenta[b]pyridine (20 mg) was added concentrated ammonia/methanol solution (1 mL), and then the mixture was stirred at 50° C. for 3 hours. After the reaction mixture was allowed to return to room temperature, the solvent was evaporated under reduced pressure. The resulting residue was purified by silica gel column chromatography and by recycling preparative gel permeation chromatography (Japan Analytical Industry, Co. Ltd., LC-9201) to give 5-[2-(pyridin-2-ylmethoxy)-6,7-dihydro-5H-cyclopenta[b]pyridin-4-yl]pyrimidin-2-amine (8 mg) as a white powder.

[MS (ESI) m/z 320.3 (M+H)⁺]

The resulting compound was dissolved in ethyl acetate, followed by the addition of 1 N HCl/Et₂O solution (0.164 mL) under ice water cooling, and the resulting precipitate was collected by filtration to give the title compound (5 mg) as a white powder.

[MS (ESI) m/z 320.3 (M+H)⁺]

### Example 260

### 2-[(4-Fluorobenzyl)oxy]-4-(2-methylpyrimidin-5-yl)-6,7-dihydro-5H-cyclopenta[b]pyridine

To 2-chloro-4-(2-methylpyrimidin-5-yl)-6,7-dihydro-5H-cyclopenta[b]pyridine (50 mg), (4-fluorophenyl)methanol (51 mL), t-Bu-X-Phos (21 mg), Pd₂ (dba)₃ (19 mg) and NaO-t-Bu (39 mg) was added toluene (2 mL), and the mixture was degassed, and then stirred under Ar atmosphere at 100° C. for 6 hours. After the solvent of the reaction mixture was evaporated under reduced pressure, the resulting residue was purified by silica gel column chromatography to give the title compound (21 mg) as a white powder.

[MS (ESI) m/z 336.3 (M+H)⁺]

### Example 261

### 4-({[4-(2-Methylpyrimidin-5-yl)-6,7-dihydro-5H-cyclopenta[b]pyridin-2-yl]oxy}methyl)benzonitrile

The title compound was prepared as a white solid according to the procedure described in Example 260 using 4-(hydroxymethyl)benzonitrile instead of (4-fluorophenyl)methanol.

[MS (ESI) m/z 343.3 (M+H)⁺]

### Example 262

### 2-[(2-Methylpyridin-4-yl)methoxy]-4-(2-methylpyrimi din-5-yl)-6,7-dihydro-5H-cyclopenta[b]pyridine

The title compound was prepared as yellow oil according to the procedure described in Example 260 using (2-methylpyridin-4-yl)methanol instead of (4-fluorophenyl)methanol.

[MS (ESI) m/z 333.6 (M+H)⁺]

### Example 263

### 5-{2-[(3-Fluorobenzyl)oxy]-6,7-dihydro-5H-cyclopenta[b]pyridin-4-yl}pyrimidin-2-amine

The title compound was prepared as a white solid according to the procedure described in Example 249, Step 2, using 5-(4,4,5,5-tetramethyl-1,3,2-dioxaborolan-2-yl)pyrimidin-2-amine instead of [5-(4,4,5,5-tetramethyl-1,3,2-dioxaborolan-2-yl)pyridin-3-yl]methanol.

[MS (ESI) m/z 337.3 (M+H)⁺]

### Example 264

### 4-({[4-(2-Aminopyrimidin-5-yl)-6,7-dihydro-5H-cyclopenta[b]pyridin-2-yl]oxy}methyl)benzonitrile

The title compound was prepared as a white solid according to the procedure described in Example 247, Step 2, using 5-(4,4,5,5-tetramethyl-1,3,2-dioxaborolan-2-yl)pyrimidin-2-amine instead of [5-(4,4,5,5-tetramethyl-1,3,2-dioxaborolan-2-yl)pyridin-3-yl]methanol.

[MS (ESI) m/z 344.3 (M+H)⁺]

### Example 265

### 3-({[4-(2-Aminopyrimidin-5-yl)-6,7-dihydro-5H-cyclopenta[b]pyridin-2-yl]oxy}methyl)benzonitrile

The title compound was prepared as a white solid according to the procedure described in Example 245, Step 3, using 5-(4,4,5,5-tetramethyl-1,3,2-dioxaborolan-2-yl)pyrimidin-2-amine instead of pyridin-3-ylboronic acid.

[MS (ESI) m/z 344.4 (M+H)⁺]

### Example 266

### 2-[(3-Fluorobenzyl)oxy]-4-(2-methylpyrimidin-5-yl)-6,7-dihydro-5H-cyclopenta[b]pyridine

The title compound was prepared as a white powder according to the procedure described in Example 260 using (3-fluorophenyl)methanol instead of (4-fluorophenyl)methanol.

[MS (ESI) m/z 336.3 (M+H)⁺]

### Example 267

### 2-({[4-(2-Methylpyrimidin-5-yl)-6,7-dihydro-5H-cyclopenta[b]pyridin-2-yl]oxy}methyl)-1,3-oxazole-4-carbonitrile

### [Step 1]

Production of ethyl [(4-chloro-6,7-dihydro-5H-cyclopenta[b]pyridin-2-yl)oxy]acetate

The title compound was prepared as a white solid according to the procedure described in Example 247, Step 1, using bromoethyl acetate instead of 4-(bromomethyl)benzonitrile

### [Step 2]

Production of [(4-chloro-6,7-dihydro-5H-cyclopenta[b]pyridin-2-yl)oxy]acetic acid

Ethyl [(4-chloro-6,7-dihydro-5H-cyclopenta[b]pyridin-2-yl)oxy]acetate was dissolved in ethanol (4 mL) and water (4 mL), followed by the addition of sodium hydroxide solution, and the mixture was stirred at 50° C. for 3 hours. After the reaction mixture was allowed to return to room temperature, the reaction mixture was made weakly acidic with hydrochloric acid, and then the mixture was added with water and ethyl acetate, and subjected to extraction. The organic layer was washed with saturated brine, dried over anhydrous sodium sulfate, and filtered off, and the filtrate was evaporated under reduced pressure. The resulting residue was purified by silica gel column chromatography to give the title compound (203 mg) as a white solid.

### [Step 3]

Production of methyl N-{[(4-chloro-6,7-dihydro-5H-cyclopenta[b]pyridin-2-yl)oxy]acetyl}serinate

To [(4-chloro-6,7-dihydro-5H-cyclopenta[b]pyridin-2-yl)oxy]acetic acid (200 mg), DL-serine methyl ester hydrochloride (205 mg), HOBt (118 mg) and triethylamine (0.184 mL) was added CH₂Cl₂ (8 mL), followed by the addition of 1-ethyl-3-(3-dimethylaminopropyl)carbodiimide (204 mg), and the mixture was stirred at room temperature overnight. The reaction mixture was added with water and ethyl acetate, and subjected to extraction. The organic layer was washed with saturated brine, dried over anhydrous magnesium sulfate, and filtered off, and the filtrate was evaporated under reduced pressure.

The resulting residue was purified by silica gel column chromatography to give the title compound (242 mg) as a white solid.

### [Step 4]

Production of methyl 2-{[(4-chloro-6,7-dihydro-5H-cyclopenta[b]pyridin-2-yl)oxy]methyl}-4,5-dihydro-1,3-oxazole-4-carboxylate

To methyl N-{[(4-chloro-6,7-dihydro-5H-cyclopenta[b]pyridin-2-yl)oxy]acetyl}serinate (240 mg) was added CH₂Cl₂ (6 mL), and the reaction mixture was degassed, and cooled to −78° C., followed by the addition of N,N-diethylaminosulfur trifluoride (176 mg) under Ar atmosphere, and then the mixture was stirred at the same temperature for 1 hour. To the reaction mixture was added potassium carbonate (151 mg), and the mixture was gradually allowed to warm up to room temperature, and then stirred at room temperature overnight. The resulting mixture was added with water and ethyl acetate, and subjected to extraction. The organic layer was washed with saturated brine, dried over anhydrous magnesium sulfate, and filtered off, and the filtrate was evaporated under reduced pressure. The resulting residue was purified by silica gel column chromatography to give the title compound (147 mg) as pale yellow oil.

### [Step 5]

Production of methyl 2-{[(4-chloro-6,7-dihydro-5H-cyclopenta[b]pyridin-2-yl)oxy]methyl}-1,3-oxazole-4-carboxylate

To methyl 2-{[(4-chloro-6,7-dihydro-5H-cyclopenta[b]pyridin-2-yl)oxy]methyl}-4,5-dihydro-1,3-oxazole-4-carboxylate (140 mg) was added CH₂Cl₂ (4 mL), followed by the addition of 1,8-diazabicyclo[5.4.0]-7-undecene (205 mg) and bromotrichloromethane (116 mg), and the mixture was stirred for 2 hours under ice water cooling. Aqueous sodium sulfite solution was added to the reaction mixture, and the reaction mixture was added with water and ethyl acetate, and subjected to extraction. The organic layer was washed with saturated brine, dried over anhydrous magnesium sulfate, and filtered off, and the filtrate was evaporated under reduced pressure. The resulting residue was purified by silica gel column chromatography to give the title compound (72 mg) as a white sol id.

### [Step 6]

Production of 2-{[(4-chloro-6,7-dihydro-5H-cyclopenta[b]pyridin-2-yl)oxy]methyl}-1,3-oxazole-4-carboxylic acid

The title compound was prepared as a white solid according to the procedure described in Example 267, Step 2, using methyl 2-{[(4-chloro-6,7-dihydro-5H-cyclopenta[b]pyridin-2-yl)oxy]methyl}-1,3-oxazole-4-carboxylate instead of ethyl [(4-chloro-6,7-dihydro-5H-cyclopenta[b]pyridin-2-yl)oxy]acetate.

### [Step 7]

Production of 2-{[(4-chloro-6,7-dihydro-5H-cyclopenta[b]pyridin-2-yl)oxy]methyl}-1,3-oxazole-4-carboxamide

The title compound was prepared as a white solid according to the procedure described in Example 267, Step 3, using 2-{[(4-chloro-6,7-dihydro-5H-cyclopenta[b]pyridin-2-yl)oxy]methyl}-1,3-oxazole-4-carboxylic acid instead of [(4-chloro-6,7-dihydro-5H-cyclopenta[b]pyridin-2-yl)oxy]acetic acid, and using ammonium chloride instead of DL-serine methyl ester hydrochloride.

### [Step 8]

Production of 2-{[(4-chloro-6,7-dihydro-5H-cyclopenta[b]pyridin-2-yl)oxy]methyl}-1,3-oxazole-4-carbonitrile

To 2-{[(4-chloro-6,7-dihydro-5H-cyclopenta[b]pyridin-2-yl)oxy]methyl}-1,3-oxazole-4-carboxamide (35 mg) was added CH₂Cl₂ (4 mL), followed by the addition of triethylamine (0.066 mL) and TFAA (0.022 mL) under ice water cooling, and the mixture was stirred at the same temperature for 1 hour. The reaction mixture was added with water and ethyl acetate, and subjected to extraction. The organic layer was washed with saturated brine, dried over anhydrous magnesium sulfate, and filtered off, and the filtrate was evaporated under reduced pressure. The resulting residue was purified by silica gel column chromatography to give the title compound (29 mg) as a white solid.

### [Step 9]

Production of 2-({[4-(2-methylpyrimidin-5-yl)-6,7-dihydro-5H-cyclopenta[b]pyridin-2-yl]oxy}methyl)-1,3-oxazole-4-carbonitrile

The title compound was prepared as a white solid according to the procedure described in Example 247, Step 2, using 2-{[(4-chloro-6,7-dihydro-5H-cyclopenta[b]pyridin-2-yl)oxy]methyl}-1,3-oxazole-4-carbonitrile instead of 4-{[(4-chloro-6,7-dihydro-5H-cyclopenta[b]pyridin-2-yl)oxy]methyl}benzonitrile, and using 2-methyl-5-(4,4,5,5-tetramethyl-1,3,2-dioxaborolan-2-yl)pyrimidine instead of [5-(4,4,5,5-tetramethyl-1,3,2-dioxaborolan-2-yl)pyridin-3-yl]methanol

[MS (ESI) m/z 334.4 (M+H)⁺]

### Example 268

### {6-[2-(Pyridin-2-ylmethoxy)-6,7-dihydro-5H-cyclopenta[b]pyridin-4-yl]pyrazin-2-yl}methanol hydrochloride

To 2-(pyridin-2-ylmethoxy)-4-(4,4,5,5-tetramethyl-1,3,2-dioxaborolan-2-yl)-6,7-dihydro-5H-cyclopenta[b]pyridine (100 mg), (6-chloropyrazin-2-yl)methanol (49 mg), Pd₂(dba)₃ (13 mg) and S-Phos (23 mg) was added 1,4-dioxane/water (3/1, 4 mL), and the mixture was degassed, and then stirred under Ar atmosphere at 100° C. for 2 hours. After the reaction mixture was allowed to return to room temperature, the mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography and by recycling preparative gel permeation chromatography (Japan Analytical Industry, Co. Ltd., LC-9201) to give {6-[2-(pyridin-2-ylmethoxy)-6,7-dihydro-5H-cyclopenta[b]pyridin-4-yl]pyrazin-2-yl}methanol (63 mg) as a white powder. The resulting compound was dissolved in ethyl acetate (5 mL), followed by the addition of 1 N HCl/Et₂O solution (0.21 mL) under ice water cooling, and the mixture was stirred at the same temperature for 0.5 hour. The resulting precipitate was collected by filtration to give the title compound (53 mg) as a white powder.

[MS (ESI) m/z 335.3 (M+H)⁺]

Elementary analysis as C₁₉H₁₈N₄O₂.HCl+0.1H₂O

Calcd. (%) C: 61.24.; H: 5.19.; N: 15.04.

Found. (%) C: 61.24.; H: 4.98.; N: 15.07.

### Example 269

### (6-{2-[(5-Fluoropyridin-2-yl)methoxy]-6,7-dihydro-5H-cyclopenta[b]pyridin-4-yl}pyrazin-2-yl)methanol

### [Step 1]

Production of 4-chloro-2-[(5-fluoropyridin-2-yl)methoxy]-6,7-dihydro-5H-cyclopenta[b]pyridine

The title compound was prepared as a white powder according to the procedure described in Example 44, Step 2, using (5-fluoropyridin-2-yl)methanol instead of (3,5-difluoropyridin-2-yl)methanol.

### [Step 2]

Production of 2-[(5-fluoropyridin-2-yl)methoxy]-6,7-dihydro-5H-cyclopenta[b]pyridin-4-ol

The title compound was prepared as a pale brown powder according to the procedure described in Example 44, Step 3, using 4-chloro-2-[(5-fluoropyridin-2-yl)methoxy]-6,7-dihydro-5H-cyclopenta[b]pyridine instead of 4-chloro-2-[(3,5-difluoropyridin-2-yl)methoxy]-6,7-dihydro-5H-cyclopenta[b]pyridine.

### [Step 3]

Production of 2-[(5-fluoropyridin-2-yl)methoxy]-6,7-dihydro-5H-cyclopenta[b]pyridin-4-yl trifluoromethanesulfonate

The title compound was prepared according to the procedure described in Example 44, Step 4, using 2-[(5-fluoropyridin-2-yl)methoxy]-6,7-dihydro-5H-cyclopenta[b]pyridin-4-ol instead of 2-[(3,5-difluoropyridin-2-yl)methoxy]-6,7-dihydro-5H-cyclopenta[b]pyridin-4-ol.

### [Step 4]

Production of 2-[(5-fluoropyridin-2-yl)methoxy]-4-(4,4,5,5-tetramethyl-1,3,2-dioxaborolan-2-yl)-6,7-dihydro-5H-cyclopenta[b]pyridine

The title compound was prepared according to the procedure described in Example 5, Step 1, using 2-[(5-fluoropyridin-2-yl)methoxy]-6,7-dihydro-5H-cyclopenta[b]pyridin-4-yl trifluoromethanesulfonate instead of 2-(pyridin-2-ylmethoxy)-6,7-dihydro-5H-cyclopenta[b]pyridin-4-yl trifluoromethanesulfonate.

### [Step 5]

Production of (6-{2-[(5-fluoropyridin-2-yl)methoxy]-6,7-dihydro-5H-cyclopenta[b]pyridin-4-yl}pyrazin-2-yl)methanol

To 2-[(5-fluoropyridin-2-yl)methoxy]-4-(4,4,5,5-tetramethyl-1,3,2-dioxaborolan-2-yl)-6,7-dihydro-5H-cyclopenta[b]pyridine (37 mg), (6-chloropyrazin-2-yl)methanol (16 mg), Pd(dppf)Cl₂.CH₂Cl₂ (8 mg) and potassium carbonate (42 mg) was added 1,4-dioxane/water (3/1, 1 mL), and the reaction mixture was degassed, and then stirred under Ar atmosphere at 80° C. for 3 hours. After the reaction mixture was allowed to return to room temperature, the mixture was added with water and ethyl acetate, and subjected to extraction. The organic layer was dried over anhydrous sodium sulfate, and filtered off, and the filtrate was evaporated under reduced pressure. The resulting residue was purified by silica gel column chromatography to give the title compound (22 mg) as a white solid.

[MS (ESI) m/z 353.1 (M+H)⁺]

### Example 270

### 3-[({4-[6-(Hydroxymethyl)pyrazin-2-yl]-6,7-dihydro-5H-cyclopenta[b]pyridin-2-yl}oxy)methyl]benzonitrile

### [Step 1]

Production of [6-(2-chloro-6,7-dihydro-5H-cyclopenta[b]pyridin-4-yl)pyrazin-2-yl]methanol

The title compound was prepared as a white solid according to the procedure described in Example 9, Step 5, using (6-chloropyrazin-2-yl)methanol instead of 5-bromopyridine-2-carbonitrile.

### [Step 2]

Production of 4-[6-({[tert-butyl(dimethyl)silyl]oxy}methyl)pyrazin-2-yl]-2-chloro-6,7-dihydro-5H-cyclopenta[b]pyridine

The title compound was prepared as a white powder according to the procedure described in Example 161, Step 1, using [6-(2-chloro-6,7-dihydro-5H-cyclopenta[b]pyridin-4-yl)pyrazin-2-yl]methanol instead of (6-bromopyridin-2-yl)methanol.

### [Step 3]

Production of 3-[({4-[6-({[tert-butyl(dimethyl)silyl]oxy}methyl)pyrazin-2-yl]-6,7-dihydro-5H-cyclopenta[b]pyridin-2-yl}oxy)methyl]benzonitrile

To 4-[6-({[tert-butyl(dimethyl)silyl]oxy}methyl)pyrazin-2-yl]-2-chloro-6,7-dihydro-5H-cyclopenta[b]pyridine (30 mg), 3-(hydroxymethyl)benzonitrile (16 mg), t-Bu-X-Phos (7 mg), Pd₂(dba)₃ (4 mg) and NaO-t-Bu (15 mg) was added 1,4-dioxane (1 mL), and the mixture was degassed, and then stirred under Ar atmosphere at 100° C. for 2 hours. The reaction mixture was purified by silica gel column chromatography to give the title compound (15 mg) as colorless oil.

### [Step 4]

Production of 3-[({4-[6-(hydroxymethyl)pyrazin-2-yl]-6,7-dihydro-5H-cyclopenta[b]pyridin-2-yl}oxy)methyl]benzonitrile

3-[({4-[6-({[tert-Butyl(dimethyl)silyl]oxy}methyl)pyrazin-2-yl]-6,7-dihydro-5H-cyclopenta[b]pyrid in-2-yl}oxy)methyl]benzonitrile (15 mg) was dissolved in THF (0.5 mL), followed by the addition of 1.0 M TBAF/THF solution (0.05 mL), and the mixture was stirred at room temperature for 1 hour. The reaction mixture was purified by silica gel column chromatography to give the title compound (10 mg) as a white solid.

[MS (ESI) m/z 359.3 (M+H)⁺]

### Example 271

### 4-(Pyridazin-3-yl)-2-(pyridin-2-ylmethoxy)-6,7-dihydro-5H-cyclopenta[b]pyridine

The title compound was prepared as a white powder according to the procedure described in Example 5, Step 2, using pyridazin-3-yl trifluoromethanesulfonate (see, for example, Tetrahedron, 2009, 65, 8969-8980) instead of 5-bromopyridin-3-ol.

[MS (ESI) m/z 305.3 (M+H)⁺]

### Example 272

2-[(3-Fluoropyridin-2-yl)methoxy]-4-(pyridazin-3-yl)-6,7-dihydro-5H-cyclopenta[b]pyridine

### [Step 1]

Production of 2-[(3-fluoropyridin-2-yl)methoxy]-4-(4,4,5,5-tetramethyl-1,3,2-dioxaborolan-2-yl)-6,7-dihydro-5H-cyclopenta[b]pyridine

The title compound was prepared as pale yellow oil according to the procedure described in Example 5, Step 1, using 4-chloro-2-[(3-fluoropyridin-2-yl)methoxy]-6,7-dihydro-5H-cyclopenta[b]pyridine instead of 2-(pyridin-2-ylmethoxy)-6,7-dihydro-5H-cyclopenta[b ]pyridin-4-yl trifluoromethanesulfonate, and using PdCl₂(PCy₃)₂ instead of Pd(dppf)Cl₂.CH₂Cl₂.

### [Step 2]

Production of 2-[(3-fluoropyridin-2-yl)methoxy]-4-(pyridazin-3-yl)-6,7-dihydro-5H-cyclopenta[b]pyridine

The title compound was prepared as a white powder according to the procedure described in Example 271 using 2-[(3-fluoropyridin-2-yl)methoxy]-4-(4,4,5,5-tetramethyl-1,3,2-dioxaborolan-2-yl)-6,7-dihydro-5H-cyclopenta[b]pyridine instead of 2-(pyridin-2-ylmethoxy)-4-(4,4,5,5-tetramethyl-1,3,2-dioxaborolan-2-yl)-6,7-dihydro-5H-cyclopenta[b]pyridine

[MS (ESI) m/z 323.3 (M+H)⁺]

### Example 273

### 4-({[4-(Pyridazin-3-yl)-6,7-dihydro-5H-cyclopenta[b]pyridin-2-yl]oxy}methyl)benzonitrile

### [Step 1]

Production of 2-chloro-4-(pyridazin-3-yl)-6,7-dihydro-5H-cyclopenta[b]pyridine

The title compound was prepared as a white solid according to the procedure described in Example 9, Step 5, using pyridazin-3-yl trifluoromethanesulfonate (see, for example, Tetrahedron, 2009, vol. 65, p. 8969-8980) instead of 5-bromopyridine-2-carbonitrile, and using THF/water (3/1) instead of 1,4-dioxane/water (3/1)

### [Step 2]

Production of 4-({[4-(pyridazin-3-yl)-6,7-dihydro-5H-cyclopenta[b]pyridin-2-yl]oxy}methyl)benzonitrile

The title compound was prepared as a white powder according to the procedure described in Example 261 using 2-chloro-4-(pyridazin-3-yl)-6,7-dihydro-5H-cyclopenta[b]pyridine instead of 2-chloro-4-(2-methylpyrimidin-5-yl)-6,7-dihydro-5H-cyclopenta[b]pyridine, and using Pd₂(dba)₃.CHCl₃ instead of Pd₂(dba)₃.

[MS (ESI) m/z 329.6 (M+H)⁺]

### Example 274

### 6-({[4-(1-Methyl-1H-pyrazol-5-yl)-6,7-dihydro-5H-cyclopenta[b]pyridin-2-yl]oxy}methyl)pyridine-3-carbo nitrile

The title compound was prepared as a white powder according to the procedure described in Example 250, Step 2, using 1-methyl-5-(4,4,5,5-tetramethyl-1,3,2-dioxaborolan-2-yl)-1H-pyrazole instead of 3-fluoro-5-(4,4,5,5-tetramethyl-1,3,2-dioxaborolan-2-yl)pyridine.

[MS (ESI) m/z 332.3 (M+H)⁺]

### Example 275

### 6-({[4-(Pyridazin-3-yl)-6,7-dihydro-5H-cyclopenta[b]pyridin-2-yl]oxy}methyl)pyridine-3-carbonitrile

The title compound was prepared as a white powder according to the procedure described in Example 273, Step 2, using 6-(hydroxymethyl)pyridine-3-carbonitrile instead of 4-(hydroxymethyl)benzonitrile.

[MS (ESI) m/z 330.3 (M+H)⁺]

### Example 276

### 6-[({4-[5-(Hydroxymethyl)pyridin-3-yl]-5,6,7,8-tetrahydroquinolin-2-yl}oxy)methyl]pyridine-3-carbonitrile

### [Step 1]

Production of 6-{[(4-chloro-5,6,7,8-tetrahydroquinolin-2-yl)oxy]m ethyl}pyridine-3-carbonitrile

The title compound was prepared as a white solid according to the procedure described in Example 250, Step 1, using 4-chloro-5,6,7,8-tetrahydroquinolin-2-ol instead of 4-chloro-6,7-dihydro-5H-cyclopenta[b]pyridin-2-ol.

### [Step 2]

Production of 6-[({4-[5-(hydroxymethyl)pyridin-3-yl]-5,6,7,8-tetrahydroquinolin-2-yl}oxy)methyl]pyridine-3-carbonitrile

The title compound was prepared as a white solid according to the procedure described in Example 247, Step 2, using 6-{[(4-chloro-5,6,7,8-tetrahydroquinolin-2-yl)oxy]m ethyl}pyridine-3-carbonitrile instead of 4-{[(4-chloro-6,7-dihydro-5H-cyclopenta[b]pyridin-2-yl)oxy]methyl}benzonitrile.

[MS (ESI) m/z 373.5 (M+H)⁺]

### Example 277

2-[(3,5-Difluoropyridin-2-yl)methoxy]-4-(pyrimidin-5-yl)-5,6,7,8-tetrahydroquinoline

The title compound was prepared as a pale yellow powder according to the procedure described in Example 150 using (3,5-difluoropyridin-2-yl)methanol instead of 6-(hydroxymethyl)pyridine-3-carbonitrile.

[MS (ESI) m/z 355.4 (M+H)⁺]

### Example 278

### 4-({[4-(Pyrimidin-5-yl)-5,6,7,8-tetrahydroquinolin-2-yl]oxy}methyl)benzonitrile

To 2-chloro-4-(pyrimidin-5-yl)-5,6,7,8-tetrahydroquinoline (50 mg), 4-(hydroxymethyl)benzonitrile (54 mL), Pd₂(dba)₃ (19 mg), t-Bu-X-Phos (21 mg) and NaO-t-Bu (39 mg) was added toluene (2 mL). The mixture was degassed, and then stirred under Ar atmosphere at 100° C. for 3 hours. After the reaction mixture was allowed to return to room temperature, diluted with ethyl acetate, and filtered off, the filtrate was evaporated under reduced pressure. The resulting residue was purified by silica gel column chromatography and by recycling preparative gel permeation chromatography (Japan Analytical Industry, Co. Ltd., LC-9201) to give the title compound (16 mg) as a cream-colored powder.

[MS (ESI) m/z 343.3 (M+H)⁺]

### Example 279

### 3-({[4-(Pyrimidin-5-yl)-5,6,7,8-tetrahydroquinolin-2-yl]oxy}methyl)benzonitrile

The title compound was prepared as a white powder according to the procedure described in Example 278 using 3-(hydroxymethyl)benzonitrile instead of 4-(hydroxymethyl)benzonitrile.

[MS (ESI) m/z 343.3 (M+H)⁺]

### Example 280

### 2-[(4-Fluorobenzyl)oxy]-4-(pyrimidin-5-yl)-5,6,7,8-tetrahydroquinoline

The title compound was prepared as a cream-colored solid according to the procedure described in Example 278 using (4-fluorophenyl)methanol instead of 4-(hydroxymethyl)benzonitrile.

[MS (ESI) m/z 336.3 (M+H)⁺]

### Example 281

### 2-[(3-Fluorobenzyl)oxy]-4-(pyrimidin-5-yl)-5,6,7,8-tetrahydroquinoline

The title compound was prepared as a white solid according to the procedure described in Example 278 using (3-fluorophenyl)methanol instead of 4-(hydroxymethyl)benzonitrile.

[MS (ESI) m/z 336.3 (M+H)⁺]

### Example 282

### 4-({[4-(Pyrimidin-5-yl)-5,6,7,8-tetrahydroquinolin-2-yl]oxy}methyl)pyridine-2-carbonitrile

### [Step 1]

Production of 4-{[(4-chloro-5,6,7,8-tetrahydroquinolin-2-yl)oxy]methyl}pyridine-2-carbonitrile

The title compound was prepared as a pale purple solid according to the procedure described in Example 276, Step 1, using 4-(bromomethyl)pyridine-2-carbonitrile instead of 6-(bromomethyl)pyridine-3-carbonitrile.

### [Step 2]

Production of 4-({[4-(pyrimidin-5-yl)-5,6,7,8-tetrahydroquinolin-2-yl]oxy}methyl)pyridine-2-carbonitrile

The title compound was prepared as a yellow powder according to the procedure described in Example 258 using 4-{[(4-chloro-5,6,7,8-tetrahydroquinolin-2-yl)oxy]methyl}pyridine-2-carbonitrile instead of 4-{[(4-chloro-6,7-dihydro-5H-cyclopenta[b]pyridin-2-yl)oxy]methyl}benzonitrile.

[MS (ESI) m/z 344.4 (M+H)⁺]

### Example 283

### 5-{2-[(6-Fluoropyridin-2-yl)methoxy]-5,6,7,8-tetrahydroquinolin-4-yl}pyrimidin-2-amine hydrochloride

### [Step 1]

Production of 2-[(6-fluoropyridin-2-yl)methoxy]-4-[(4-methoxybenzyl)oxy]-5,6,7,8-tetrahydroquinoline

The title compound was prepared as a yellow solid according to the procedure described in Example 278 using 2-chloro-4-[(4-methoxybenzyl)oxy]-5,6,7,8-tetrahydroquinoline instead of 2-chloro-4-(pyrimidin-5-yl)-5,6,7,8-tetrahydroquino line and using (6-fluoropyridin-2-yl)methanol instead of 4-(hydroxymethyl)benzonitrile.

### [Step 2]

Production of 2-[(6-fluoropyridin-2-yl)methoxy]-5,6,7,8-tetrahydroquinolin-4-ol

The title compound was prepared as a white powder according to the procedure described in Example 191, Step 3, using 2-[(6-fluoropyridin-2-yl)methoxy]-4-[(4-methoxybenzyl) oxy]-5,6,7,8-tetrahydroquinoline instead of 2-[(3-fluoropyridin-2-yl)methoxy]-4-[(4-methoxybenzyl) oxy]-5,6,7,8-tetrahydroquinoline.

### [Step 3]

Production of 2-[(6-fluoropyridin-2-yl)methoxy]-5,6,7,8-tetrahydroquinolin-4-yl trifluoromethanesulfonate

The title compound was prepared as colorless oil according to the procedure described in Example 191, Step 4, using 2-[(6-fluoropyridin-2-yl)methoxy]-5,6,7,8-tetrahydroquinolin-4-ol instead of 2-[(3-fluoropyridin-2-yl)methoxy]-5,6,7,8-tetrahydroquinolin-4-ol.

### [Step 4]

Production of 5-{2-[(6-fluoropyridin-2-yl)methoxy]-5,6,7,8-tetrahydroquinolin-4-yl}pyrimidin-2-amine hydrochloride

The title compound was prepared as a white powder according to the procedure described in Example 191, Step 5, using 2-[(6-fluoropyridin-2-yl)methoxy]-5,6,7,8-tetrahydroquinolin-4-yl trifluoromethanesulfonate instead of 2-[(3-fluoropyridin-2-yl)methoxy]-5,6,7,8-tetrahydroquinolin-4-yl trifluoromethanesulfonate.

[MS (ESI) m/z 352.3 (M+H)⁺]

Elementary analysis as C₁₉H₁₈FN₅O HCl+H₂O

Calcd. (%) C: 56.23.; H: 5.22.; N: 17.23.

Found. (%) C: 55.90.; H: 4.76.; N: 17.11.

### Example 284

### 3-({[4-(2-Methylpyrimidin-5-yl)-5,6,7,8-tetrahydroquinolin-2-yl]oxy}methyl)benzonitrile

To 2-chloro-4-(2-methylpyrimidin-5-yl)-5,6,7,8-tetrahydroquinoline (50 mg), 3-(hydroxymethyl)benzonitrile (51 mL), Pd₂(dba)₃ (18 mg), t-Bu-X-Phos (20 mg), and NaO-t-Bu (37 mg) was added toluene (2 mL), and the mixture was degassed, and then stirred under Ar atmosphere at 100° C. for 3 hours. After the solvent of the reaction mixture was evaporated under reduced pressure, the resulting residue was purified by silica gel column chromatography to give the title compound (57 mg) as yellow oil.

[MS (ESI) m/z 357.3 (M+H)⁺]

### Example 285

### 4-({[4-(2-Methylpyrimidin-5-yl)-5,6,7,8-tetrahydroquinolin-2-yl]oxy}methyl)benzonitrile

The title compound was prepared as a pale yellow solid according to the procedure described in Example 284 using 4-(hydroxymethyl)benzonitrile instead of 3-(hydroxymethyl)benzonitrile.

[MS (ESI) m/z 357.3 (M+H)⁺]

### Example 286

### 2-[(2-Methylpyridin-4-yl)methoxy]-4-(2-methylpyrimidin-5-yl)-5,6,7,8-tetrahydroquinoline

The title compound was prepared as yellow oil according to the procedure described in Example 284 using (2-methylpyridin-4-yl)methanol instead of 3-(hydroxymethyl)benzonitrile.

[MS (ESI) m/z 347.6 (M+H)⁺]

### Example 287

### 4-({[4-(2-Methylpyrimidin-5-yl)-5,6,7,8-tetrahydroquinolin-2-yl]oxy}methyl)pyridine-2-carbonitrile

The title compound was prepared as a pale brown powder according to the procedure described in Example 282, Step 2, using 2-methyl-5-(4,4,5,5-tetramethyl-1,3,2-dioxaborolan-2-yl)pyrimidine instead of pyrimidin-5-ylboronic acid.

[MS (ESI) m/z 358.4 (M+H)⁺]

### Example 288

### 6-[({4-[6-(Hydroxymethyl)pyrazin-2-yl]-5,6,7,8-tetrahydroquinolin-2-yl}oxy)methyl]pyridine-3-carbonitrile

### [Step 1]

Production of 6-({[4-(4,4,5,5-tetramethyl-1,3,2-dioxaborolan-2-yl)-5,6,7,8-tetrahydroquinolin-2-yl]oxy}methyl)pyridine-3-carbonitrile

The title compound was prepared according to the procedure described in Example 5, Step 1, using 6-{[(4-chloro-5,6,7,8-tetrahydroquinolin-2-yl)oxy]m ethyl}pyridine-3-carbonitrile instead of 2-(pyridin-2-ylmethoxy)-6,7-dihydro-5H-cyclopenta[b]pyridin-4-yl trifluoromethanesulfonate, and using PdCl₂(PCy₃)₂ instead of Pd(dppf)Cl₂.CH₂Cl₂.

### [Step 2]

Production of 6-[({4-[6-(hydroxymethyl)pyrazin-2-yl]-5,6,7,8-tetrahydroquinolin-2-yl}oxy)methyl]pyridine-3-carbonitrile

The title compound was prepared as a white powder according to the procedure described in Example 269, Step 5, using 6-({[4-(4,4,5,5-tetramethyl-1,3,2-dioxaborolan-2-yl)-5,6,7,8-tetrahydroquinolin-2-yl]oxy}methyl)pyridine-3-carbonitrile instead of 2-[(5-fluoropyridin-2-yl)methoxy]-4-(4,4,5,5-tetramethyl-1,3,2-dioxaborolan-2-yl)-6,7-dihydro-5H-cyclopenta[b]pyridine and using Pd(dtbpf)Cl₂ instead of Pd(dppf) Cl₂.CH₂Cl₂.

[MS (ESI) m/z 374.4 (M+H)⁺]

### Example 289

### 4-(Pyridazin-3-yl)-2-(pyridin-2-ylmethoxy)-5,6,7,8-tetrahydroquinoline

The title compound was prepared as colorless oil according to the procedure described in Example 108, Step 2, using pyridazin-3-yl trifluoromethanesulfonate (see, for example, Tetrahedron, 2009, vol. 65, p. 8969-8980) instead of 3-bromo-2-fluoro-5-methylpyridine and using THF/water (1/1) instead of 1,4-dioxane/water (3/1).

[MS (ESI) m/z 319.3 (M+H)⁺]

### Test Example 1

### Experiment for Receptor Function of mGluR5

Stable expression cells in which the human mGluR5 gene had been introduced into a Chinese hamster ovary cell (CHO-K1) were used for evaluation. The cells were suspended in a growth medium (MEM-α (D8042, available from Sigma) containing 10% inactivated FBS (Cat. No. 171012, available from CCB) and 2 mM glutamax (Cat. No. 35050, available from GIBCO)) and seeded on a black clear-bottom 96-well plate at 100,000 cells per well. One day after seeding, the medium was replaced by a medium (Hanks' balanced salt solution (HBSS buffer solution) (20 mM HEPES, 2.5 mM probencide (165-15472, available from Wako Pure Chemical Industries, Ltd.), and 10×HBSS (14065-056, available from Gibco) diluted 10 times, pH 7.4)) containing a calcium-sensitive dye Fluo-4 AM at a final concentration of 1 M (F312, available from Dojindo Laboratories) and Pluronic F-127 at a final concentration of 0.0145% (P-2443, available from Sigma), followed by incubation at 37° C. for one hour, thereby loading Fluo-4 AM into the cells.

Subsequently, the cells were washed twice with an HBSS buffer solution, to which was subsequently added the same buffer solution. Twenty minutes thereafter, the plate was transferred into a fluorescence screening system (FLIPR TETRA, available from Molecular Devices, LLC) and measured for a Ca²⁺-dependent fluorescence change in the cells.

First of all, a fluorescence baseline was measured, and thereafter, fluorescence measurement was performed for 2 minutes while adding a test substance, an excessive amount of 6-methyl-2-(phenylethynyl)pyridine (hereinafter referred to as “MPEP”) (final concentration: 10 μM), or the medium. Thereafter, fluorescence measurement was carried out for one minute while adding L-glutamic acid (final concentration: 10 μM).

While taking a fluorescence intensity caused by L-glutamic acid as 100% and a fluorescence intensity caused by L-glutamic acid in the presence of MPEP as 0%, respectively, function inhibitory activity against human mGluR5 was evaluated from the fluorescence intensity caused by L-glutamic acid in the presence of the test substance at each concentration, and an IC50 value was estimated according to a logistic equation (SAS System). The obtained results are shown in Tables 1 and 2.

TABLE 1

| Test substance | Experiment for receptor function |
|---|---|
| (Example No.) | of human mGluR5 IC50 (nM) |
| 1 | 144 |
| 2 | 5.26 |
| 3 | 12.0 |
| 4 | 147 |
| 18 | 105 |
| 23 | 39 |
| 24 | 36 |
| 25 | 60.6 |
| 26 | 73.6 |
| 28 | 104 |
| 32 | 31 |
| 34 | 15 |
| 35 | 58 |
| 36 | 71 |
| 37 | 79 |
| 38 | 45 |
| 39 | 122 |
| 40 | 78 |
| 41 | 30 |
| 44 | 166 |
| 46 | 207 |
| 48 | 72 |
| 49 | 55 |
| 50 | 176 |
| 53 | 11.4 |
| 54 | 20 |
| 55 | 348 |
| 56 | 495 |
| 58 | 55.6 |
| 63 | 59.9 |
| 64 | 297 |
| 66 | 108 |
| 68 | 92 |
| 69 | 94 |
| 73 | 16.2 |
| 82 | 153 |
| 83 | 16.2 |
| 84 | 112 |
| 85 | 178 |
| 86 | 15.1 |
| 89 | 41.4 |
| 90 | 152 |
| 91 | 16.9 |
| 92 | 40.1 |
| 94 | 75.0 |
| 95 | 42 |
| 96 | 56.3 |
| 101 | 23.4 |
| 102 | 51.2 |
| 104 | 51.7 |
| 107 | 81.2 |
| 113 | 68.8 |
| 115 | 98.7 |
| 116 | 133 |
| 120 | 206 |
| 122 | 72.7 |
| 123 | 58.8 |
| 124 | 24.8 |
| 125 | 45.6 |
| 126 | 99.8 |
| 138 | 95 |
| 143 | 38.0 |
| 144 | 19.4 |
| 145 | 74 |
| 146 | 176 |
| 147 | 19.7 |
| 148 | 22 |
| 149 | 19.3 |
| 150 | 63 |
| 151 | 439 |
| 152 | 44.8 |
| 153 | 316 |
| 154 | 280 |
| 157 | 333 |
| 158 | 253 |
| 160 | 152 |
| 161 | 359 |
| 164 | 27.9 |
| 165 | 37.9 |
| 167 | 20.4 |
| 168 | 153 |
| 169 | 13.9 |
| 173 | 161 |
| 176 | 70.8 |
| 182 | 123 |
| 183 | 148 |
| 185 | 26 |
| 186 | 26 |
| 187 | 36 |
| 191 | 36 |
| 194 | 11.4 |
| 195 | 24.3 |
| 196 | 170 |
| 197 | 93.7 |
| 198 | 56.3 |
| 199 | 24.5 |
| 200 | 144 |
| 201 | 36.2 |
| 202 | 43 |
| 204 | 230 |
| 205 | 93 |
| 206 | 36.5 |
| 207 | 346 |
| 208 | 125 |
| 214 | 131 |
| 223 | 47.3 |
| 224 | 25.6 |
| 225 | 102 |
| 226 | 52.1 |
| 227 | 36.2 |
| 231 | 304 |
| 232 | 28.9 |
| 233 | 10.9 |
| 234 | 41.4 |
| 237 | 34.5 |
| 238 | 23 |
| 240 | 24 |

TABLE 2

| Test substance | Experiment for receptor function |
|---|---|
| (Example No.) | of human mGluR5 IC50 (nM) |
| 245 | 39 |
| 246 | 42 |
| 247 | 52 |
| 248 | 95 |
| 249 | 53 |
| 250 | 10.6 |
| 251 | 94.3 |
| 252 | 13.5 |
| 253 | 80 |
| 256 | 62.8 |
| 257 | 63.5 |
| 258 | 10.1 |
| 259 | 27.3 |
| 260 | 79 |
| 261 | 64 |
| 262 | 82 |
| 263 | 111 |
| 264 | 126 |
| 265 | 46.8 |
| 266 | 71 |
| 267 | 55 |
| 269 | 67 |
| 270 | 186 |
| 271 | 92.0 |
| 272 | 117 |
| 273 | 62 |
| 274 | 61 |
| 275 | 82.0 |
| 276 | 28.0 |
| 278 | 87 |
| 279 | 108 |
| 280 | 92 |
| 281 | 94 |
| 282 | 66 |
| 286 | 95 |
| 287 | 146 |
| 288 | 119 |

As described in Test Example 1, the compound of the present invention or a pharmaceutically acceptable salt thereof exhibits high mGluR5 inhibitory activity.

### Test Example 2

### Action Against Formalin-Induced Pain Behavior in Mice

4 to 6-week-old male Slc:ICR mice (available from Japan SLC, Inc.) were used. A medium or a test substance (1 mg/kg or 3 mg/kg) was intravenously administered to each mouse, and immediately thereafter, 20 μL of 1% or 5% formalin was subcutaneously administered to a right hind paw of the mouse. Thereafter, the mice were quickly put into an observation cage and measured for the total time (sec) of pain-related behavior (licking or biting behavior against the right hindlimb) during 6 minutes, and an average value of the pain-related behavior time of each group was calculated.

The intensity of an analgesic action of the test substance was evaluated in terms of percent inhibition (%) of pain-related behavior time which is calculated as {(A−B)/A×100}, wherein A is an average value of the pain-related behavior time of a medium-administered group, and B is an average value of the pain-related behavior time of a test substance-administered group. The obtained results are shown in Table 3.

TABLE 3

| | | Percent inhibition |
|---|---|---|
| Test substance | Concentration | of pain-related |
| (Example No.) | of formalin | behavior time (%) |
| 2 | 1% | 47.0 *¹ |
| 3 | 1% | 25.1 *¹ |
| 9 | 5% | 39.1 *¹ |
| 12 | 1% | 66.8 *¹ |
| 15 | 5% | 64.0 *¹ |
| 23 | 5% | 47.9 *¹ |
| 26 | 1% | 71.9 *¹ |
| 82 | 1% | 59.8 *² |
| 90 | 5% | 54.3 *¹ |
| 163 | 5% | 37.2 *² |
| 164 | 1% | 42.2 *¹ |
| 165 | 1% | 52.3 *¹ |
| 173 | 5% | 65.4 *¹ |
| 187 | 5% | 59.4 *¹ |
| 198 | 1% | 24.7 *¹ |
| 214 | 5% | 55.3 *¹ |
| 216 | 5% | 41.2 *¹ |
| 226 | 1% | 26.8 *¹ |
| 227 | 1% | 25.6 *¹ |
| 231 | 5% | 66.1 *¹ |
| 233 | 1% | 59.5 *¹ |
| 238 | 5% | 77.9 *¹ |
| 246 | 5% | 22.8 *¹ |
| 257 | 5% | 51.1 *¹ |
| 260 | 5% | 42.9 *¹ |
| 268 | 5% | 53.9 *¹ |
| 272 | 5% | 40.5 *¹ |
| 277 | 5% | 60.9 *¹ |
| 283 | 5% | 38.8 *¹ |

*¹ A dosage of a test substance is 1 mg/kg.
*² A dosage of a test substance is 3 mg/kg.

### Test Example 3

### Action Against Pain-Related Behavior and Micturition Cycle in Mice with Cyclophosphamide (CP)-Induced Acute Cystitis

An action of a test substance against visceral pain and frequent urination associated with cystitis, which was induced by intraperitoneal administration (20 mL/kg) of CP (300 mg/kg) to 4 to 5-week-old male Slc:ICR mice (available from Japan SLC, Inc.), was investigated. This experiment was performed according to a method described in European Journal of Pharmacology, 676 (2012), pp. 75-80.

Three hours after the administration of CP, a test substance (1 mg/kg or 3 mg/kg) was intravenously administered, and observation by visual inspection and video recording were performed for 15 minutes immediately thereafter. In the observation by visual inspection, four pain-related behaviors (eye opening, rounded back, leg dragging, and behavior during urination) were scored, each rated on a scale of 0-10, according to the following criteria.

Eye opening (normal: 0 point, sometimes closed or half-open eyes: 5 points, completely closed: 10 points)

Rounded back (normal: 0 point, sometimes crouched: 5 points, crouched at almost all times: 10 points)

Leg dragging (normal: 0 point, leg dragging: 10 points)

Behavior during urination (normal: 0 point, shaking hip in micturition: 10 points)

In addition, the video was played back, and the frequency of licking behavior around the abdomen as a pain-related behavior was scored, rated on a scale of 0-10, according to the following criteria.

Licking behavior around the abdomen (0 to 3 times: 0 point, 4 times to 10 times: 5 points, 11 times or more: 10 points)

Furthermore, the video was played back, and the frequency of urination (urination marks) on a filter paper was counted and taken as an index for micturition cycle.

An average value of the pain-related behavior score of each group (total score of the five items: 50 points at maximum) and an average value of the frequency of urination (urination marks) on the filter paper were determined.

The strength of an analgesic action of the test substance against the CP-induced visceral pain was evaluated in terms of percent inhibition (%) of pain-related behavior score which is calculated as {(A−B)/A×100}, wherein A is an average value of the pain-related behavior score of a medium-administered group, and B is an average value of the pain-related behavior score of a test substance-administered group.

In addition, the strength of a frequent urination inhibitory action of the test substance against the CP-induced frequent urination was evaluated in terms of percent inhibition (%) of the frequency of urination which is calculated as {(C−D)/C×100}, wherein C is an average value of the frequency of urination of a medium-administered group, and D is an average value of the frequency of urination of a test substance-administered group. The obtained results are shown in Table 4.

TABLE 4

| | Percent inhibition | Percent inhibition |
|---|---|---|
| Test substance | of pain-related | of the frequency |
| (Example No.) | behavior score (%) | of urination (%) |
| 9 | 23.9 *¹ | 36.3 *¹ |
| 26 | 60.5 *² | 57.3 *² |
| 36 | 40.4 *² | 31.3 *² |
| 43 | 31.0 *² | 41.9 *² |
| 45 | 34.1 *² | 33.8 *² |
| 46 | 22.0 *¹ | 31.5 *¹ |
| 143 | 52.2 *² | 47.7 *² |
| 147 | 38.8 *² | 21.1 *² |
| 148 | 45.7 *² | 31.5 *² |
| 173 | 63.3 *² | 52.6 *² |
| 182 | 26.0 *² | 43.2 *² |
| 183 | 37.0 *¹ | 22.7 *¹ |
| 187 | 22.9 *¹ | 51.6 *¹ |
| 191 | 20.5 *² | 36.5 *² |
| 198 | 40.4 *² | 37.6 *² |
| 214 | 44.2 *² | 25.3 *² |
| 216 | 28.2 *² | 30.1 *² |
| 217 | 38.3 *² | 40.4 *² |
| 255 | 31.0 *² | 26.4 *² |
| 277 | 40.4 *² | 24.8 *² |

*¹ A dosage of a test substance is 1 mg/kg.
*² A dosage of a test substance is 3 mg/kg.

### Formulation Example 1

### Tablet (Oral Tablet)

In an 80 mg tablet of the formulation:

Compound of the invention of Example 1: 5.0 mg

Corn starch: 46.6 mg

Crystalline cellulose: 24.0 mg

Methyl cellulose: 4.0 mg

Magnesium stearate: 0.4 mg

A mixed powder of this proportion is press-molded to form an oral tablet by an ordinary method.

### INDUSTRIAL APPLICABILITY

In view of the fact that the compound of the present invention or a pharmaceutically acceptable salt thereof exhibits mGluR5 inhibitory activity, in particular, it is useful as a preventive agent or therapeutic agent for pain (for example, acute pain, chronic pain, inflammatory pain, neuropathic pain, hyperalgesia, thermal hyperalgesia, allodynia, pain by noxious thermal stimulation, pain by noxious mechanical stimulation, pain in the lower urinary tract or reproductive organs or migraine), pruritus, a lower urinary tract symptom or lower urinary tract dysfunction, a lower urinary tract symptom or lower urinary tract dysfunction, gastroesophageal reflux disease (GERD), gastroesophageal reflux disease associated with transient lower esophageal sphincter relaxation (TLESR), or central nervous system disease.

1. A compound represented by the following formula [1] or a pharmaceutically acceptable salt thereof: 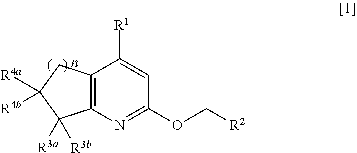
wherein
R¹ represents phenyl, benzo[d][1,3]dioxolyl or heteroaryl;
R¹, when it is a heteroaryl, is bound through a carbon atom on the ring;
the phenyl, benzo[d][1,3]dioxolyl or heteroaryl of R¹ may be substituted at a substitutable arbitrary position(s) with one or two same or different groups selected from the group consisting of
(i) halogen,
(ii) cyano,
(iii) hydroxy,
(iv) nitro,
(v) alkoxy,
(vi) cycloalkyl,
(vii) alkylsulfonate,
(viii) alkyl,
(ix) hydroxyalkyl,
(x) alkoxyalkyl,
(xi) monohalogenoalkyl,
(xii) dihalogenoalkyl,
(xiii) trihalogenoalkyl,
(xiv) amino which may be substituted at a substitutable arbitrary position(s) with one or two same or different groups selected from the group consisting of alkyl, aminoalkyl, alkoxycarbonylaminoalkyl, monoalkylaminoalkyl, dialkylaminoalkyl, and alkylsulfonyl,
(xv) saturated cyclic amino which may be substituted at a substitutable arbitrary position(s) with one or two oxos,
(xvi) alkylcarbonyl,
(xvii) alkoxycarbonyl,
(xviii) hydroxycarbonyl,
(xix) carbamoyl which may be substituted at a substitutable arbitrary position(s) with one or two same or different groups selected from the group consisting of alkyl, cycloalkyl, and (cycloalkyl)alkyl, and
(xx) a group represented by the following formula [2]: 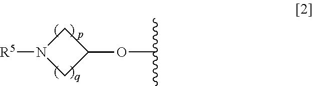
wherein R⁵ represents hydrogen, alkyl or alkylcarbonyl; p and q may be the same as or different from each other and each represents 1 or 2; and
when a nitrogen atom is contained as the ring constituting atom in the heteroaryl of R¹, an oxygen atom may coordinate to the nitrogen atom;
R² represents phenyl or heteroaryl;
R², when it is a heteroaryl, is bound through a carbon atom on the ring;
the phenyl or heteroaryl of R² may be substituted at a substitutable arbitrary position(s) with one or two same or different groups selected from the group consisting of cyano, halogen, cycloalkyl, alkoxy, carbamoyl, alkenyl, alkyl, hydroxyalkyl, alkoxyalkyl, monohalogenoalkyl, dihalogenoalkyl, trihalogenoalkyl, amino, monoalkylamino and dialkylamino; and
when a nitrogen atom is contained as the ring constituting atom in the heteroaryl of R², an oxygen atom may coordinate to the nitrogen atom;
R^{3a} represents hydrogen, and R^{3b} represents hydrogen, alkyl, hydroxy, halogen, alkoxy or alkylcarbonyloxy, or R^{3a} and R^{3b} taken together with the adjacent carbon atom represent a group represented by the following formula [3] or [4]: 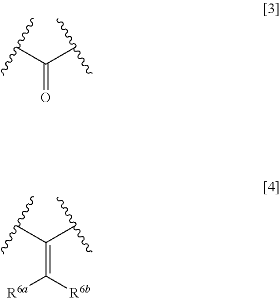
wherein R^{6a} and R^{6b} may be the same as or different from each other and each represents hydrogen or alkyl;
R^{4a} and R^{4b} may be the same as or different from each other and each represents hydrogen or alkyl; and
n represents an integer of 1 to 3.

2. The compound or a pharmaceutically acceptable salt thereof according to claim 1, wherein R¹ is phenyl or heteroaryl.

3. The compound or a pharmaceutically acceptable salt thereof according to claim 1, wherein R¹ is phenyl, pyridyl, pyrimidyl, pyrazinyl or pyridazinyl.

4. The compound or a pharmaceutically acceptable salt thereof according to claim 1, wherein R¹ is phenyl, pyridyl, pyrimidyl, pyrazinyl or pyridazinyl, and R¹ may be substituted with one or two groups selected from the group consisting of alkyl, halogen, cyano, amino, monoalkylamino, dialkylamino, hydroxyalkyl and alkylsulfonate.

5. The compound or a pharmaceutically acceptable salt thereof according to claim 1, wherein R² is phenyl, pyridyl, pyrazinyl, pyrimidyl, oxazolyl, imidazolyl or thiazolyl.

6. The compound or a pharmaceutically acceptable salt thereof according to claim 1, wherein R² is phenyl, pyridyl, pyrazinyl, pyrimidyl, oxazolyl, imidazolyl or thiazolyl, and R² may be substituted with one or two groups selected from the group consisting of alkyl, hydroxyalkyl, alkoxyalkyl, halogen and cyano.

7. The compound or a pharmaceutically acceptable salt thereof according to claim 1, wherein R^{3a} is hydrogen, and R^{3b} is hydrogen, alkyl, halogen, hydroxy or alkoxy.

8. The compound or a pharmaceutically acceptable salt thereof according to claim 1, wherein R^{3a}, R^{3b}, R^{4a} and R^{4b} are each hydrogen.

9. The compound or a pharmaceutically acceptable salt thereof according to claim 1, wherein:
R¹ is phenyl, pyridyl, pyrimidyl, pyrazinyl or pyridazinyl;
R² is phenyl, pyridyl, pyrazinyl, pyrimidyl, oxazolyl, imidazolyl or thiazolyl; and
R^{3a}, R^{3b}, R^{4a} and R^{4b} are each hydrogen.

10. The compound or a pharmaceutically acceptable salt thereof according to claim 1, wherein:
R¹ is phenyl, pyridyl, pyrimidyl, pyrazinyl or pyridazinyl, and R¹ may be substituted with one or two groups selected from the group consisting of alkyl, halogen, cyano, amino, monoalkylamino, dialkylamino, hydroxyalkyl and alkylsulfonate;
R² is phenyl, pyridyl, pyrazinyl, pyrimidyl, oxazolyl, imidazolyl or thiazolyl, and R² may be substituted with one or two groups selected from the group consisting of alkyl, hydroxyalkyl, alkoxyalkyl, halogen and cyano; and
R^{3a}, R^{3b}, R^{4a} and R^{4b} are each hydrogen.

11. A compound according to any one of the following (1) to (95):
(1) 4-(pyridin-3-yl)-2-(pyridin-2-ylmethoxy)-6,7-dihydro-5H-cyclopenta[b]pyridine,
(2) 3-[2-(pyridin-2-ylmethoxy)-6,7-dihydro-5H-cyclopenta[b]pyridin-4-yl]pyridine-2-carbonitrile,
(3) 5-[2-(pyridin-2-ylmethoxy)-6,7-dihydro-5H-cyclopenta[b]pyridin-4-yl]pyridin-3-yl methanesulfonate,
(4) 5-{2-[(5-fluoropyridin-2-yl)methoxy]-6,7-dihydro-5H-cyclopenta[b]pyridin-4-yl}pyridine-2-carbonitrile,
(5) 2-(benzyloxy)-4-(pyrimidin-5-yl)-6,7-dihydro-5H-cyclopenta[b]pyridine,
(6) 3-({[4-(pyrimidin-5-yl)-6,7-dihydro-5H-cyclopenta[b]pyridin-2-yl]oxy}methyl)benzonitrile,
(7) 2-[(3-fluoropyridin-2-yl)methoxy]-4-(pyrimidin-5-yl)-6,7-dihydro-5H-cyclopenta[b]pyridine,
(8) 2-[(3,5-difluoropyridin-2-yl)methoxy]-4-(pyrimidin-5-yl)-6,7-dihydro-5H-cyclopenta[b]pyridine,
(9) 2-[(6-methylpyridin-3-yl)methoxy]-4-(pyrimidin-5-yl)-6,7-dihydro-5H-cyclopenta[b]pyridine,
(10) 4-(2-methylpyrimidin-5-yl)-2-(pyridin-2-ylmethoxy)-6,7-dihydro-5H-cyclopenta[b]pyridine,
(11) 5-[2-(pyridin-2-ylmethoxy)-6,7-dihydro-5H-cyclopenta[b]pyridin-4-yl]pyrimidine-2-carbonitrile,
(12) 2-[(3-fluoropyridin-2-yl)methoxy]-4-(2-methylpyrimidin-5-yl)-6,7-dihydro-5H-cyclopenta[b]pyridine,
(13) 2-[(3,5-difluoropyridin-2-yl)methoxy]-4-(2-methylpyrimidin-5-yl)-6,7-dihydro-5H-cyclopenta[b]pyridine,
(14) 2-[(3,6-difluoropyridin-2-yl)methoxy]-4-(2-methylpyrimidin-5-yl)-6,7-dihydro-5H-cyclopenta[b]pyridine,
(15) 5-{2-[(3,5-difluoropyridin-2-yl)methoxy]-6,7-dihydro-5H-cyclopenta[b]pyridin-4-yl}pyrimidine-2-carbonitrile,
(16) 2-[(6-methylpyridin-2-yl)methoxy]-4-(2-methylpyrimidin-5-yl)-6,7-dihydro-5H-cyclopenta[b]pyridine,
(17) 6-({[4-(2-methylpyrimidin-5-yl)-6,7-dihydro-5H-cyclopenta[b]pyridin-2-yl]oxy}methyl)pyridine-2-carbonitrile,
(18) 6-({[4-(2-methylpyrimidin-5-yl)-6,7-dihydro-5H-cyclopenta[b]pyridin-2-yl]oxy}methyl)pyridine-3-carbonitrile,
(19) 5-{2-[(3-fluoropyridin-2-yl)methoxy]-6,7-dihydro-5H-cyclopenta[b]pyridin-4-yl}pyrimidine-2-carbonitrile,
(20) 2-fluoro-4-[2-(pyridin-2-ylmethoxy)-6,7-dihydro-5H-cyclopenta[b]pyridin-4-yl]benzonitrile,
(21) 3-fluoro-5-[2-(pyridin-2-ylmethoxy)-6,7-dihydro-5H-cyclopenta[b]pyridin-4-yl]phenyl methanesulfonate,
(22) 4-(pyridin-3-yl)-2-(pyrimidin-2-ylmethoxy)-5,6,7,8-tetrahydroquinoline,
(23) 2-[(4-methyl-1,3-oxazol-2-yl)methoxy]-4-(pyridin-3-yl)-5,6,7,8-tetrahydroquinoline,
(24) 4-(pyridin-3-yl)-2-(1,3-thiazol-2-ylmethoxy)-5,6,7,8-tetrahydroquinoline,
(25) 2-[(1-methyl-1H-imidazol-2-yl)methoxy]-4-(pyridin-3-yl)-5,6,7,8-tetrahydroquinoline,
(26) 4-(4-methylpyridin-3-yl)-2-(pyridin-2-ylmethoxy)-5,6,7,8-tetrahydroquinoline,
(27) 5-[2-(pyridin-2-ylmethoxy)-5,6,7,8-tetrahydroquinolin-4-yl]pyridine-2-carbonitrile,
(28) {5-[2-(pyridin-2-ylmethoxy)-5,6,7,8-tetrahydroquinolin-4-yl]pyridin-3-yl}methanol,
(29)N-methyl-5-[2-(pyridin-2-ylmethoxy)-5,6,7,8-tetrahydroquinolin-4-yl]pyridin-3-amine,
(30) N,N-dimethyl-5-[2-(pyridin-2-ylmethoxy)-5,6,7,8-tetrahydroquinolin-4-yl]pyridin-3-amine,
(31) 5-{2-[(3-fluoropyridin-2-yl)methoxy]-5,6,7,8-tetrahydroquinolin-4-yl}pyridine-2-carbonitrile,
(32) 2-(pyridin-2-ylmethoxy)-4-(pyrimidin-5-yl)-5,6,7,8-tetrahydroquinoline,
(33) 2-[(3-fluoropyridin-2-yl)methoxy]-4-(pyrimidin-5-yl)-5,6,7,8-tetrahydroquinoline,
(34) 2-[(3,6-difluoropyridin-2-yl)methoxy]-4-(pyrimidin-5-yl)-5,6,7,8-tetrahydroquinoline,
(35) 6-({[4-(pyrimidin-5-yl)-5,6,7,8-tetrahydroquinolin-2-yl]oxy}methyl)pyridine-2-carbontrile,
(36) 2-[(6-methylpyridin-2-yl)methoxy]-4-(pyrimidin-5-yl)-5,6,7,8-tetrahydroquinoline,
(37) [6-({[4-(pyrimidin-5-yl)-5,6,7,8-tetrahydroquinolin-2-yl]oxy}methyl)pyridin-2-yl]methanol,
(38) 5-[2-(pyridin-2-ylmethoxy)-5,6,7,8-tetrahydroquinolin-4-yl]pyrimidin-2-amine,
(39) 5-[2-(pyridin-2-ylmethoxy)-5,6,7,8-tetrahydroquinolin-4-yl]pyrimidine-2-carbonitrile,
(40) 2-[(6-methylpyridin-2-yl)methoxy]-4-(2-methylpyrimidin-5-yl)-5,6,7,8-tetrahydroquinoline,
(41) 6-({[4-(2-methylpyrimidin-5-yl)-5,6,7,8-tetrahydroquinolin-2-yl]oxy}methyl)pyridine-2-carbonitrile,
(42) 6-({[4-(2-methylpyrimidin-5-yl)-5,6,7,8-tetrahydroquinolin-2-yl]oxy}methyl)pyridine-3-carbonitrile,
(43) 2-[(5-fluoropyridin-2-yl)methoxy]-4-(2-methylpyrimidin-5-yl)-5,6,7,8-tetrahydroquinoline,
(44) 2-[(3-fluoropyridin-2-yl)methoxy]-4-(2-methylpyrimidin-5-yl)-5,6,7,8-tetrahydroquinoline,
(45) 2-[(3,5-difluoropyridin-2-yl)methoxy]-4-(2-methylpyrimidin-5-yl)-5,6,7,8-tetrahydroquinoline,
(46) 5-{2-[(3-fluoropyridin-2-yl)methoxy]-5,6,7,8-tetrahydroquinolin-4-yl}pyrimidin-2-amine,
(47) 3-[2-(pyridin-2-ylmethoxy)-5,6,7,8-tetrahydroquinolin-4-yl]pyrazine-2-carbonitrile,
(48) (6-{2-[(5-fluoropyridin-2-yl)methoxy]-5,6,7,8-tetrahydroquinolin-4-yl}pyrazin-2-yl)methanol,
(49) (6-{2-[(3-fluoropyridin-2-yl)methoxy]-5,6,7,8-tetrahydroquinolin-4-yl}pyrazin-2-yl)methanol,
(50) 7-ethyl-2-(pyridin-2-ylmethoxy)-4-(pyrimidin-5-yl)-6,7-dihydro-5H-cyclopenta[b]pyridine,
(51) 3-({[4-(pyridin-3-yl)-6,7-dihydro-5H-cyclopenta[b]pyridin-2-yl]oxy}methyl)benzonitrile,
(52) 5-{2-[(3,5-difluoropyridin-2-yl)methoxy]-6,7-dihydro-5H-cyclopenta[b]pyridin-4-yl}pyridine-2-carbonitrile,
(53) 4-[({4-[5-(hydroxymethyl)pyridin-3-yl]-6,7-dihydro-5H-cyclopenta[b]pyridin-2-yl}oxy)methyl]benzonitrile,
(54) (5-{2-[(4-fluorobenzyl)oxy]-6,7-dihydro-5H-cyclopenta[b]pyridin-4-yl}pyridin-3-yl)methanol,
(55) (5-{2-[(3-fluorobenzyl)oxy]-6,7-dihydro-5H-cyclopenta[b]pyridin-4-yl}pyridin-3-yl)methanol,
(56) 6-({[4-(5-fluoropyridin-3-yl)-6,7-dihydro-5H-cyclopenta[b]pyridin-2-yl]oxy}methyl)pyridine-3-carbonitrile,
(57) 3-({[4-(2-fluoropyridin-4-yl)-6,7-dihydro-5H-cyclopenta[b]pyridin-2-yl]oxy}methyl)benzonitrile,
(58) 6-({[4-(2-fluoropyridin-4-yl)-6,7-dihydro-5H-cyclopenta[b]pyridin-2-yl]oxy}methyl)pyridine-3-carbonitrile,
(59) 3-[({4-[5-(hydroxymethyl)pyridin-3-yl]-6,7-dihydro-5H-cyclopenta[b]pyridin-2-yl}oxy)methyl]benzonitrile,
(60) 6-({[4-(pyrimidin-5-yl)-6,7-dihydro-5H-cyclopenta[b]pyridin-2-yl]oxy}methyl)pyridine-3-carbonitrile,
(61) 2-[(3,6-difluoropyridin-2-yl)methoxy]-4-(pyrimidin-5-yl)-6,7-dihydro-5H-cyclopenta[b]pyridine,
(62) 2-[(2-methylpyridin-4-yl)methoxy]-4-(pyrimidin-5-yl)-6,7-dihydro-5H-cyclopenta[b]pyridine,
(63) 2-[(4-fluorobenzyl)oxy]-4-(pyrimidin-5-yl)-6,7-dihydro-5H-cyclopenta[b]pyridine,
(64) 4-({[4-(pyrimidin-5-yl)-6,7-dihydro-5H-cyclopenta[b]pyridin-2-yl]oxy}methyl)benzonitrile,
(65) 5-[2-(pyridin-2-ylmethoxy)-6,7-dihydro-5H-cyclopenta[b]pyridin-4-yl]pyrimidin-2-amine,
(66) 2-[(4-fluorobenzyl)oxy]-4-(2-methylpyrimidin-5-yl)-6,7-dihydro-5H-cyclopenta[b]pyridine,
(67) 4-({[4-(2-methylpyrimidin-5-yl)-6,7-dihydro-5H-cyclopenta[b]pyridin-2-yl]oxy}methyl)benzonitrile,
(68) 2-[(2-methylpyridin-4-yl)methoxy]-4-(2-methylpyrimidin-5-yl)-6,7-dihydro-5H-cyclopenta[b]pyridine,
(69) 5-{2-[(3-fluorobenzyl)oxy]-6,7-dihydro-5H-cyclopenta[b]pyridin-4-yl}pyrimidine-2-amine,
(70) 4-({[4-(2-aminopyrimidin-5-yl)-6,7-dihydro-5H-cyclopenta[b]pyridin-2-yl]oxy}methyl)benzonitrile,
(71) 3-({[4-(2-aminopyrimidin-5-yl)-6,7-dihydro-5H-cyclopenta[b]pyridin-2-yl]oxy}methyl)benzonitrile,
(72) 2-[(3-fluorobenzyl)oxy]-4-(2-methylpyrimidin-5-yl)-6,7-dihydro-5H-cyclopenta[b]pyridine,
(73) 2-({[4-(2-methylpyrimidin-5-yl)-6,7-dihydro-5H-cyclopenta[b]pyridin-2-yl]oxy}methyl)-1,3-oxazole-4-carbonitrile,
(74) {6-[2-(pyridin-2-ylmethoxy)-6,7-dihydro-5H-cyclopenta[b]pyridin-4-yl]pyrazin-2-yl}methanol,
(75) (6-{2-[(5-fluoropyridin-2-yl)methoxy]-6,7-dihydro-5H-cyclopenta[b]pyridin-4-yl}pyrazin-2-yl)methanol,
(76) 3-[({4-[6-(hydroxymethyl)pyrazin-2-yl]-6,7-dihydro-5H-cyclopenta[b]pyridin-2-yl}oxy)methyl]benzonitrile,
(77) 4-(pyridazin-3-yl)-2-(pyridin-2-ylmethoxy)-6,7-dihydro-5H-cyclopenta[b]pyridine,
(78) 2-[(3-fluoropyridin-2-yl)methoxy]-4-(pyridazin-3-yl)-6,7-dihydro-5H-cyclopenta[b]pyridine,
(79) 4-({[4-(pyridazin-3-yl)-6,7-dihydro-5H-cyclopenta[b]pyridin-2-yl]oxy}methyl)benzonitrile,
(80) 6-({[4-(1-methyl-1H-pyrazol-5-yl)-6,7-dihydro-5H-cyclopenta[b]pyridin-2-yl]oxy}methyl)pyridine-3-carbonitrile,
(81) 6-({[4-(pyridazin-3-yl)-6,7-dihydro-5H-cyclopenta[b]pyridin-2-yl]oxy}methyl)pyridine-3-carbonitrile,
(82) 6-[({4-[5-(hydroxymethyl)pyridin-3-yl]-5,6,7,8-tetrahydroquinolin-2-yl}oxy)methyl]pyridine-3-carbonitrile,
(83) 2-[(3,5-difluoropyridin-2-yl)methoxy]-4-(pyrimidin-5-yl)-5,6,7,8-tetrahydroquinoline,
(84) 4-({[4-(pyrimidin-5-yl)-5,6,7,8-tetrahydroquinolin-2-yl]oxy}methyl)benzonitrile,
(85) 3-({[4-(pyrimidin-5-yl)-5,6,7,8-tetrahydroquinolin-2-yl]oxy}methyl)benzonitrile,
(86) 2-[(4-fluorobenzyl)oxy]-4-(pyrimidin-5-yl)-5,6,7,8-tetrahydroquinoline,
(87) 2-[(3-fluorobenzyl)oxy]-4-(pyrimidin-5-yl)-5,6,7,8-tetrahydroquinoline,
(88) 4-({[4-(pyrimidin-5-yl)-5,6,7,8-tetrahydroquinolin-2-yl]oxy}methyl)pyridine-2-carbonitrile,
(89) 5-{2-[(6-fluoropyridin-2-yl)methoxy]-5,6,7,8-tetrahydroquinolin-4-yl}pyrimidin-2-amine,
(90) 3-({[4-(2-methylpyrimidin-5-yl)-5,6,7,8-tetrahydroquinolin-2-yl]oxy}methyl)benzonitrile,
(91) 4-({[4-(2-methylpyrimidin-5-yl)-5,6,7,8-tetrahydroquinolin-2-yl]oxy}methyl)benzonitrile,
(92) 2-[(2-methylpyridin-4-yl)methoxy]-4-(2-methylpyrimidin-5-yl)-5,6,7,8-tetrahydroquinoline,
(93) 4-({[4-(2-methylpyrimidin-5-yl)-5,6,7,8-tetrahydroquinolin-2-yl]oxy}methyl)pyridine-2-carbonitrile,
(94) 6-[({4-[6-(hydroxymethyl)pyrazin-2-yl]-5,6,7,8-tetrahydroquinolin-2-yl}oxy)methyl]pyridine-3-carbonitrile, and
(95) 4-(pyridazin-3-yl)-2-(pyridin-2-ylmethoxy)-5,6,7,8-tetrahydroquinoline;
or a pharmaceutically acceptable salt thereof.

12. A pharmaceutical composition comprising the compound or a pharmaceutically acceptable salt thereof according to claim 1 as an active ingredient.

13. An mGluR5 inhibitor comprising the compound or a pharmaceutically acceptable salt thereof according to claim 1 as an active ingredient.

14. A preventive agent or therapeutic agent for pain, comprising the compound or a pharmaceutically acceptable salt thereof according to claim 1 as an active ingredient.

15. The preventive agent or therapeutic agent according to claim 14, wherein the pain is acute pain, chronic pain, inflammatory pain, neuropathic pain, hyperalgesia, thermal hyperalgesia, allodynia, pain by noxious thermal stimulation, pain by noxious mechanical stimulation, pain in the lower urinary tract or reproductive organs or migraine.

16-25. (canceled)

26. A preventive agent or therapeutic agent for pruritus, comprising the compound or a pharmaceutically acceptable salt thereof according to claim 1 as an active ingredient.

27. A preventive agent or therapeutic agent for a lower urinary tract symptom or lower urinary tract dysfunction, comprising the compound or a pharmaceutically acceptable salt thereof according to claim 1 as an active ingredient.

28. The preventive agent or therapeutic agent according to claim 27, wherein the pain in the lower urinary tract symptom or lower urinary tract dysfunction is frequent urination.

29. (canceled)

30. A preventive agent or therapeutic agent for gastroesophageal reflux disease (GERD) or gastroesophageal reflux disease associated with transient lower esophageal sphincter relaxation (TLESR), comprising the compound or a pharmaceutically acceptable salt thereof according to claim 1 as an active ingredient.

31. A preventive agent or therapeutic agent for central nervous system disease, comprising the compound or a pharmaceutically acceptable salt thereof according to claim 1 as an active ingredient.

32. (canceled)
